(19) United States
(12) Patent Application Publication
(10) Pub. No.: US 2025/0169357 A1
(43) Pub. Date: May 22, 2025

(54) COMPOUND, ORGANIC LIGHT EMITTING DEVICE, AND DISPLAY DEVICE

(71) Applicant: SK Inc., Seoul (KR)

(72) Inventors: Jeongmi KIM, Sejong-si (KR); Jongho LEE, Sejong-si (KR); Kiseon PARK, Seoul (KR)

(73) Assignee: SK Inc., Seoul (KR)

(21) Appl. No.: 19/008,324

(22) Filed: Jan. 2, 2025

Related U.S. Application Data
(63) Continuation of application No. 16/966,982, filed on Aug. 3, 2020.

(30) Foreign Application Priority Data
Mar. 2, 2018 (KR) 10-2018-0025274

(51) Int. Cl. H10K 85/60; C07D 401/04; C07D 401/10; C07D 401/14; C07D 495/04; H10K 50/11; H10K 50/15; H10K 50/16; H10K 50/17; H10K 50/18; H10K 59/12

(52) U.S. Cl. CPC H10K 85/654; C07D 401/04; C07D 401/10; C07D 401/14; C07D 495/04; H10K 85/6572; H10K 50/11; H10K 50/15; H10K 50/16; H10K 50/17; H10K 50/171; H10K 50/18; H10K 59/12

(57) ABSTRACT

Disclosed are: a compound applicable to an electron transport layer of an organic light emitting diode; an organic light emitting diode employing the compound; and an organic EL display device comprising the organic light emitting diode. The organic light emitting device includes a first electrode, and a second electrode facing the first electrode, and an organic material layer interposed between the first electrode and the second electrode.

### CROSS-REFERENCE TO PRIOR APPLICATIONS

This application is a Continuation Application of U.S. patent application Ser. No. 16/966,982 (filed on Aug. 3, 2020), which is a National Stage patent application of PCT International Patent Application No. PCT/KR2019/002398 (filed on Feb. 27, 2019), which claims priority to Korean Patent Application No. 10-2018-0025274 (filed on Mar. 2, 2018), which are all hereby incorporated by reference in their entirety.

### BACKGROUND

The present invention relates to a compound, an organic light emitting device, and an organic EL display device.

Generally, an organic light emitting phenomenon refers to a phenomenon in which electrical energy is converted into light energy in an organic material. An organic light emitting device employing the organic light emitting phenomenon typically has a structure including an anode, a cathode and an organic layer therebetween. In order to increase the efficiency and stability of the organic light emitting device, the organic layer may have a multi-layered structure composed of different materials, and, for example, may include a hole injection layer, a hole transport layer, a light emitting layer, an electron transport layer, and an electron injection layer or the like.

When a voltage is applied between two electrodes in the structure of the organic light emitting device, holes are injected into the light emitting layer through the hole injection layer and the hole transport layer from the anode, and electrons are injected into the light emitting layer through the electron injection layer and the electron transport layer from the cathode, and excitons are formed by recombination of the injected holes and electrons, and light is emitted when the excitons fall back to the ground state.

As for the electron transporting material, an organometallic complex which is an organic single-molecule material and has a relatively good electron transporting rate and stability to electrons is desired. Among them, Alq₃ having excellent stability and high electron affinity has been reported to be the best, but when used in a blue light emitting device, color purity is deteriorated due to light emission by exciton diffusion. That is, if the holes move faster than the electrons and the excitons generated in the light emitting layer are leaked into the electron transporting layer, a charge unbalance in the light-emitting layer is induced, thereby emitting of light at the interface of the electron transporting layer occurs. When light is emitted at the interface of the electron transport layer, the color purity and efficiency of the organic electroluminescent device are deteriorated, and particularly when the organic light emitting device is manufactured using it, the lifetime of the organic light emitting device is shortened.

Also known as other electron-transporting materials are Flavon derivatives, or germanium and silicon cyclopentadiene derivatives, and the like. In addition, organic single molecule materials such as PBD (2-biphenyl-4-yl-5-(4-t-butylphenyl)-1,3,4-oxidiazole) derivative bonded to a Spiro compound, and TPBI (2,2′,2″-(benzene-1, 3,5-triyl)-tris(1-phenyl-1H-benzimidazole) having both hole blocking capability and excellent electron transport capability are known. In particular, benzo imidazole derivatives are well known in their superior durability.

However, the organic light emitting device using such a material as an electron transport layer has a short lifetime, low storage durability and reliability, and there is a need for an improvement in terms of efficiency and driving voltage.

### SUMMARY

The objective of the present invention is to provide an organic light emitting device having high electron mobility and excellent hole blocking ability, high efficiency and low driving voltage, and a display device using the same.

According to an aspect of the present invention, provided is a compound represented by chemical formula 1,

A₁ [symbol] L [symbol] A₂ <Chemical Formula 1>
wherein A₁ is a group represented by any one of the following structures:

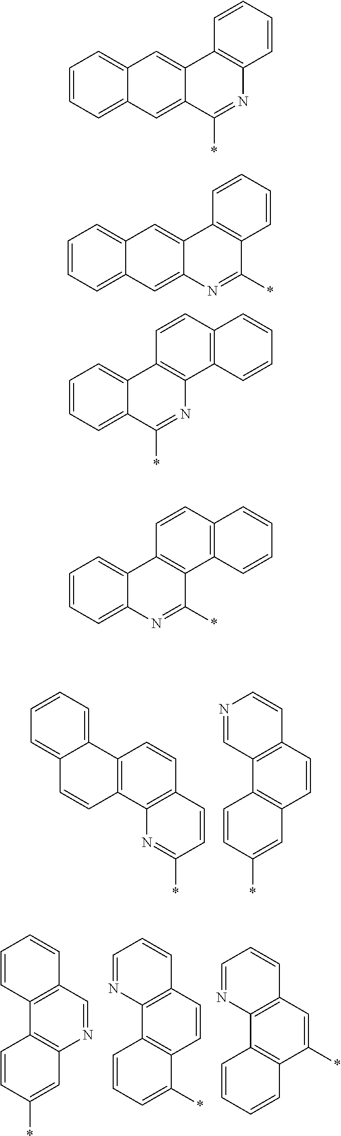

L is a direct bond; a substituted or unsubstituted arylene group; or a substituted or unsubstituted heteroarylene group; or a substituted or unsubstituted C₉-C₆₀ fused polycyclic group,
A₂ is hydrogen; deuterium; a halogen group; a nitrile group; a nitro group; a hydroxy group; a carbonyl group; a ester group; an imide group; an amino group; a substituted or unsubstituted silyl group; a substituted or unsubstituted boron group; a substituted or unsubstituted alkyl group; a substituted or unsubstituted alkylsulfoxy group; a substituted or unsubstituted arylsulfoxy group; a substituted or unsubstituted alkenyl group; a substituted or unsubstituted aralkyl group; a substituted or unsubstituted aralkenyl group; a substituted or unsubstituted alkylaryl group; a substituted or unsubstituted alkylamine group; a substituted or unsubstituted aralkyl group; a substituted or unsubstituted heteroarylamine group; a substituted or unsubstituted arylamine group; a substituted or unsubstituted arylphosphinic group; a substituted or unsubstituted phosphine oxide group; a substituted or unsubstituted aryl group; or a substituted or unsubstituted heterocyclic group.

The compounds of the present invention have high electron mobility and excellent hole blocking ability. In addition, the organic light emitting device using the compound of the present invention has a high efficiency and a low driving voltage.

### BRIEF DESCRIPTION OF THE DRAWINGS

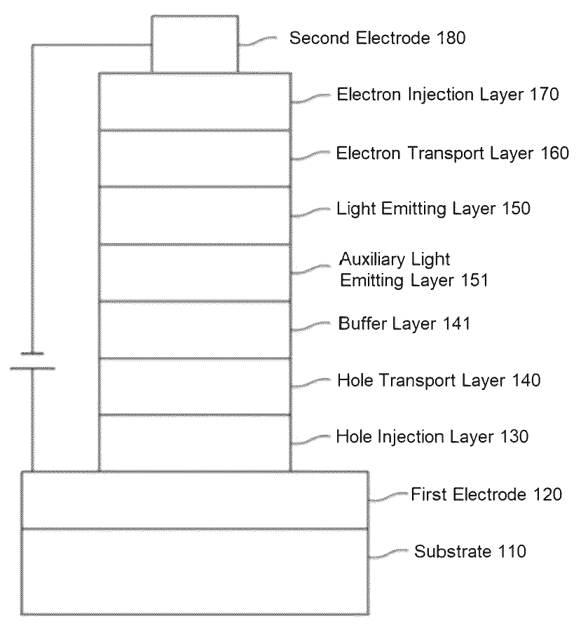

The attached FIGURE (hereinafter referred to as “FIG. 1”) is an illustration of an organic light emitting device according to an embodiment of the present invention.

### DETAILED DESCRIPTION

Hereinafter, embodiments of the present invention will be described in detail with reference to the accompanying drawings. In designation of reference numerals to components in respective drawings, it should be noted that the same components are given the same reference numerals even though they are shown in different FIGURES. Further, in the following description the present invention, a detailed description of known configurations or functions related thereto will be omitted when it is considered that it may make the subject matter of the present invention rather unclear.

It should be noted that if it is described in the specification that one component is “connected”, “coupled” or “joined” to another component, a third component may be “connected”, “coupled”, or “joined” between the first and second components, although the first component may be directly connected, coupled or joined to the second component.

As used in this specification and the accompanying claims, unless otherwise stated, the meaning of the following terms is as follows:

The term “halo” or “halogen” as used herein includes fluorine (F), bromine (Br), chlorine (Cl), or iodine (I), unless otherwise indicated.

The term “alkyl” or “alkyl group” as used herein, has a single bond of 1 to 60 carbon atoms unless otherwise indicated, and means aliphatic functional radicals including a linear alkyl group, a branched chain alkyl group, a cycloalkyl group (alicyclic), or an alkyl-substituted cycloalkyl group, a cycloalkyl-substituted alkyl group. Specific examples of alkyl groups include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohextylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like.

The term “haloalkyl group” or “halogen alkyl group” as used herein, means an alkyl group substituted with a halogen unless otherwise indicated.

The term “heteroalkyl group” as used herein, means an alkyl group of which at least one of carbon atoms is substituted with a hetero atom.

Unless otherwise stated, the term “alkenyl” or “alkynyl” as used herein has, but not limited to, double or triple bonds of 2 to 60 carbon atoms, and includes a linear alkyl group, or a branched chain alkyl group. Specific examples include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl) vinyl-1-yl, 2,2-bis(diphenyl-1-yl) vinyl-1-yl, a stilbenyl group, a styrenyl group, and the like, but are not limited thereto.

The term “cycloalkyl” as used herein, refers to alkyl that forms a ring having 3 to 60 carbon atoms unless otherwise indicated, but is not limited thereto. Specific examples include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl,3,4,5-trimethylcyclohexyl, 4-tert-butylcyclohexyl, cycloheptyl, cyclooctyl, and the like, but are not limited thereto.

The term “alkoxyl group”, “alkoxy group” or “alkyloxy group” as used herein, means an alkyl group to which an oxygen radical is attached, and, unless otherwise indicated, has from 1 to 60 carbon atoms, and is not limited thereto.

The term “alkenoxyl group”, “alkenoxy group”, “alkenyloxyl group”, or “alkenyloxy group” as used herein, means an alkenyl group to which oxygen radical is attached, and, unless otherwise stated, has 2 to 60 carbon atoms, but is not limited thereto.

The term “aryloxyl group” or “aryloxy group” as used herein means an aryl group to which oxygen radical is attached, and, unless otherwise indicated, has 6 to 60 carbon atoms, but is not limited thereto.

The terms “aryl group” and “arylene group” as used herein, unless otherwise indicated, have a carbon number of 6 to 60, respectively, and are not limited thereto.

The aryl group or arylene group herein means a monocyclic or polycyclic aromatic group, and includes an aromatic ring that is formed in conjunction with an adjacent substituent linked thereto or participating in the reaction. Examples of the aryl group may include as a monocyclic aryl group, a phenyl group, a biphenylyl group, and a terphenylyl group, and may include as a polycyclic aryl group, a naphthyl group, an anthracenyl group, a phenanthryl group, a pyrenyl group, a perylenyl group, a chrysenyl group, a fluorenyl group, and a spirofluorenyl group.

The fluorenyl group herein can be substituted and two substituents can be bonded together to form a spiro structure. When the fluorenyl group is substituted, it may have the following structure, but is not limited thereto.

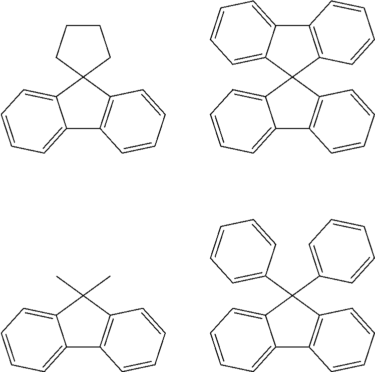

The prefix “aryl” or “Ar” refers to a radical substituted with an aryl group. For example, an arylalkyl group is an alkyl group substituted with an aryl group, and an arylalkenyl group is an alkenyl group substituted with an aryl group, and a radical substituted with an aryl group has the carbon number defined as herein.

Also, when the prefix is subsequently named, it means that substituents are listed in the order described first. For example, an arylalkoxy group refers to an alkoxy group substituted with an aryl group, an alkoxylcarbonyl group means a carbonyl group substituted with an alkoxyl group, and an arylcarbonylalkenyl group means an alkenyl group substituted with an arylcarbonyl group, wherein the arylcarbonyl group is a carbonyl group substituted with an aryl group.

Unless otherwise stated, the term “heteroaryl group” or “heteroarylene group” as used herein means, but not limited to, an aryl or arylene group having 2 to 60 carbon atoms and containing one or more heteroatoms, includes at least one of monocyclic and polycyclic rings, and may also be formed in conjunction with an adjacent functional group.

Unless otherwise stated, the term “heterocyclic group” as used herein contains one or more heteroatoms, has 2 to 60 carbon atoms, includes at least one of monocyclic and polycyclic rings, and includes a heteroaliphatic ring and a heteroaromatic ring. It may be formed in conjunction with adjacent functional groups. “Heteroatom” refers to N, O, S, P, or Si unless otherwise indicated. A “heterocyclic group” may also include a ring containing SO₂ instead of carbon forming the ring.

Examples of heterocyclic groups include a thiophene group, a furan group, a pyrrole group, an imidazole group, a thiazole group, an oxazole group, an oxadiazole group, a triazole group, a pyridyl group, a pyrimidyl group, a triazine group, a triazole group, an acridyl group, a pyridazine group, a pyrazinyl group, a quinolinyl group, a quinazoline group, a quinoxalinyl group, a phthalazinyl group, a pyrido pyrimidinyl group, a pyrido pyrazinyl group, a pyrazino pyrazinyl group, an isoquinoline group, an indole group, a carbazole group, a benzooxazole group, a benzoimidazole group, a benzothiazole group, a benzocarbazole group, a benzothiophene group, a dibenzothiophene group, a benzofuranyl group, a phenanthroline group, a thiazolyl group, an isooxazolyl group, an oxadiazolyl group, a thiadiazolyl group, a benzothiazolyl group, a phenothiazinyl group, and a dibenzofuranyl group, but are not limited thereto.

Unless otherwise indicated, the term “aliphatic” as used herein refers to aliphatic hydrocarbons having from 1 to 60 carbon atoms, and “aliphatic ring” means an aliphatic hydrocarbon ring having from 3 to 60 carbon atoms.

Unless otherwise stated, the term “ring” means an aliphatic ring having 3 to 60 carbon atoms, an aromatic ring having 6 to 60 carbon atoms, a hetero ring having 2 to 60 carbon atoms, or a fused ring formed by the combination of them, and includes a saturated or unsaturated ring.

Other heterocompounds or heteroradicals other than the foregoing heterocompounds include, but are not limited to, one or more heteroatoms.

Unless otherwise stated, the term “carbonyl” as used herein is represented by —COR′, wherein R′ may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “ether” as used herein is represented by —R—O—R′, wherein R or R′ independently may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these, respectively.

Unless otherwise stated, the term “substituted or unsubstituted” as used herein means that substitution is carried out by at least one substituent selected from the group consisting of, but not limited to, deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopene group, a C₆-C₂₀ arylthiopene group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group.

Otherwise specified, the formulas used in the present invention are defined as in the index definition of the substituent of the following Formula.

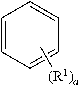

Wherein, when a is an integer of zero, the substituent R¹ is absent, when a is an integer of 1, the sole substituent R¹ is linked to any one of the carbon atoms constituting the benzene ring, when a is an integer of 2 or 3, the R¹s may be the same and different, and are linked to the benzene ring as follows. When a is an integer of 4 to 6, the R¹s may be the same and different, and are linked to the benzene ring in a similar manner to that when a is an integer of 2 or 3. Hydrogen atoms linked to carbon constituents of the benzene ring are omitted.

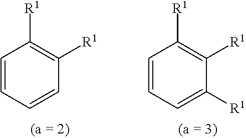

FIG. 1 illustrates an organic light emitting device according to an embodiment of the present invention.

Referring to FIG. 1, an organic light emitting device 100 according to an embodiment of the present invention includes a first electrode 120 formed on a substrate 110, a second electrode 180, and an organic material layer formed between the first electrode 120 and the second electrode 180, wherein the organic layer includes a compound according to the present invention. The first electrode 120 may be an anode (positive electrode), and the second electrode 180 may be a cathode (negative electrode). In the case of an inverted organic light emitting device, the first electrode may be a cathode, and the second electrode may be an anode.

The anode is desirably formed of a material having a high work function so that hole injection into the organic material layer can be facilitated. Specific examples of materials for the anode that may be used in the present invention include metals such as vanadium, chromium, copper, zinc, gold, or alloys thereof; metal oxides such as zinc oxide, indium oxide, indium tin oxide (ITO), indium zinc oxide (IZO); combinations of metals and oxides such as ZnO:Al or SNO₂:Sb; conductive polymers such as poly(3-methylthiophene), poly[3,4-(ethylene-1,2-dioxy)thiophene] (PEDOT), polypyrrole, and polyaniline, but are not limited thereto.

It is desired that the cathode is formed of a material having a small work function to facilitate electron injection into the organic material layer. Specific examples of cathode materials include, but are not limited to, metals such as magnesium, calcium, sodium, potassium, titanium, indium, yttrium, lithium, gadolinium, aluminum, silver, tin, and lead, or alloys thereof; multi-layered structure materials such as LiF/Al or LiO₂/Al and the like.

The organic material layer may include a hole injection layer 130, a hole transport layer 140, a light emitting layer 150, an electron transport layer 160, and an electron injection layer 170 formed in sequence on the first electrode 120. Here, some of the layers included in the organic material layer, except for the light emitting layer 150, may not be formed.

The hole injection layer 130 is a layer that facilitates injection of holes from the first electrode 120, and is desirably formed of a compound having excellent hole injection effect from the anode and thin film formation capability. For these reasons, it is preferred that the highest occupied molecular orbital (HOMO) of the hole injecting material falls between the work function of the anode material and the HOMO of the surrounding organic material layer. Examples of the hole-injecting material include, but are not limited to, a metal porphyrin, an oligothiophene, an arylamine-based organic material, a hexanitrilehexaazatriphenylene-based organic material, a quinacridone-based organic material, a perylene-based organic material, an anthraquinone, and polyaniline-based and polythiophene-based conductive polymer.

The hole transport layer 140 receives holes from the hole injection layer 130 and transports holes to the light emitting layer 150, and the hole transport material is preferably a material having a high mobility for holes. Specific examples include, but are not limited to, an arylamine-based organic material, a conductive polymer, and a block copolymer having both a conjugated part and a non-conjugated part.

The light emitting layer 150 is a layer emitting light in the visible light region by receiving holes and electrons from the hole transport layer 140 and the electron transport layer 160, respectively, and recombining them. The light-emitting material is preferably a material having good quantum efficiency for fluorescence or phosphorescence. Specific examples include, but are not limited to, 8-hydroxy-quinoline aluminum complex (Alq₃); carbazole based compounds; dimerized styryl compounds; BAlq; 10-hydroxybenzo quinoline-metal compounds; benzoxazole, benzthiazole and benzimidazole-based compounds; poly(p-phenylenevinylene) (PPV)-based polymers; spiro compounds; polyfluorenes, rubrene, and the like.

The light emitting layer 150 may include a host material and a dopant material. The host material may be a fused aromatic ring derivative or a heterocyclic-containing compound. Specifically, the fused aromatic ring derivative includes an anthracene derivative, a pyrene derivative, a naphthalene derivative, a pentacene derivative, a phenanthrene compound, a fluoranthene compound, and the like. The heterocyclic-containing compound includes, but is not limited to, a carbazole derivative, a dibenzofuran derivative, a ladder-type furan compound, a pyrimidine derivative, and the like.

The dopant material includes an aromatic amine derivative, a styrylamine compound, a boron complex, a fluoranthene compound, a metal complex, and the like. Specifically, the aromatic amine derivative is a fused aromatic ring derivative having a substituted or unsubstituted arylamino group, and includes a pyrene, an anthracene, a chrysene, and a periflanthene, each of which has an arylamino group. The styrylamine compound is a compound in which at least one arylvinyl group is substituted in a substituted or unsubstituted arylamine, in which at least one or two substituent selected from the group consisting of an aryl group, a silyl group, an alkyl group, a cycloalkyl group, and an arylamino group is substituted or unsubstituted. Specific examples include, but are not limited to, styrylamine, styryldiamine, styryltriamine, styryltetraamine, and the like. In addition, the metal complex includes, but is not limited to, a iridium complex, a platinum complex, and the like.

The electron transport layer 160 receives electrons from the electron injection layer 170 and transports electrons to the light emitting layer 150, and the electron transport material is preferably a material having a high mobility for electrons. Specific examples include, but are not limited to, Al complexes of 8-hydroxyquinoline; complexes including Alq₃; organic radical compounds; hydroxyflavone-metal complexes; and the like. The electron transporting materials of the present invention are described below.

The electron injection layer 170 is a layer that facilitates the injection of electrons from the second electrode 180, and it is preferable that the electron injection layer 170 has the ability to transport electrons, and the electron injection effect from the cathode electrode and the ability to form the thin film are excellent. Examples include, but are not limited to, fluorenone, anthraquinodimethane, diphenoquinone, thiopyran dioxide, oxazole, oxadiazole, triazole, imidazole, perylenetetracarboxylic acid, preorenylidene methane, anthrone, and derivatives thereof, metal complex compounds, and nitrogen-containing 5-membered ring derivatives. Metal complex compounds include 8-hydroxyquinolinato lithium, bis(8-hydroxyquinolinato) Zinc, bis(8-hydroxyquinolinato) Copper, bis(8-hydroxyquinolinato) Manganese, tris(8-hydroxyquinolinato) Aluminum, tris(2-methyl-8-hydroxyquinolinato) Aluminum, tris(8-hydroxyquinolinato) Gallium, bis(10-hydroxybenzo [h] quinolinato) beryllium, bis(10-hydroxybenzo[h]quinolinato) zinc, bis(2-methyl-8-quinolinato) chlorogallium, bis(2-methyl-8-quinolinato) (o-cresolato) gallium, bis(2-methyl-8-quinolinato) (1-naphtholato) aluminum, bis(2-methyl-8-quinolinato) (2-naphtholato) gallium, and the like, but are not limited thereto.

In addition to the hole injection layer 130, the hole transport layer 140, the light emitting layer 150, the electron transport layer 160, and the electron injection layer 170, the organic material layer may further include a hole blocking layer, an electron blocking layer, an auxiliary light emitting layer 151, a buffer layer 141, and the like, and the electron transport layer 160 or the like may serve as a hole blocking layer.

In addition, although not shown, the organic light emitting device according to the present invention may further include a protective layer or a light efficiency improving layer (capping layer) formed on at least one of sides of the first electrode 120 and the second electrode 180, which is opposite to the organic material layer.

Although the compound according to the present invention is mainly used in an electron transporting region such as the electron injection layer 170, the electron transport layer 160, a hole blocking layer, etc. the present invention is not limited thereto, and may be used as a hole transport region such as the hole injection layer 130, the hole transport layer 140, a host or a dopant of the light emitting layer 150, or a capping layer.

The organic light emitting device according to an embodiment of the present invention can be manufactured using a physical vapor deposition (PVD) method such as vacuum evaporation or sputtering. For example, the device can be manufactured by depositing a metal or a conductive metal oxide or a mixture thereof on a substrate to form the anode 120, forming an organic material layer including the hole injection layer 130, the hole transport layer 140, the light emitting layer 150, the electron transport layer 160, and the electron injection layer 170 on the anode 120, and then depositing a material that can be used as the cathode 180 thereon.

In addition, the organic material layer may be formed of a smaller number of layers by using a soluble process or a solvent process, such as a spin coating process, a nozzle printing process, an inkjet printing process, a slot coating process, a dip coating process, a roll-to-roll process, a doctor blading process, a screen printing process, or a thermal transfer method. Since the organic layer according to the present invention can be formed by various methods, the scope of the present invention is not limited by the formation method thereof.

Depending on a used material, the organic light emitting device according to the present invention can be of a top emission type, a bottom emission type, or a dual emission type.

A WOLED (White Organic Light Emitting Device) readily allows for the formation of ultra-high definition images, and is of excellent processability as well as enjoying the advantage of being produced using conventional color filter technologies for LCDs. In this regard, various structures for WOLEDs, usually used as back light units, have been suggested and patented. Representative among the structures are a parallel side-by-side arrangement of R (Red), G (Green), B (Blue) light-emitting units, a vertical stack arrangement of RGB light-emitting units, and a color conversion material (CCM) structure in which electroluminescence from a blue (B) organic light emitting layer and photoluminescence from an inorganic phosphor using light from the blue (B) organic light emitting layer are combined. The present invention is applicable to these WOLEDs.

Another embodiment of the present invention provides an electronic device including a display device, which includes the above described organic light emitting device and a control unit for controlling the display device. Here, the electronic device may be a wired/wireless communication terminal which is currently used or will be used in the future, and includes all kinds of electronic devices including a mobile communication terminal such as a cellular phone, a personal digital assistant (PDA), an electronic dictionary, a point-to-multipoint (PMP), a remote controller, a navigation device, a game player, various kinds of TVs, and various kinds of computers.

Hereinafter, a compound according to an aspect of the present invention will be described.

According to an aspect of the present invention, provided is a compound represented by chemical formula 1,

A₁ [symbol] L [symbol] A₂ <Chemical Formula 1>
wherein A₁ is a group represented by any one of the following structures:

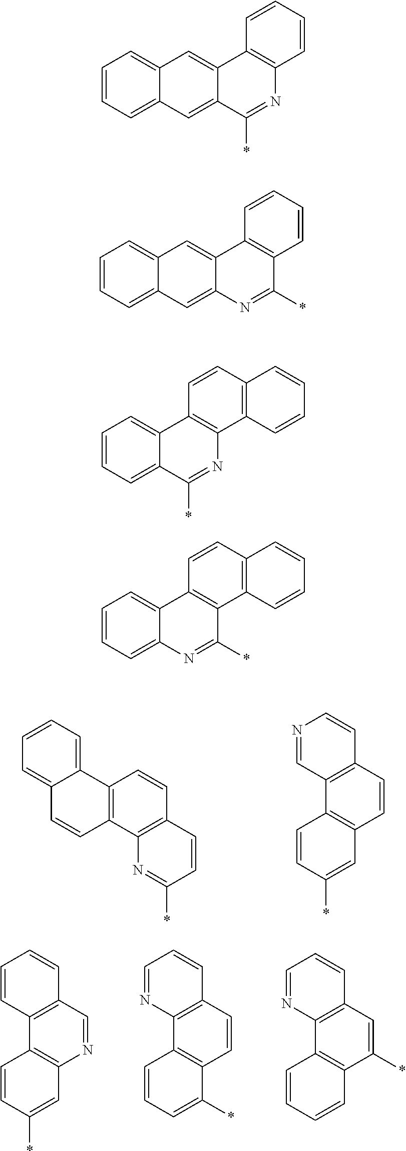

L is a direct bond; a substituted or unsubstituted arylene group; or a substituted or unsubstituted heteroarylene group; or a substituted or unsubstituted C₉-C₆₀ fused polycyclic group,
A₂ is hydrogen; deuterium; a halogen group; a nitrile group; a nitro group; a hydroxy group; a carbonyl group; a ester group; an imide group; an amino group; a substituted or unsubstituted silyl group; a substituted or unsubstituted boron group; a substituted or unsubstituted alkyl group; a substituted or unsubstituted alkylsulfoxy group; a substituted or unsubstituted arylsulfoxy group; a substituted or unsubstituted alkenyl group; a substituted or unsubstituted aralkyl group; a substituted or unsubstituted aralkenyl group; a substituted or unsubstituted alkylaryl group; a substituted or unsubstituted alkylamine group; a substituted or unsubstituted aralkyl group; a substituted or unsubstituted heteroarylamine group; a substituted or unsubstituted arylamine group; a substituted or unsubstituted arylphosphinic group; a substituted or unsubstituted phosphine oxide group; a substituted or unsubstituted aryl group; or a substituted or unsubstituted heterocyclic group.

Furthermore, L of the compound may have the following structure, wherein L₁ to L₃ are each independently a direct bond; a substituted or unsubstituted arylene group; or a substituted or unsubstituted heteroarylene group; or a substituted or unsubstituted C₉-C₆₀ fused polycyclic group.

-L₁-L₂-L₃-

Furthermore, L of chemical formula 1 may have the following structure.

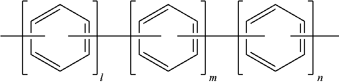

wherein L, M, and n are each independently 0 or 1.

Furthermore, A₂ of the compound may be any one of the following structures, wherein X₁ to X₃ are each independently C or N, at least one of X₁ to X₃ is N, and Ar₁ and Ar₂ are each independently hydrogen, deuterium, a halogen group, a cyano group, a substituted or unsubstituted C₁ to C₆₀ alkyl group, a substituted or unsubstituted C₃ to C₁₀ cycloalkyl group, a substituted or unsubstituted C₆ to C₆₀ aryl group, a substituted or unsubstituted C₁ to C₆₀ heteroaryl group.

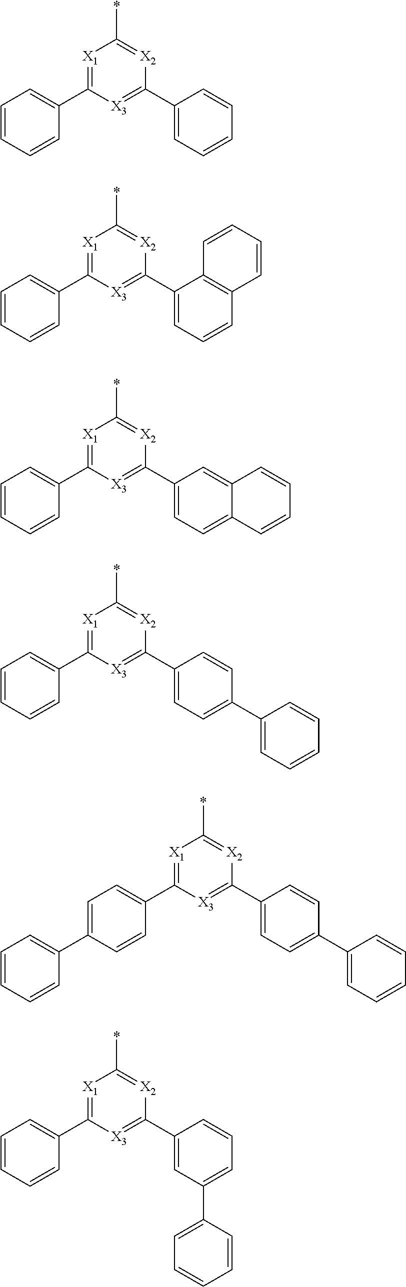

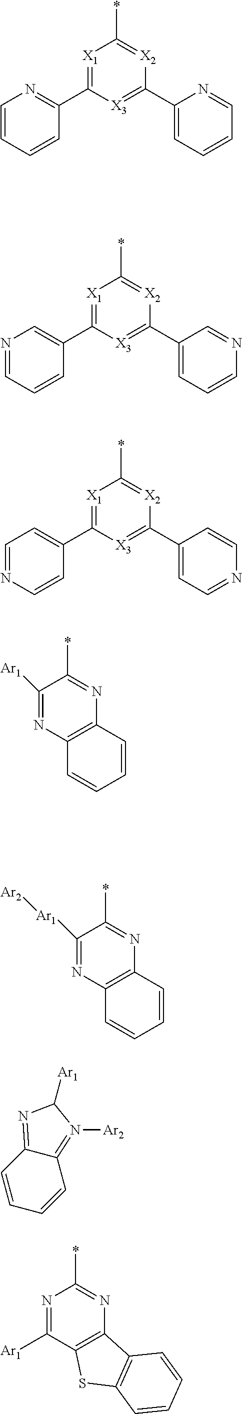

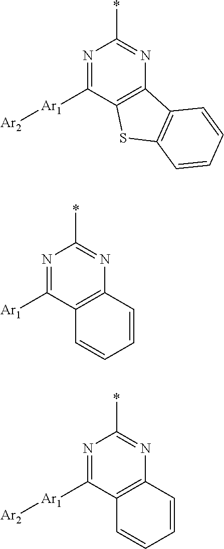

A₂ of the compound may be represented by the following structural formula:

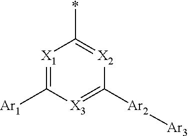

wherein X₁ to X₃ are each independently C or N, at least one of X₁ to X₃ is N, Ar₁ and Ar₂ are the same or different, each being independently hydrogen, deuterium, a halogen group, a cyano group, a substituted or unsubstituted C₁ to C₆₀ alkyl group, a substituted or unsubstituted C₃ to C₁₀ cycloalkyl group, a substituted or unsubstituted C₆ to C₆₀ aryl group, a substituted or unsubstituted C₆ to C₆₀ arylene group, a substituted or unsubstituted C₁ to C₆₀ heteroaryl group and, Ar3 is hydrogen, deuterium, a halogen group, a cyano group, a substituted or unsubstituted C₁ to C₆₀ alkyl group, a substituted or unsubstituted C₃ to C₁₀ cycloalkyl group, a substituted or unsubstituted C₆ to C₆₀ aryl group, a substituted or unsubstituted C₁ to C₆₀ heteroaryl group.

Furthermore, the compound of chemical formula 1 may be any one of the following compounds.

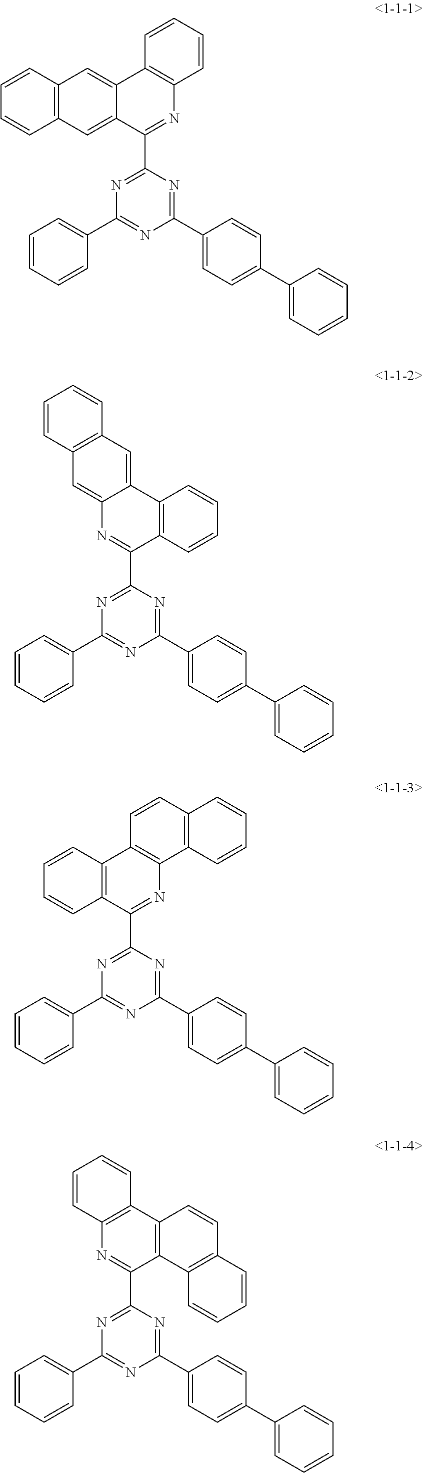 [SMILES: c1ccc(-c2ccc(-c3nc(-c4ccccc4)nc(-c4nc5ccccc5c5cc6ccccc6cc45)n3)cc2)cc1 ; c1ccc(-c2ccc(-c3nc(-c4ccccc4)nc(-c4nc5cc6ccccc6cc5c5ccccc45)n3)cc2)cc1 ; c1ccc(-c2ccc(-c3nc(-c4ccccc4)nc(-c4nc5c6ccccc6ccc5c5ccccc45)n3)cc2)cc1 ; c1ccc(-c2ccc(-c3nc(-c4ccccc4)nc(-c4nc5ccccc5c5ccc6ccccc6c45)n3)cc2)cc1]

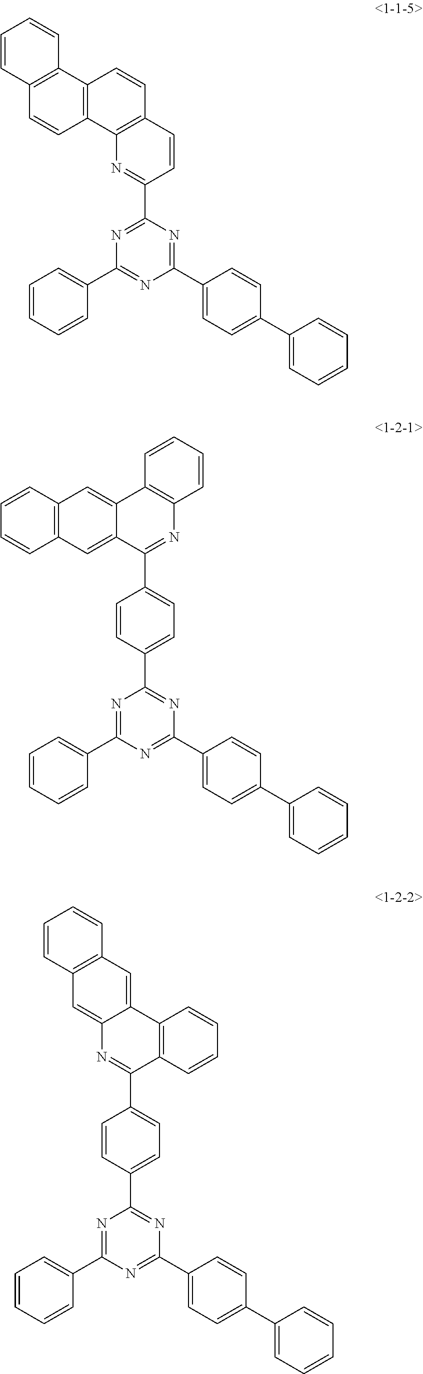 [SMILES: c1ccc(-c2ccc(-c3nc(-c4ccccc4)nc(-c4ccc5ccc6c7ccccc7ccc6c5n4)n3)cc2)cc1 ; c1ccc(-c2ccc(-c3nc(-c4ccccc4)nc(-c4ccc(-c5nc6ccccc6c6cc7ccccc7cc56)cc4)n3)cc2)cc1 ; c1ccc(-c2ccc(-c3nc(-c4ccccc4)nc(-c4ccc(-c5nc6cc7ccccc7cc6c6ccccc56)cc4)n3)cc2)cc1]

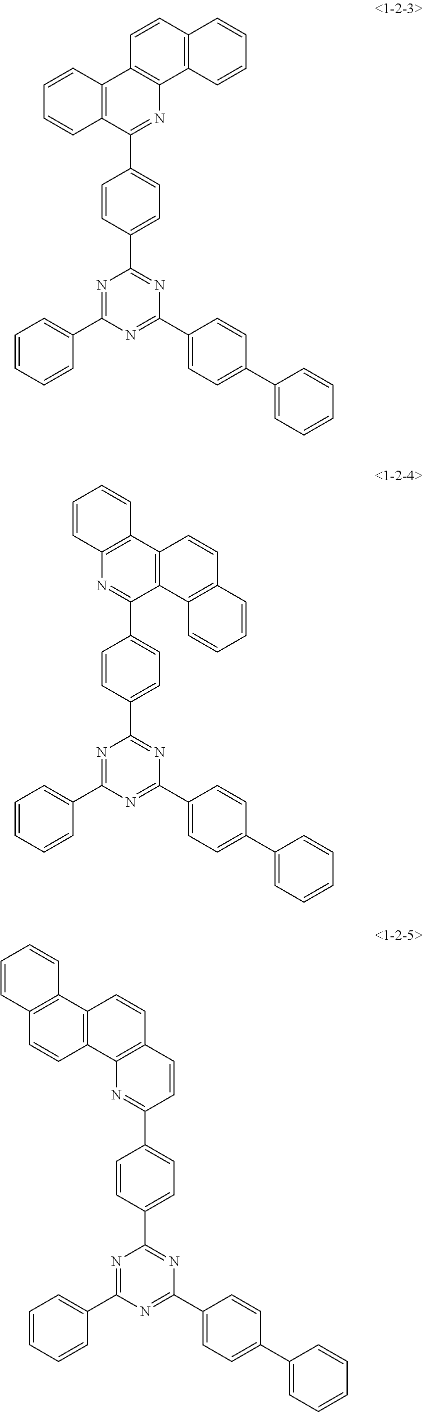 [SMILES: c1ccc(-c2ccc(-c3nc(-c4ccccc4)nc(-c4ccc(-c5nc6c7ccccc7ccc6c6ccccc56)cc4)n3)cc2)cc1 ; c1ccc(-c2ccc(-c3nc(-c4ccccc4)nc(-c4ccc(-c5nc6ccccc6c6ccc7ccccc7c56)cc4)n3)cc2)cc1 ; c1ccc(-c2ccc(-c3nc(-c4ccccc4)nc(-c4ccc(-c5ccc6ccc7c8ccccc8ccc7c6n5)cc4)n3)cc2)cc1]

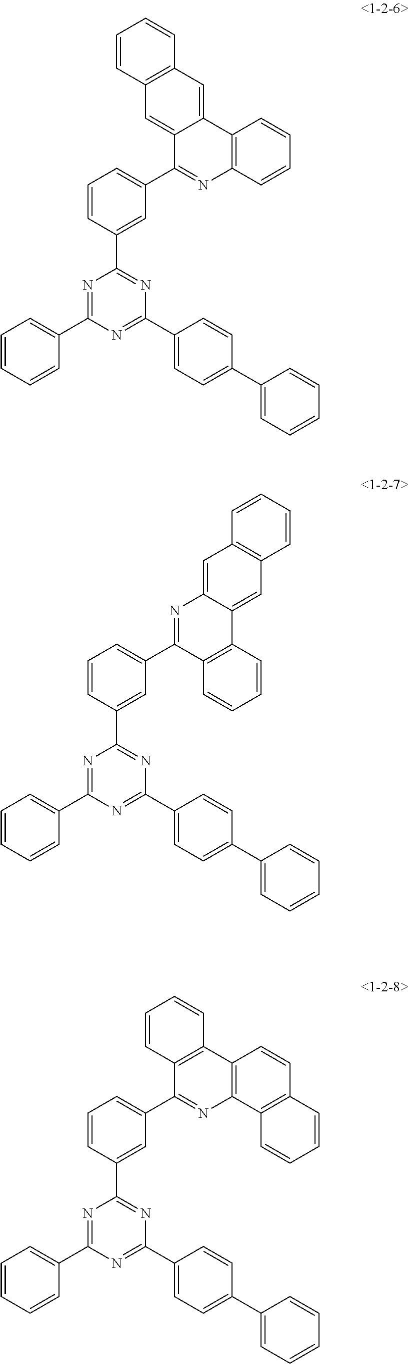 [SMILES: c1ccc(-c2ccc(-c3nc(-c4ccccc4)nc(-c4cccc(-c5nc6ccccc6c6cc7ccccc7cc56)c4)n3)cc2)cc1 ; c1ccc(-c2ccc(-c3nc(-c4ccccc4)nc(-c4cccc(-c5nc6cc7ccccc7cc6c6ccccc56)c4)n3)cc2)cc1 ; c1ccc(-c2ccc(-c3nc(-c4ccccc4)nc(-c4cccc(-c5nc6c7ccccc7ccc6c6ccccc56)c4)n3)cc2)cc1]

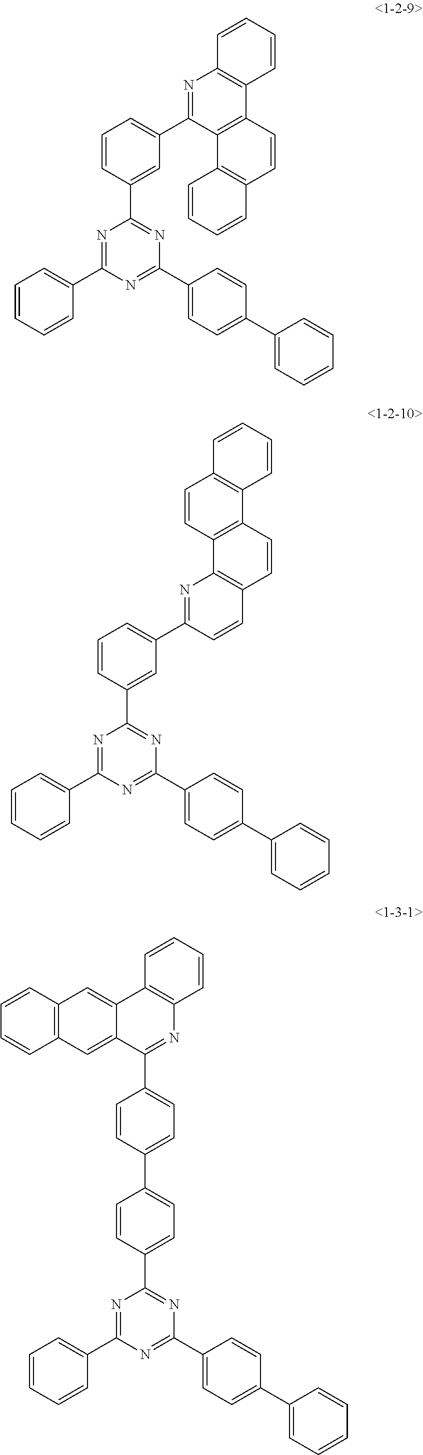 [SMILES: c1ccc(-c2ccc(-c3nc(-c4ccccc4)nc(-c4cccc(-c5nc6ccccc6c6ccc7ccccc7c56)c4)n3)cc2)cc1 ; c1ccc(-c2ccc(-c3nc(-c4ccccc4)nc(-c4cccc(-c5ccc6ccc7c8ccccc8ccc7c6n5)c4)n3)cc2)cc1 ; c1ccc(-c2ccc(-c3nc(-c4ccccc4)nc(-c4ccc(-c5ccc(-c6nc7ccccc7c7cc8ccccc8cc67)cc5)cc4)n3)cc2)cc1]

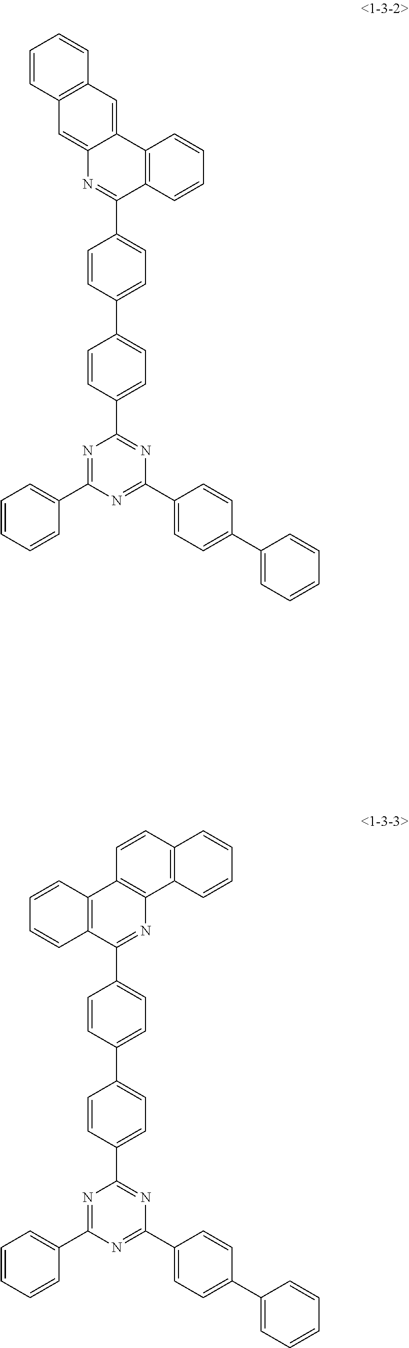 [SMILES: c1ccc(-c2ccc(-c3nc(-c4ccccc4)nc(-c4ccc(-c5ccc(-c6nc7cc8ccccc8cc7c7ccccc67)cc5)cc4)n3)cc2)cc1 ; c1ccc(-c2ccc(-c3nc(-c4ccccc4)nc(-c4ccc(-c5ccc(-c6nc7c8ccccc8ccc7c7ccccc67)cc5)cc4)n3)cc2)cc1]

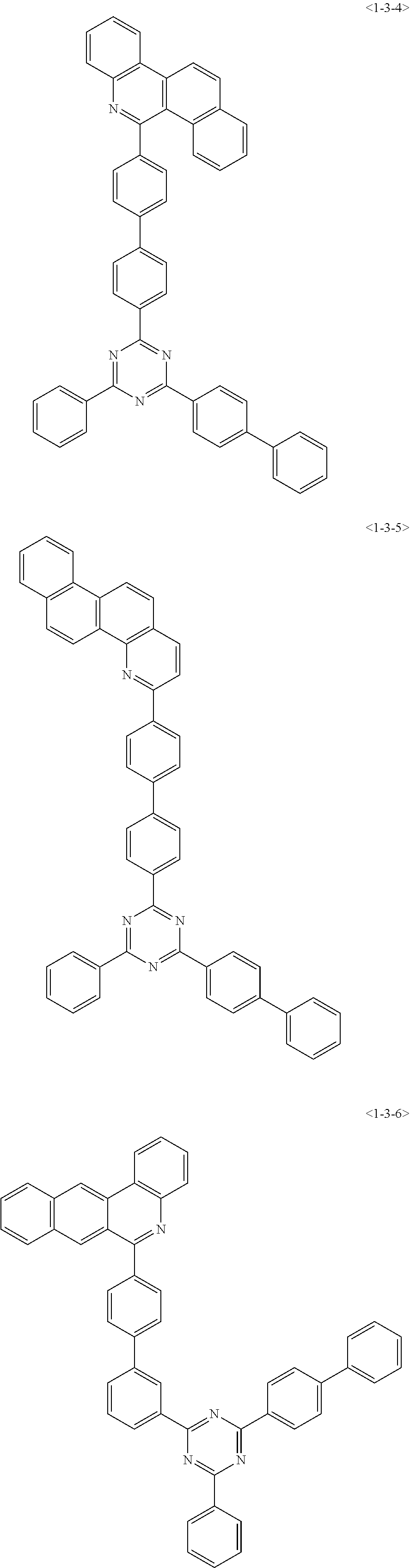 [SMILES: c1ccc(-c2ccc(-c3nc(-c4ccccc4)nc(-c4ccc(-c5ccc(-c6nc7ccccc7c7ccc8ccccc8c67)cc5)cc4)n3)cc2)cc1 ; c1ccc(-c2ccc(-c3nc(-c4ccccc4)nc(-c4ccc(-c5ccc(-c6ccc7ccc8c9ccccc9ccc8c7n6)cc5)cc4)n3)cc2)cc1 ; c1ccc(-c2ccc(-c3nc(-c4ccccc4)nc(-c4cccc(-c5ccc(-c6nc7ccccc7c7cc8ccccc8cc67)cc5)c4)n3)cc2)cc1]

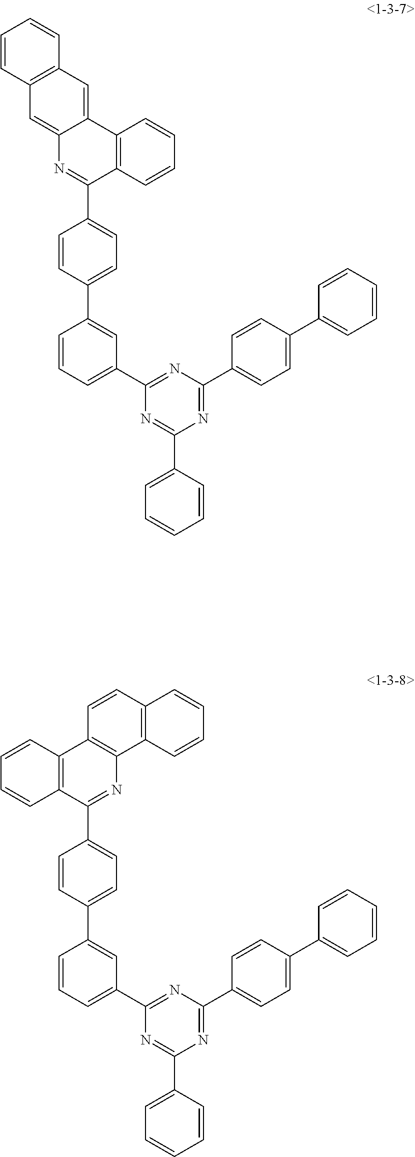 [SMILES: c1ccc(-c2ccc(-c3nc(-c4ccccc4)nc(-c4cccc(-c5ccc(-c6nc7cc8ccccc8cc7c7ccccc67)cc5)c4)n3)cc2)cc1 ; c1ccc(-c2ccc(-c3nc(-c4ccccc4)nc(-c4cccc(-c5ccc(-c6nc7c8ccccc8ccc7c7ccccc67)cc5)c4)n3)cc2)cc1]

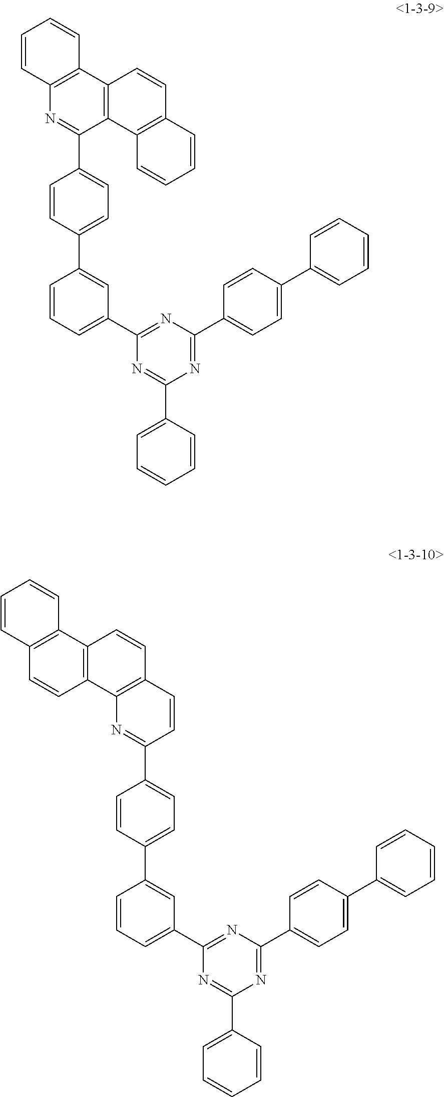 [SMILES: c1ccc(-c2ccc(-c3nc(-c4ccccc4)nc(-c4cccc(-c5ccc(-c6nc7ccccc7c7ccc8ccccc8c67)cc5)c4)n3)cc2)cc1 ; c1ccc(-c2ccc(-c3nc(-c4ccccc4)nc(-c4cccc(-c5ccc(-c6ccc7ccc8c9ccccc9ccc8c7n6)cc5)c4)n3)cc2)cc1]

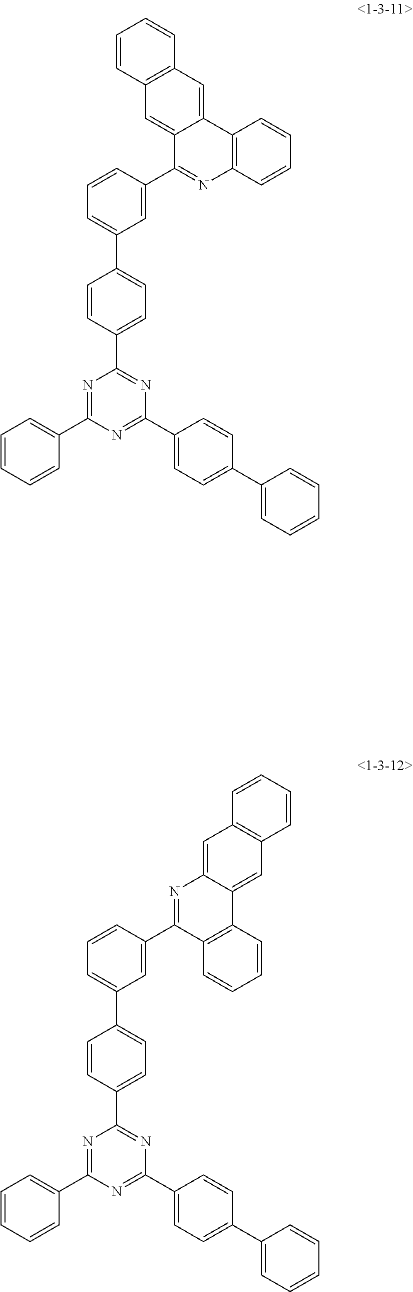 [SMILES: c1ccc(-c2ccc(-c3nc(-c4ccccc4)nc(-c4ccc(-c5cccc(-c6nc7ccccc7c7cc8ccccc8cc67)c5)cc4)n3)cc2)cc1 ; c1ccc(-c2ccc(-c3nc(-c4ccccc4)nc(-c4ccc(-c5cccc(-c6nc7cc8ccccc8cc7c7ccccc67)c5)cc4)n3)cc2)cc1]

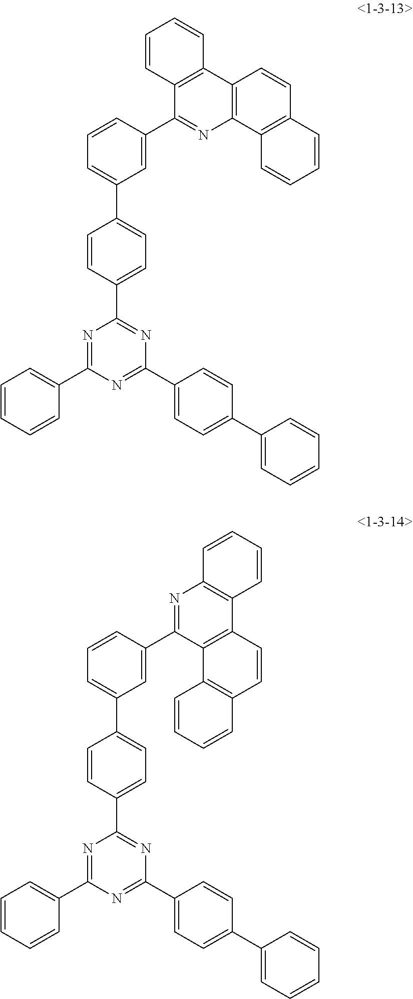 [SMILES: c1ccc(-c2ccc(-c3nc(-c4ccccc4)nc(-c4ccc(-c5cccc(-c6nc7c8ccccc8ccc7c7ccccc67)c5)cc4)n3)cc2)cc1 ; c1ccc(-c2ccc(-c3nc(-c4ccccc4)nc(-c4ccc(-c5cccc(-c6nc7ccccc7c7ccc8ccccc8c67)c5)cc4)n3)cc2)cc1]

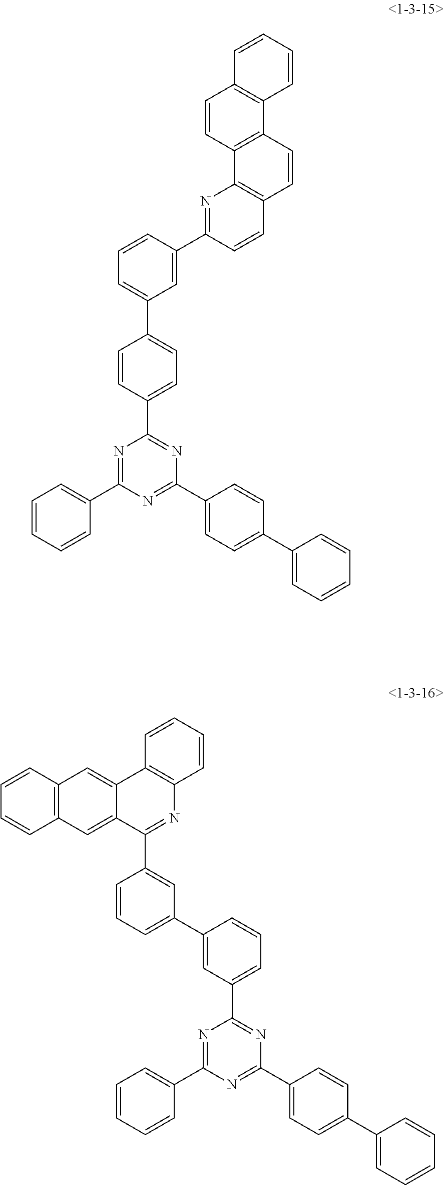 [SMILES: c1ccc(-c2ccc(-c3nc(-c4ccccc4)nc(-c4ccc(-c5cccc(-c6ccc7ccc8c9ccccc9ccc8c7n6)c5)cc4)n3)cc2)cc1 ; c1ccc(-c2ccc(-c3nc(-c4ccccc4)nc(-c4cccc(-c5cccc(-c6nc7ccccc7c7cc8ccccc8cc67)c5)c4)n3)cc2)cc1]

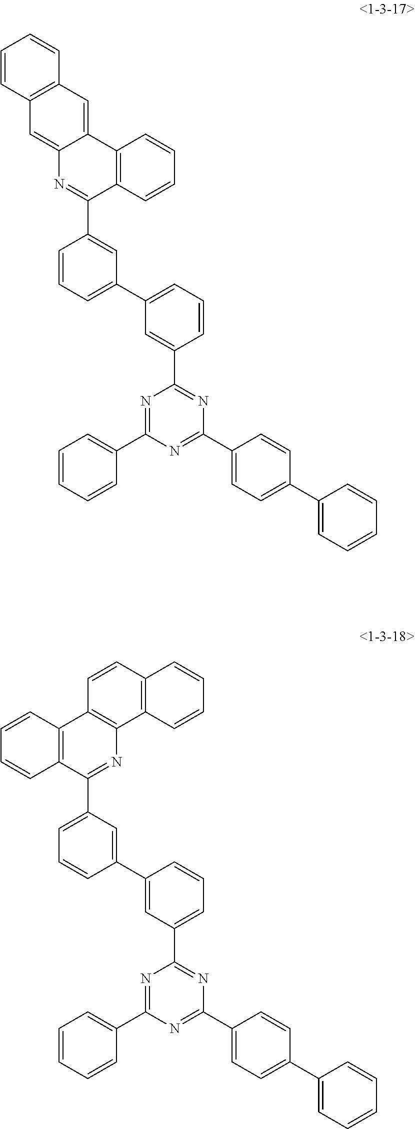 [SMILES: c1ccc(-c2ccc(-c3nc(-c4ccccc4)nc(-c4cccc(-c5cccc(-c6nc7cc8ccccc8cc7c7ccccc67)c5)c4)n3)cc2)cc1 ; c1ccc(-c2ccc(-c3nc(-c4ccccc4)nc(-c4cccc(-c5cccc(-c6nc7c8ccccc8ccc7c7ccccc67)c5)c4)n3)cc2)cc1]

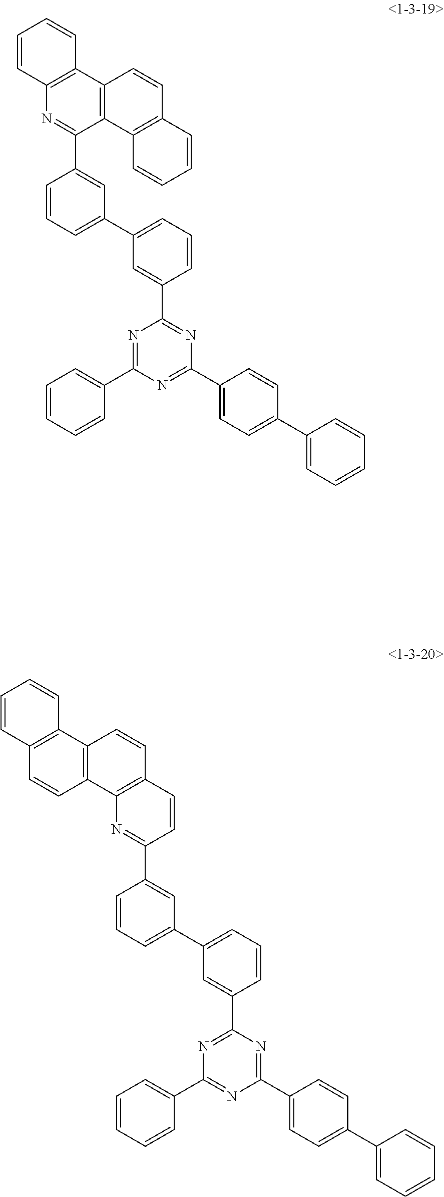 [SMILES: c1ccc(-c2ccc(-c3nc(-c4ccccc4)nc(-c4cccc(-c5cccc(-c6nc7ccccc7c7ccc8ccccc8c67)c5)c4)n3)cc2)cc1 ; c1ccc(-c2ccc(-c3nc(-c4ccccc4)nc(-c4cccc(-c5cccc(-c6ccc7ccc8c9ccccc9ccc8c7n6)c5)c4)n3)cc2)cc1]

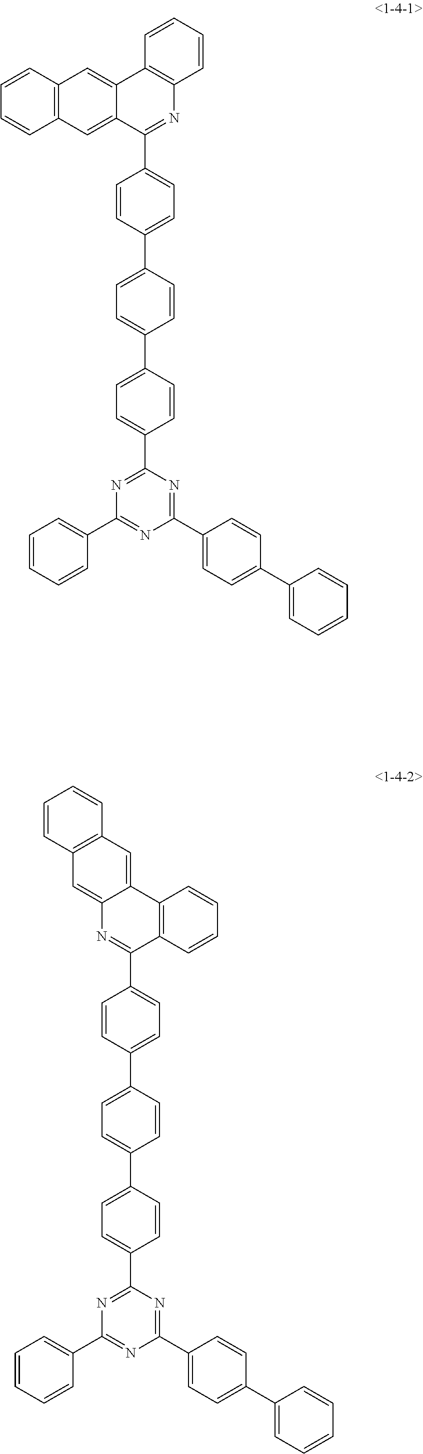 [SMILES: c1ccc(-c2ccc(-c3nc(-c4ccccc4)nc(-c4ccc(-c5ccc(-c6ccc(-c7nc8ccccc8c8cc9ccccc9cc78)cc6)cc5)cc4)n3)cc2)cc1 ; c1ccc(-c2ccc(-c3nc(-c4ccccc4)nc(-c4ccc(-c5ccc(-c6ccc(-c7nc8cc9ccccc9cc8c8ccccc78)cc6)cc5)cc4)n3)cc2)cc1]

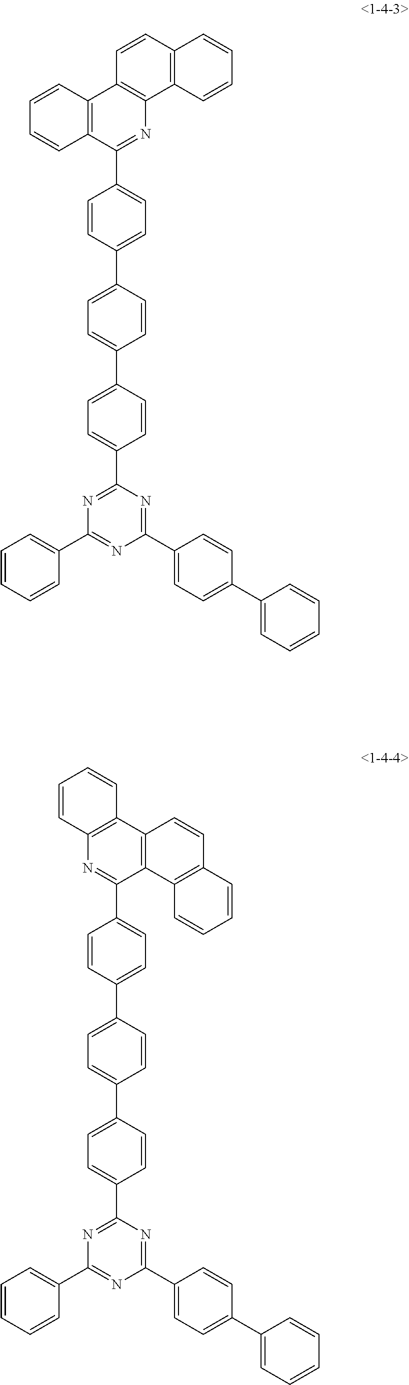 [SMILES: c1ccc(-c2ccc(-c3nc(-c4ccccc4)nc(-c4ccc(-c5ccc(-c6ccc(-c7nc8c9ccccc9ccc8c8ccccc78)cc6)cc5)cc4)n3)cc2)cc1 ; c1ccc(-c2ccc(-c3nc(-c4ccccc4)nc(-c4ccc(-c5ccc(-c6ccc(-c7nc8ccccc8c8ccc9ccccc9c78)cc6)cc5)cc4)n3)cc2)cc1]

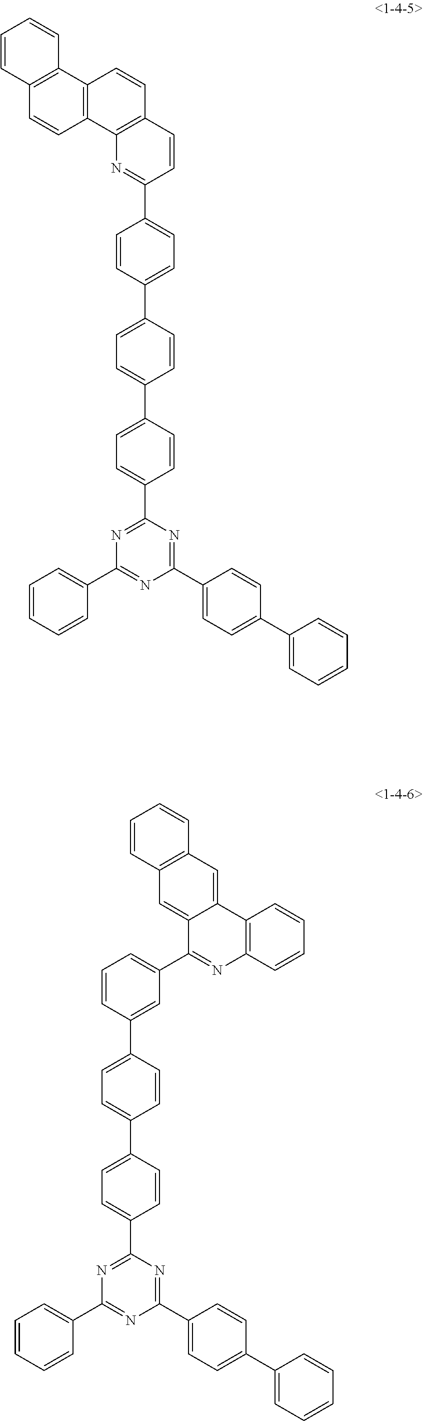 [SMILES: c1ccc(-c2ccc(-c3nc(-c4ccccc4)nc(-c4ccc(-c5ccc(-c6ccc(-c7ccc8ccc9c%10ccccc%10ccc9c8n7)cc6)cc5)cc4)n3)cc2)cc1 ; c1ccc(-c2ccc(-c3nc(-c4ccccc4)nc(-c4ccc(-c5ccc(-c6cccc(-c7nc8ccccc8c8cc9ccccc9cc78)c6)cc5)cc4)n3)cc2)cc1]

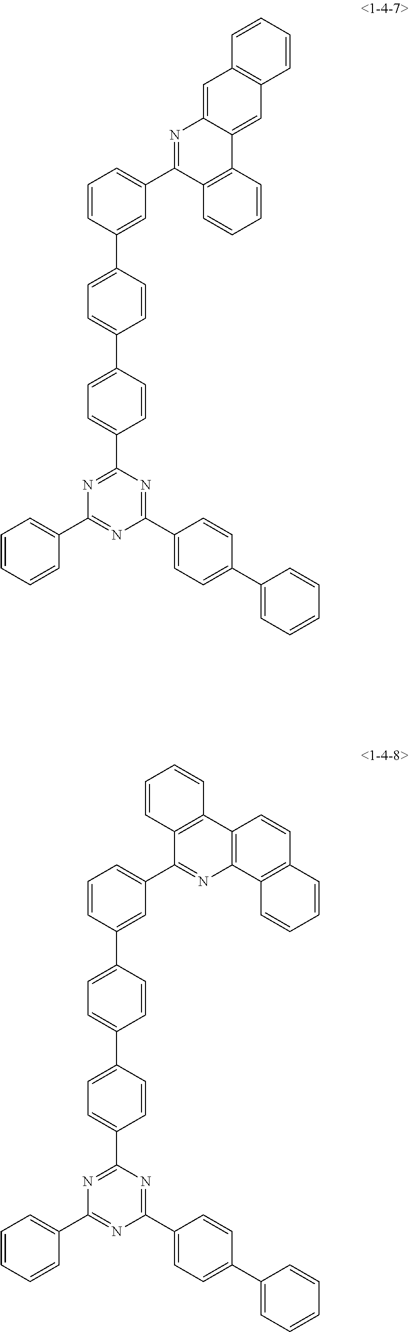 [SMILES: c1ccc(-c2ccc(-c3nc(-c4ccccc4)nc(-c4ccc(-c5ccc(-c6cccc(-c7nc8cc9ccccc9cc8c8ccccc78)c6)cc5)cc4)n3)cc2)cc1 ; c1ccc(-c2ccc(-c3nc(-c4ccccc4)nc(-c4ccc(-c5ccc(-c6cccc(-c7nc8c9ccccc9ccc8c8ccccc78)c6)cc5)cc4)n3)cc2)cc1]

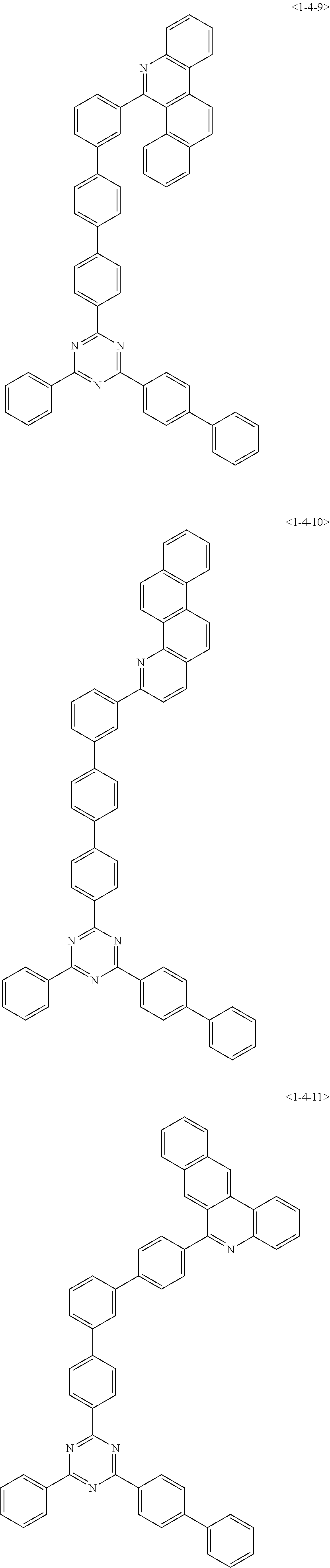 [SMILES: c1ccc(-c2ccc(-c3nc(-c4ccccc4)nc(-c4ccc(-c5ccc(-c6cccc(-c7nc8ccccc8c8ccc9ccccc9c78)c6)cc5)cc4)n3)cc2)cc1 ; c1ccc(-c2ccc(-c3nc(-c4ccccc4)nc(-c4ccc(-c5ccc(-c6cccc(-c7ccc8ccc9c%10ccccc%10ccc9c8n7)c6)cc5)cc4)n3)cc2)cc1 ; c1ccc(-c2ccc(-c3nc(-c4ccccc4)nc(-c4ccc(-c5cccc(-c6ccc(-c7nc8ccccc8c8cc9ccccc9cc78)cc6)c5)cc4)n3)cc2)cc1]

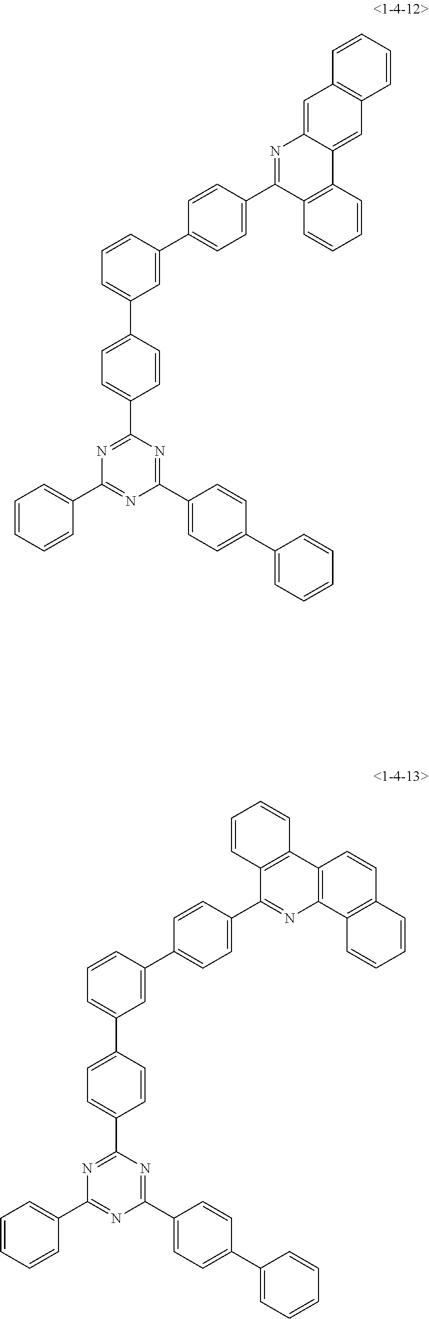 [SMILES: c1ccc(-c2ccc(-c3nc(-c4ccccc4)nc(-c4ccc(-c5cccc(-c6ccc(-c7nc8cc9ccccc9cc8c8ccccc78)cc6)c5)cc4)n3)cc2)cc1 ; c1ccc(-c2ccc(-c3nc(-c4ccccc4)nc(-c4ccc(-c5cccc(-c6ccc(-c7nc8c9ccccc9ccc8c8ccccc78)cc6)c5)cc4)n3)cc2)cc1]

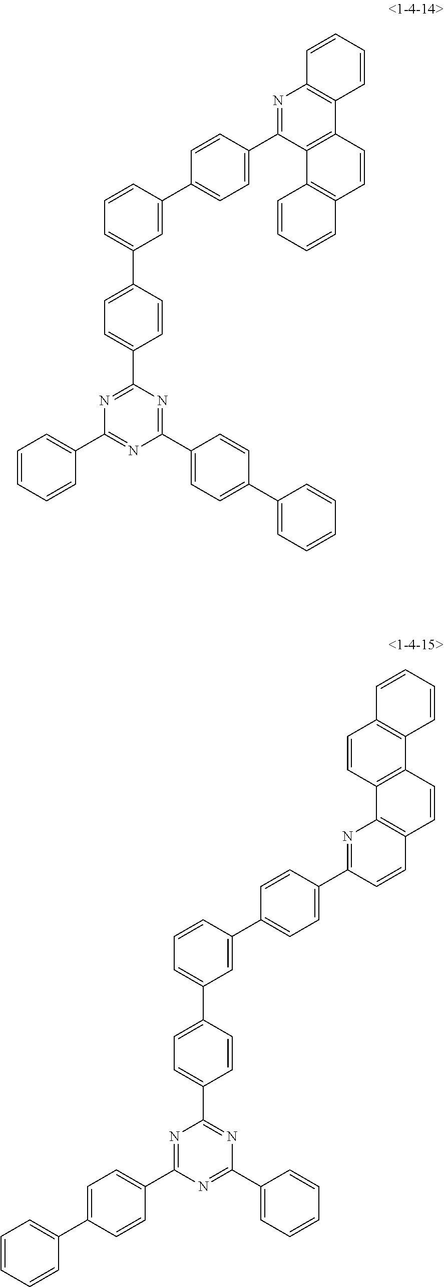 [SMILES: c1ccc(-c2ccc(-c3nc(-c4ccccc4)nc(-c4ccc(-c5cccc(-c6ccc(-c7nc8ccccc8c8ccc9ccccc9c78)cc6)c5)cc4)n3)cc2)cc1 ; c1ccc(-c2ccc(-c3nc(-c4ccccc4)nc(-c4ccc(-c5cccc(-c6ccc(-c7ccc8ccc9c%10ccccc%10ccc9c8n7)cc6)c5)cc4)n3)cc2)cc1]

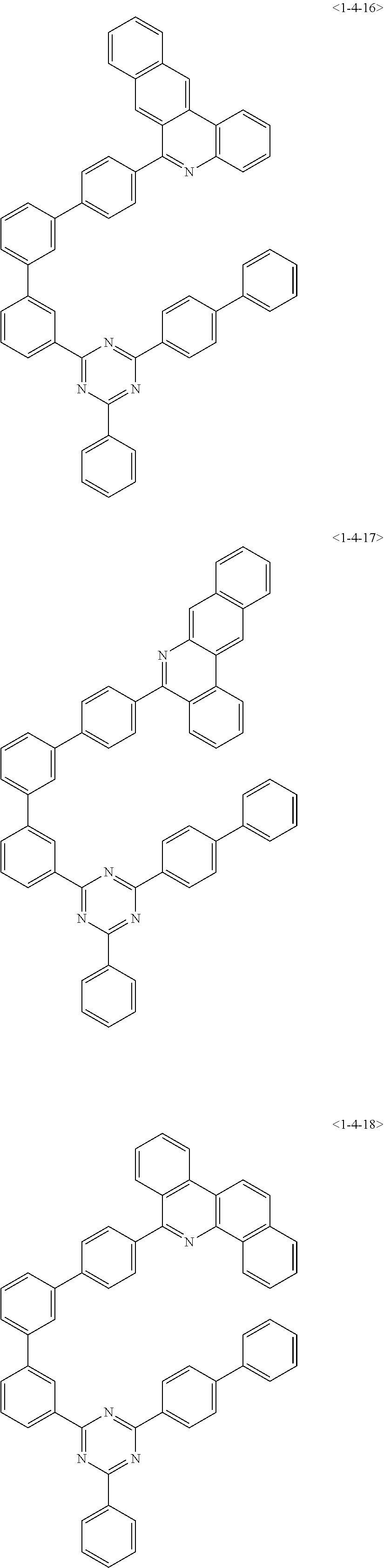 [SMILES: c1ccc(-c2ccc(-c3nc(-c4ccccc4)nc(-c4cccc(-c5cccc(-c6ccc(-c7nc8ccccc8c8cc9ccccc9cc78)cc6)c5)c4)n3)cc2)cc1 ; c1ccc(-c2ccc(-c3nc(-c4ccccc4)nc(-c4cccc(-c5cccc(-c6ccc(-c7nc8cc9ccccc9cc8c8ccccc78)cc6)c5)c4)n3)cc2)cc1 ; c1ccc(-c2ccc(-c3nc(-c4ccccc4)nc(-c4cccc(-c5cccc(-c6ccc(-c7nc8c9ccccc9ccc8c8ccccc78)cc6)c5)c4)n3)cc2)cc1]

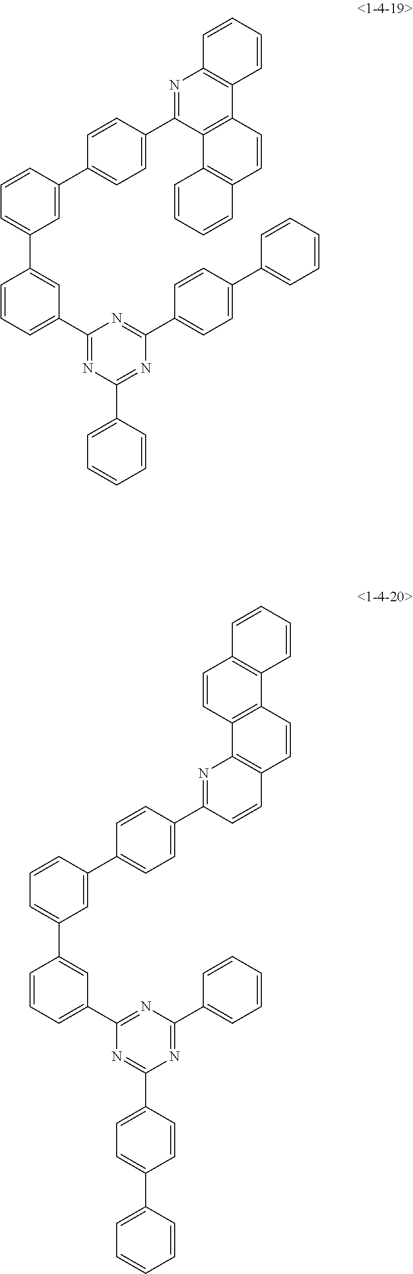 [SMILES: c1ccc(-c2ccc(-c3nc(-c4ccccc4)nc(-c4cccc(-c5cccc(-c6ccc(-c7nc8ccccc8c8ccc9ccccc9c78)cc6)c5)c4)n3)cc2)cc1 ; c1ccc(-c2ccc(-c3nc(-c4ccccc4)nc(-c4cccc(-c5cccc(-c6ccc(-c7ccc8ccc9c%10ccccc%10ccc9c8n7)cc6)c5)c4)n3)cc2)cc1]

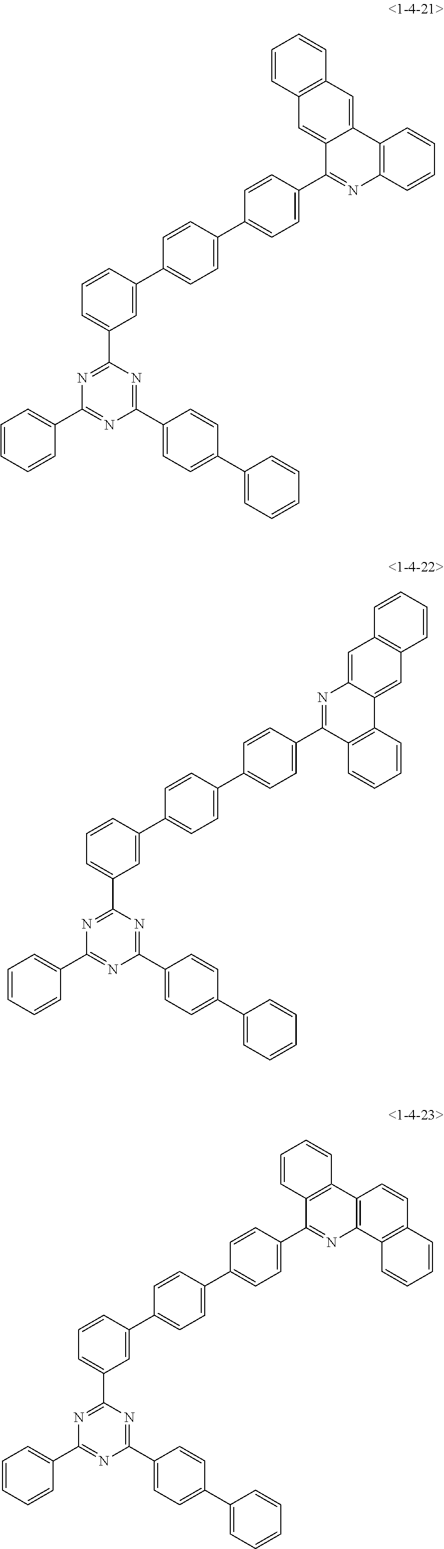 [SMILES: c1ccc(-c2ccc(-c3nc(-c4ccccc4)nc(-c4cccc(-c5ccc(-c6ccc(-c7nc8ccccc8c8cc9ccccc9cc78)cc6)cc5)c4)n3)cc2)cc1 ; c1ccc(-c2ccc(-c3nc(-c4ccccc4)nc(-c4cccc(-c5ccc(-c6ccc(-c7nc8cc9ccccc9cc8c8ccccc78)cc6)cc5)c4)n3)cc2)cc1 ; c1ccc(-c2ccc(-c3nc(-c4ccccc4)nc(-c4cccc(-c5ccc(-c6ccc(-c7nc8c9ccccc9ccc8c8ccccc78)cc6)cc5)c4)n3)cc2)cc1]

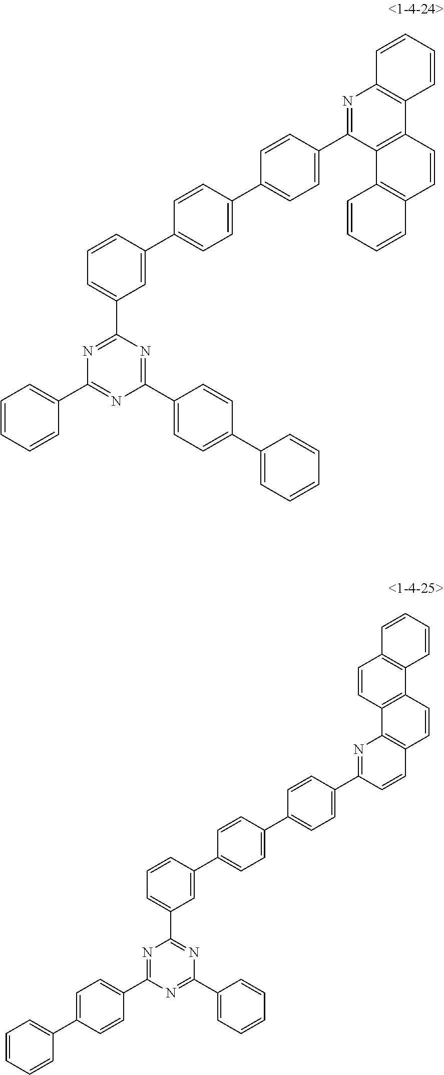 [SMILES: c1ccc(-c2ccc(-c3nc(-c4ccccc4)nc(-c4cccc(-c5ccc(-c6ccc(-c7nc8ccccc8c8ccc9ccccc9c78)cc6)cc5)c4)n3)cc2)cc1 ; c1ccc(-c2ccc(-c3nc(-c4ccccc4)nc(-c4cccc(-c5ccc(-c6ccc(-c7ccc8ccc9c%10ccccc%10ccc9c8n7)cc6)cc5)c4)n3)cc2)cc1]

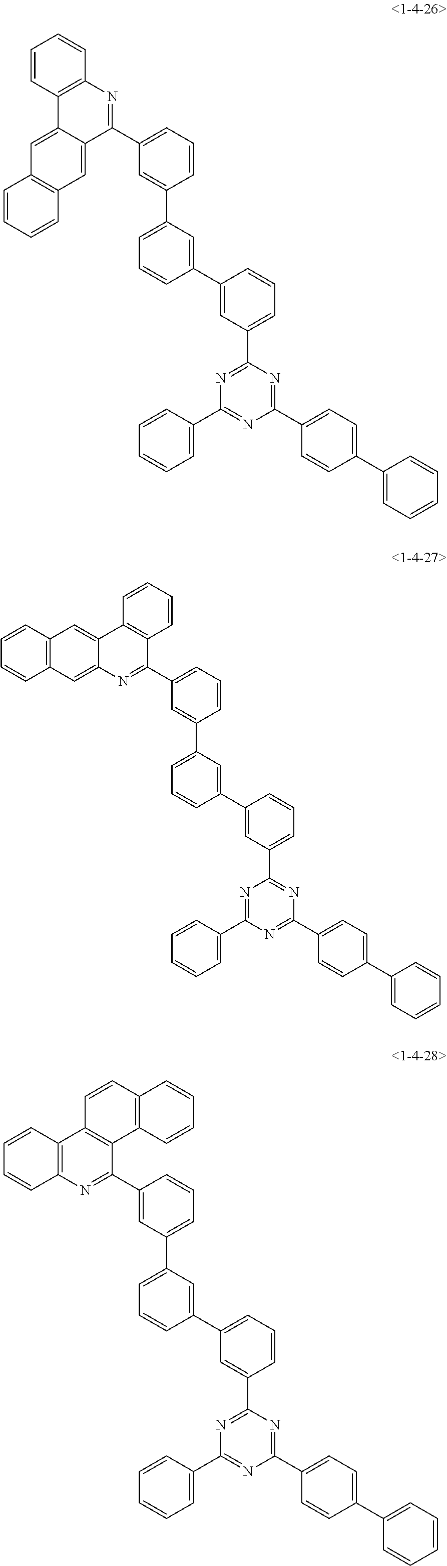 [SMILES: c1ccc(-c2ccc(-c3nc(-c4ccccc4)nc(-c4cccc(-c5cccc(-c6cccc(-c7nc8ccccc8c8cc9ccccc9cc78)c6)c5)c4)n3)cc2)cc1 ; c1ccc(-c2ccc(-c3nc(-c4ccccc4)nc(-c4cccc(-c5cccc(-c6cccc(-c7nc8cc9ccccc9cc8c8ccccc78)c6)c5)c4)n3)cc2)cc1 ; c1ccc(-c2ccc(-c3nc(-c4ccccc4)nc(-c4cccc(-c5cccc(-c6cccc(-c7nc8ccccc8c8ccc9ccccc9c78)c6)c5)c4)n3)cc2)cc1]

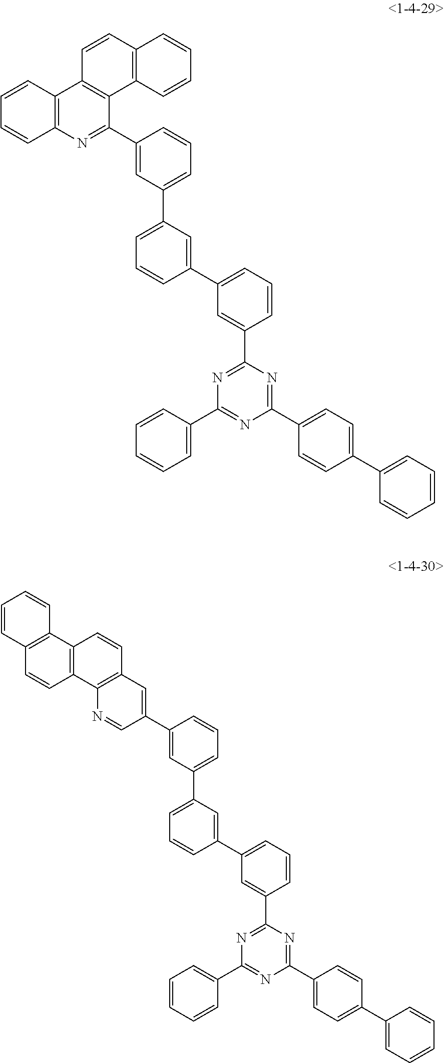 [SMILES: c1ccc(-c2ccc(-c3nc(-c4ccccc4)nc(-c4cccc(-c5cccc(-c6cccc(-c7nc8ccccc8c8ccc9ccccc9c78)c6)c5)c4)n3)cc2)cc1 ; c1ccc(-c2ccc(-c3nc(-c4ccccc4)nc(-c4cccc(-c5cccc(-c6cccc(-c7cnc8c(ccc9c%10ccccc%10ccc98)c7)c6)c5)c4)n3)cc2)cc1]

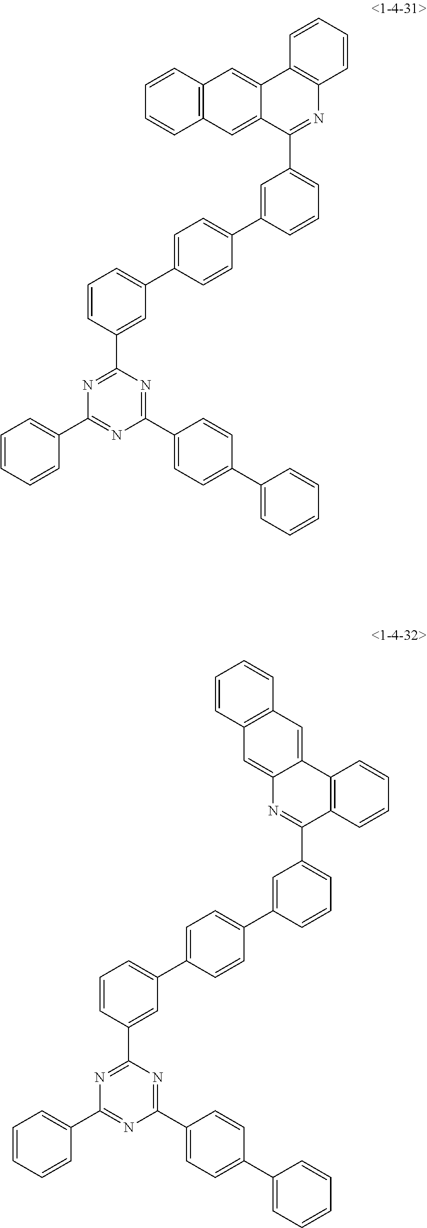 [SMILES: c1ccc(-c2ccc(-c3nc(-c4ccccc4)nc(-c4cccc(-c5ccc(-c6cccc(-c7nc8ccccc8c8cc9ccccc9cc78)c6)cc5)c4)n3)cc2)cc1 ; c1ccc(-c2ccc(-c3nc(-c4ccccc4)nc(-c4cccc(-c5ccc(-c6cccc(-c7nc8cc9ccccc9cc8c8ccccc78)c6)cc5)c4)n3)cc2)cc1]

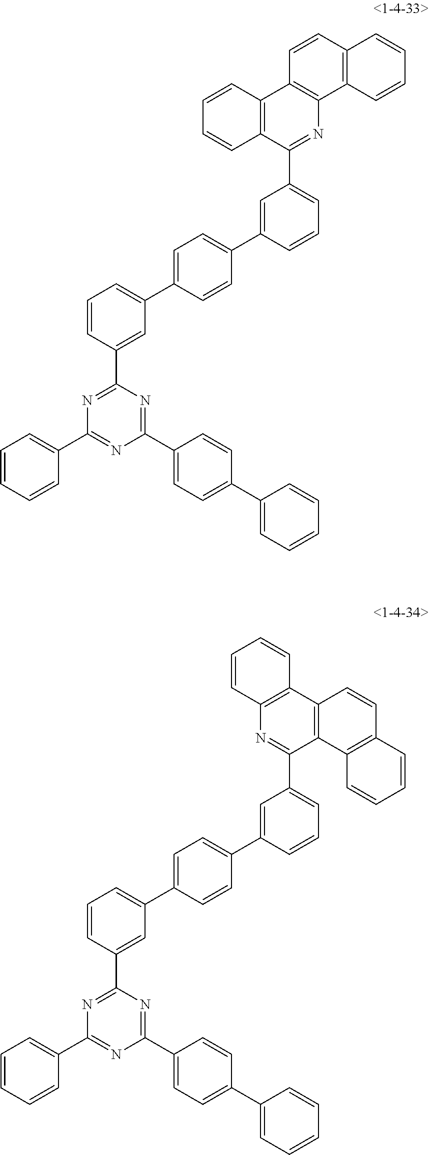 [SMILES: c1ccc(-c2ccc(-c3nc(-c4ccccc4)nc(-c4cccc(-c5ccc(-c6cccc(-c7nc8c9ccccc9ccc8c8ccccc78)c6)cc5)c4)n3)cc2)cc1 ; c1ccc(-c2ccc(-c3nc(-c4ccccc4)nc(-c4cccc(-c5ccc(-c6cccc(-c7nc8ccccc8c8ccc9ccccc9c78)c6)cc5)c4)n3)cc2)cc1]

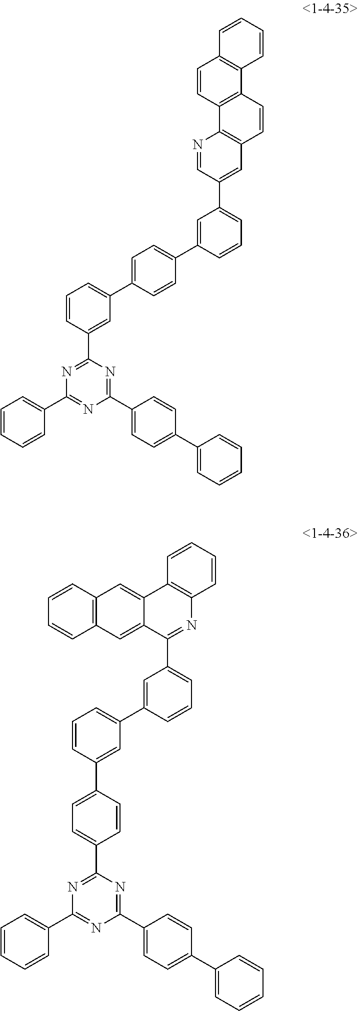 [SMILES: c1ccc(-c2ccc(-c3nc(-c4ccccc4)nc(-c4cccc(-c5ccc(-c6cccc(-c7cnc8c(ccc9c%10ccccc%10ccc98)c7)c6)cc5)c4)n3)cc2)cc1 ; c1ccc(-c2ccc(-c3nc(-c4ccccc4)nc(-c4ccc(-c5cccc(-c6cccc(-c7nc8ccccc8c8cc9ccccc9cc78)c6)c5)cc4)n3)cc2)cc1]

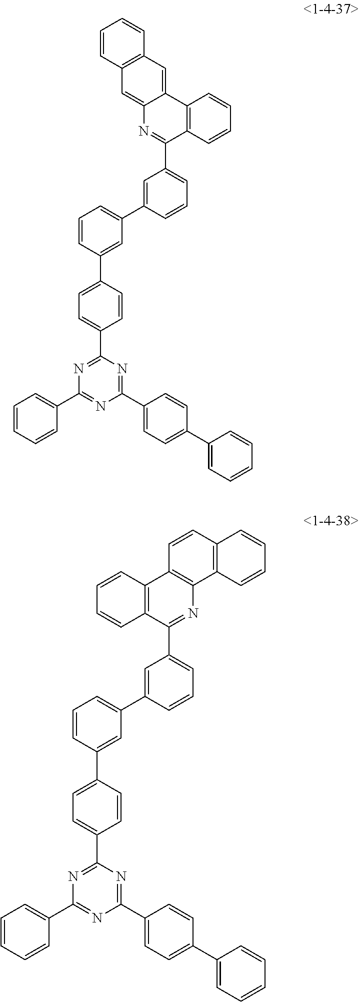 [SMILES: c1ccc(-c2ccc(-c3nc(-c4ccccc4)nc(-c4ccc(-c5cccc(-c6cccc(-c7nc8cc9ccccc9cc8c8ccccc78)c6)c5)cc4)n3)cc2)cc1 ; c1ccc(-c2ccc(-c3nc(-c4ccccc4)nc(-c4ccc(-c5cccc(-c6cccc(-c7nc8c9ccccc9ccc8c8ccccc78)c6)c5)cc4)n3)cc2)cc1]

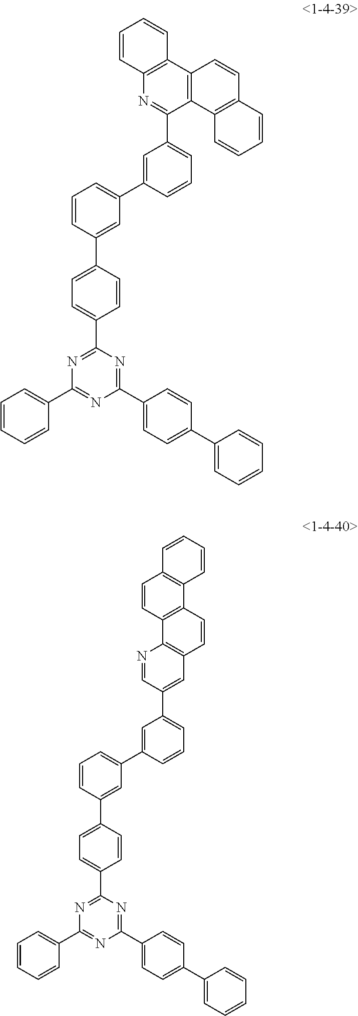 [SMILES: c1ccc(-c2ccc(-c3nc(-c4ccccc4)nc(-c4ccc(-c5cccc(-c6cccc(-c7nc8ccccc8c8ccc9ccccc9c78)c6)c5)cc4)n3)cc2)cc1 ; c1ccc(-c2ccc(-c3nc(-c4ccccc4)nc(-c4ccc(-c5cccc(-c6cccc(-c7cnc8c(ccc9c%10ccccc%10ccc98)c7)c6)c5)cc4)n3)cc2)cc1]

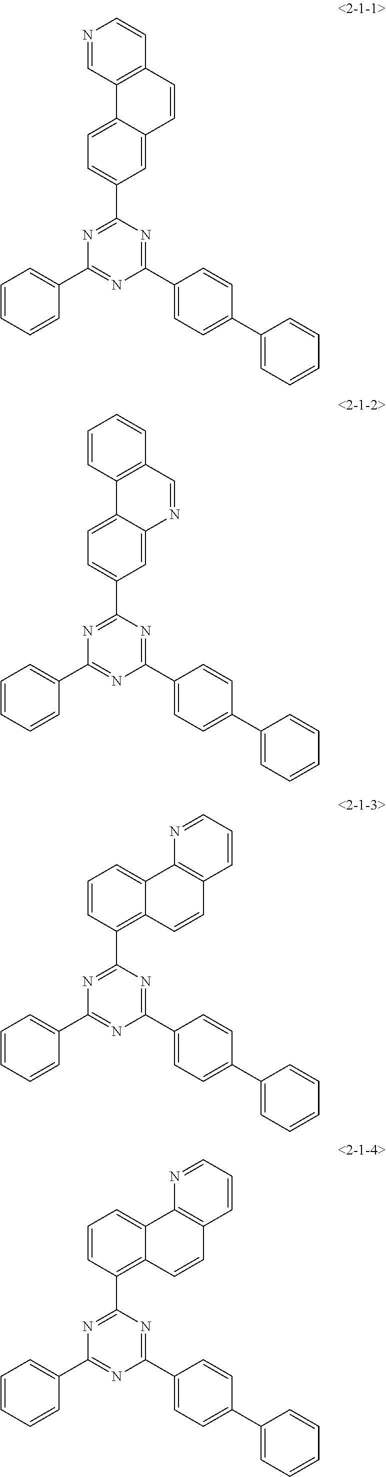 [SMILES: c1ccc(-c2ccc(-c3nc(-c4ccccc4)nc(-c4ccc5c(ccc6ccncc65)c4)n3)cc2)cc1 ; c1ccc(-c2ccc(-c3nc(-c4ccccc4)nc(-c4ccc5c(c4)ncc4ccccc45)n3)cc2)cc1 ; c1ccc(-c2ccc(-c3nc(-c4ccccc4)nc(-c4cccc5c4ccc4cccnc45)n3)cc2)cc1 ; c1ccc(-c2ccc(-c3nc(-c4ccccc4)nc(-c4cccc5c4ccc4cccnc45)n3)cc2)cc1]

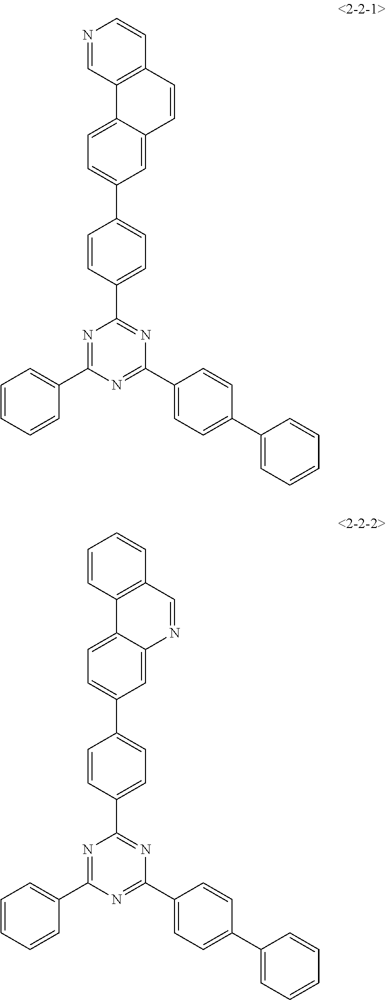 [SMILES: c1ccc(-c2ccc(-c3nc(-c4ccccc4)nc(-c4ccc(-c5ccc6c(ccc7ccncc76)c5)cc4)n3)cc2)cc1 ; c1ccc(-c2ccc(-c3nc(-c4ccccc4)nc(-c4ccc(-c5ccc6c(c5)ncc5ccccc56)cc4)n3)cc2)cc1]

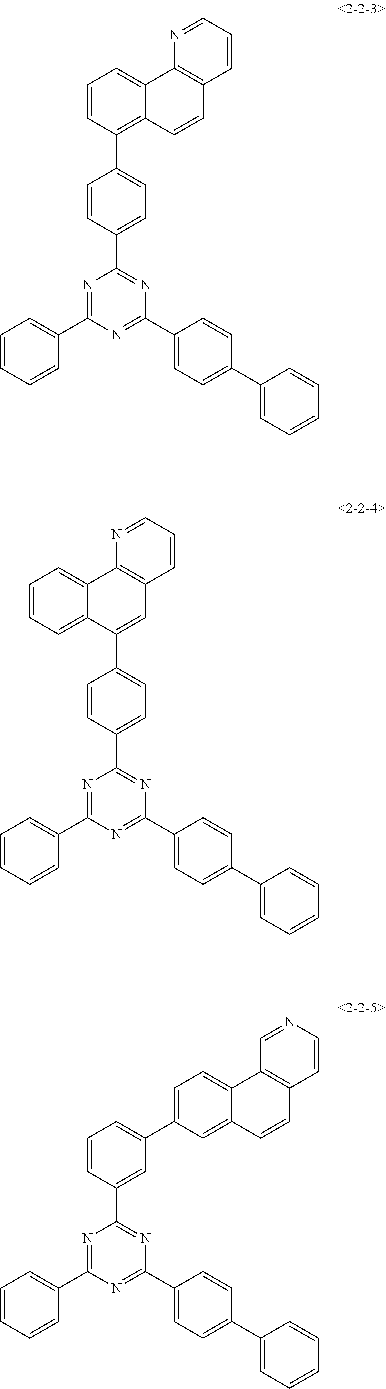 [SMILES: c1ccc(-c2ccc(-c3nc(-c4ccccc4)nc(-c4ccc(-c5cccc6c5ccc5cccnc56)cc4)n3)cc2)cc1 ; c1ccc(-c2ccc(-c3nc(-c4ccccc4)nc(-c4ccc(-c5cc6cccnc6c6ccccc56)cc4)n3)cc2)cc1 ; c1ccc(-c2ccc(-c3nc(-c4ccccc4)nc(-c4cccc(-c5ccc6c(ccc7ccncc76)c5)c4)n3)cc2)cc1]

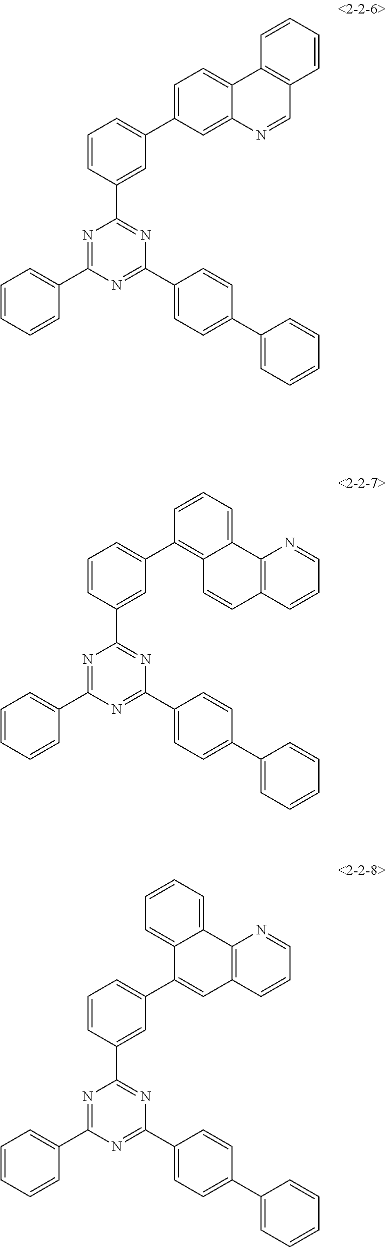 [SMILES: c1ccc(-c2ccc(-c3nc(-c4ccccc4)nc(-c4cccc(-c5ccc6c(c5)ncc5ccccc56)c4)n3)cc2)cc1 ; c1ccc(-c2ccc(-c3nc(-c4ccccc4)nc(-c4cccc(-c5cccc6c5ccc5cccnc56)c4)n3)cc2)cc1 ; c1ccc(-c2ccc(-c3nc(-c4ccccc4)nc(-c4cccc(-c5cc6cccnc6c6ccccc56)c4)n3)cc2)cc1]

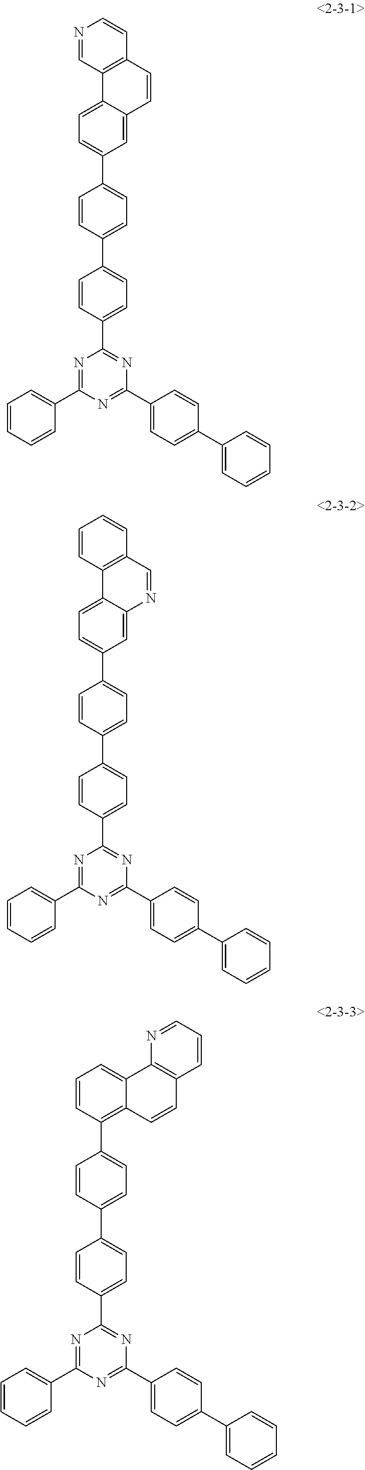 [SMILES: c1ccc(-c2ccc(-c3nc(-c4ccccc4)nc(-c4ccc(-c5ccc(-c6ccc7c(ccc8ccncc87)c6)cc5)cc4)n3)cc2)cc1 ; c1ccc(-c2ccc(-c3nc(-c4ccccc4)nc(-c4ccc(-c5ccc(-c6ccc7c(c6)ncc6ccccc67)cc5)cc4)n3)cc2)cc1 ; c1ccc(-c2ccc(-c3nc(-c4ccccc4)nc(-c4ccc(-c5ccc(-c6cccc7c6ccc6cccnc67)cc5)cc4)n3)cc2)cc1]

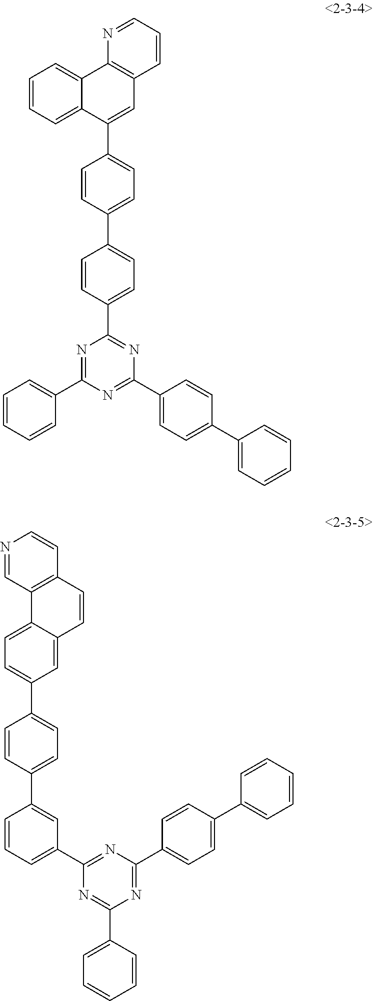 [SMILES: c1ccc(-c2ccc(-c3nc(-c4ccccc4)nc(-c4ccc(-c5ccc(-c6cc7cccnc7c7ccccc67)cc5)cc4)n3)cc2)cc1 ; c1ccc(-c2ccc(-c3nc(-c4ccccc4)nc(-c4cccc(-c5ccc(-c6ccc7c(ccc8ccncc87)c6)cc5)c4)n3)cc2)cc1]

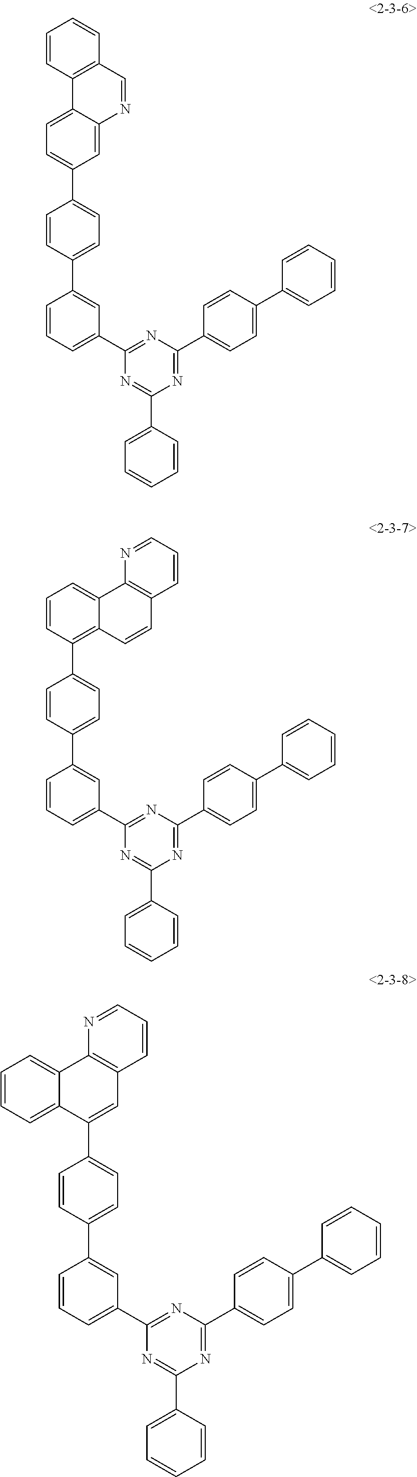 [SMILES: c1ccc(-c2ccc(-c3nc(-c4ccccc4)nc(-c4cccc(-c5ccc(-c6ccc7c(c6)ncc6ccccc67)cc5)c4)n3)cc2)cc1 ; c1ccc(-c2ccc(-c3nc(-c4ccccc4)nc(-c4cccc(-c5ccc(-c6cccc7c6ccc6cccnc67)cc5)c4)n3)cc2)cc1 ; c1ccc(-c2ccc(-c3nc(-c4ccccc4)nc(-c4cccc(-c5ccc(-c6cc7cccnc7c7ccccc67)cc5)c4)n3)cc2)cc1]

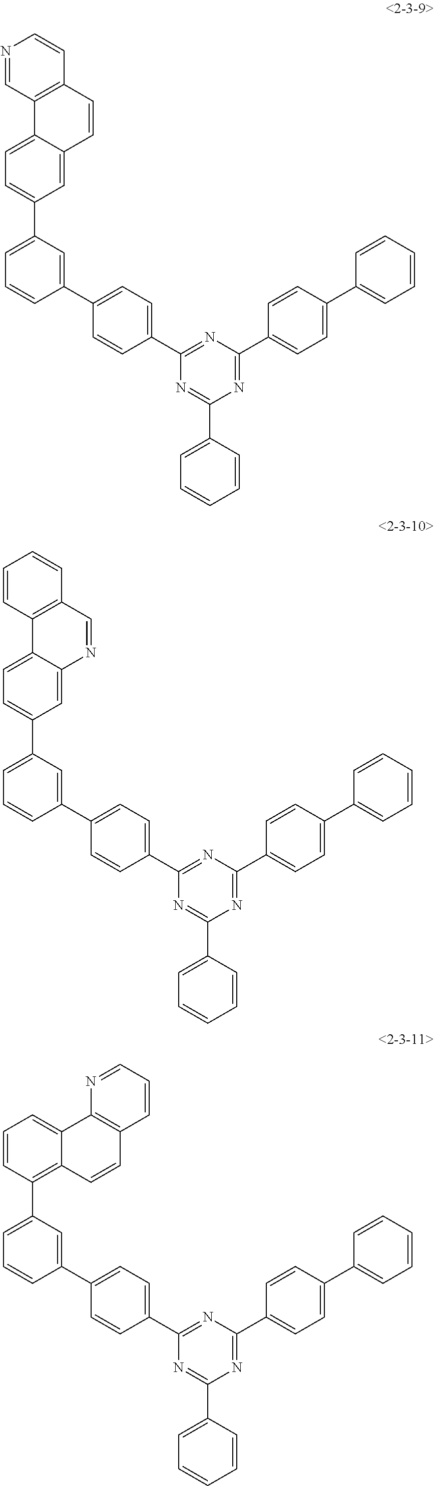 [SMILES: c1ccc(-c2ccc(-c3nc(-c4ccccc4)nc(-c4ccc(-c5cccc(-c6ccc7c(ccc8ccncc87)c6)c5)cc4)n3)cc2)cc1 ; c1ccc(-c2ccc(-c3nc(-c4ccccc4)nc(-c4ccc(-c5cccc(-c6ccc7c(c6)ncc6ccccc67)c5)cc4)n3)cc2)cc1 ; c1ccc(-c2ccc(-c3nc(-c4ccccc4)nc(-c4ccc(-c5cccc(-c6cccc7c6ccc6cccnc67)c5)cc4)n3)cc2)cc1]

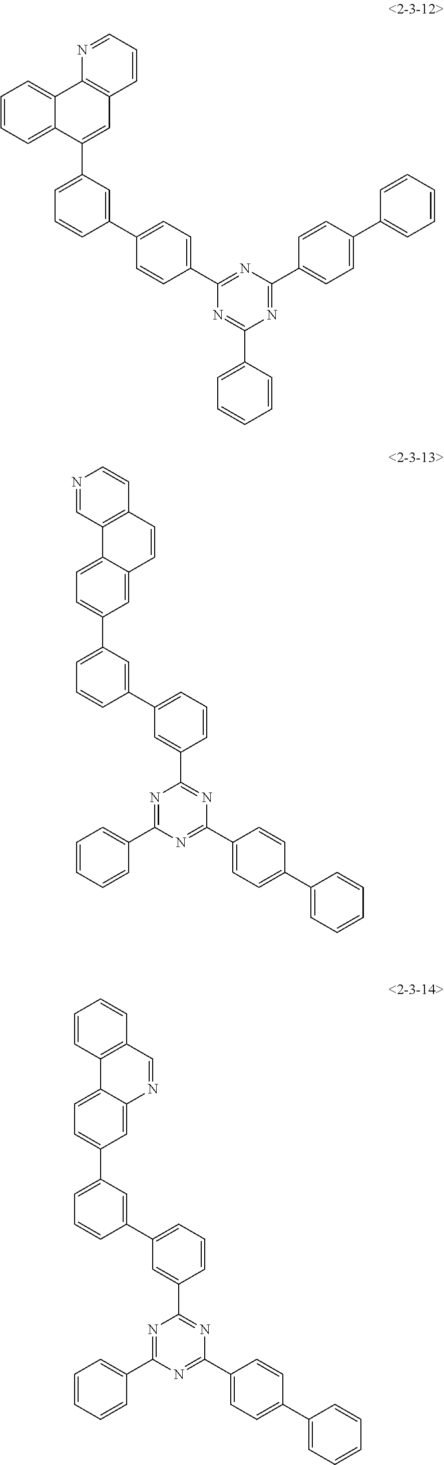 [SMILES: c1ccc(-c2ccc(-c3nc(-c4ccccc4)nc(-c4ccc(-c5cccc(-c6cc7cccnc7c7ccccc67)c5)cc4)n3)cc2)cc1 ; c1ccc(-c2ccc(-c3nc(-c4ccccc4)nc(-c4cccc(-c5cccc(-c6ccc7c(ccc8ccncc87)c6)c5)c4)n3)cc2)cc1 ; c1ccc(-c2ccc(-c3nc(-c4ccccc4)nc(-c4cccc(-c5cccc(-c6ccc7c(c6)ncc6ccccc67)c5)c4)n3)cc2)cc1]

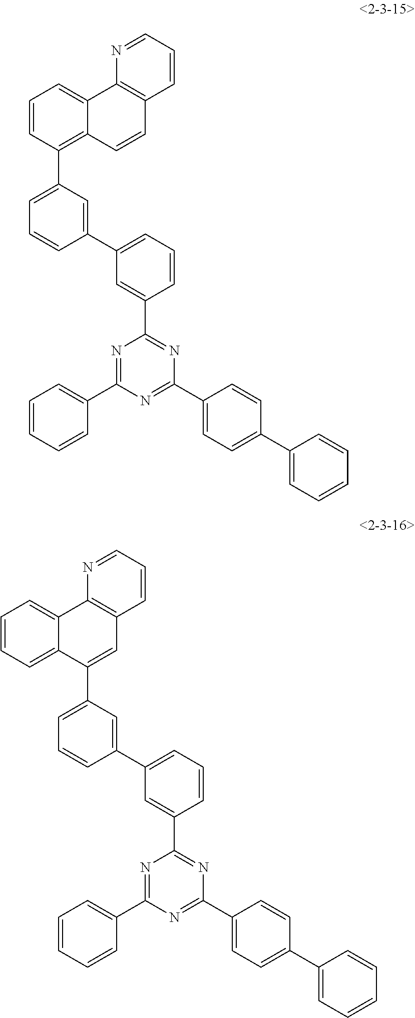 [SMILES: c1ccc(-c2ccc(-c3nc(-c4ccccc4)nc(-c4cccc(-c5cccc(-c6cccc7c6ccc6cccnc67)c5)c4)n3)cc2)cc1 ; c1ccc(-c2ccc(-c3nc(-c4ccccc4)nc(-c4cccc(-c5cccc(-c6cc7cccnc7c7ccccc67)c5)c4)n3)cc2)cc1]

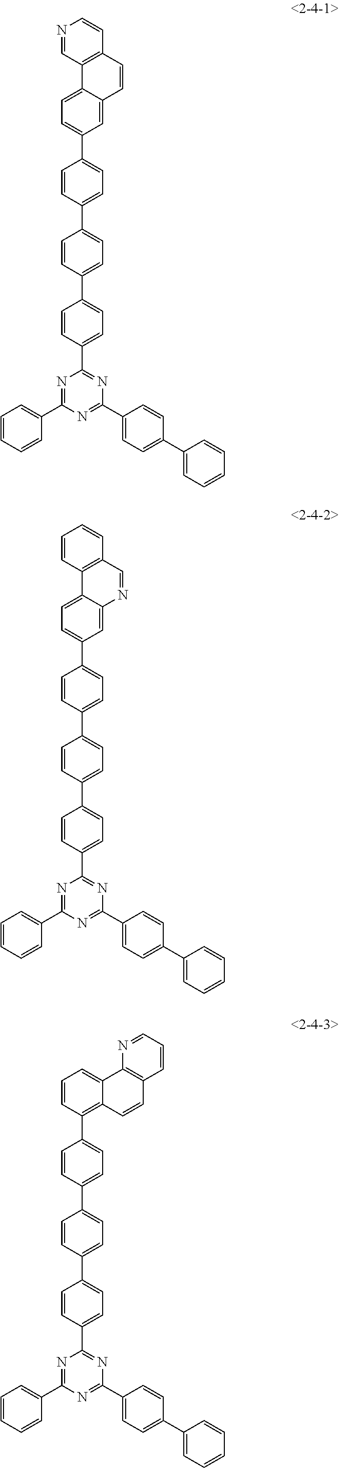 [SMILES: c1ccc(-c2ccc(-c3nc(-c4ccccc4)nc(-c4ccc(-c5ccc(-c6ccc(-c7ccc8c(ccc9ccncc98)c7)cc6)cc5)cc4)n3)cc2)cc1 ; c1ccc(-c2ccc(-c3nc(-c4ccccc4)nc(-c4ccc(-c5ccc(-c6ccc(-c7ccc8c(c7)ncc7ccccc78)cc6)cc5)cc4)n3)cc2)cc1 ; c1ccc(-c2ccc(-c3nc(-c4ccccc4)nc(-c4ccc(-c5ccc(-c6ccc(-c7cccc8c7ccc7cccnc78)cc6)cc5)cc4)n3)cc2)cc1]

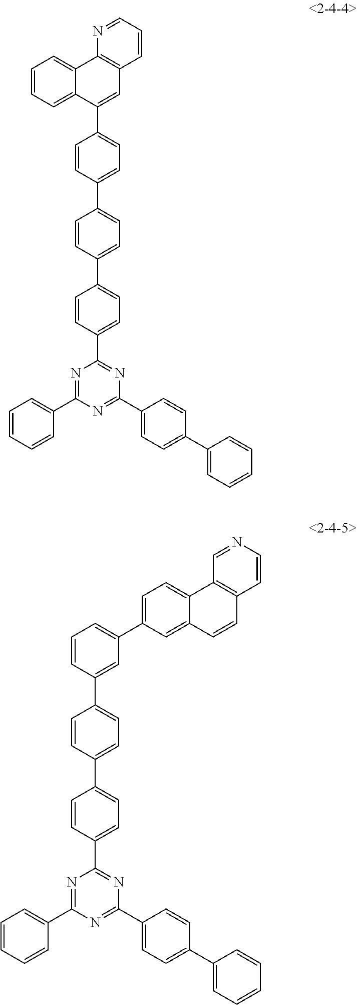 [SMILES: c1ccc(-c2ccc(-c3nc(-c4ccccc4)nc(-c4ccc(-c5ccc(-c6ccc(-c7cc8cccnc8c8ccccc78)cc6)cc5)cc4)n3)cc2)cc1 ; c1ccc(-c2ccc(-c3nc(-c4ccccc4)nc(-c4ccc(-c5ccc(-c6cccc(-c7ccc8c(ccc9ccncc98)c7)c6)cc5)cc4)n3)cc2)cc1]

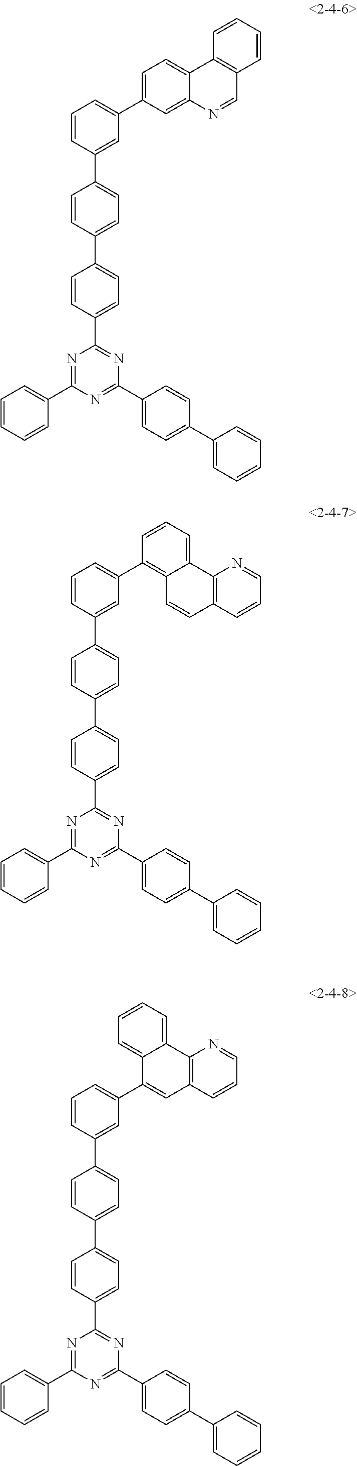 [SMILES: c1ccc(-c2ccc(-c3nc(-c4ccccc4)nc(-c4ccc(-c5ccc(-c6cccc(-c7ccc8c(c7)ncc7ccccc78)c6)cc5)cc4)n3)cc2)cc1 ; c1ccc(-c2ccc(-c3nc(-c4ccccc4)nc(-c4ccc(-c5ccc(-c6cccc(-c7cccc8c7ccc7cccnc78)c6)cc5)cc4)n3)cc2)cc1 ; c1ccc(-c2ccc(-c3nc(-c4ccccc4)nc(-c4ccc(-c5ccc(-c6cccc(-c7cc8cccnc8c8ccccc78)c6)cc5)cc4)n3)cc2)cc1]

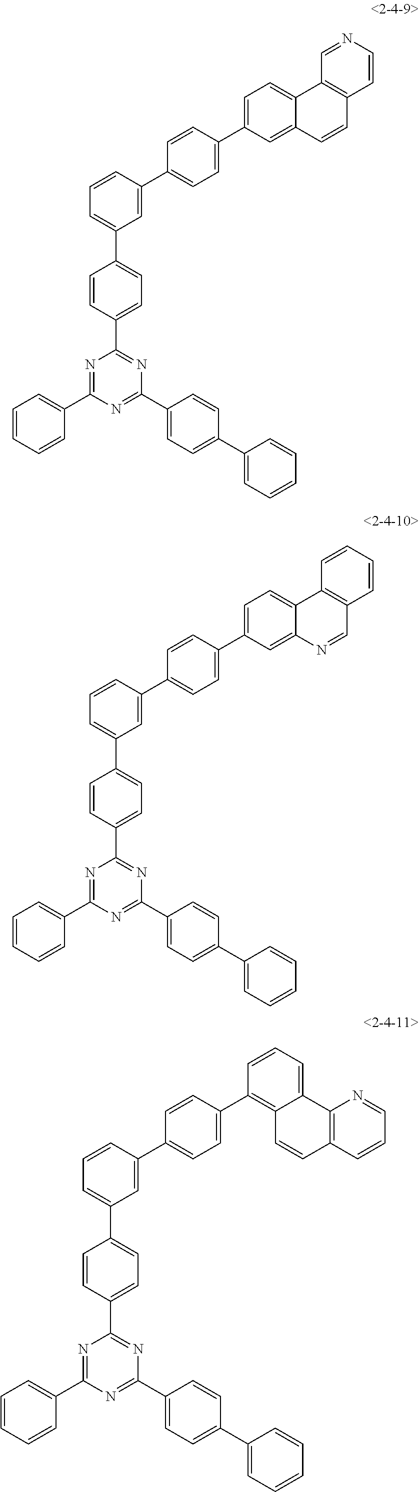 [SMILES: c1ccc(-c2ccc(-c3nc(-c4ccccc4)nc(-c4ccc(-c5cccc(-c6ccc(-c7ccc8c(ccc9ccncc98)c7)cc6)c5)cc4)n3)cc2)cc1 ; c1ccc(-c2ccc(-c3nc(-c4ccccc4)nc(-c4ccc(-c5cccc(-c6ccc(-c7ccc8c(c7)ncc7ccccc78)cc6)c5)cc4)n3)cc2)cc1 ; c1ccc(-c2ccc(-c3nc(-c4ccccc4)nc(-c4ccc(-c5cccc(-c6ccc(-c7cccc8c7ccc7cccnc78)cc6)c5)cc4)n3)cc2)cc1]

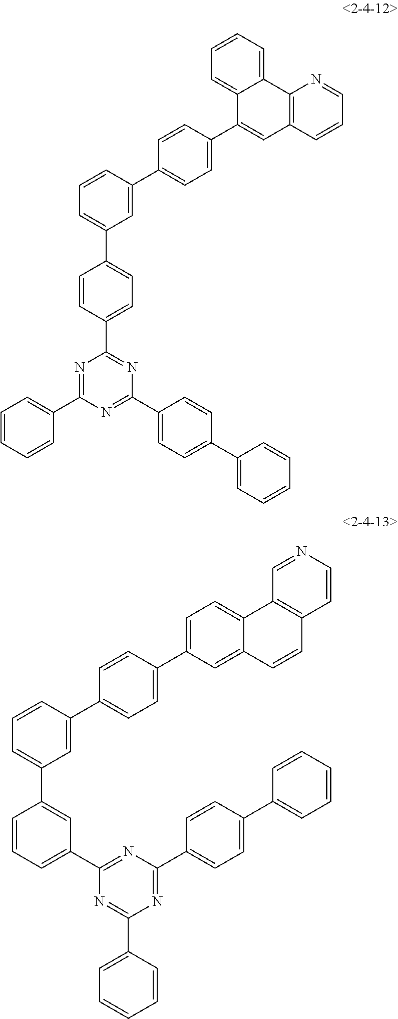 [SMILES: c1ccc(-c2ccc(-c3nc(-c4ccccc4)nc(-c4ccc(-c5cccc(-c6ccc(-c7cc8cccnc8c8ccccc78)cc6)c5)cc4)n3)cc2)cc1 ; c1ccc(-c2ccc(-c3nc(-c4ccccc4)nc(-c4cccc(-c5cccc(-c6ccc(-c7ccc8c(ccc9ccncc98)c7)cc6)c5)c4)n3)cc2)cc1]

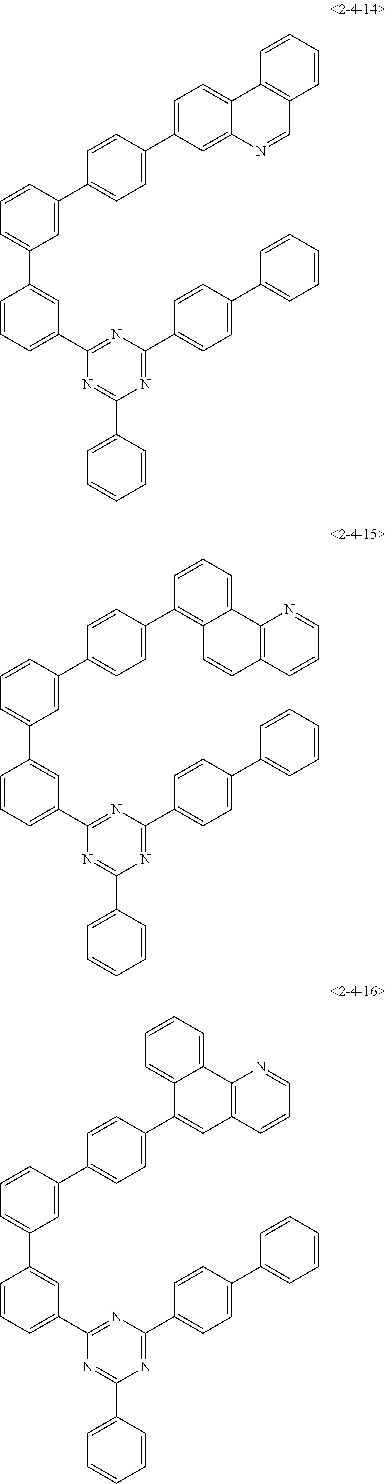 [SMILES: c1ccc(-c2ccc(-c3nc(-c4ccccc4)nc(-c4cccc(-c5cccc(-c6ccc(-c7ccc8c(c7)ncc7ccccc78)cc6)c5)c4)n3)cc2)cc1 ; c1ccc(-c2ccc(-c3nc(-c4ccccc4)nc(-c4cccc(-c5cccc(-c6ccc(-c7cccc8c7ccc7cccnc78)cc6)c5)c4)n3)cc2)cc1 ; c1ccc(-c2ccc(-c3nc(-c4ccccc4)nc(-c4cccc(-c5cccc(-c6ccc(-c7cc8cccnc8c8ccccc78)cc6)c5)c4)n3)cc2)cc1]

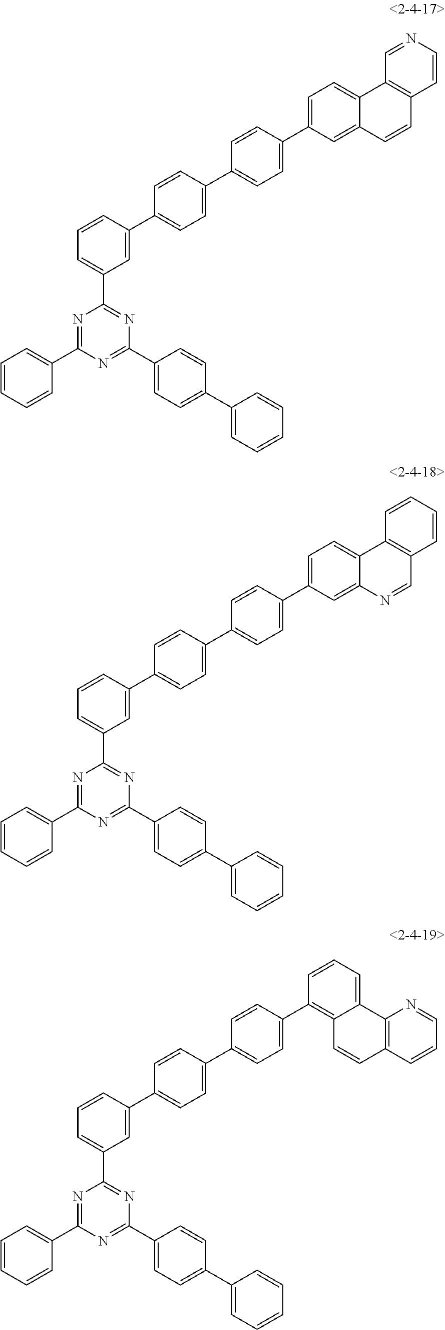 [SMILES: c1ccc(-c2ccc(-c3nc(-c4ccccc4)nc(-c4cccc(-c5ccc(-c6ccc(-c7ccc8c(ccc9ccncc98)c7)cc6)cc5)c4)n3)cc2)cc1 ; c1ccc(-c2ccc(-c3nc(-c4ccccc4)nc(-c4cccc(-c5ccc(-c6ccc(-c7ccc8c(c7)ncc7ccccc78)cc6)cc5)c4)n3)cc2)cc1 ; c1ccc(-c2ccc(-c3nc(-c4ccccc4)nc(-c4cccc(-c5ccc(-c6ccc(-c7cccc8c7ccc7cccnc78)cc6)cc5)c4)n3)cc2)cc1]

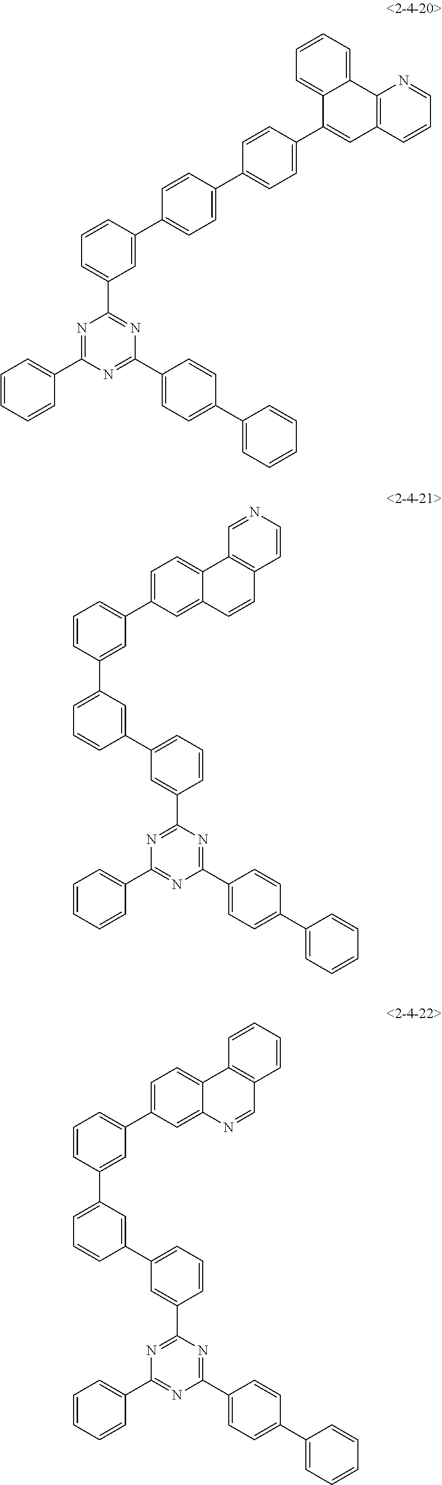 [SMILES: c1ccc(-c2ccc(-c3nc(-c4ccccc4)nc(-c4cccc(-c5ccc(-c6ccc(-c7cc8cccnc8c8ccccc78)cc6)cc5)c4)n3)cc2)cc1 ; c1ccc(-c2ccc(-c3nc(-c4ccccc4)nc(-c4cccc(-c5cccc(-c6cccc(-c7ccc8c(ccc9ccncc98)c7)c6)c5)c4)n3)cc2)cc1 ; c1ccc(-c2ccc(-c3nc(-c4ccccc4)nc(-c4cccc(-c5cccc(-c6cccc(-c7ccc8c(c7)ncc7ccccc78)c6)c5)c4)n3)cc2)cc1]

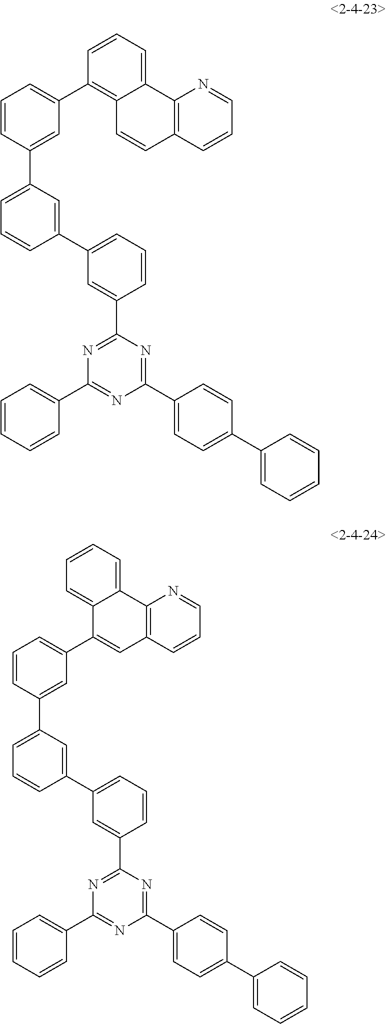 [SMILES: c1ccc(-c2ccc(-c3nc(-c4ccccc4)nc(-c4cccc(-c5cccc(-c6cccc(-c7cccc8c7ccc7cccnc78)c6)c5)c4)n3)cc2)cc1 ; c1ccc(-c2ccc(-c3nc(-c4ccccc4)nc(-c4cccc(-c5cccc(-c6cccc(-c7cc8cccnc8c8ccccc78)c6)c5)c4)n3)cc2)cc1]

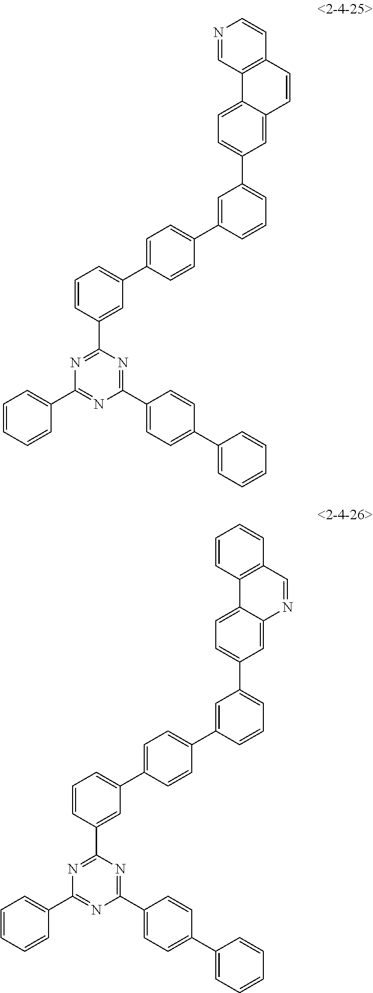

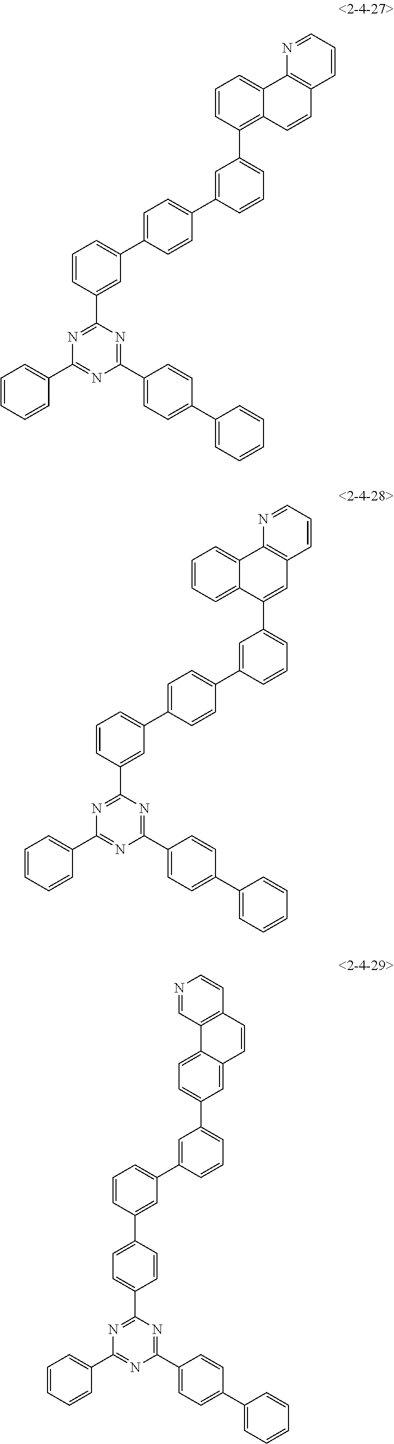 [SMILES: c1ccc(-c2ccc(-c3nc(-c4ccccc4)nc(-c4cccc(-c5ccc(-c6cccc(-c7cccc8c7ccc7cccnc78)c6)cc5)c4)n3)cc2)cc1 ; c1ccc(-c2ccc(-c3nc(-c4ccccc4)nc(-c4cccc(-c5ccc(-c6cccc(-c7cc8cccnc8c8ccccc78)c6)cc5)c4)n3)cc2)cc1 ; c1ccc(-c2ccc(-c3nc(-c4ccccc4)nc(-c4ccc(-c5cccc(-c6cccc(-c7ccc8c(ccc9ccncc98)c7)c6)c5)cc4)n3)cc2)cc1]

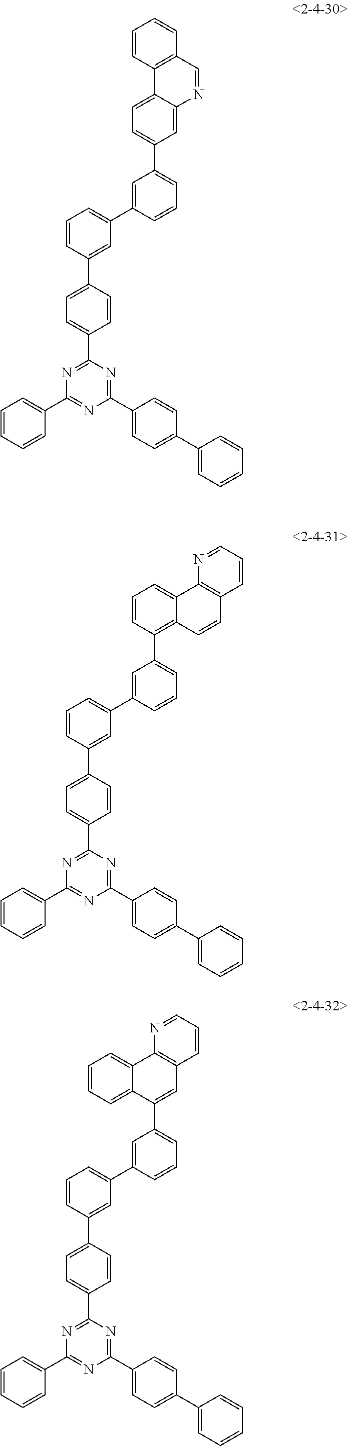 [SMILES: c1ccc(-c2ccc(-c3nc(-c4ccccc4)nc(-c4ccc(-c5cccc(-c6cccc(-c7ccc8c(c7)ncc7ccccc78)c6)c5)cc4)n3)cc2)cc1 ; c1ccc(-c2ccc(-c3nc(-c4ccccc4)nc(-c4ccc(-c5cccc(-c6cccc(-c7cccc8c7ccc7cccnc78)c6)c5)cc4)n3)cc2)cc1 ; c1ccc(-c2ccc(-c3nc(-c4ccccc4)nc(-c4ccc(-c5cccc(-c6cccc(-c7cc8cccnc8c8ccccc78)c6)c5)cc4)n3)cc2)cc1]

Furthermore, the compound of chemical formula 1 may be any one of the following compounds.

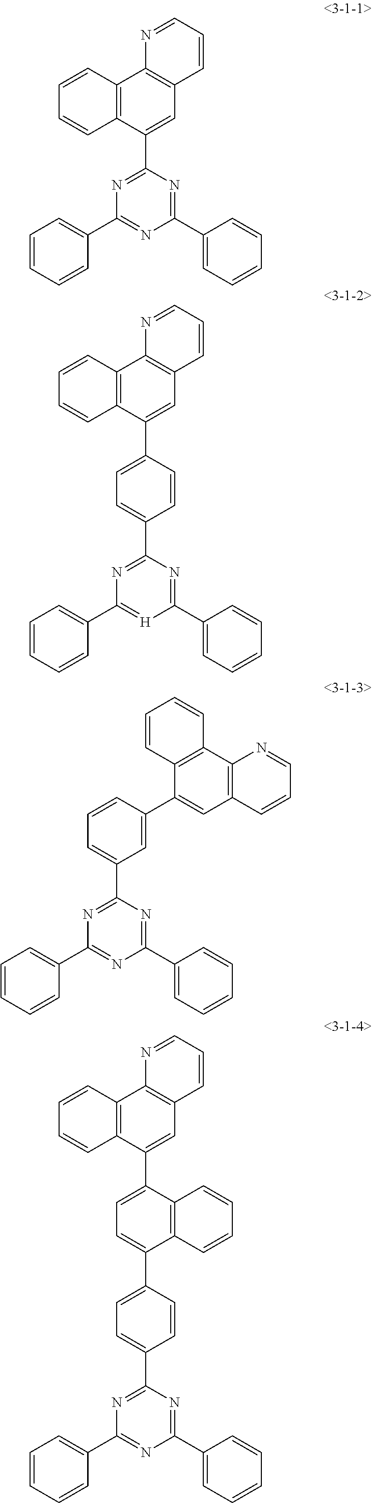

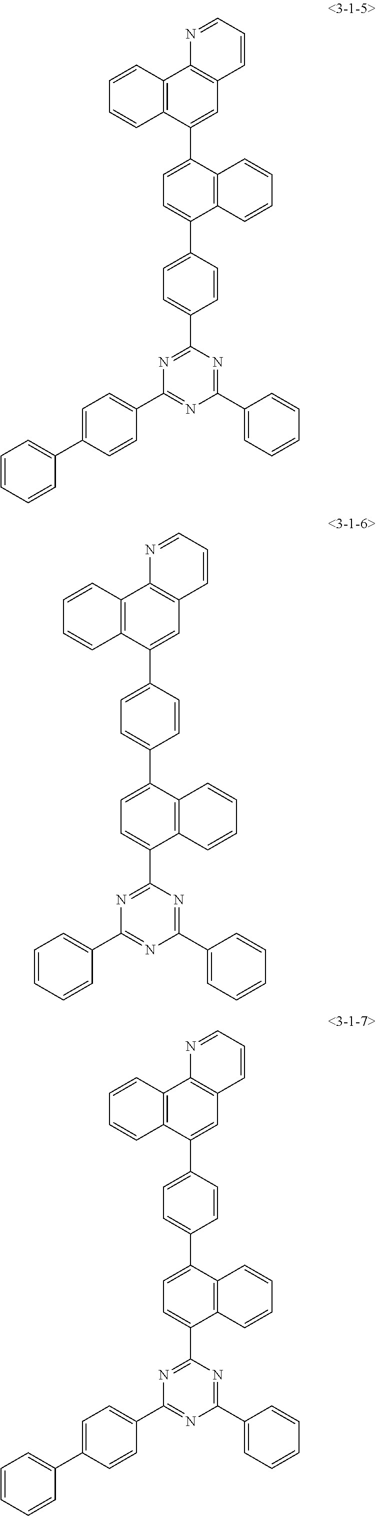

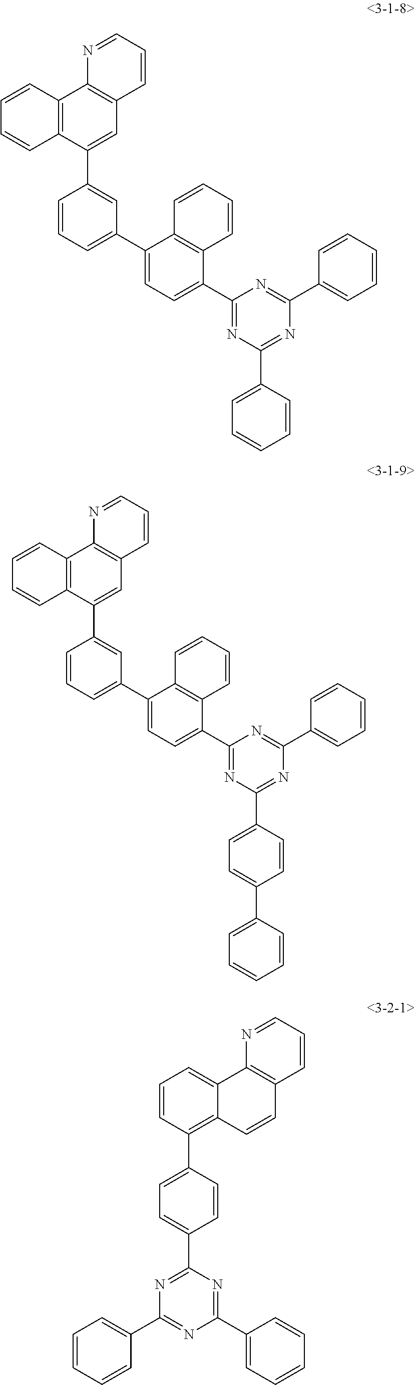

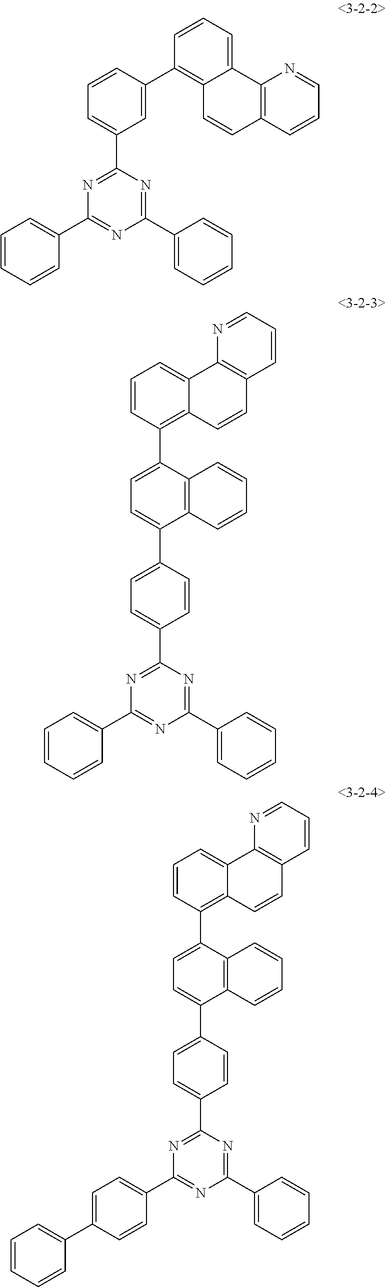

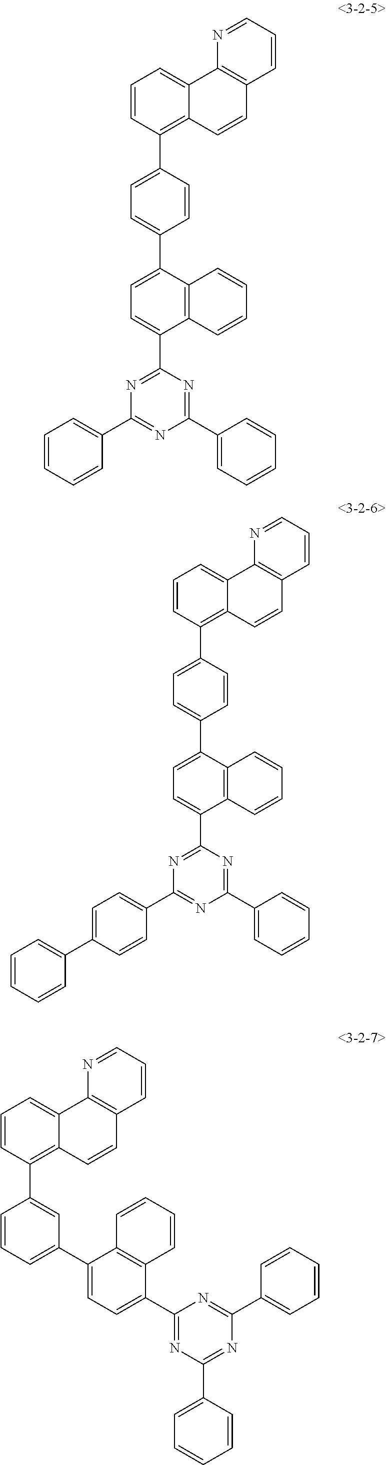 [SMILES: c1ccc(-c2nc(-c3ccccc3)nc(-c3ccc(-c4ccc(-c5cccc6c5ccc5cccnc56)cc4)c4ccccc34)n2)cc1 ; c1ccc(-c2ccc(-c3nc(-c4ccccc4)nc(-c4ccc(-c5ccc(-c6cccc7c6ccc6cccnc67)cc5)c5ccccc45)n3)cc2)cc1 ; c1ccc(-c2nc(-c3ccccc3)nc(-c3ccc(-c4cccc(-c5cccc6c5ccc5cccnc56)c4)c4ccccc34)n2)cc1]

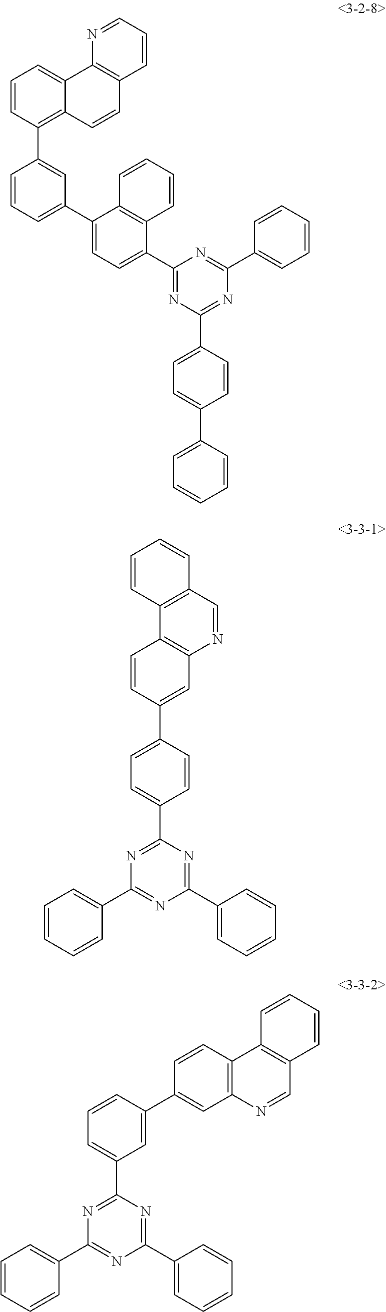 [SMILES: c1ccc(-c2ccc(-c3nc(-c4ccccc4)nc(-c4ccc(-c5cccc(-c6cccc7c6ccc6cccnc67)c5)c5ccccc45)n3)cc2)cc1 ; c1ccc(-c2nc(-c3ccccc3)nc(-c3ccc(-c4ccc5c(c4)ncc4ccccc45)cc3)n2)cc1 ; c1ccc(-c2nc(-c3ccccc3)nc(-c3cccc(-c4ccc5c(c4)ncc4ccccc45)c3)n2)cc1]

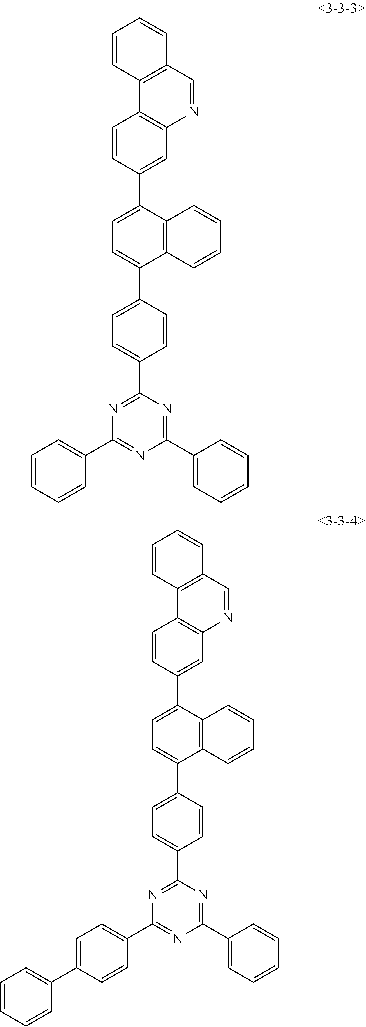 [SMILES: c1ccc(-c2nc(-c3ccccc3)nc(-c3ccc(-c4ccc(-c5ccc6c(c5)ncc5ccccc56)c5ccccc45)cc3)n2)cc1 ; c1ccc(-c2ccc(-c3nc(-c4ccccc4)nc(-c4ccc(-c5ccc(-c6ccc7c(c6)ncc6ccccc67)c6ccccc56)cc4)n3)cc2)cc1]

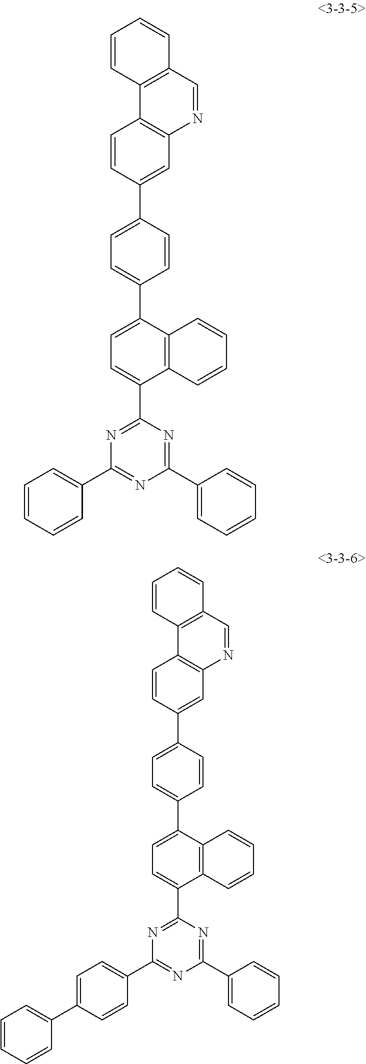 [SMILES: c1ccc(-c2nc(-c3ccccc3)nc(-c3ccc(-c4ccc(-c5ccc6c(c5)ncc5ccccc56)cc4)c4ccccc34)n2)cc1 ; c1ccc(-c2ccc(-c3nc(-c4ccccc4)nc(-c4ccc(-c5ccc(-c6ccc7c(c6)ncc6ccccc67)cc5)c5ccccc45)n3)cc2)cc1]

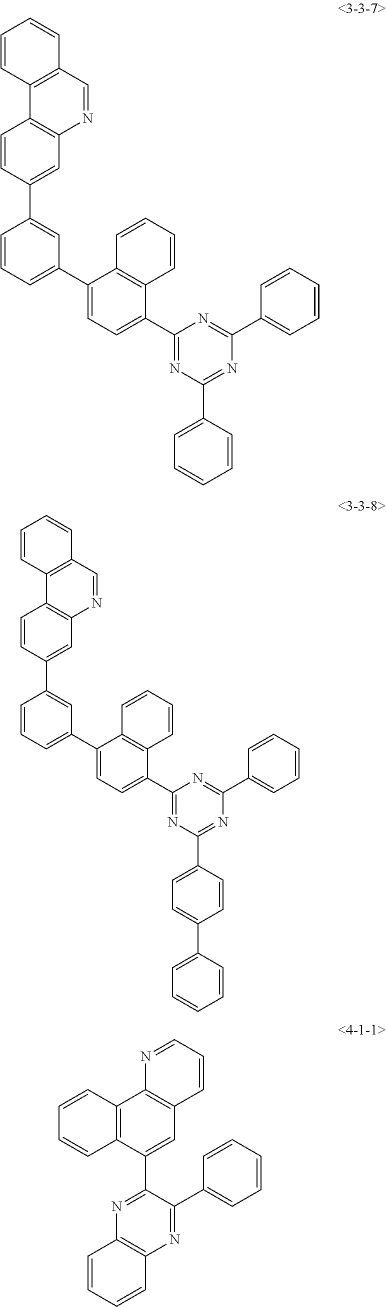

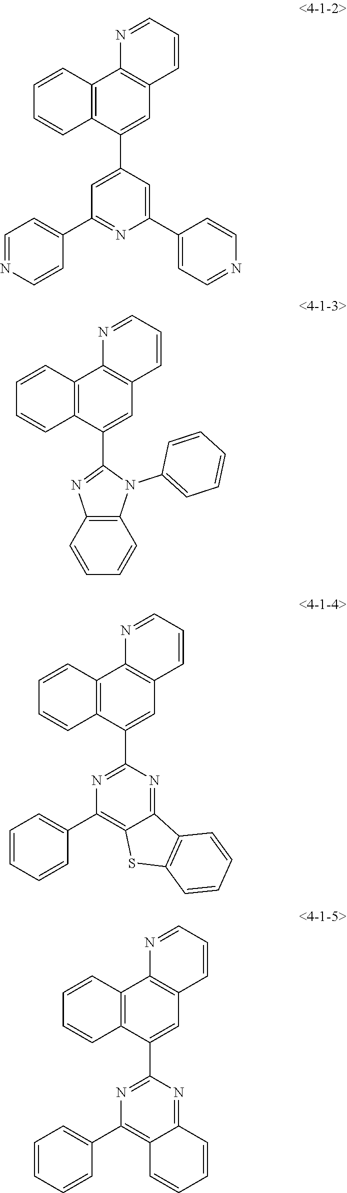

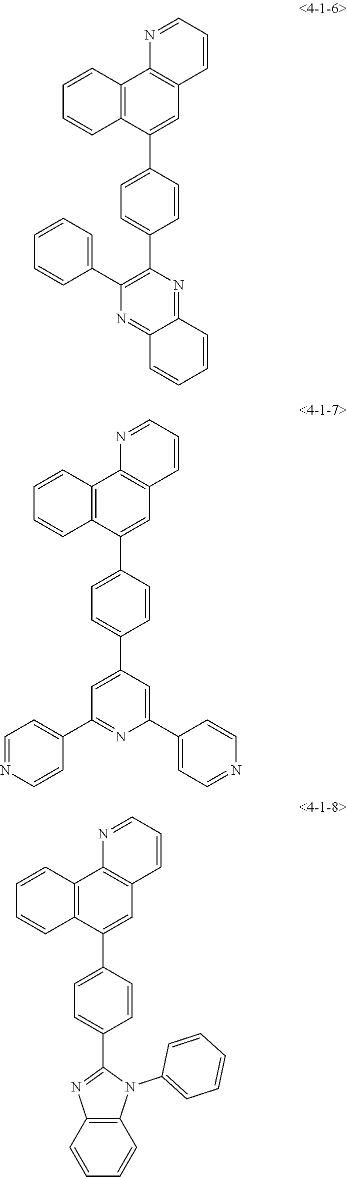

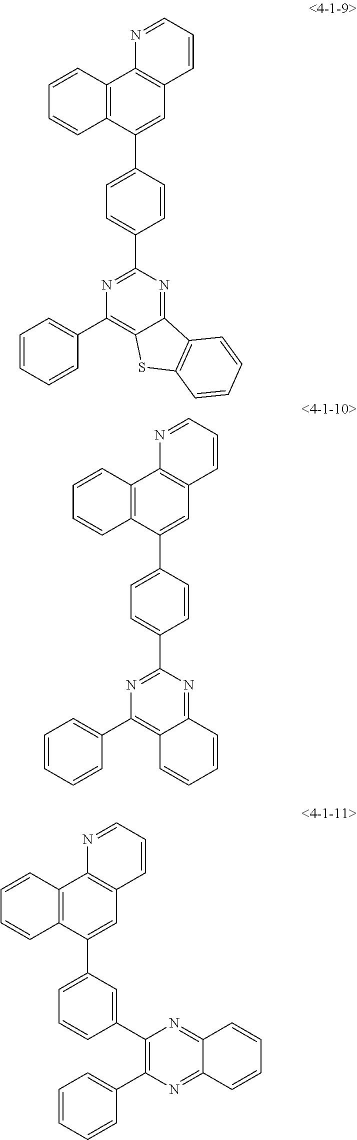

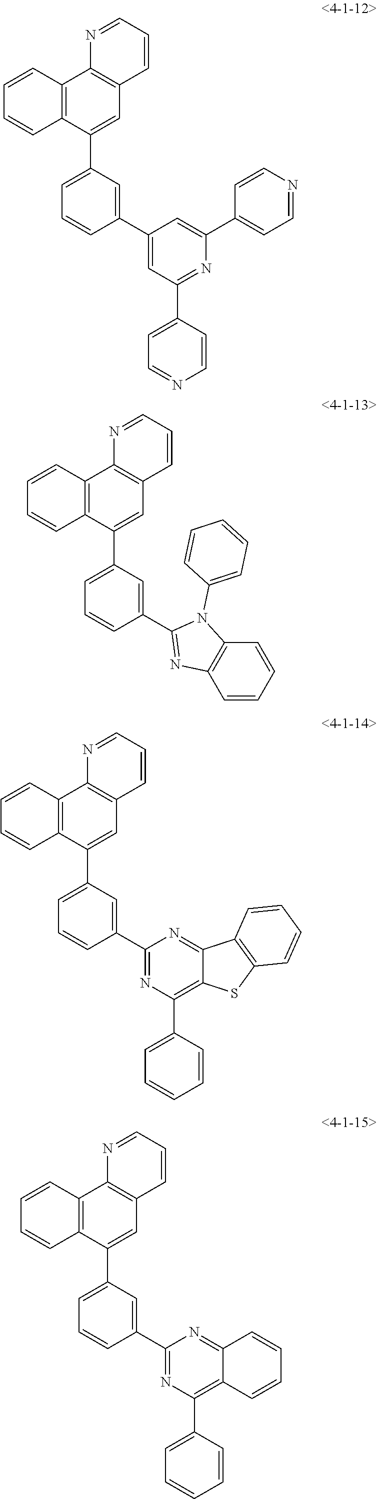

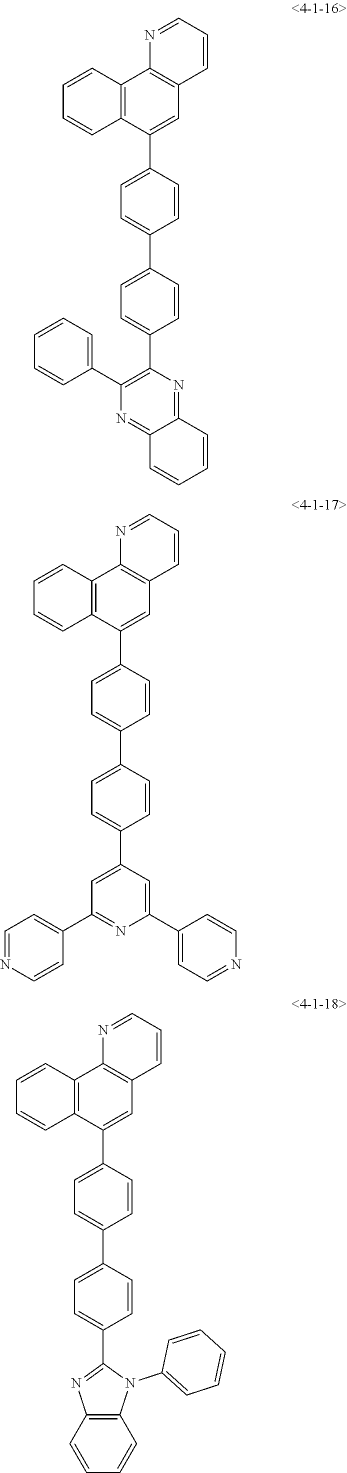

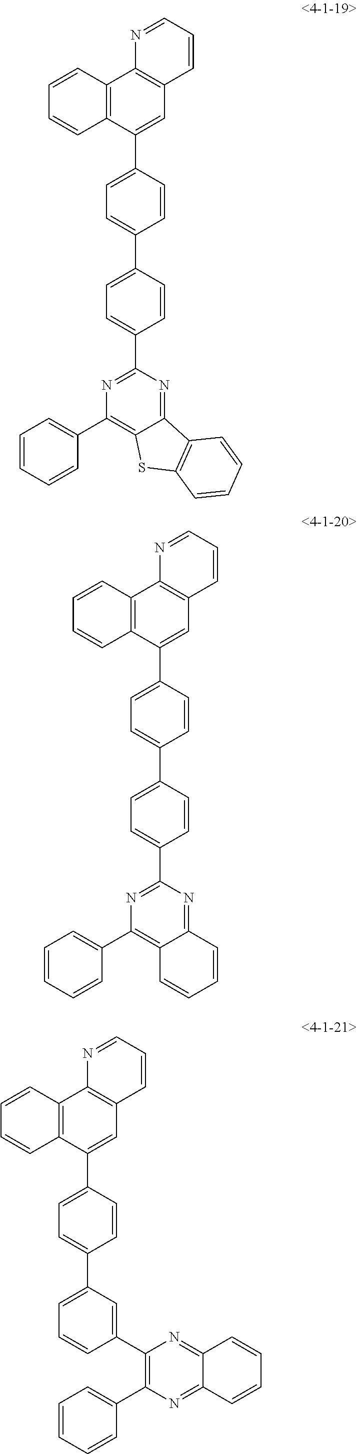

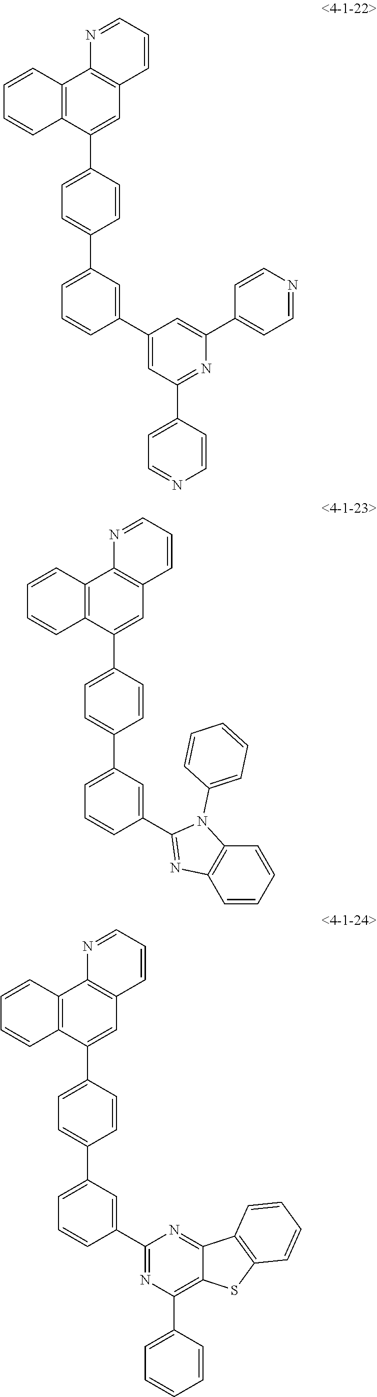 [SMILES: c1cc(-c2ccc(-c3cc4cccnc4c4ccccc34)cc2)cc(-c2cc(-c3ccncc3)nc(-c3ccncc3)c2)c1 ; c1ccc(-n2c(-c3cccc(-c4ccc(-c5cc6cccnc6c6ccccc56)cc4)c3)nc3ccccc32)cc1 ; c1ccc(-c2nc(-c3cccc(-c4ccc(-c5cc6cccnc6c6ccccc56)cc4)c3)nc3c2sc2ccccc23)cc1]

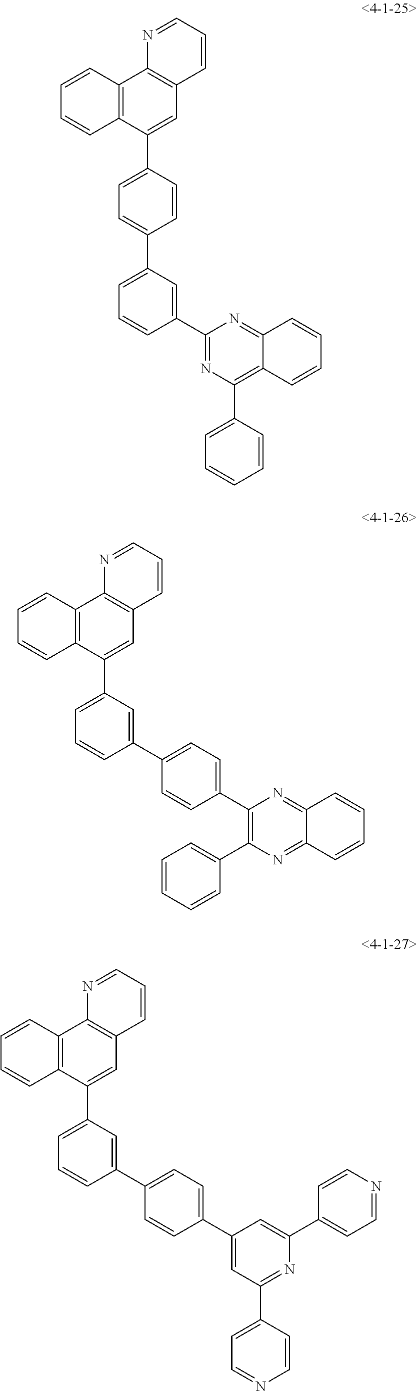 [SMILES: c1ccc(-c2nc(-c3cccc(-c4ccc(-c5cc6cccnc6c6ccccc56)cc4)c3)nc3ccccc23)cc1 ; c1ccc(-c2nc3ccccc3nc2-c2ccc(-c3cccc(-c4cc5cccnc5c5ccccc45)c3)cc2)cc1 ; c1cc(-c2ccc(-c3cc(-c4ccncc4)nc(-c4ccncc4)c3)cc2)cc(-c2cc3cccnc3c3ccccc23)c1]

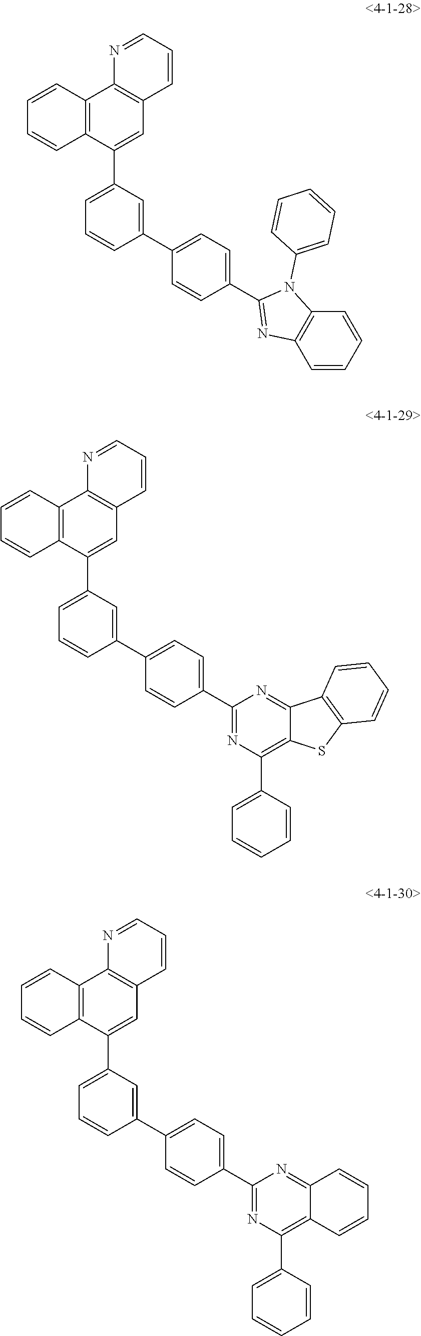 [SMILES: c1ccc(-n2c(-c3ccc(-c4cccc(-c5cc6cccnc6c6ccccc56)c4)cc3)nc3ccccc32)cc1 ; c1ccc(-c2nc(-c3ccc(-c4cccc(-c5cc6cccnc6c6ccccc56)c4)cc3)nc3c2sc2ccccc23)cc1 ; c1ccc(-c2nc(-c3ccc(-c4cccc(-c5cc6cccnc6c6ccccc56)c4)cc3)nc3ccccc23)cc1]

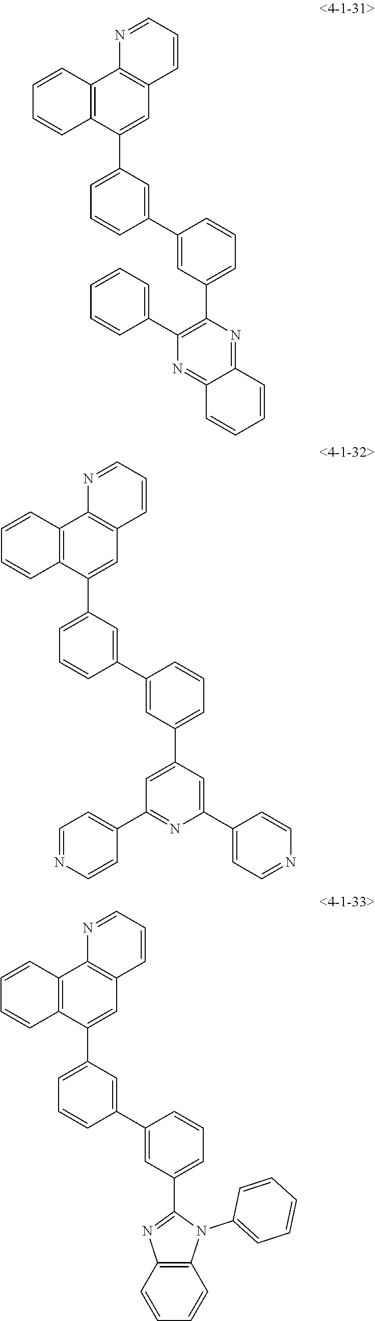

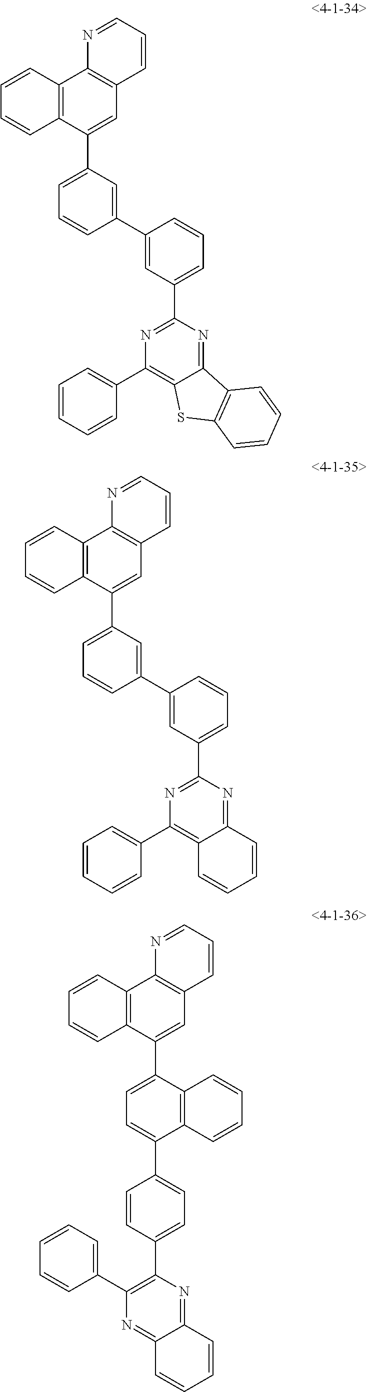 [SMILES: c1ccc(-c2nc(-c3cccc(-c4cccc(-c5cc6cccnc6c6ccccc56)c4)c3)nc3c2sc2ccccc23)cc1 ; c1ccc(-c2nc(-c3cccc(-c4cccc(-c5cc6cccnc6c6ccccc56)c4)c3)nc3ccccc23)cc1 ; c1ccc(-c2nc3ccccc3nc2-c2ccc(-c3ccc(-c4cc5cccnc5c5ccccc45)c4ccccc34)cc2)cc1]

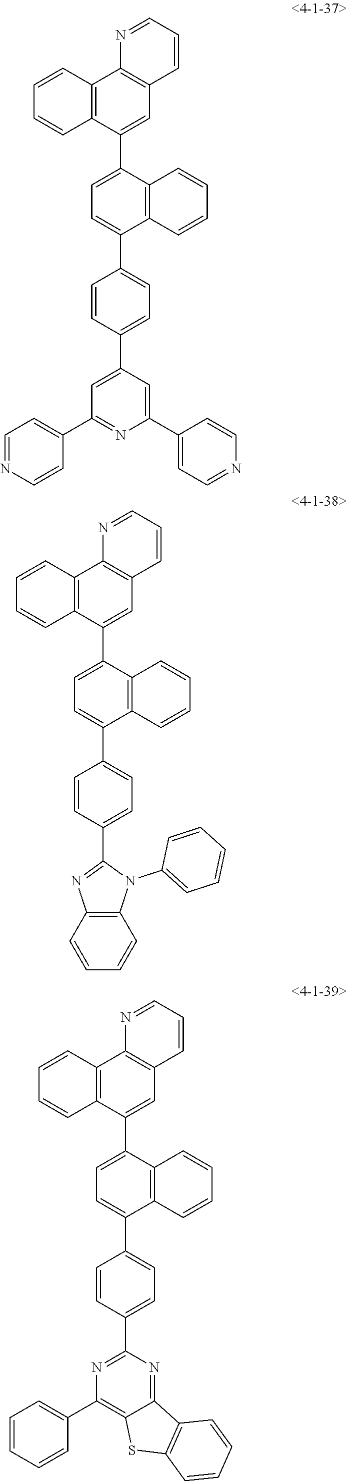

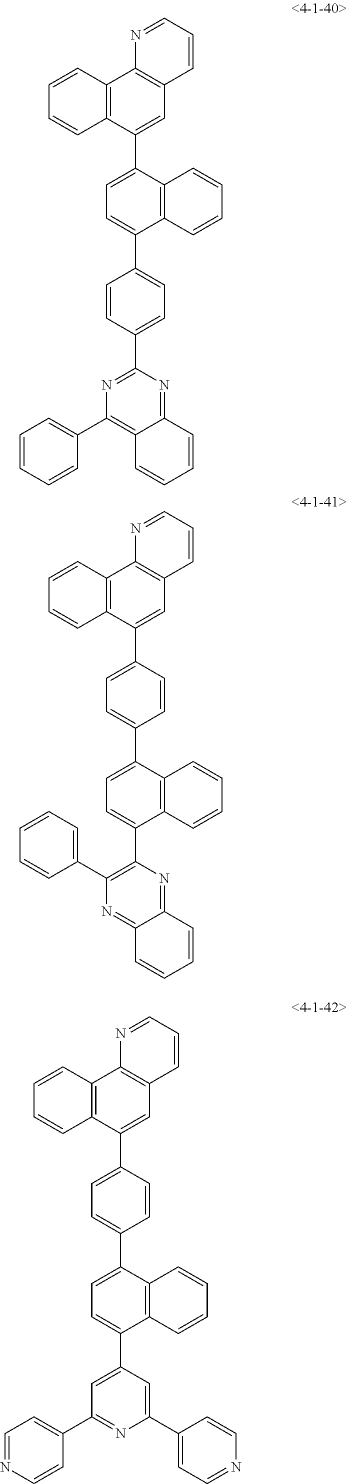

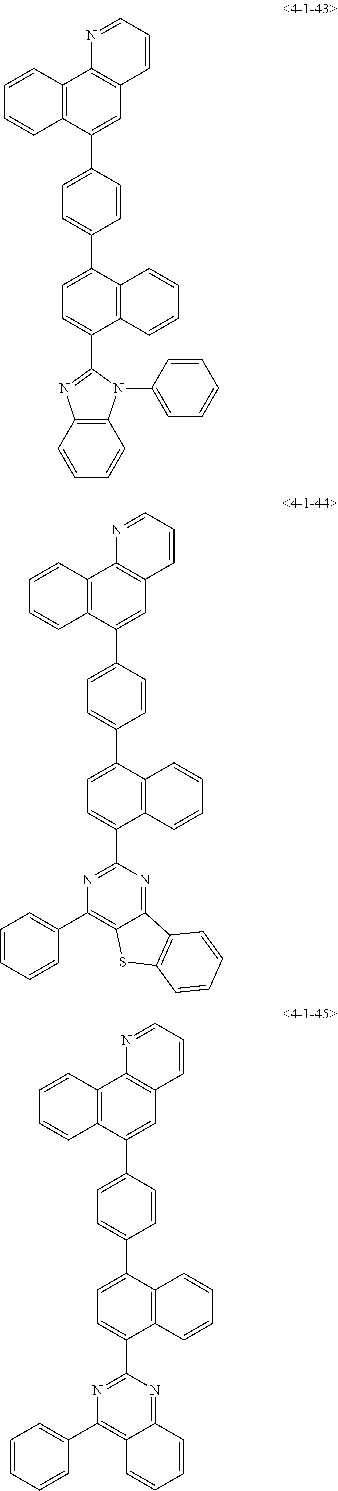

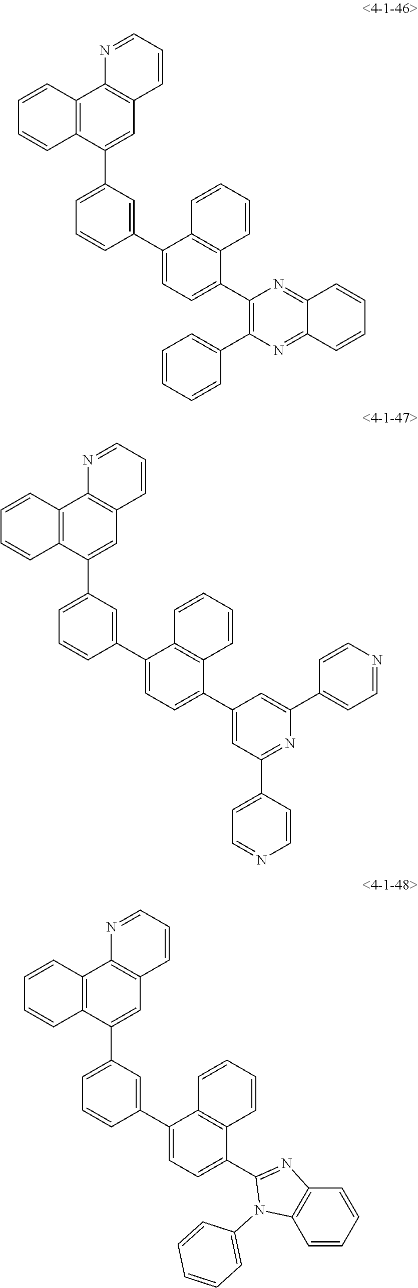 [SMILES: c1ccc(-c2nc3ccccc3nc2-c2ccc(-c3cccc(-c4cc5cccnc5c5ccccc45)c3)c3ccccc23)cc1 ; c1cc(-c2ccc(-c3cc(-c4ccncc4)nc(-c4ccncc4)c3)c3ccccc23)cc(-c2cc3cccnc3c3ccccc23)c1 ; c1ccc(-n2c(-c3ccc(-c4cccc(-c5cc6cccnc6c6ccccc56)c4)c4ccccc34)nc3ccccc32)cc1]

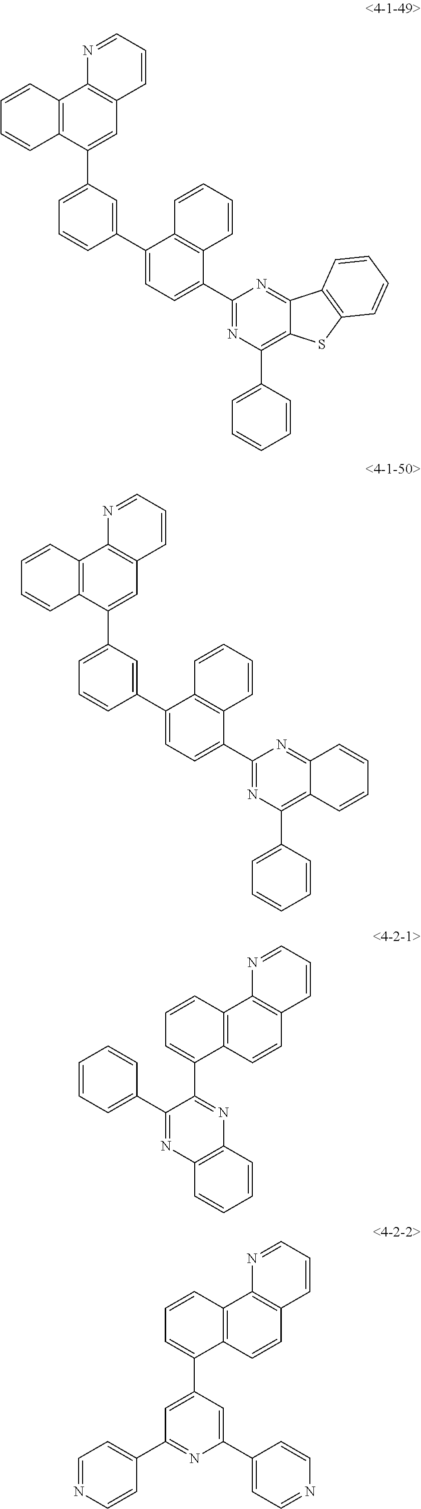

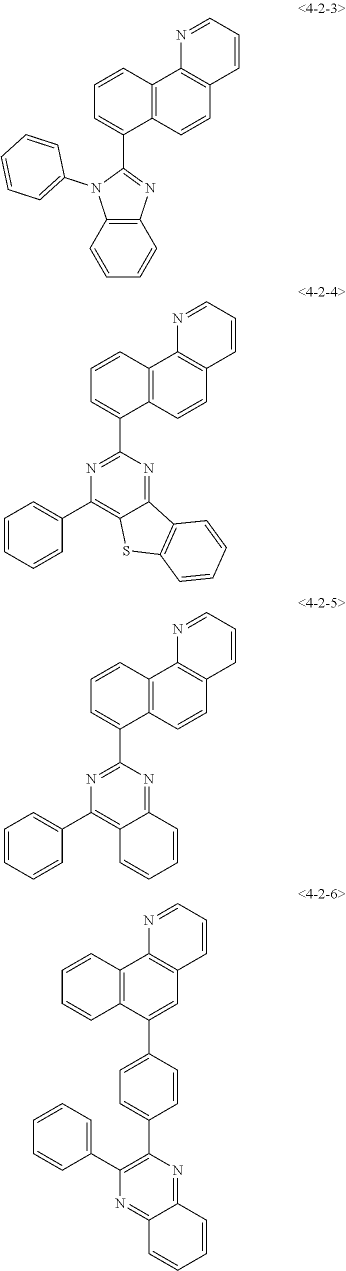

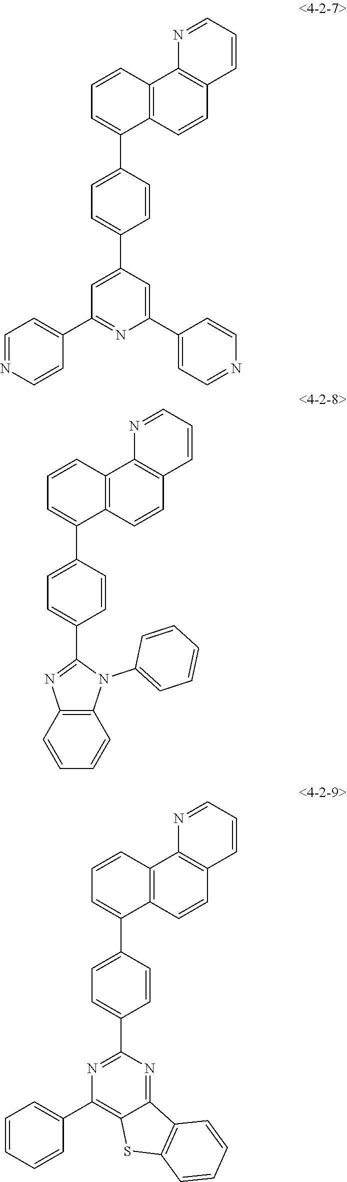

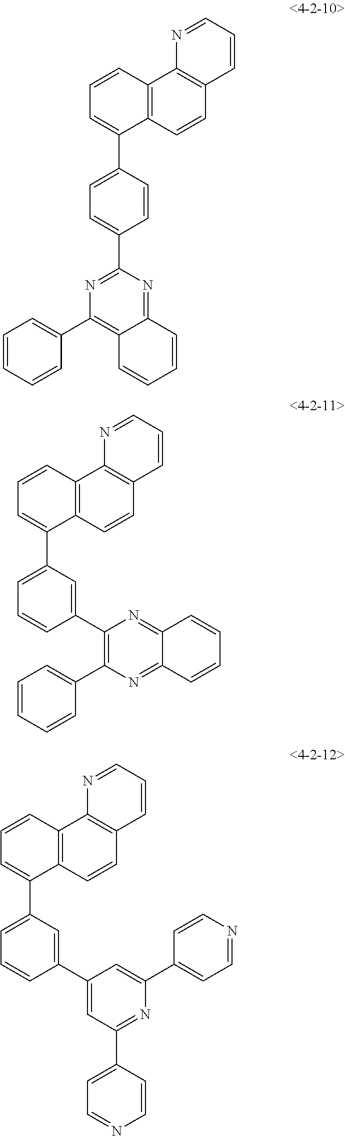

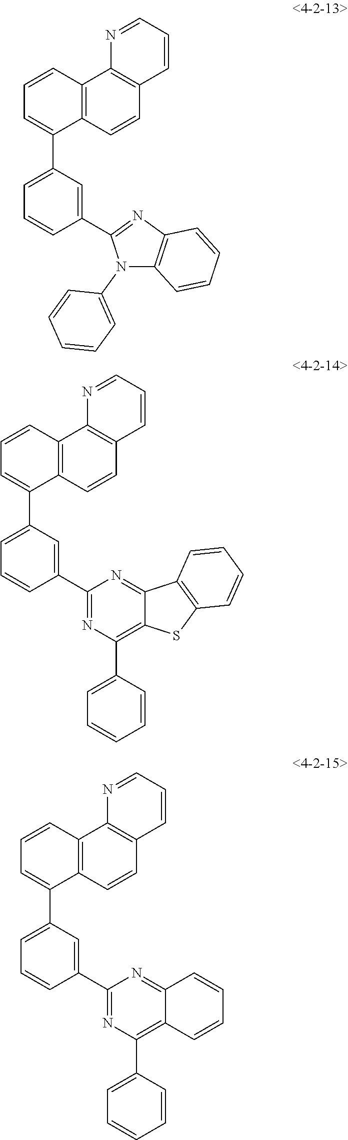

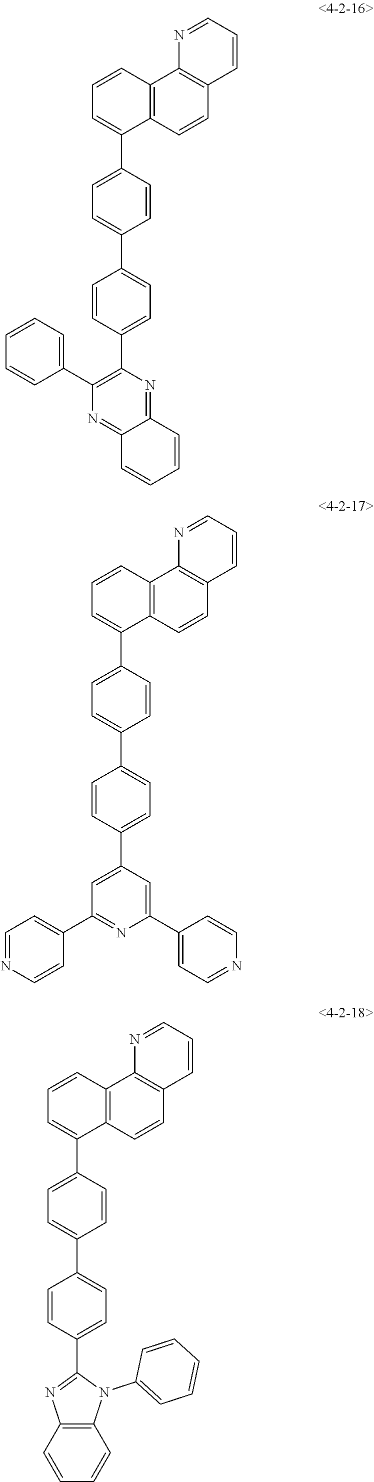 [SMILES: c1ccc(-c2nc3ccccc3nc2-c2ccc(-c3ccc(-c4cccc5c4ccc4cccnc45)cc3)cc2)cc1 ; c1cnc2c(c1)ccc1c(-c3ccc(-c4ccc(-c5cc(-c6ccncc6)nc(-c6ccncc6)c5)cc4)cc3)cccc12 ; c1ccc(-n2c(-c3ccc(-c4ccc(-c5cccc6c5ccc5cccnc56)cc4)cc3)nc3ccccc32)cc1]

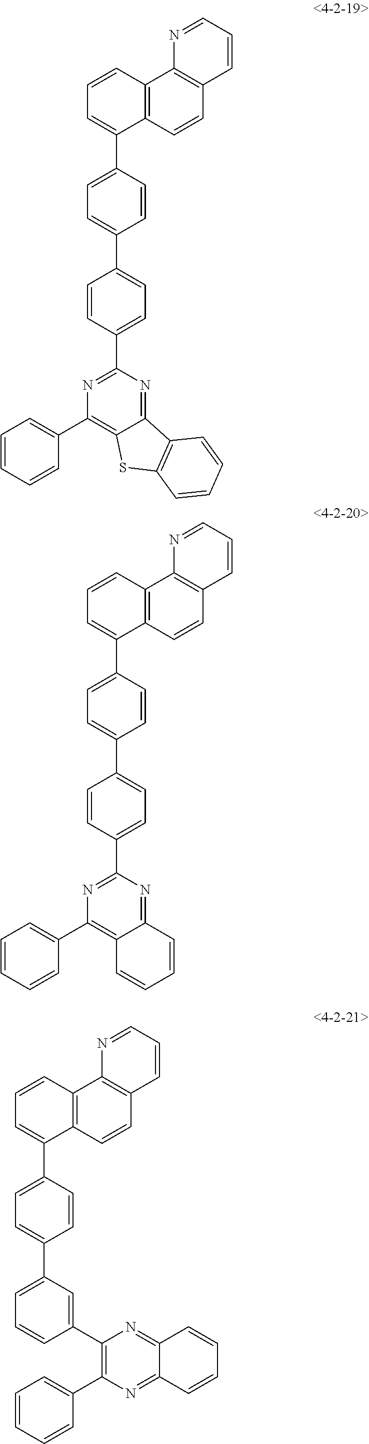

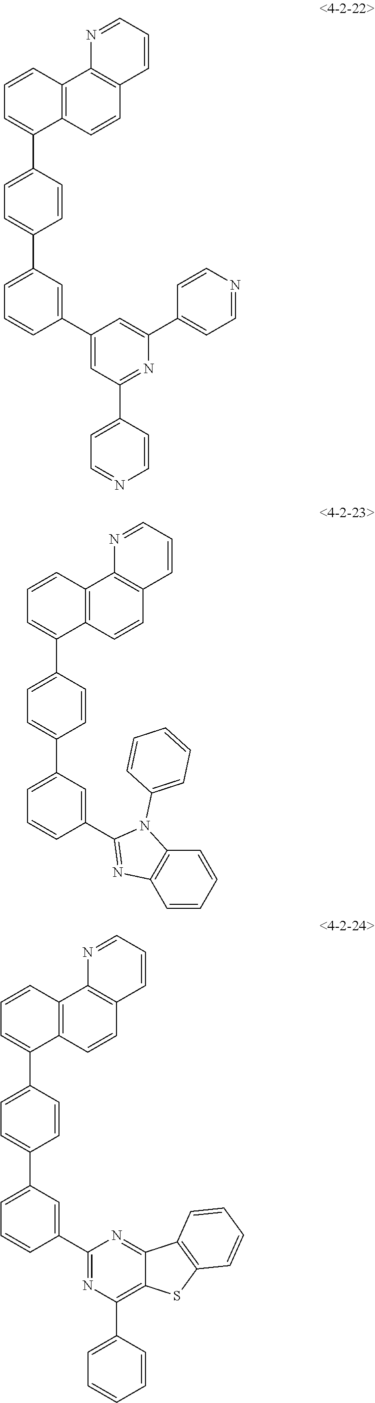

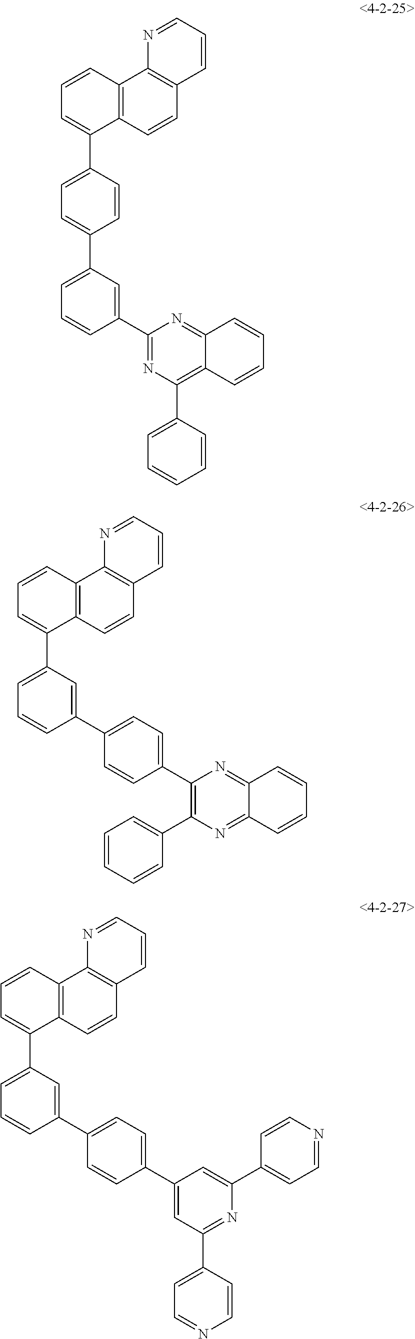

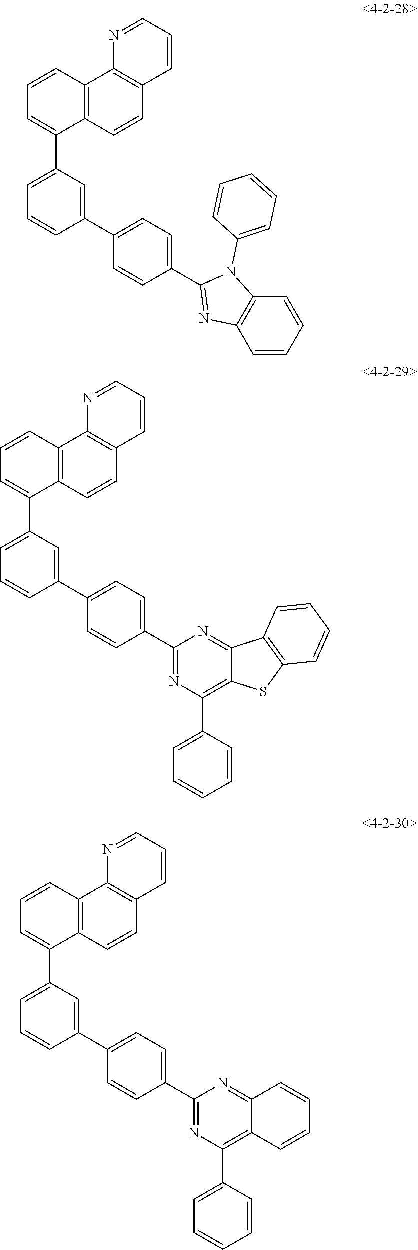 [SMILES: c1ccc(-n2c(-c3ccc(-c4cccc(-c5cccc6c5ccc5cccnc56)c4)cc3)nc3ccccc32)cc1 ; c1ccc(-c2nc(-c3ccc(-c4cccc(-c5cccc6c5ccc5cccnc56)c4)cc3)nc3c2sc2ccccc23)cc1 ; c1ccc(-c2nc(-c3ccc(-c4cccc(-c5cccc6c5ccc5cccnc56)c4)cc3)nc3ccccc23)cc1]

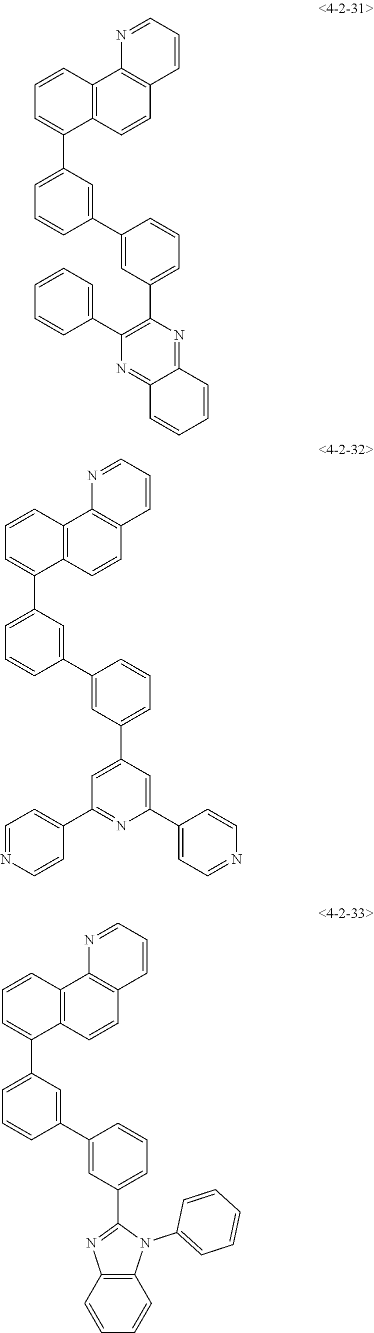 [SMILES: c1ccc(-c2nc3ccccc3nc2-c2cccc(-c3cccc(-c4cccc5c4ccc4cccnc45)c3)c2)cc1 ; c1cc(-c2cccc(-c3cccc4c3ccc3cccnc34)c2)cc(-c2cc(-c3ccncc3)nc(-c3ccncc3)c2)c1 ; c1ccc(-n2c(-c3cccc(-c4cccc(-c5cccc6c5ccc5cccnc56)c4)c3)nc3ccccc32)cc1]

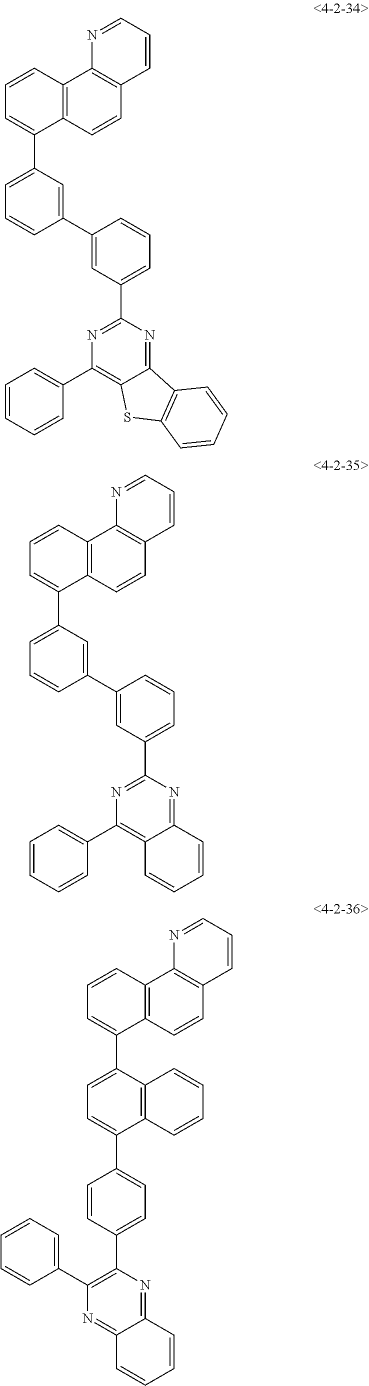

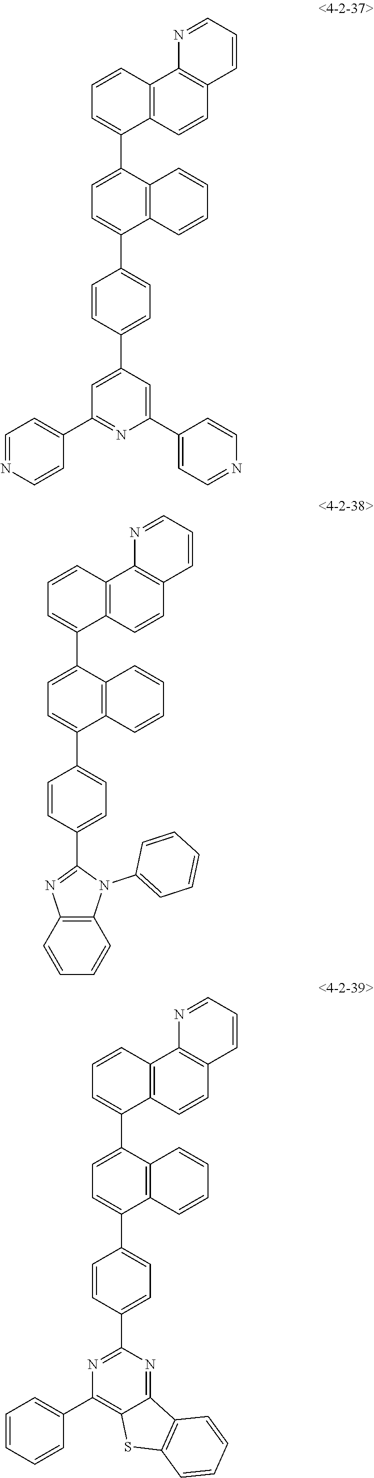 [SMILES: c1cnc2c(c1)ccc1c(-c3ccc(-c4ccc(-c5cc(-c6ccncc6)nc(-c6ccncc6)c5)cc4)c4ccccc34)cccc12 ; c1ccc(-n2c(-c3ccc(-c4ccc(-c5cccc6c5ccc5cccnc56)c5ccccc45)cc3)nc3ccccc32)cc1 ; c1ccc(-c2nc(-c3ccc(-c4ccc(-c5cccc6c5ccc5cccnc56)c5ccccc45)cc3)nc3c2sc2ccccc23)cc1]

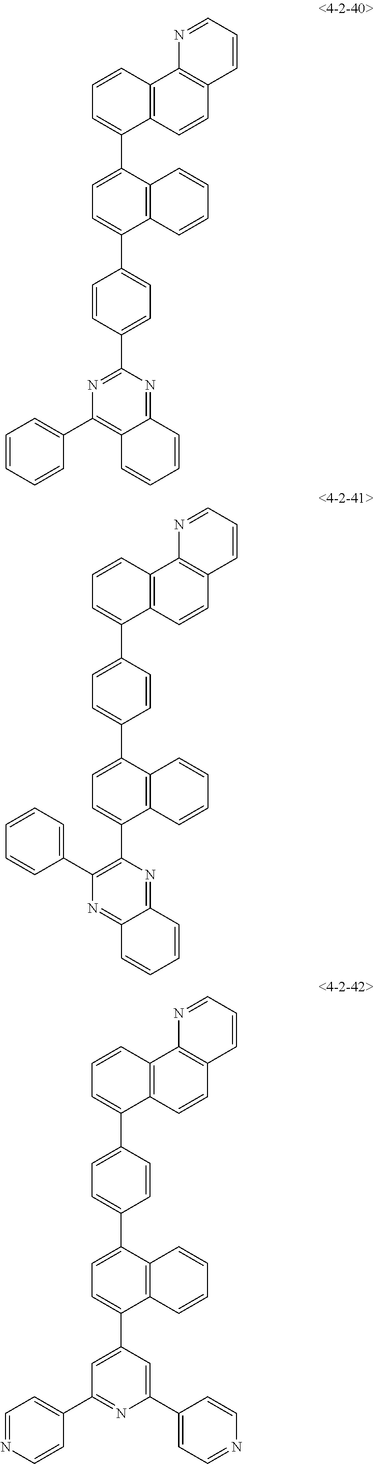 [SMILES: c1ccc(-c2nc(-c3ccc(-c4ccc(-c5cccc6c5ccc5cccnc56)c5ccccc45)cc3)nc3ccccc23)cc1 ; c1ccc(-c2nc3ccccc3nc2-c2ccc(-c3ccc(-c4cccc5c4ccc4cccnc45)cc3)c3ccccc23)cc1 ; c1cnc2c(c1)ccc1c(-c3ccc(-c4ccc(-c5cc(-c6ccncc6)nc(-c6ccncc6)c5)c5ccccc45)cc3)cccc12]

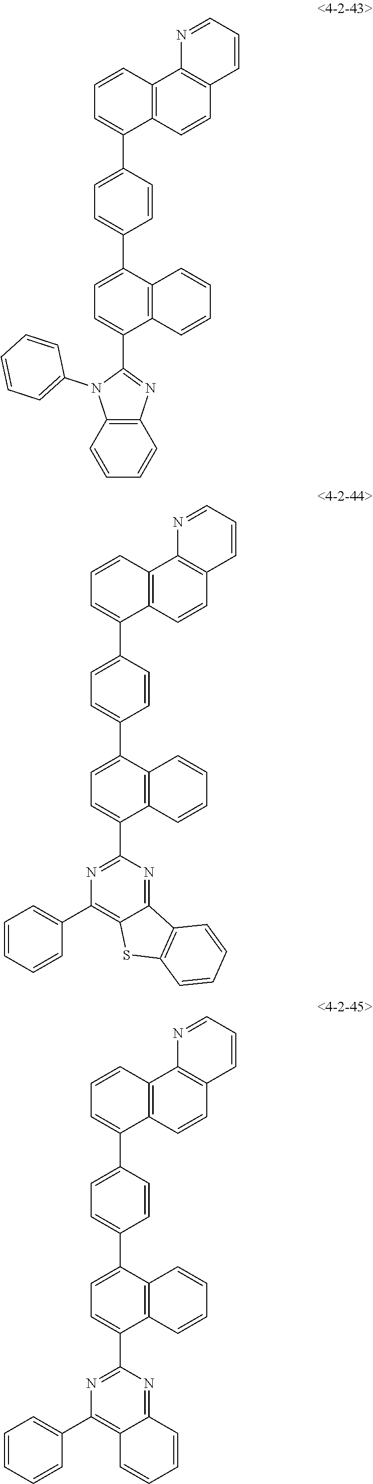

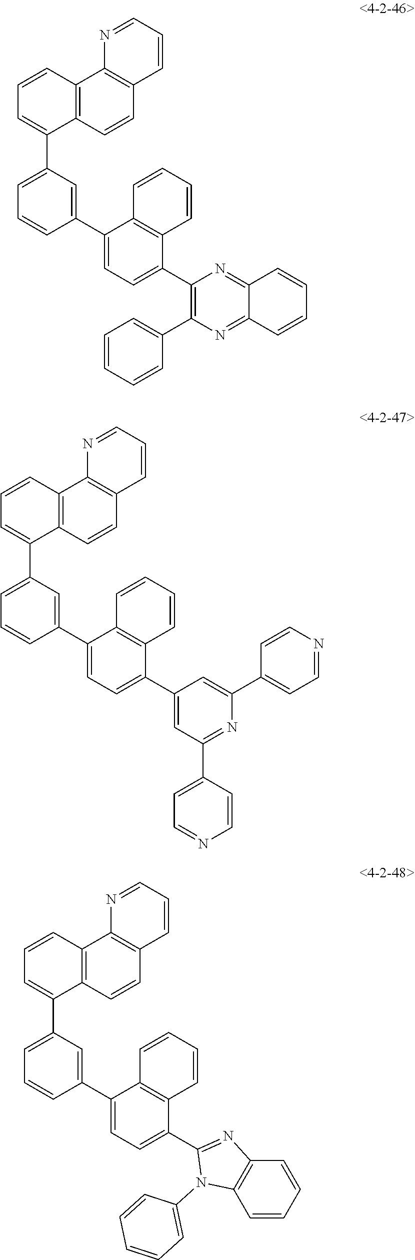

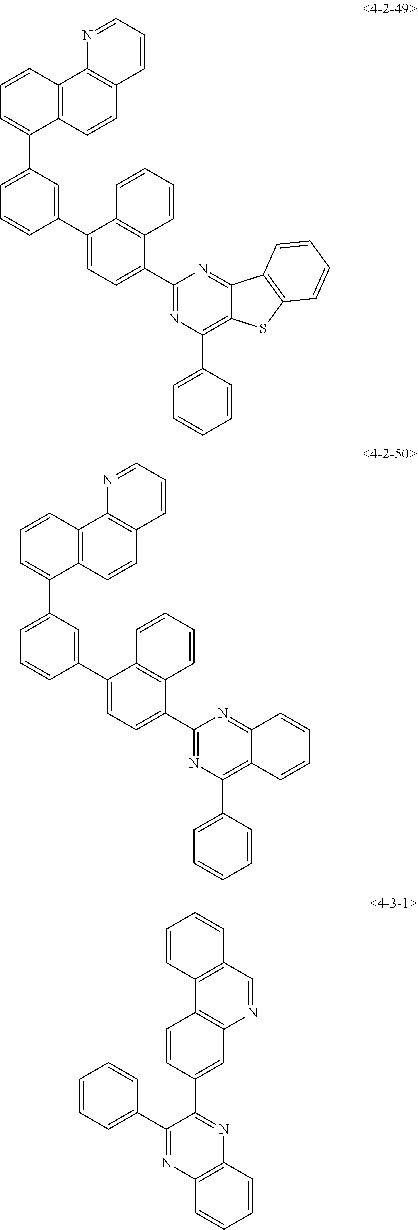

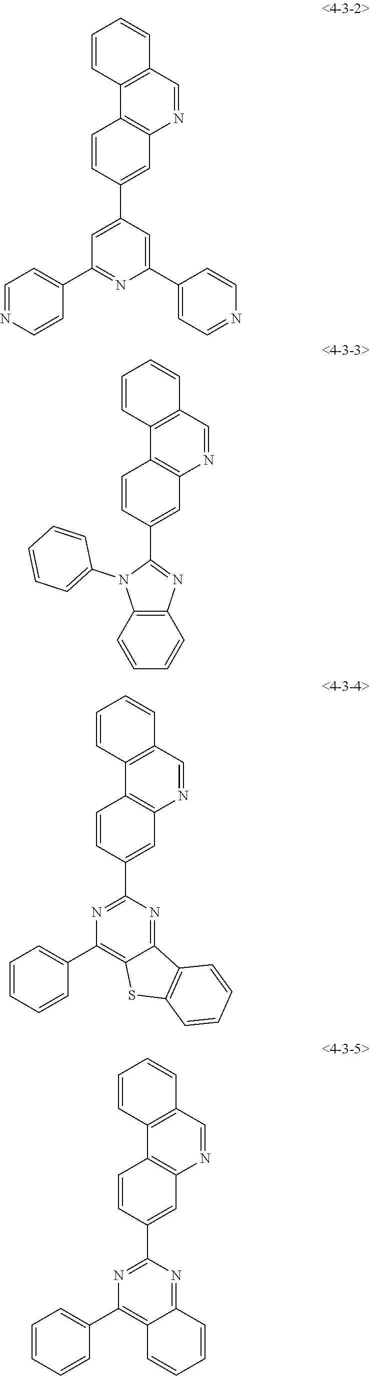

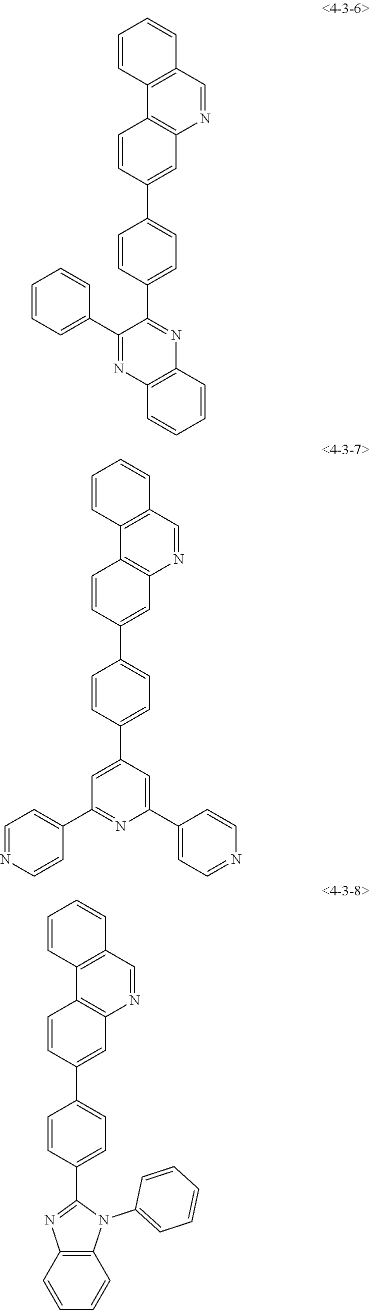

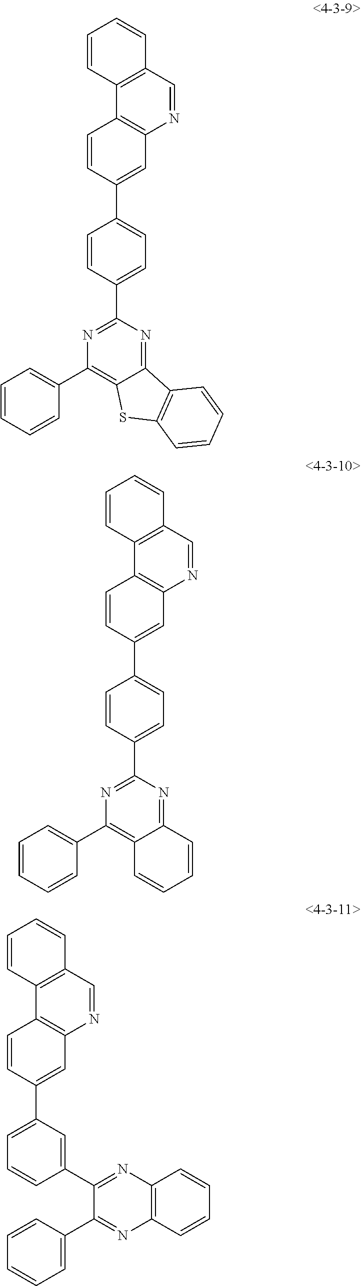

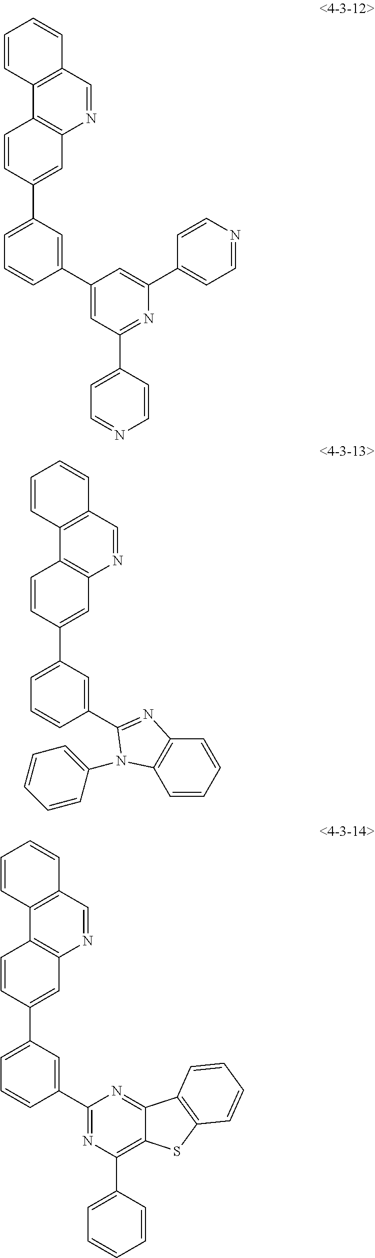

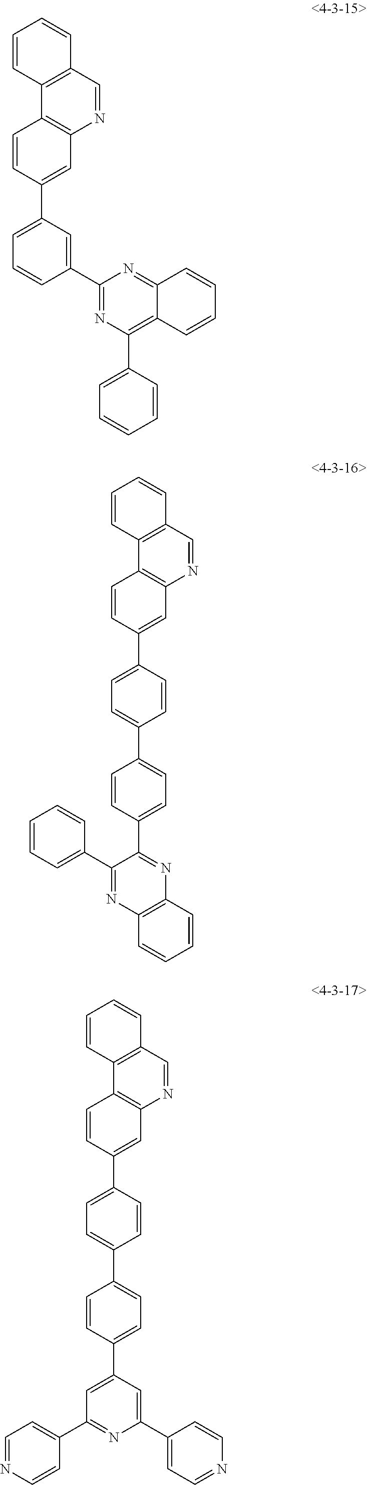

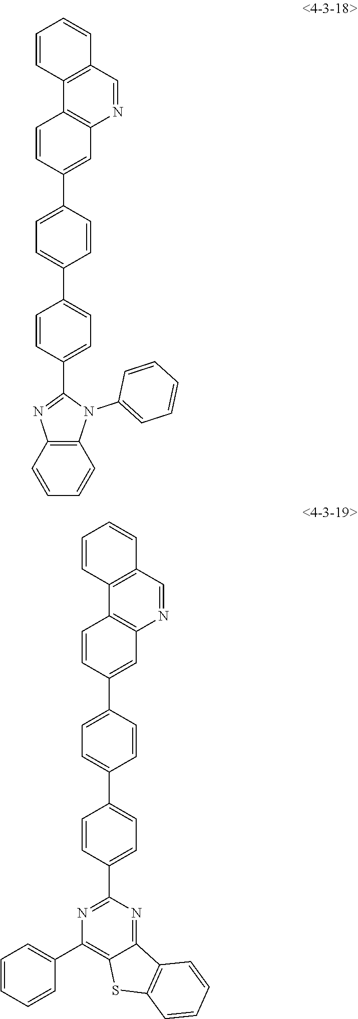

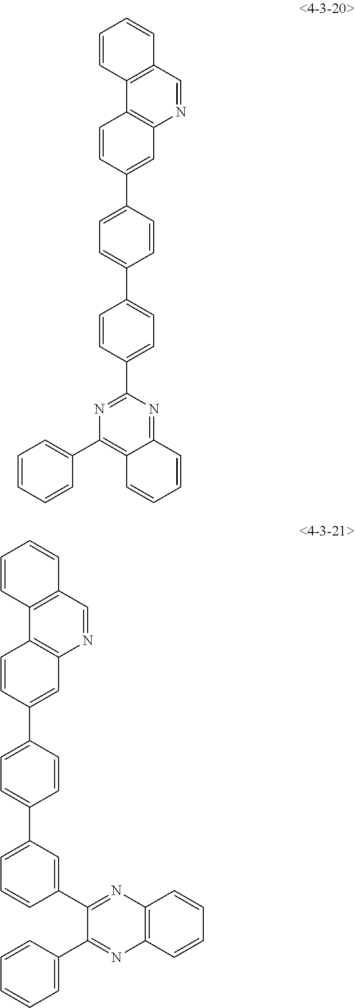

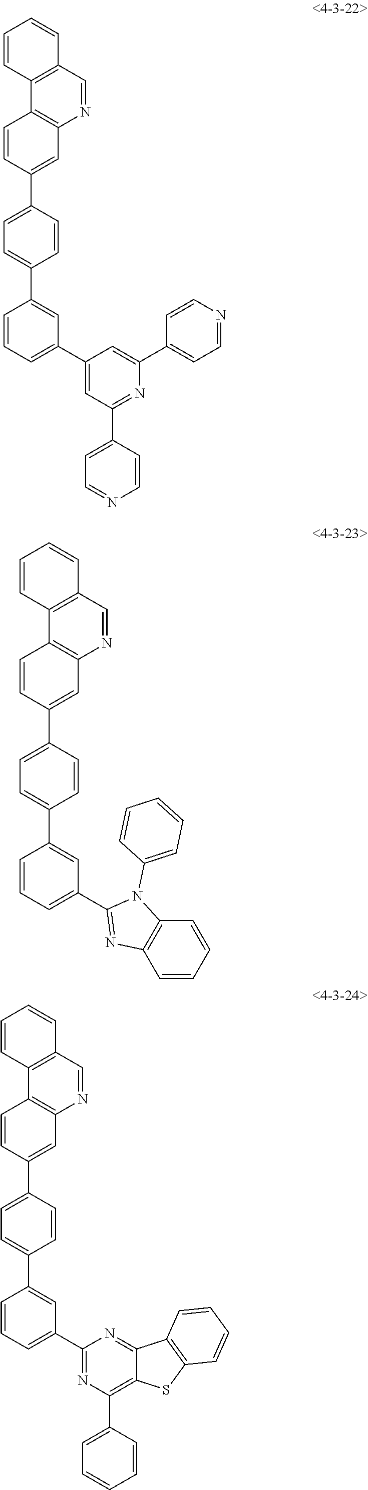 [SMILES: c1cc(-c2ccc(-c3ccc4c(c3)ncc3ccccc34)cc2)cc(-c2cc(-c3ccncc3)nc(-c3ccncc3)c2)c1 ; c1ccc(-n2c(-c3cccc(-c4ccc(-c5ccc6c(c5)ncc5ccccc56)cc4)c3)nc3ccccc32)cc1 ; c1ccc(-c2nc(-c3cccc(-c4ccc(-c5ccc6c(c5)ncc5ccccc56)cc4)c3)nc3c2sc2ccccc23)cc1]

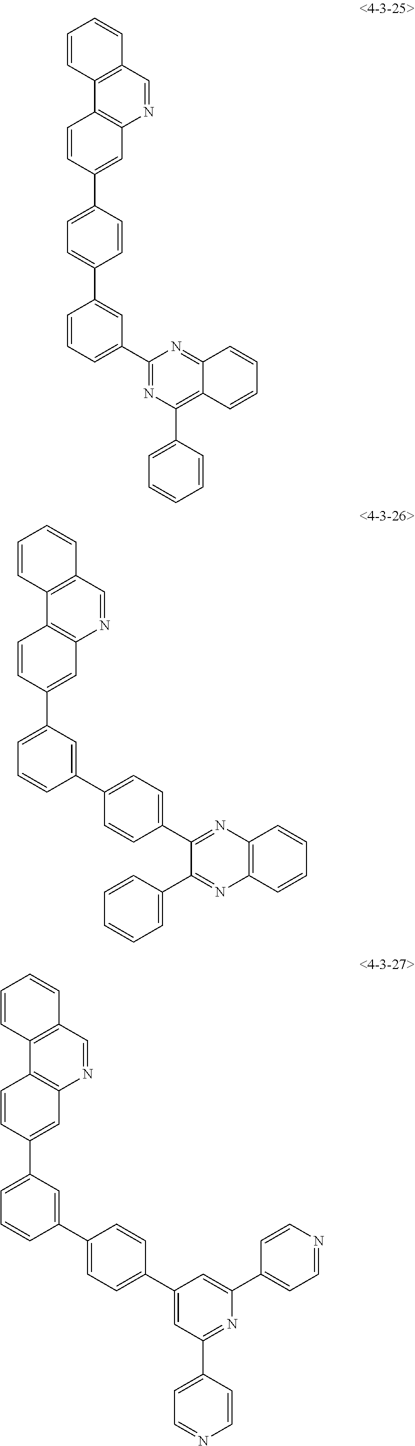 [SMILES: c1ccc(-c2nc(-c3cccc(-c4ccc(-c5ccc6c(c5)ncc5ccccc56)cc4)c3)nc3ccccc23)cc1 ; c1ccc(-c2nc3ccccc3nc2-c2ccc(-c3cccc(-c4ccc5c(c4)ncc4ccccc45)c3)cc2)cc1 ; c1cc(-c2ccc(-c3cc(-c4ccncc4)nc(-c4ccncc4)c3)cc2)cc(-c2ccc3c(c2)ncc2ccccc23)c1]

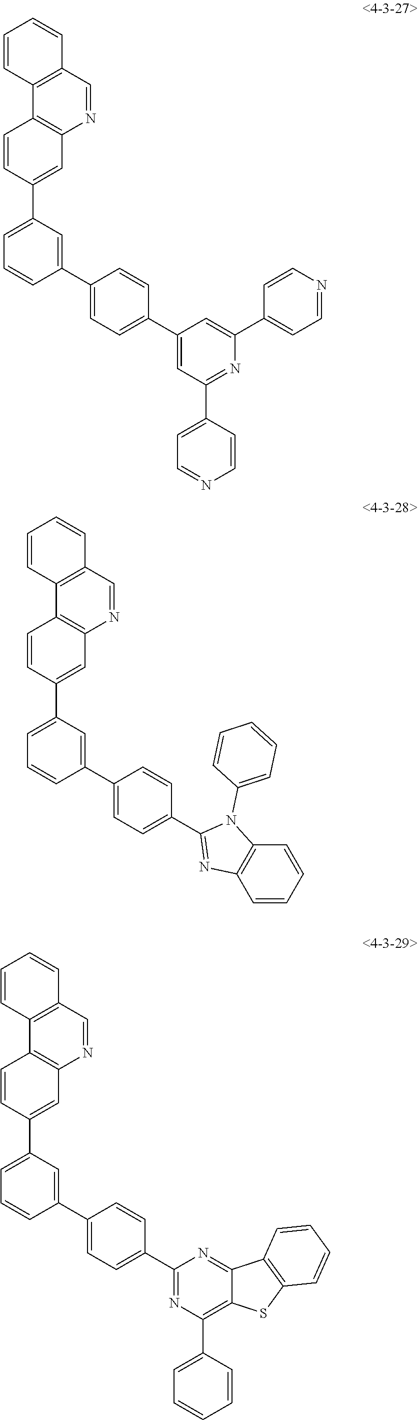 [SMILES: c1cc(-c2ccc(-c3cc(-c4ccncc4)nc(-c4ccncc4)c3)cc2)cc(-c2ccc3c(c2)ncc2ccccc23)c1 ; c1ccc(-n2c(-c3ccc(-c4cccc(-c5ccc6c(c5)ncc5ccccc56)c4)cc3)nc3ccccc32)cc1 ; c1ccc(-c2nc(-c3ccc(-c4cccc(-c5ccc6c(c5)ncc5ccccc56)c4)cc3)nc3c2sc2ccccc23)cc1]

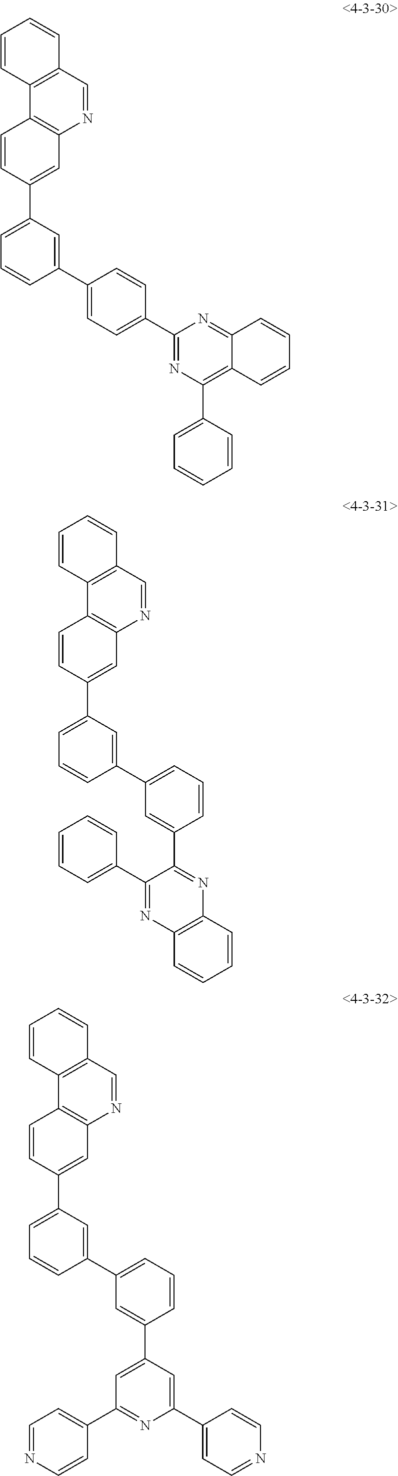 [SMILES: c1ccc(-c2nc(-c3ccc(-c4cccc(-c5ccc6c(c5)ncc5ccccc56)c4)cc3)nc3ccccc23)cc1 ; c1ccc(-c2nc3ccccc3nc2-c2cccc(-c3cccc(-c4ccc5c(c4)ncc4ccccc45)c3)c2)cc1 ; c1cc(-c2cccc(-c3ccc4c(c3)ncc3ccccc34)c2)cc(-c2cc(-c3ccncc3)nc(-c3ccncc3)c2)c1]

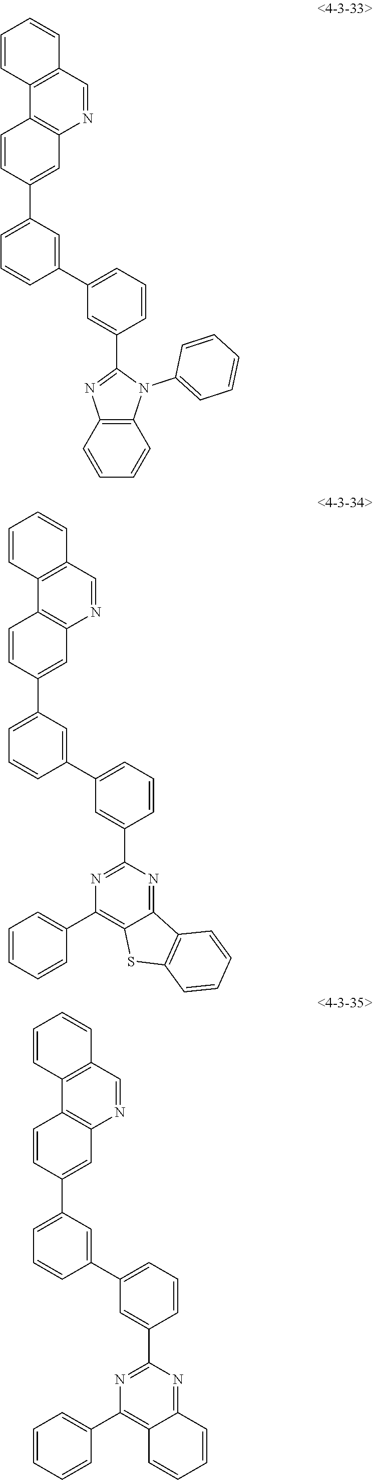

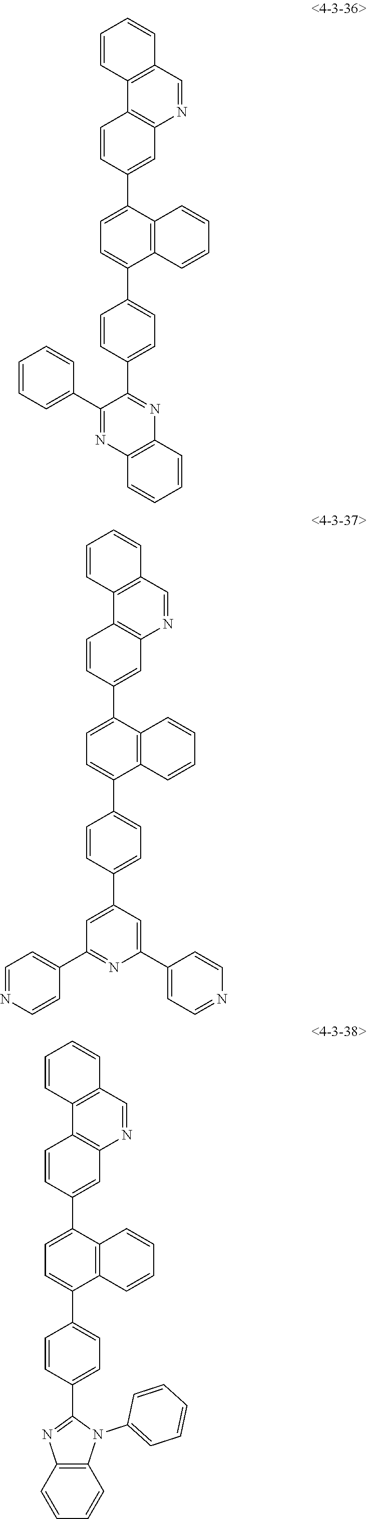 [SMILES: c1ccc(-c2nc3ccccc3nc2-c2ccc(-c3ccc(-c4ccc5c(c4)ncc4ccccc45)c4ccccc34)cc2)cc1 ; c1ccc2c(c1)cnc1cc(-c3ccc(-c4ccc(-c5cc(-c6ccncc6)nc(-c6ccncc6)c5)cc4)c4ccccc34)ccc12 ; c1ccc(-n2c(-c3ccc(-c4ccc(-c5ccc6c(c5)ncc5ccccc56)c5ccccc45)cc3)nc3ccccc32)cc1]

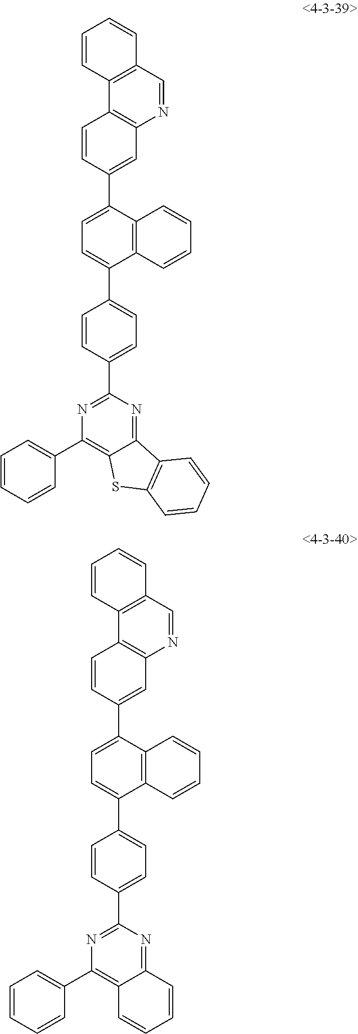 [SMILES: c1ccc(-c2nc(-c3ccc(-c4ccc(-c5ccc6c(c5)ncc5ccccc56)c5ccccc45)cc3)nc3c2sc2ccccc23)cc1 ; c1ccc(-c2nc(-c3ccc(-c4ccc(-c5ccc6c(c5)ncc5ccccc56)c5ccccc45)cc3)nc3ccccc23)cc1]

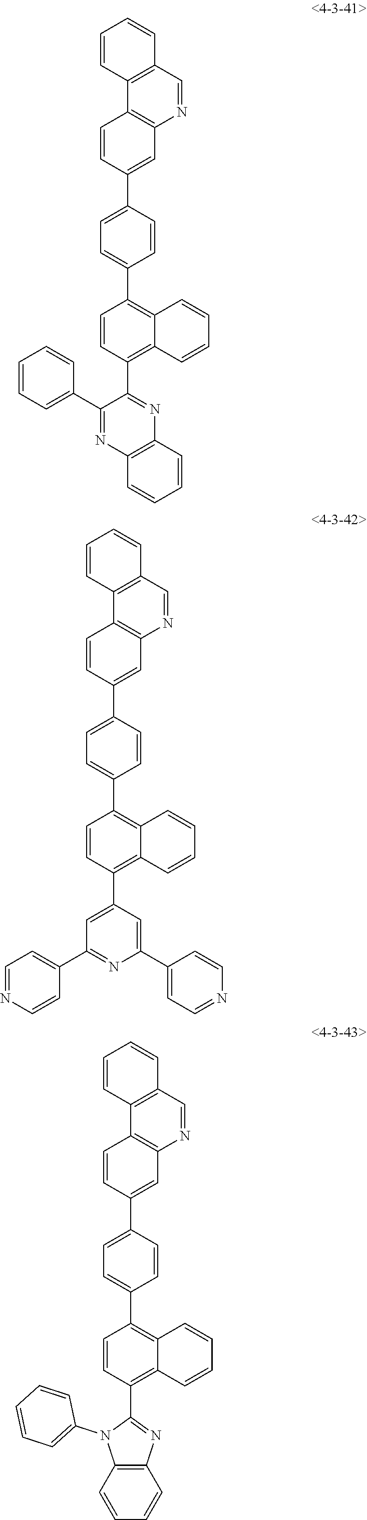 [SMILES: c1ccc(-c2nc3ccccc3nc2-c2ccc(-c3ccc(-c4ccc5c(c4)ncc4ccccc45)cc3)c3ccccc23)cc1 ; c1ccc2c(c1)cnc1cc(-c3ccc(-c4ccc(-c5cc(-c6ccncc6)nc(-c6ccncc6)c5)c5ccccc45)cc3)ccc12 ; c1ccc(-n2c(-c3ccc(-c4ccc(-c5ccc6c(c5)ncc5ccccc56)cc4)c4ccccc34)nc3ccccc32)cc1]

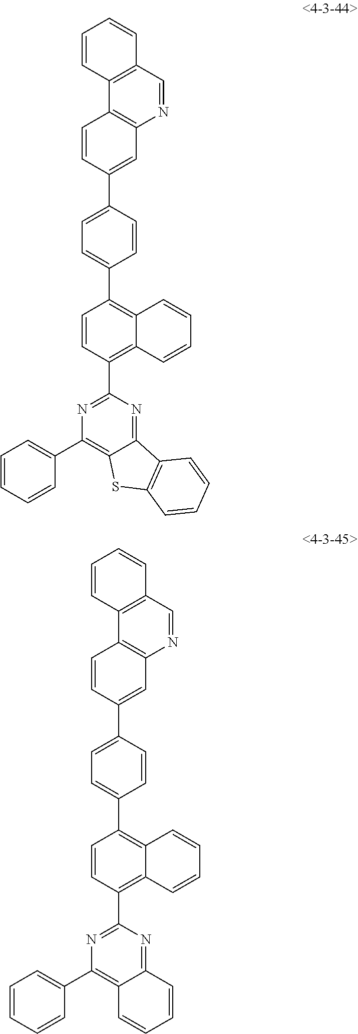 [SMILES: c1ccc(-c2nc(-c3ccc(-c4ccc(-c5ccc6c(c5)ncc5ccccc56)cc4)c4ccccc34)nc3c2sc2ccccc23)cc1 ; c1ccc(-c2nc(-c3ccc(-c4ccc(-c5ccc6c(c5)ncc5ccccc56)cc4)c4ccccc34)nc3ccccc23)cc1]

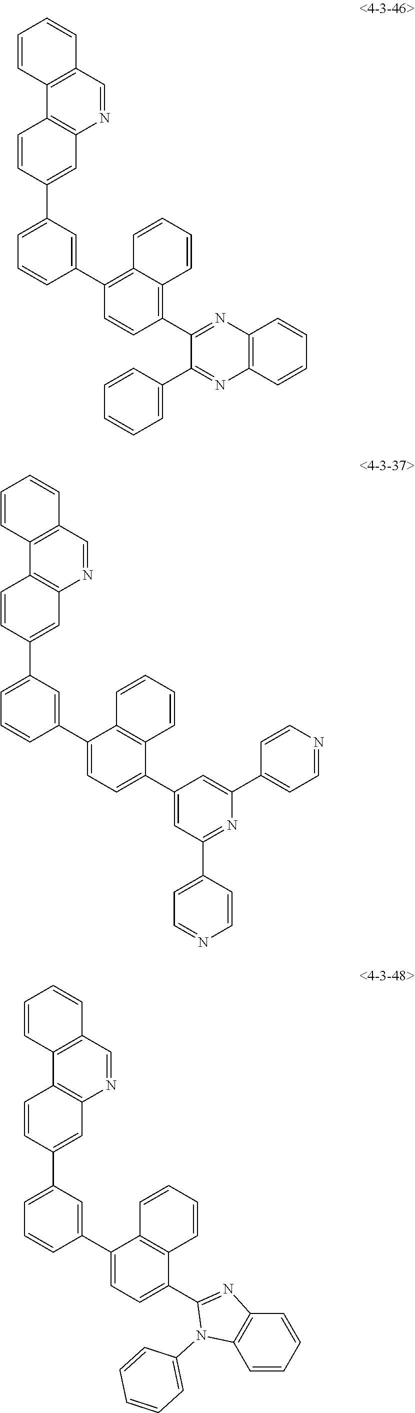 [SMILES: c1ccc(-c2nc3ccccc3nc2-c2ccc(-c3cccc(-c4ccc5c(c4)ncc4ccccc45)c3)c3ccccc23)cc1 ; c1cc(-c2ccc3c(c2)ncc2ccccc23)cc(-c2ccc(-c3cc(-c4ccncc4)nc(-c4ccncc4)c3)c3ccccc23)c1 ; c1ccc(-n2c(-c3ccc(-c4cccc(-c5ccc6c(c5)ncc5ccccc56)c4)c4ccccc34)nc3ccccc32)cc1]

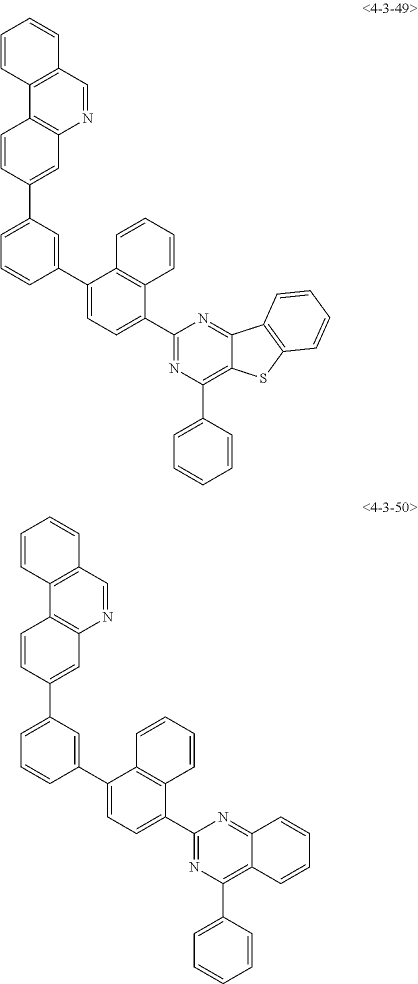 [SMILES: c1ccc(-c2nc(-c3ccc(-c4cccc(-c5ccc6c(c5)ncc5ccccc56)c4)c4ccccc34)nc3c2sc2ccccc23)cc1 ; c1ccc(-c2nc(-c3ccc(-c4cccc(-c5ccc6c(c5)ncc5ccccc56)c4)c4ccccc34)nc3ccccc23)cc1]

Furthermore, the compound of chemical formula 1 may be any one of the following compounds.

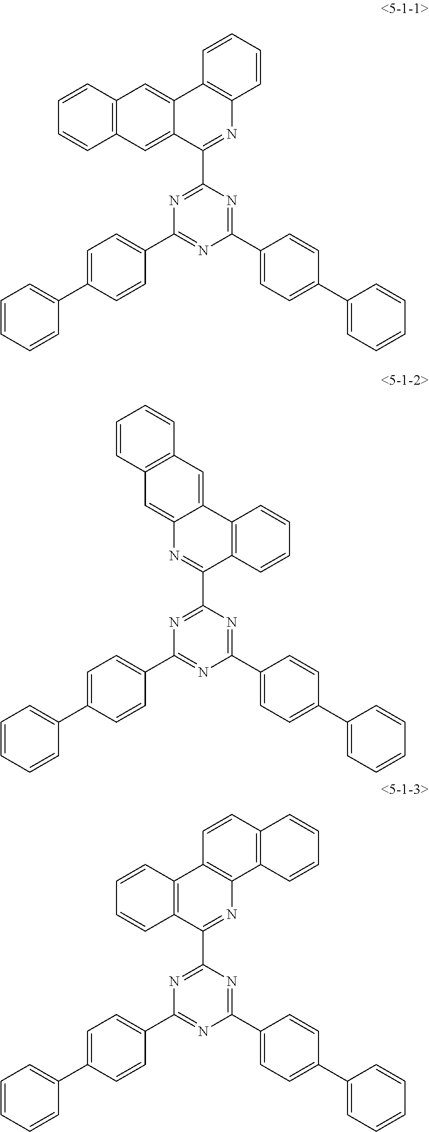 [SMILES: c1ccc(-c2ccc(-c3nc(-c4ccc(-c5ccccc5)cc4)nc(-c4nc5ccccc5c5cc6ccccc6cc45)n3)cc2)cc1 ; c1ccc(-c2ccc(-c3nc(-c4ccc(-c5ccccc5)cc4)nc(-c4nc5cc6ccccc6cc5c5ccccc45)n3)cc2)cc1 ; c1ccc(-c2ccc(-c3nc(-c4ccc(-c5ccccc5)cc4)nc(-c4nc5c6ccccc6ccc5c5ccccc45)n3)cc2)cc1]

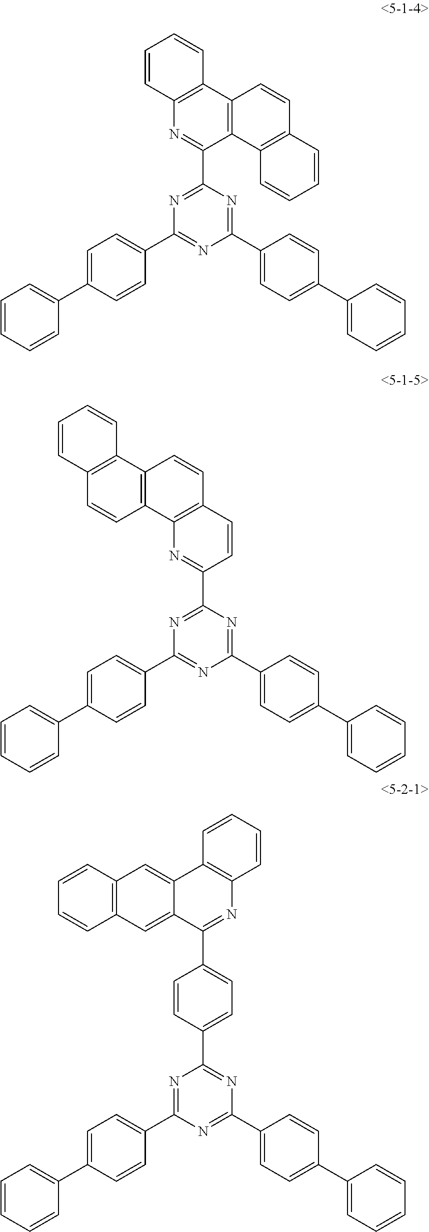 [SMILES: c1ccc(-c2ccc(-c3nc(-c4ccc(-c5ccccc5)cc4)nc(-c4nc5ccccc5c5ccc6ccccc6c45)n3)cc2)cc1 ; c1ccc(-c2ccc(-c3nc(-c4ccc(-c5ccccc5)cc4)nc(-c4ccc5ccc6c7ccccc7ccc6c5n4)n3)cc2)cc1 ; c1ccc(-c2ccc(-c3nc(-c4ccc(-c5ccccc5)cc4)nc(-c4ccc(-c5nc6ccccc6c6cc7ccccc7cc56)cc4)n3)cc2)cc1]

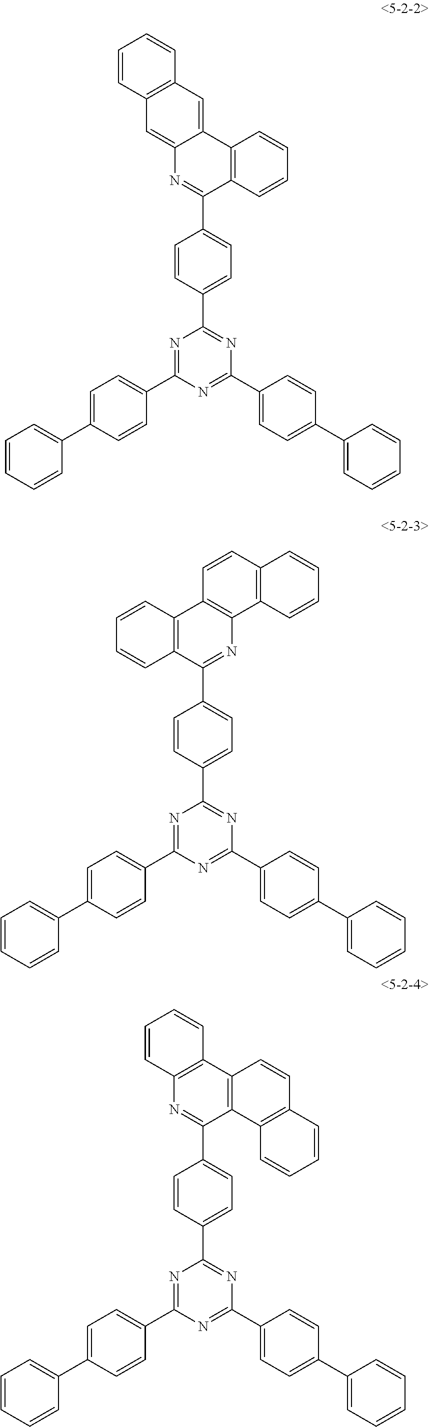 [SMILES: c1ccc(-c2ccc(-c3nc(-c4ccc(-c5ccccc5)cc4)nc(-c4ccc(-c5nc6cc7ccccc7cc6c6ccccc56)cc4)n3)cc2)cc1 ; c1ccc(-c2ccc(-c3nc(-c4ccc(-c5ccccc5)cc4)nc(-c4ccc(-c5nc6c7ccccc7ccc6c6ccccc56)cc4)n3)cc2)cc1 ; c1ccc(-c2ccc(-c3nc(-c4ccc(-c5ccccc5)cc4)nc(-c4ccc(-c5nc6ccccc6c6ccc7ccccc7c56)cc4)n3)cc2)cc1]

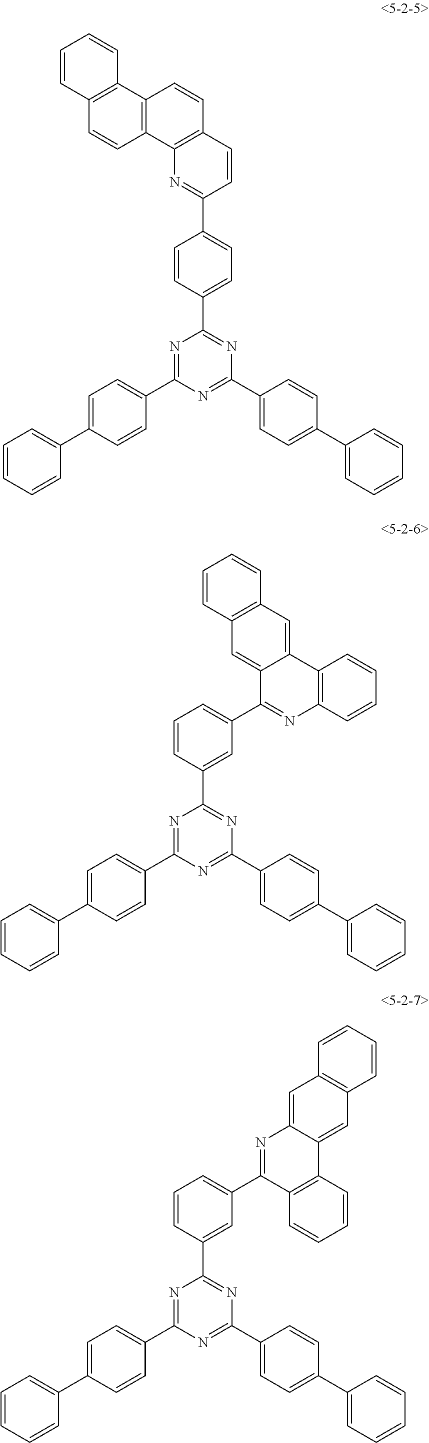 [SMILES: c1ccc(-c2ccc(-c3nc(-c4ccc(-c5ccccc5)cc4)nc(-c4ccc(-c5ccc6ccc7c8ccccc8ccc7c6n5)cc4)n3)cc2)cc1 ; c1ccc(-c2ccc(-c3nc(-c4ccc(-c5ccccc5)cc4)nc(-c4cccc(-c5nc6ccccc6c6cc7ccccc7cc56)c4)n3)cc2)cc1 ; c1ccc(-c2ccc(-c3nc(-c4ccc(-c5ccccc5)cc4)nc(-c4cccc(-c5nc6cc7ccccc7cc6c6ccccc56)c4)n3)cc2)cc1]

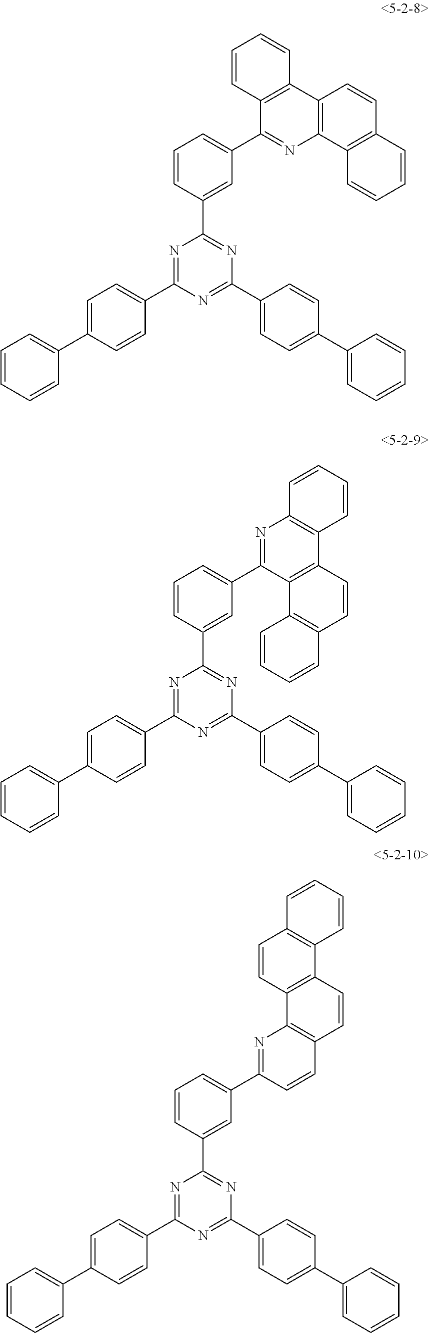 [SMILES: c1ccc(-c2ccc(-c3nc(-c4ccc(-c5ccccc5)cc4)nc(-c4cccc(-c5nc6c7ccccc7ccc6c6ccccc56)c4)n3)cc2)cc1 ; c1ccc(-c2ccc(-c3nc(-c4ccc(-c5ccccc5)cc4)nc(-c4cccc(-c5nc6ccccc6c6ccc7ccccc7c56)c4)n3)cc2)cc1 ; c1ccc(-c2ccc(-c3nc(-c4ccc(-c5ccccc5)cc4)nc(-c4cccc(-c5ccc6ccc7c8ccccc8ccc7c6n5)c4)n3)cc2)cc1]

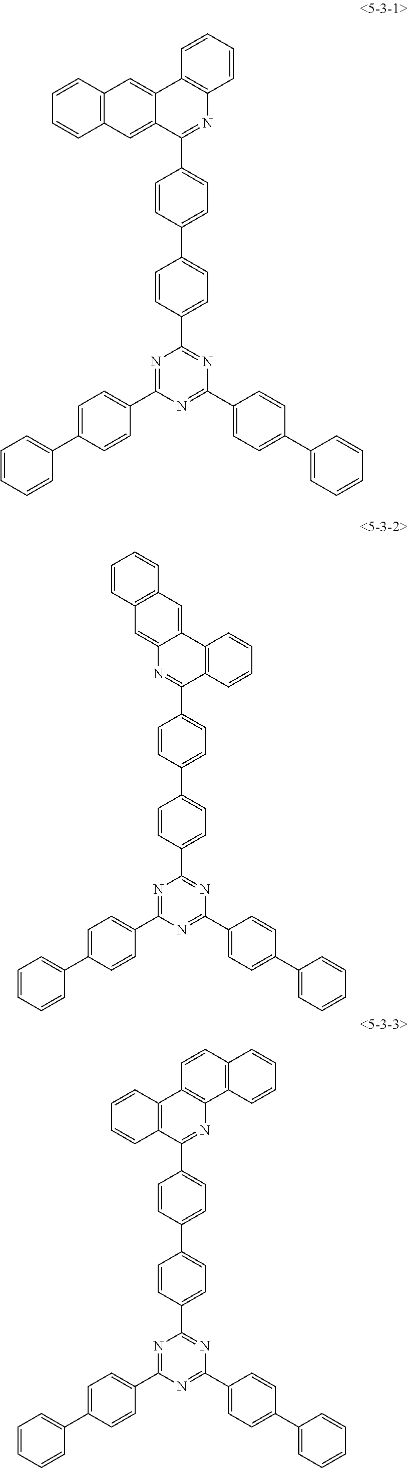 [SMILES: c1ccc(-c2ccc(-c3nc(-c4ccc(-c5ccccc5)cc4)nc(-c4ccc(-c5ccc(-c6nc7ccccc7c7cc8ccccc8cc67)cc5)cc4)n3)cc2)cc1 ; c1ccc(-c2ccc(-c3nc(-c4ccc(-c5ccccc5)cc4)nc(-c4ccc(-c5ccc(-c6nc7cc8ccccc8cc7c7ccccc67)cc5)cc4)n3)cc2)cc1 ; c1ccc(-c2ccc(-c3nc(-c4ccc(-c5ccccc5)cc4)nc(-c4ccc(-c5ccc(-c6nc7c8ccccc8ccc7c7ccccc67)cc5)cc4)n3)cc2)cc1]

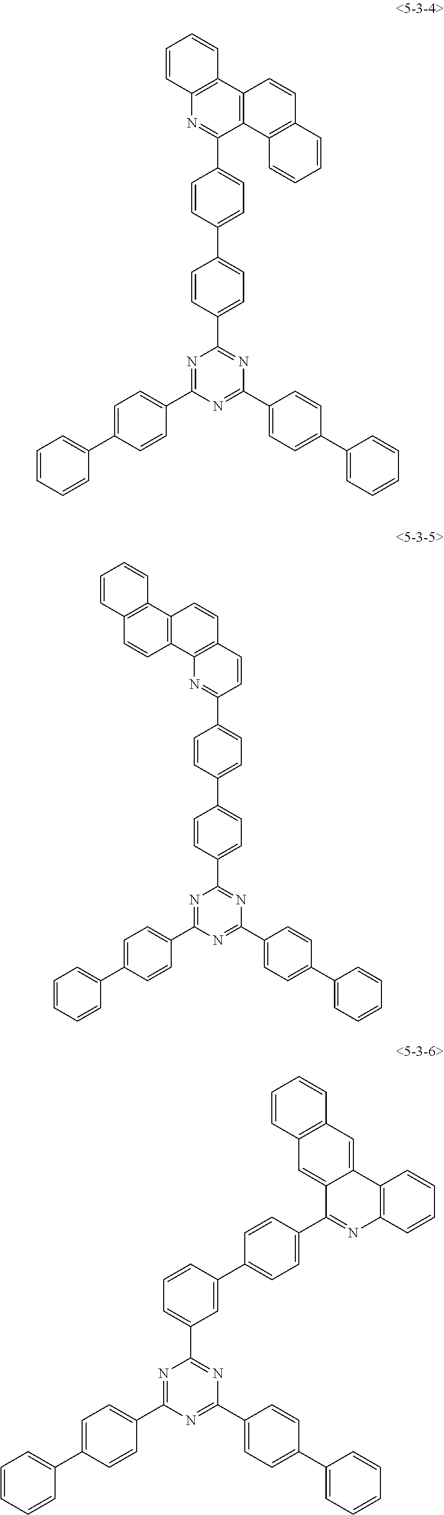 [SMILES: c1ccc(-c2ccc(-c3nc(-c4ccc(-c5ccccc5)cc4)nc(-c4ccc(-c5ccc(-c6nc7ccccc7c7ccc8ccccc8c67)cc5)cc4)n3)cc2)cc1 ; c1ccc(-c2ccc(-c3nc(-c4ccc(-c5ccccc5)cc4)nc(-c4ccc(-c5ccc(-c6ccc7ccc8c9ccccc9ccc8c7n6)cc5)cc4)n3)cc2)cc1 ; c1ccc(-c2ccc(-c3nc(-c4ccc(-c5ccccc5)cc4)nc(-c4cccc(-c5ccc(-c6nc7ccccc7c7cc8ccccc8cc67)cc5)c4)n3)cc2)cc1]

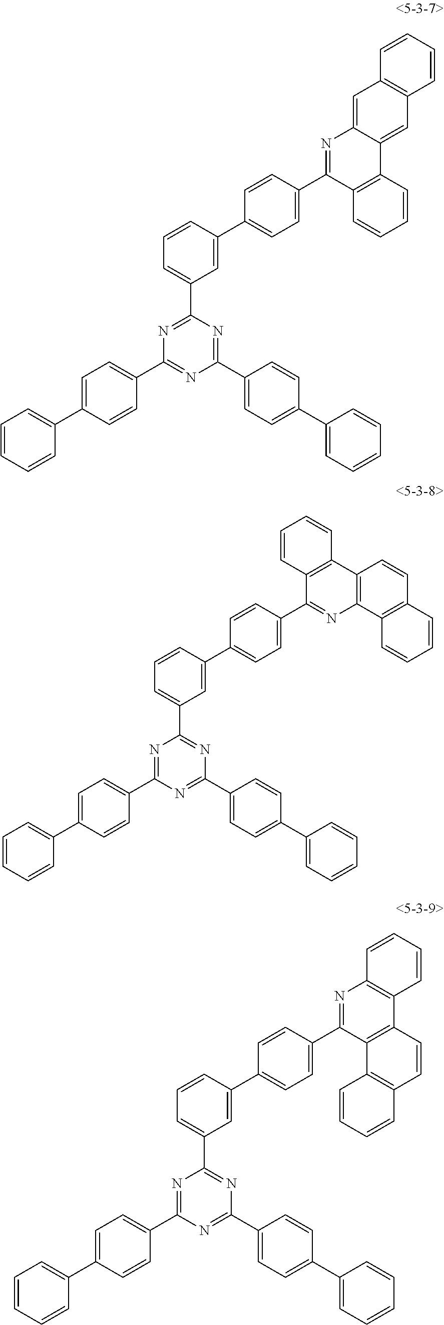 [SMILES: c1ccc(-c2ccc(-c3nc(-c4ccc(-c5ccccc5)cc4)nc(-c4cccc(-c5ccc(-c6nc7cc8ccccc8cc7c7ccccc67)cc5)c4)n3)cc2)cc1 ; c1ccc(-c2ccc(-c3nc(-c4ccc(-c5ccccc5)cc4)nc(-c4cccc(-c5ccc(-c6nc7c8ccccc8ccc7c7ccccc67)cc5)c4)n3)cc2)cc1 ; c1ccc(-c2ccc(-c3nc(-c4ccc(-c5ccccc5)cc4)nc(-c4cccc(-c5ccc(-c6nc7ccccc7c7ccc8ccccc8c67)cc5)c4)n3)cc2)cc1]

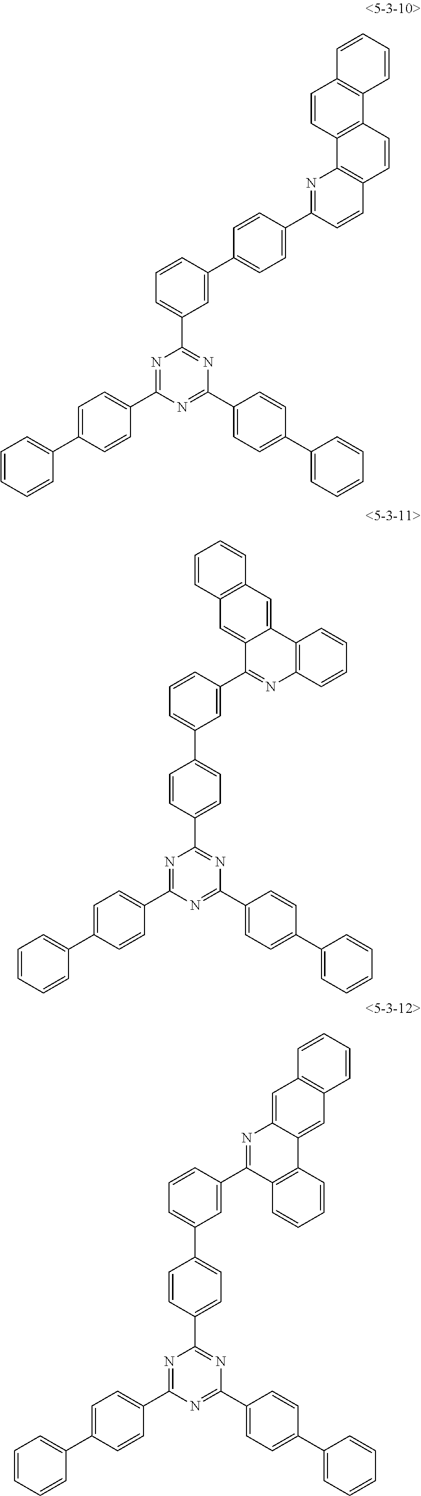 [SMILES: c1ccc(-c2ccc(-c3nc(-c4ccc(-c5ccccc5)cc4)nc(-c4cccc(-c5ccc(-c6ccc7ccc8c9ccccc9ccc8c7n6)cc5)c4)n3)cc2)cc1 ; c1ccc(-c2ccc(-c3nc(-c4ccc(-c5ccccc5)cc4)nc(-c4ccc(-c5cccc(-c6nc7ccccc7c7cc8ccccc8cc67)c5)cc4)n3)cc2)cc1 ; c1ccc(-c2ccc(-c3nc(-c4ccc(-c5ccccc5)cc4)nc(-c4ccc(-c5cccc(-c6nc7cc8ccccc8cc7c7ccccc67)c5)cc4)n3)cc2)cc1]

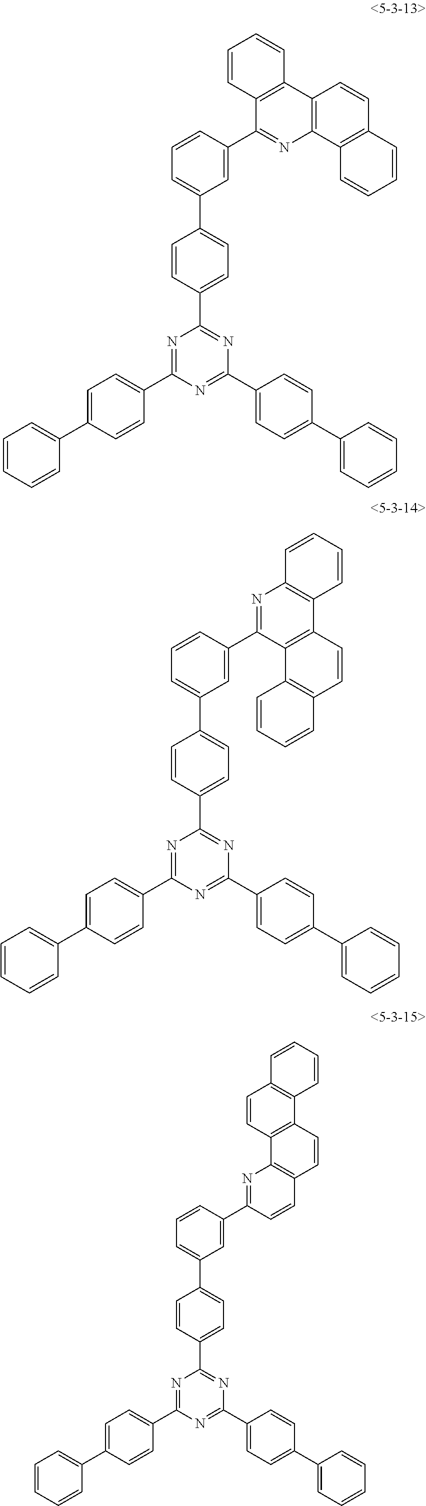 [SMILES: c1ccc(-c2ccc(-c3nc(-c4ccc(-c5ccccc5)cc4)nc(-c4ccc(-c5cccc(-c6nc7c8ccccc8ccc7c7ccccc67)c5)cc4)n3)cc2)cc1 ; c1ccc(-c2ccc(-c3nc(-c4ccc(-c5ccccc5)cc4)nc(-c4ccc(-c5cccc(-c6nc7ccccc7c7ccc8ccccc8c67)c5)cc4)n3)cc2)cc1 ; c1ccc(-c2ccc(-c3nc(-c4ccc(-c5ccccc5)cc4)nc(-c4ccc(-c5cccc(-c6ccc7ccc8c9ccccc9ccc8c7n6)c5)cc4)n3)cc2)cc1]

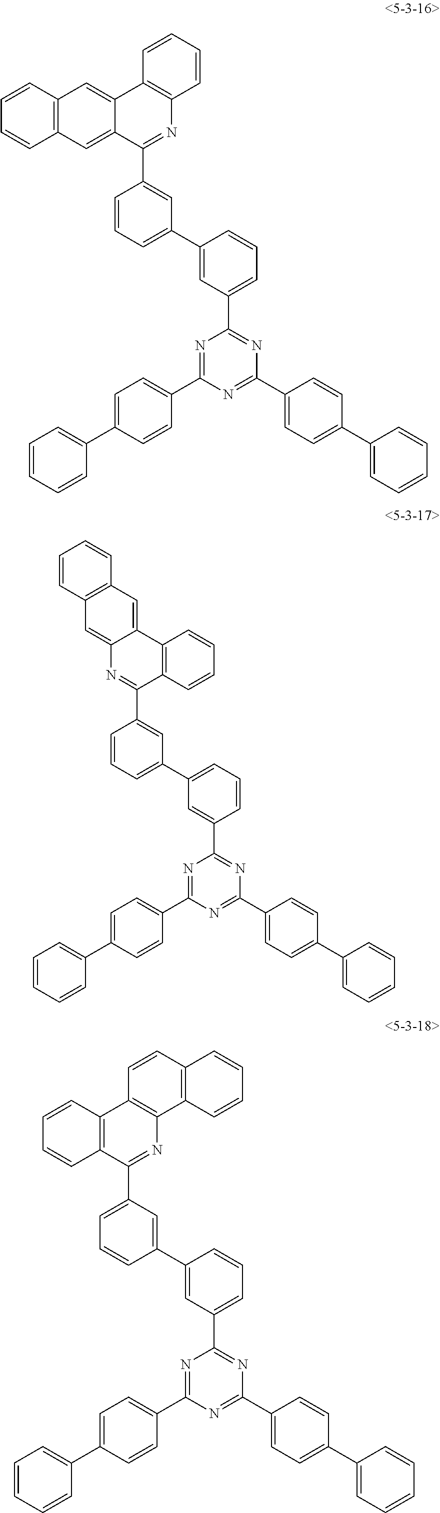 [SMILES: c1ccc(-c2ccc(-c3nc(-c4ccc(-c5ccccc5)cc4)nc(-c4cccc(-c5cccc(-c6nc7ccccc7c7cc8ccccc8cc67)c5)c4)n3)cc2)cc1 ; c1ccc(-c2ccc(-c3nc(-c4ccc(-c5ccccc5)cc4)nc(-c4cccc(-c5cccc(-c6nc7cc8ccccc8cc7c7ccccc67)c5)c4)n3)cc2)cc1 ; c1ccc(-c2ccc(-c3nc(-c4ccc(-c5ccccc5)cc4)nc(-c4cccc(-c5cccc(-c6nc7c8ccccc8ccc7c7ccccc67)c5)c4)n3)cc2)cc1]

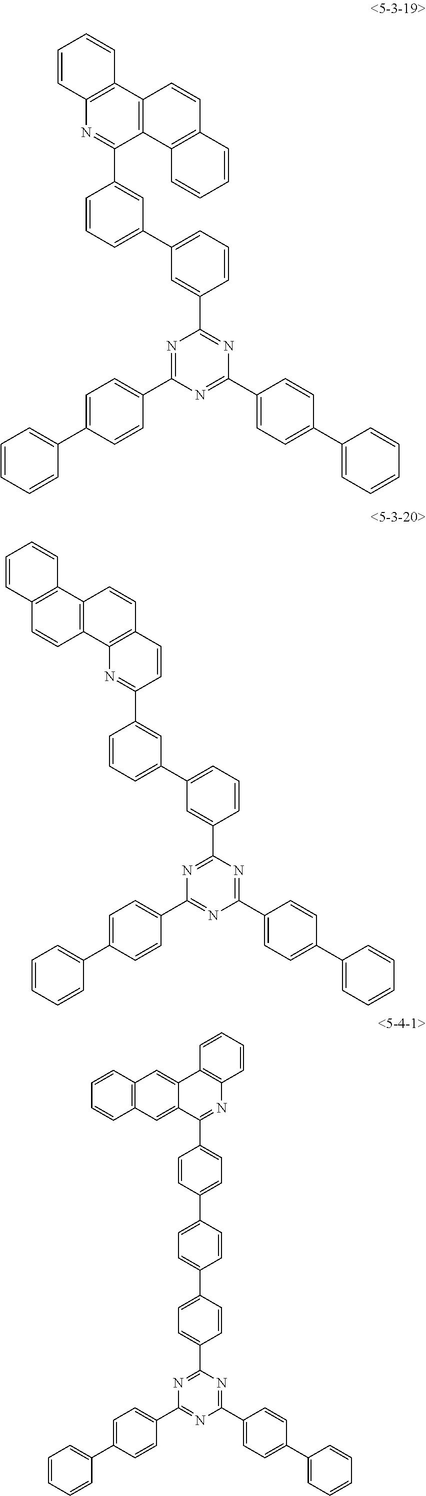 [SMILES: c1ccc(-c2ccc(-c3nc(-c4ccc(-c5ccccc5)cc4)nc(-c4cccc(-c5cccc(-c6nc7ccccc7c7ccc8ccccc8c67)c5)c4)n3)cc2)cc1 ; c1ccc(-c2ccc(-c3nc(-c4ccc(-c5ccccc5)cc4)nc(-c4cccc(-c5cccc(-c6ccc7ccc8c9ccccc9ccc8c7n6)c5)c4)n3)cc2)cc1 ; c1ccc(-c2ccc(-c3nc(-c4ccc(-c5ccccc5)cc4)nc(-c4ccc(-c5ccc(-c6ccc(-c7nc8ccccc8c8cc9ccccc9cc78)cc6)cc5)cc4)n3)cc2)cc1]

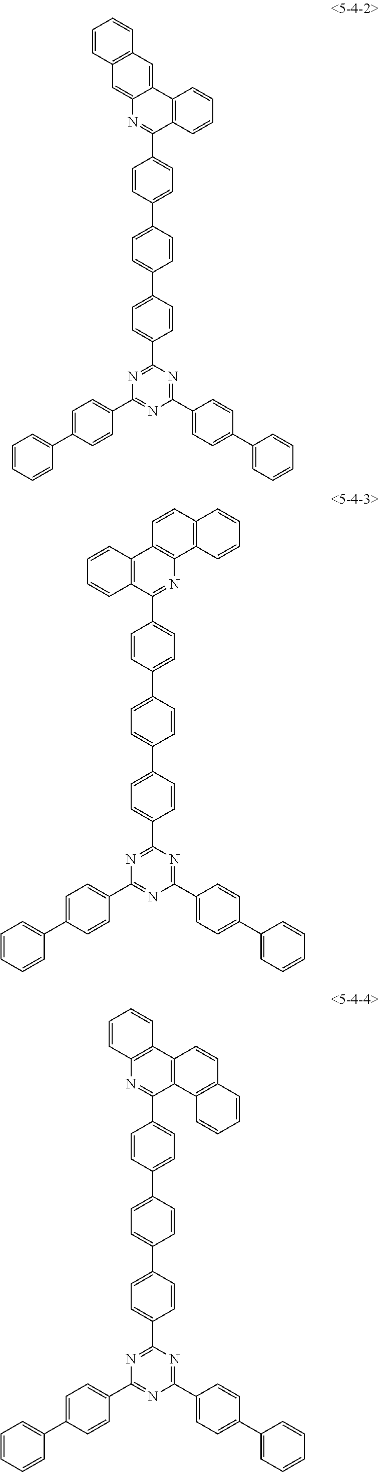 [SMILES: c1ccc(-c2ccc(-c3nc(-c4ccc(-c5ccccc5)cc4)nc(-c4ccc(-c5ccc(-c6ccc(-c7nc8cc9ccccc9cc8c8ccccc78)cc6)cc5)cc4)n3)cc2)cc1 ; c1ccc(-c2ccc(-c3nc(-c4ccc(-c5ccccc5)cc4)nc(-c4ccc(-c5ccc(-c6ccc(-c7nc8c9ccccc9ccc8c8ccccc78)cc6)cc5)cc4)n3)cc2)cc1 ; c1ccc(-c2ccc(-c3nc(-c4ccc(-c5ccccc5)cc4)nc(-c4ccc(-c5ccc(-c6ccc(-c7nc8ccccc8c8ccc9ccccc9c78)cc6)cc5)cc4)n3)cc2)cc1]

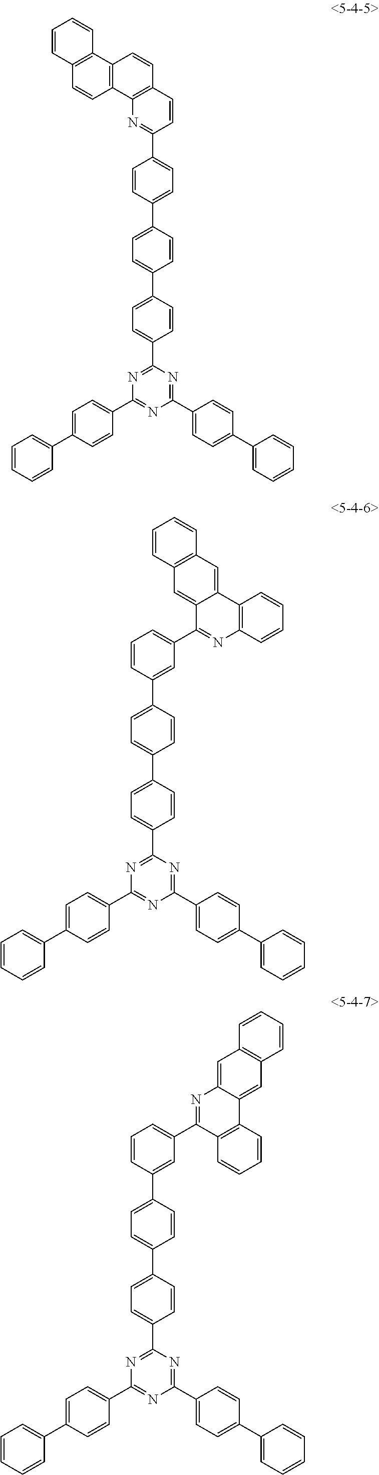 [SMILES: c1ccc(-c2ccc(-c3nc(-c4ccc(-c5ccccc5)cc4)nc(-c4ccc(-c5ccc(-c6ccc(-c7ccc8ccc9c%10ccccc%10ccc9c8n7)cc6)cc5)cc4)n3)cc2)cc1 ; c1ccc(-c2ccc(-c3nc(-c4ccc(-c5ccccc5)cc4)nc(-c4ccc(-c5ccc(-c6cccc(-c7nc8ccccc8c8cc9ccccc9cc78)c6)cc5)cc4)n3)cc2)cc1 ; c1ccc(-c2ccc(-c3nc(-c4ccc(-c5ccccc5)cc4)nc(-c4ccc(-c5ccc(-c6cccc(-c7nc8cc9ccccc9cc8c8ccccc78)c6)cc5)cc4)n3)cc2)cc1]

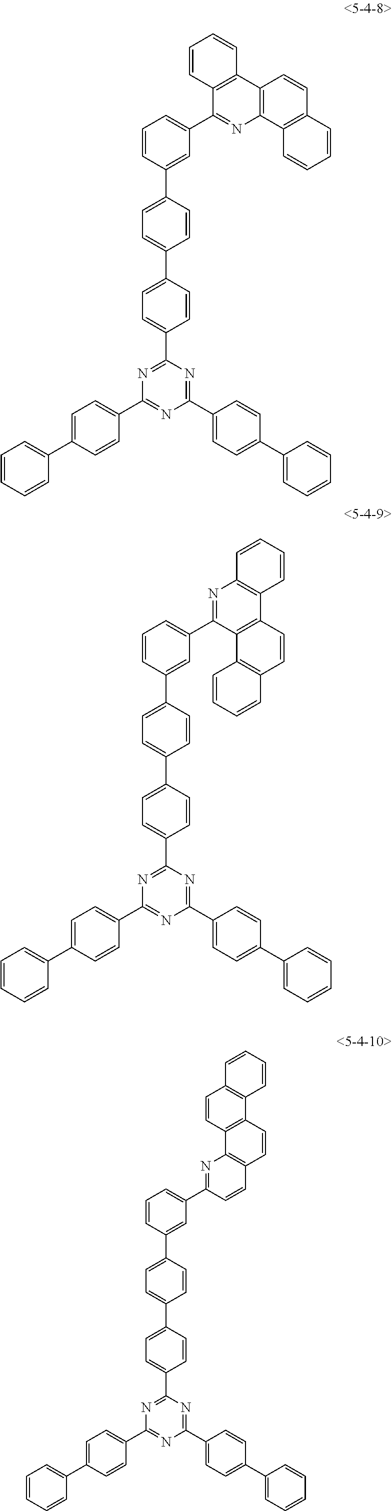 [SMILES: c1ccc(-c2ccc(-c3nc(-c4ccc(-c5ccccc5)cc4)nc(-c4ccc(-c5ccc(-c6cccc(-c7nc8c9ccccc9ccc8c8ccccc78)c6)cc5)cc4)n3)cc2)cc1 ; c1ccc(-c2ccc(-c3nc(-c4ccc(-c5ccccc5)cc4)nc(-c4ccc(-c5ccc(-c6cccc(-c7nc8ccccc8c8ccc9ccccc9c78)c6)cc5)cc4)n3)cc2)cc1 ; c1ccc(-c2ccc(-c3nc(-c4ccc(-c5ccccc5)cc4)nc(-c4ccc(-c5ccc(-c6cccc(-c7ccc8ccc9c%10ccccc%10ccc9c8n7)c6)cc5)cc4)n3)cc2)cc1]

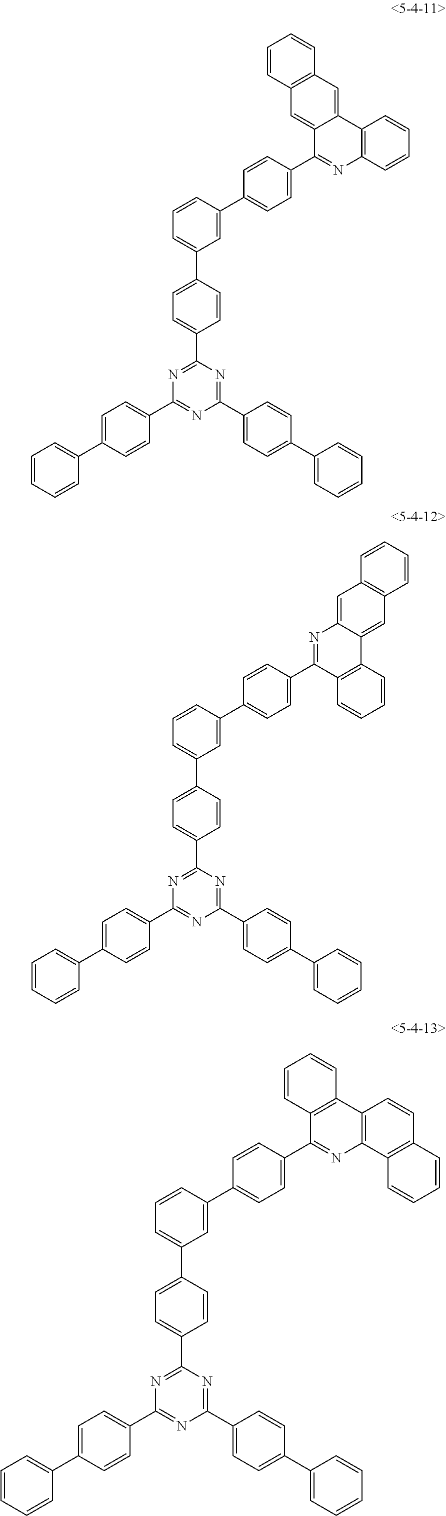 [SMILES: c1ccc(-c2ccc(-c3nc(-c4ccc(-c5ccccc5)cc4)nc(-c4ccc(-c5cccc(-c6ccc(-c7nc8ccccc8c8cc9ccccc9cc78)cc6)c5)cc4)n3)cc2)cc1 ; c1ccc(-c2ccc(-c3nc(-c4ccc(-c5ccccc5)cc4)nc(-c4ccc(-c5cccc(-c6ccc(-c7nc8cc9ccccc9cc8c8ccccc78)cc6)c5)cc4)n3)cc2)cc1 ; c1ccc(-c2ccc(-c3nc(-c4ccc(-c5ccccc5)cc4)nc(-c4ccc(-c5cccc(-c6ccc(-c7nc8c9ccccc9ccc8c8ccccc78)cc6)c5)cc4)n3)cc2)cc1]

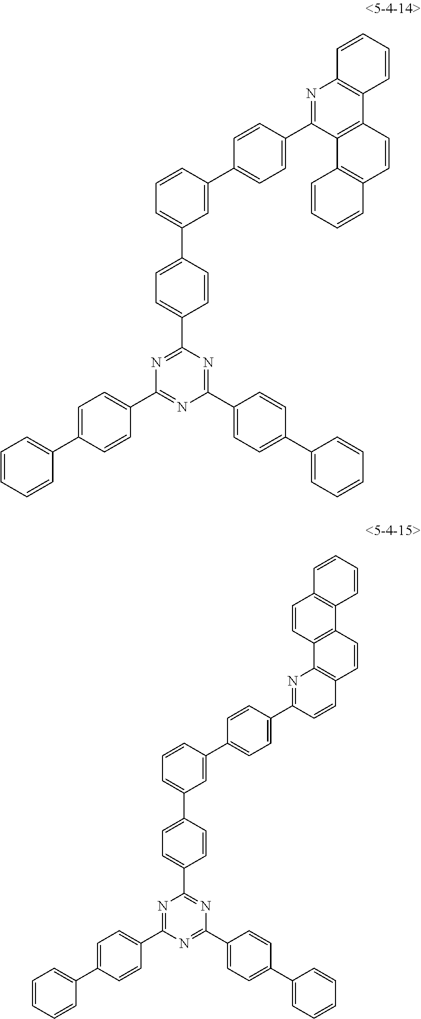 [SMILES: c1ccc(-c2ccc(-c3nc(-c4ccc(-c5ccccc5)cc4)nc(-c4ccc(-c5cccc(-c6ccc(-c7nc8ccccc8c8ccc9ccccc9c78)cc6)c5)cc4)n3)cc2)cc1 ; c1ccc(-c2ccc(-c3nc(-c4ccc(-c5ccccc5)cc4)nc(-c4ccc(-c5cccc(-c6ccc(-c7ccc8ccc9c%10ccccc%10ccc9c8n7)cc6)c5)cc4)n3)cc2)cc1]

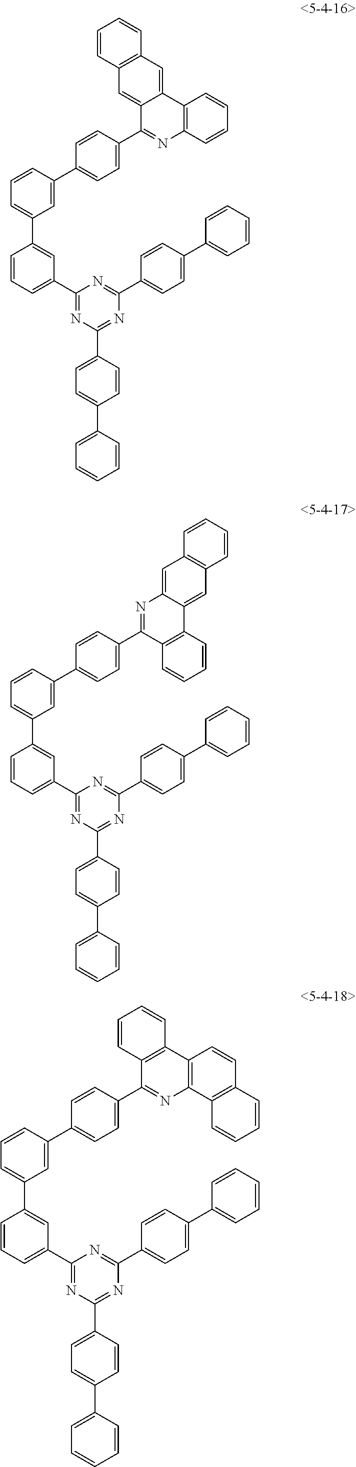 [SMILES: c1ccc(-c2ccc(-c3nc(-c4ccc(-c5ccccc5)cc4)nc(-c4cccc(-c5cccc(-c6ccc(-c7nc8ccccc8c8cc9ccccc9cc78)cc6)c5)c4)n3)cc2)cc1 ; c1ccc(-c2ccc(-c3nc(-c4ccc(-c5ccccc5)cc4)nc(-c4cccc(-c5cccc(-c6ccc(-c7nc8cc9ccccc9cc8c8ccccc78)cc6)c5)c4)n3)cc2)cc1 ; c1ccc(-c2ccc(-c3nc(-c4ccc(-c5ccccc5)cc4)nc(-c4cccc(-c5cccc(-c6ccc(-c7nc8c9ccccc9ccc8c8ccccc78)cc6)c5)c4)n3)cc2)cc1]

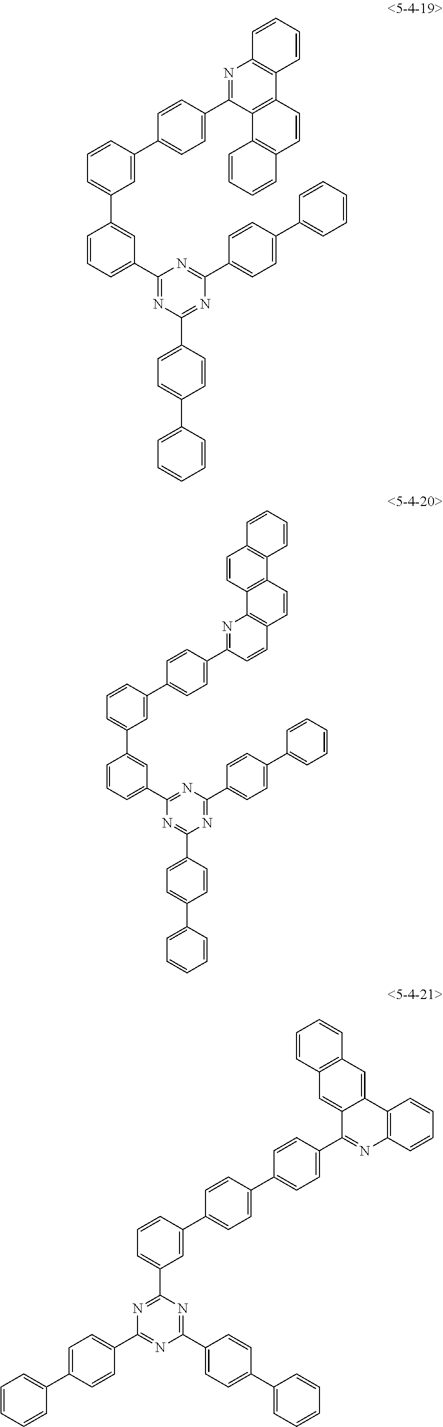 [SMILES: c1ccc(-c2ccc(-c3nc(-c4ccc(-c5ccccc5)cc4)nc(-c4cccc(-c5cccc(-c6ccc(-c7nc8ccccc8c8ccc9ccccc9c78)cc6)c5)c4)n3)cc2)cc1 ; c1ccc(-c2ccc(-c3nc(-c4ccc(-c5ccccc5)cc4)nc(-c4cccc(-c5cccc(-c6ccc(-c7ccc8ccc9c%10ccccc%10ccc9c8n7)cc6)c5)c4)n3)cc2)cc1 ; c1ccc(-c2ccc(-c3nc(-c4ccc(-c5ccccc5)cc4)nc(-c4cccc(-c5ccc(-c6ccc(-c7nc8ccccc8c8cc9ccccc9cc78)cc6)cc5)c4)n3)cc2)cc1]

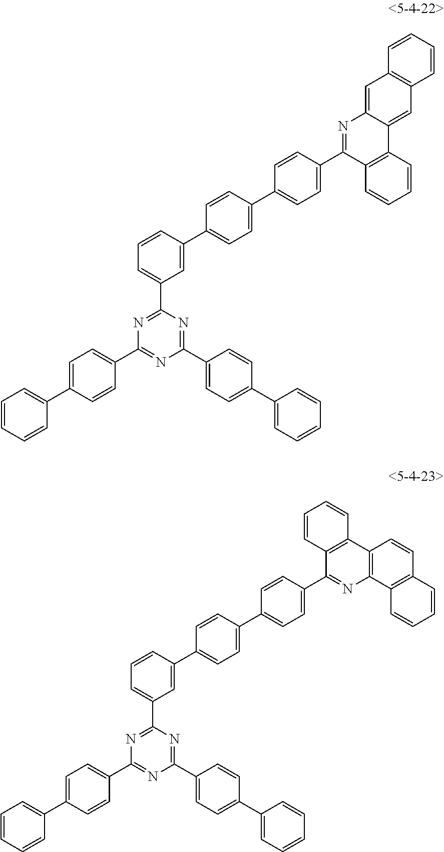 [SMILES: c1ccc(-c2ccc(-c3nc(-c4ccc(-c5ccccc5)cc4)nc(-c4cccc(-c5ccc(-c6ccc(-c7nc8cc9ccccc9cc8c8ccccc78)cc6)cc5)c4)n3)cc2)cc1 ; c1ccc(-c2ccc(-c3nc(-c4ccc(-c5ccccc5)cc4)nc(-c4cccc(-c5ccc(-c6ccc(-c7nc8c9ccccc9ccc8c8ccccc78)cc6)cc5)c4)n3)cc2)cc1]

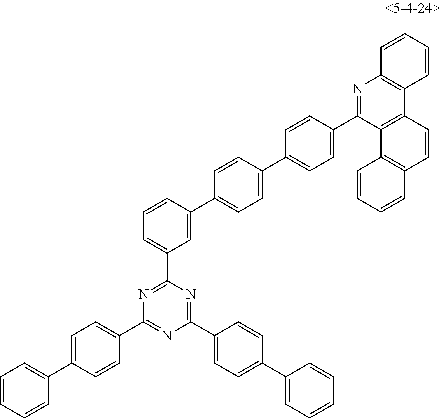 [SMILES: c1ccc(-c2ccc(-c3nc(-c4ccc(-c5ccccc5)cc4)nc(-c4cccc(-c5ccc(-c6ccc(-c7nc8ccccc8c8ccc9ccccc9c78)cc6)cc5)c4)n3)cc2)cc1]

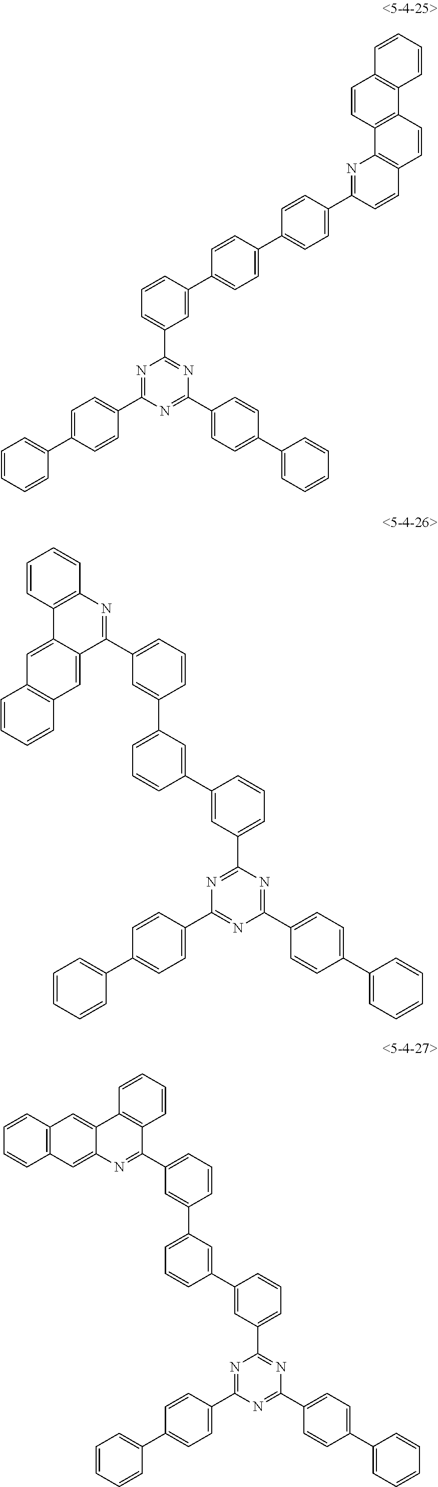 [SMILES: c1ccc(-c2ccc(-c3nc(-c4ccc(-c5ccccc5)cc4)nc(-c4cccc(-c5ccc(-c6ccc(-c7ccc8ccc9c%10ccccc%10ccc9c8n7)cc6)cc5)c4)n3)cc2)cc1 ; c1ccc(-c2ccc(-c3nc(-c4ccc(-c5ccccc5)cc4)nc(-c4cccc(-c5cccc(-c6cccc(-c7nc8ccccc8c8cc9ccccc9cc78)c6)c5)c4)n3)cc2)cc1 ; c1ccc(-c2ccc(-c3nc(-c4ccc(-c5ccccc5)cc4)nc(-c4cccc(-c5cccc(-c6cccc(-c7nc8cc9ccccc9cc8c8ccccc78)c6)c5)c4)n3)cc2)cc1]

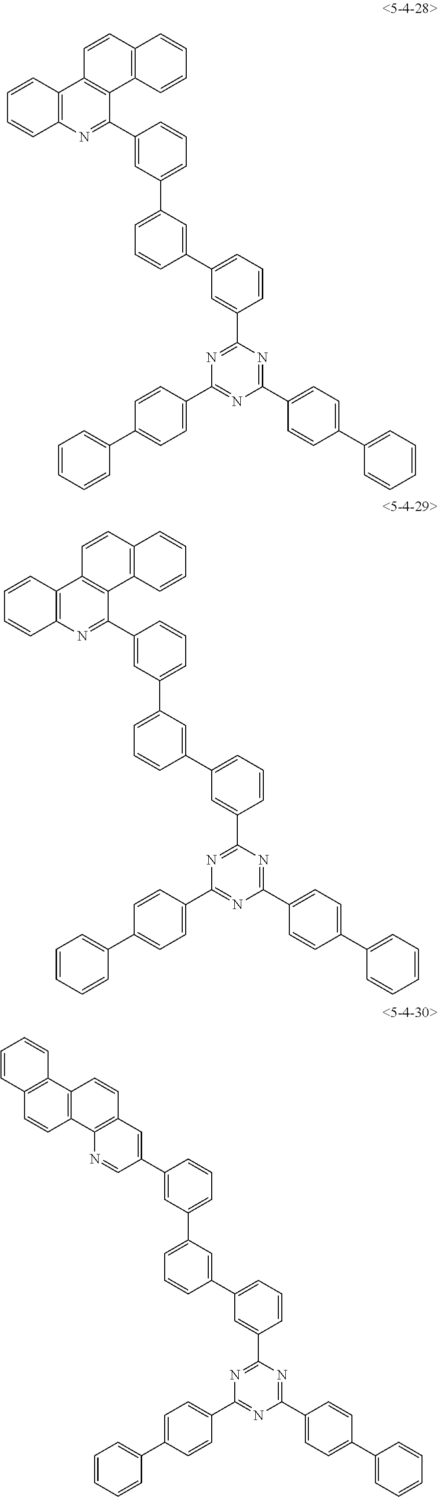 [SMILES: c1ccc(-c2ccc(-c3nc(-c4ccc(-c5ccccc5)cc4)nc(-c4cccc(-c5cccc(-c6cccc(-c7nc8ccccc8c8ccc9ccccc9c78)c6)c5)c4)n3)cc2)cc1 ; c1ccc(-c2ccc(-c3nc(-c4ccc(-c5ccccc5)cc4)nc(-c4cccc(-c5cccc(-c6cccc(-c7nc8ccccc8c8ccc9ccccc9c78)c6)c5)c4)n3)cc2)cc1 ; c1ccc(-c2ccc(-c3nc(-c4ccc(-c5ccccc5)cc4)nc(-c4cccc(-c5cccc(-c6cccc(-c7cnc8c(ccc9c%10ccccc%10ccc98)c7)c6)c5)c4)n3)cc2)cc1]

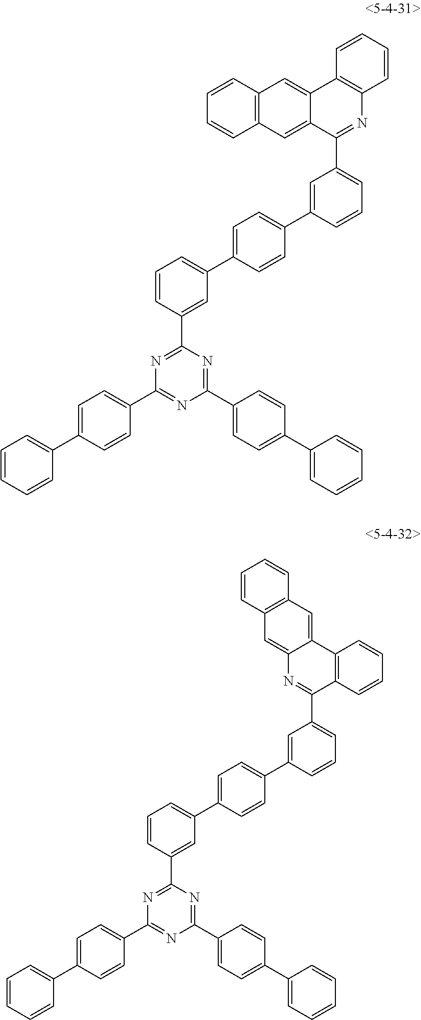 [SMILES: c1ccc(-c2ccc(-c3nc(-c4ccc(-c5ccccc5)cc4)nc(-c4cccc(-c5ccc(-c6cccc(-c7nc8ccccc8c8cc9ccccc9cc78)c6)cc5)c4)n3)cc2)cc1 ; c1ccc(-c2ccc(-c3nc(-c4ccc(-c5ccccc5)cc4)nc(-c4cccc(-c5ccc(-c6cccc(-c7nc8cc9ccccc9cc8c8ccccc78)c6)cc5)c4)n3)cc2)cc1]

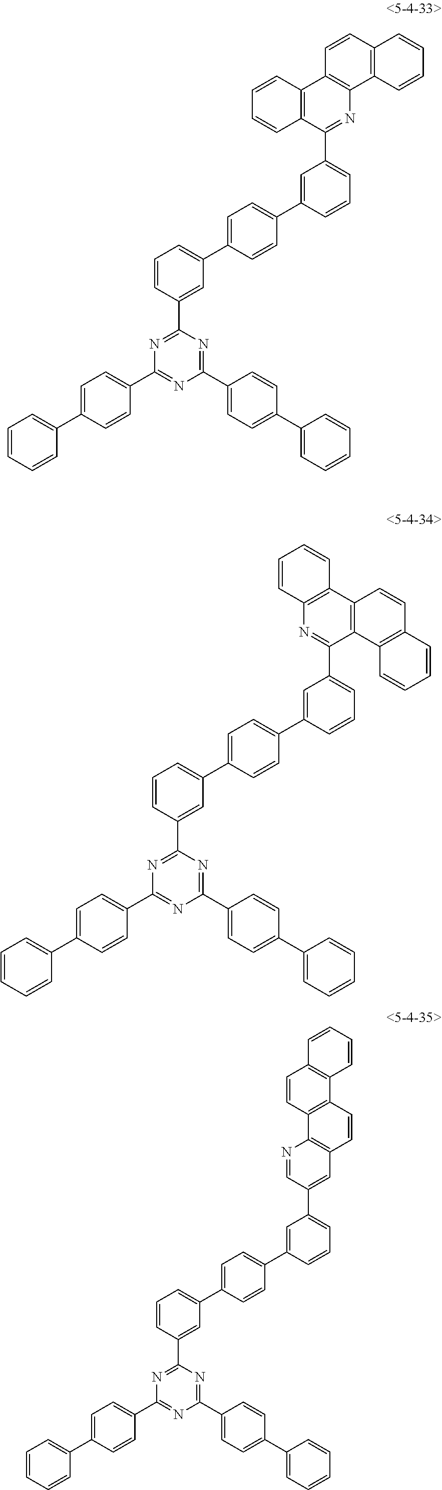

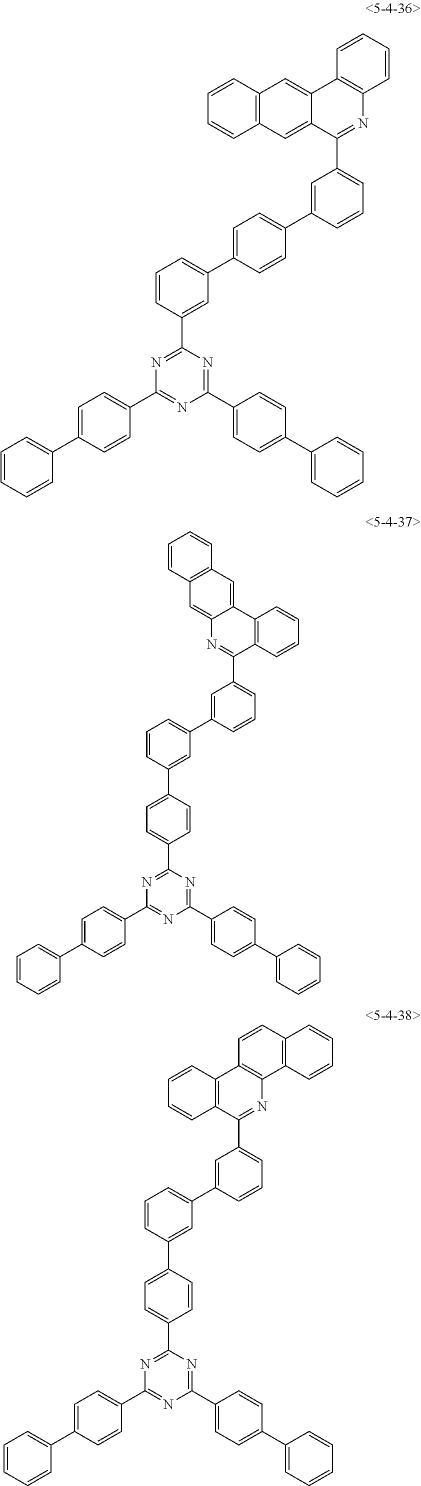

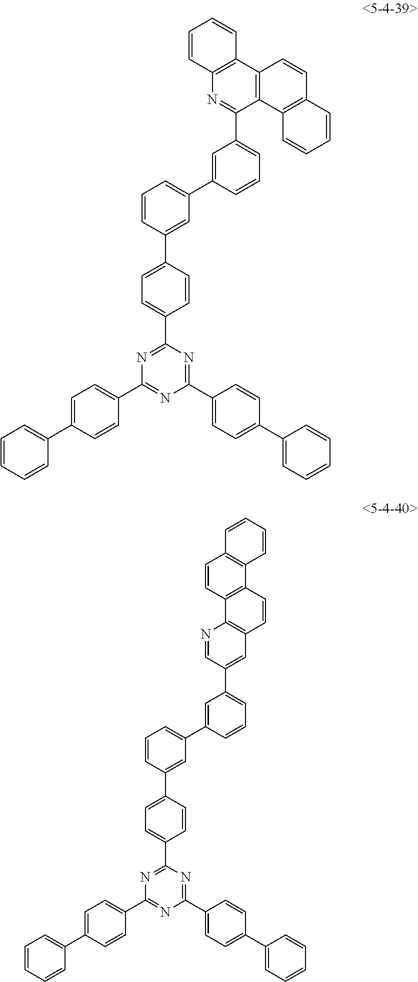 [SMILES: c1ccc(-c2ccc(-c3nc(-c4ccc(-c5ccccc5)cc4)nc(-c4ccc(-c5cccc(-c6cccc(-c7nc8ccccc8c8ccc9ccccc9c78)c6)c5)cc4)n3)cc2)cc1 ; c1ccc(-c2ccc(-c3nc(-c4ccc(-c5ccccc5)cc4)nc(-c4ccc(-c5cccc(-c6cccc(-c7cnc8c(ccc9c%10ccccc%10ccc98)c7)c6)c5)cc4)n3)cc2)cc1]

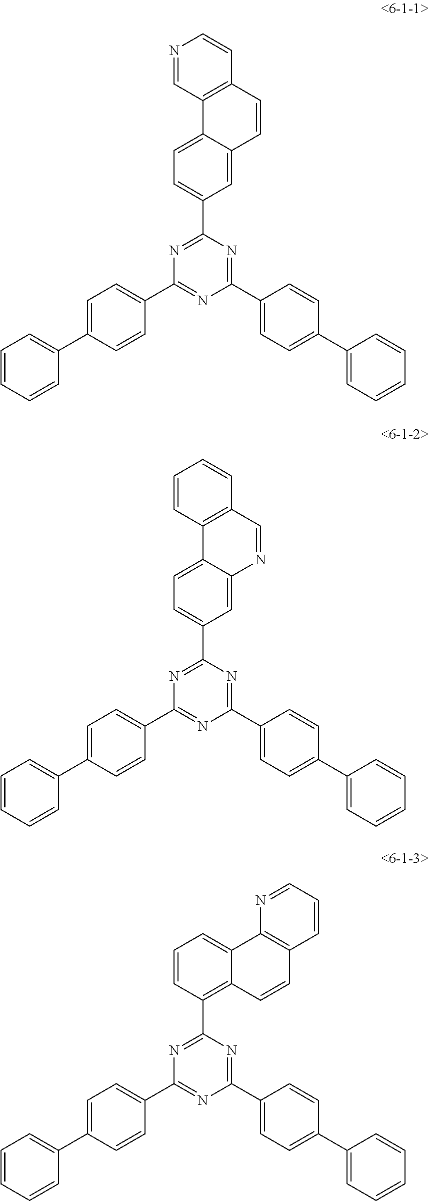 [SMILES: c1ccc(-c2ccc(-c3nc(-c4ccc(-c5ccccc5)cc4)nc(-c4ccc5c(ccc6ccncc65)c4)n3)cc2)cc1 ; c1ccc(-c2ccc(-c3nc(-c4ccc(-c5ccccc5)cc4)nc(-c4ccc5c(c4)ncc4ccccc45)n3)cc2)cc1 ; c1ccc(-c2ccc(-c3nc(-c4ccc(-c5ccccc5)cc4)nc(-c4cccc5c4ccc4cccnc45)n3)cc2)cc1]

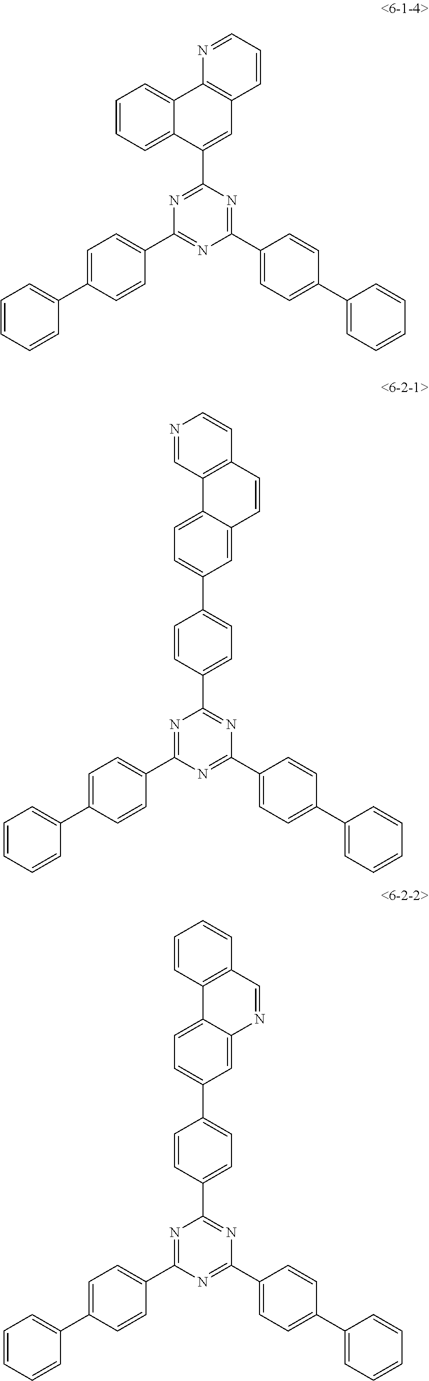 [SMILES: c1ccc(-c2ccc(-c3nc(-c4ccc(-c5ccccc5)cc4)nc(-c4cc5cccnc5c5ccccc45)n3)cc2)cc1 ; c1ccc(-c2ccc(-c3nc(-c4ccc(-c5ccccc5)cc4)nc(-c4ccc(-c5ccc6c(ccc7ccncc76)c5)cc4)n3)cc2)cc1 ; c1ccc(-c2ccc(-c3nc(-c4ccc(-c5ccccc5)cc4)nc(-c4ccc(-c5ccc6c(c5)ncc5ccccc56)cc4)n3)cc2)cc1]

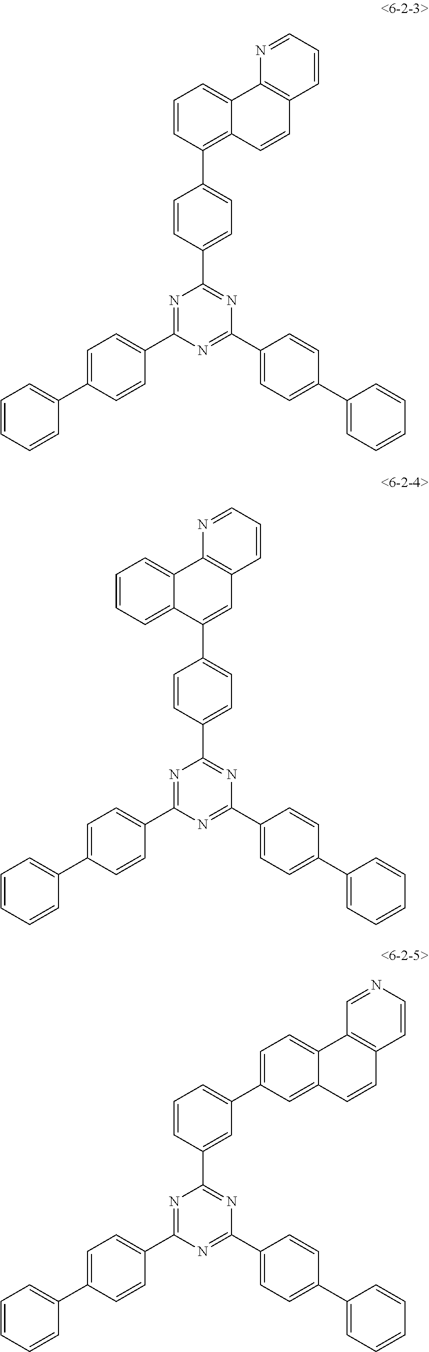 [SMILES: c1ccc(-c2ccc(-c3nc(-c4ccc(-c5ccccc5)cc4)nc(-c4ccc(-c5cccc6c5ccc5cccnc56)cc4)n3)cc2)cc1 ; c1ccc(-c2ccc(-c3nc(-c4ccc(-c5ccccc5)cc4)nc(-c4ccc(-c5cc6cccnc6c6ccccc56)cc4)n3)cc2)cc1 ; c1ccc(-c2ccc(-c3nc(-c4ccc(-c5ccccc5)cc4)nc(-c4cccc(-c5ccc6c(ccc7ccncc76)c5)c4)n3)cc2)cc1]

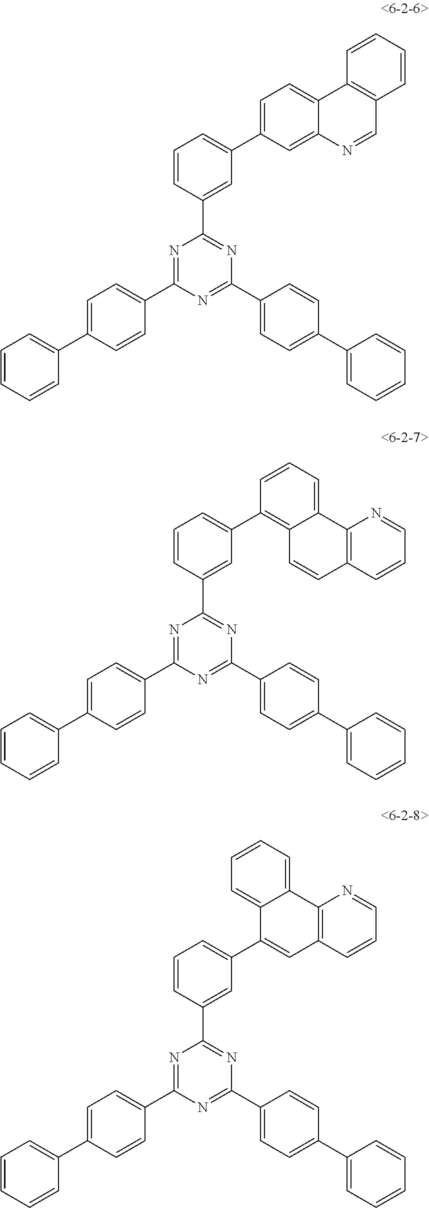 [SMILES: c1ccc(-c2ccc(-c3nc(-c4ccc(-c5ccccc5)cc4)nc(-c4cccc(-c5ccc6c(c5)ncc5ccccc56)c4)n3)cc2)cc1 ; c1ccc(-c2ccc(-c3nc(-c4ccc(-c5ccccc5)cc4)nc(-c4cccc(-c5cccc6c5ccc5cccnc56)c4)n3)cc2)cc1 ; c1ccc(-c2ccc(-c3nc(-c4ccc(-c5ccccc5)cc4)nc(-c4cccc(-c5cc6cccnc6c6ccccc56)c4)n3)cc2)cc1]

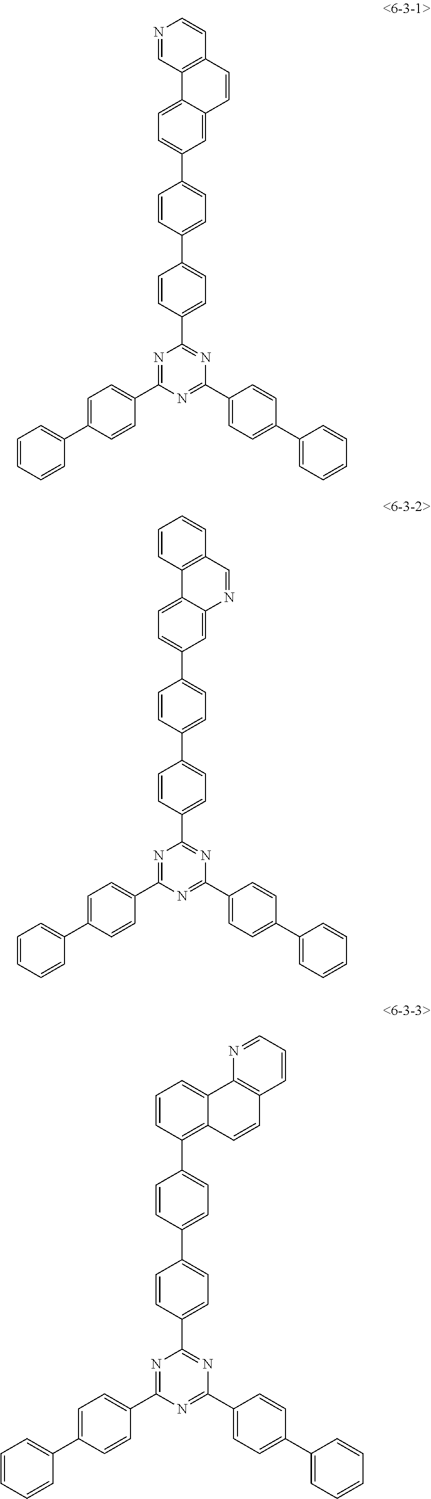 [SMILES: c1ccc(-c2ccc(-c3nc(-c4ccc(-c5ccccc5)cc4)nc(-c4ccc(-c5ccc(-c6ccc7c(ccc8ccncc87)c6)cc5)cc4)n3)cc2)cc1 ; c1ccc(-c2ccc(-c3nc(-c4ccc(-c5ccccc5)cc4)nc(-c4ccc(-c5ccc(-c6ccc7c(c6)ncc6ccccc67)cc5)cc4)n3)cc2)cc1 ; c1ccc(-c2ccc(-c3nc(-c4ccc(-c5ccccc5)cc4)nc(-c4ccc(-c5ccc(-c6cccc7c6ccc6cccnc67)cc5)cc4)n3)cc2)cc1]

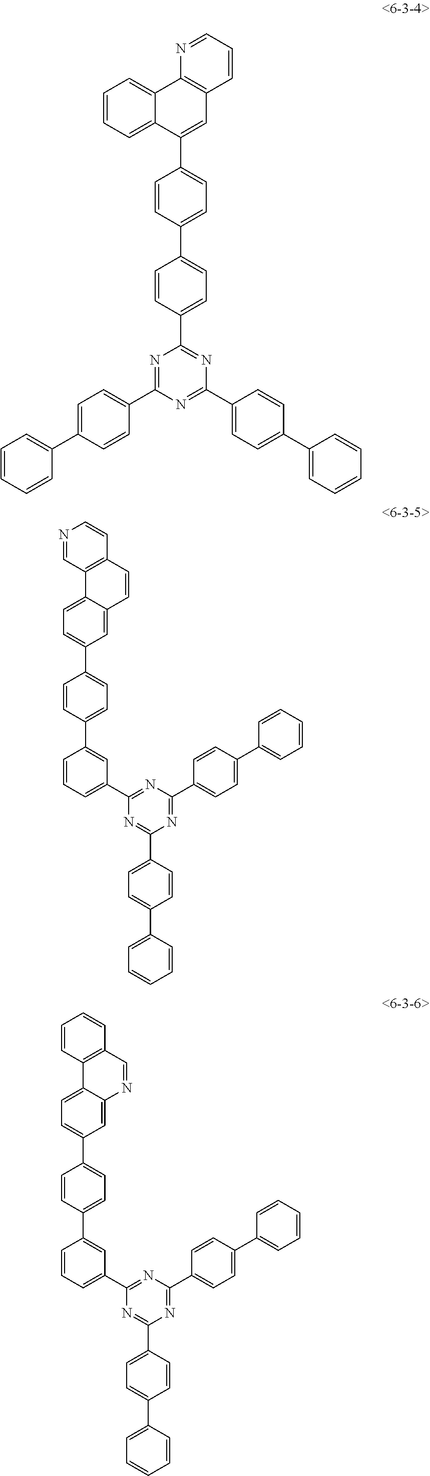 [SMILES: c1ccc(-c2ccc(-c3nc(-c4ccc(-c5ccccc5)cc4)nc(-c4ccc(-c5ccc(-c6cc7cccnc7c7ccccc67)cc5)cc4)n3)cc2)cc1 ; c1ccc(-c2ccc(-c3nc(-c4ccc(-c5ccccc5)cc4)nc(-c4cccc(-c5ccc(-c6ccc7c(ccc8ccncc87)c6)cc5)c4)n3)cc2)cc1 ; c1ccc(-c2ccc(-c3nc(-c4ccc(-c5ccccc5)cc4)nc(-c4cccc(-c5ccc(-c6ccc7c(c6)ncc6ccccc67)cc5)c4)n3)cc2)cc1]

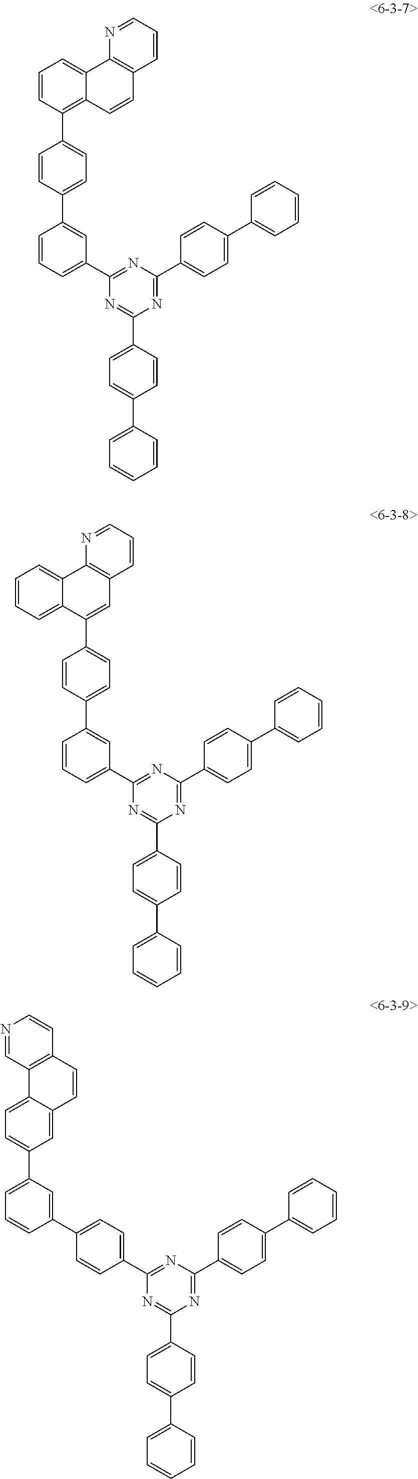 [SMILES: c1ccc(-c2ccc(-c3nc(-c4ccc(-c5ccccc5)cc4)nc(-c4cccc(-c5ccc(-c6cccc7c6ccc6cccnc67)cc5)c4)n3)cc2)cc1 ; c1ccc(-c2ccc(-c3nc(-c4ccc(-c5ccccc5)cc4)nc(-c4cccc(-c5ccc(-c6cc7cccnc7c7ccccc67)cc5)c4)n3)cc2)cc1 ; c1ccc(-c2ccc(-c3nc(-c4ccc(-c5ccccc5)cc4)nc(-c4ccc(-c5cccc(-c6ccc7c(ccc8ccncc87)c6)c5)cc4)n3)cc2)cc1]

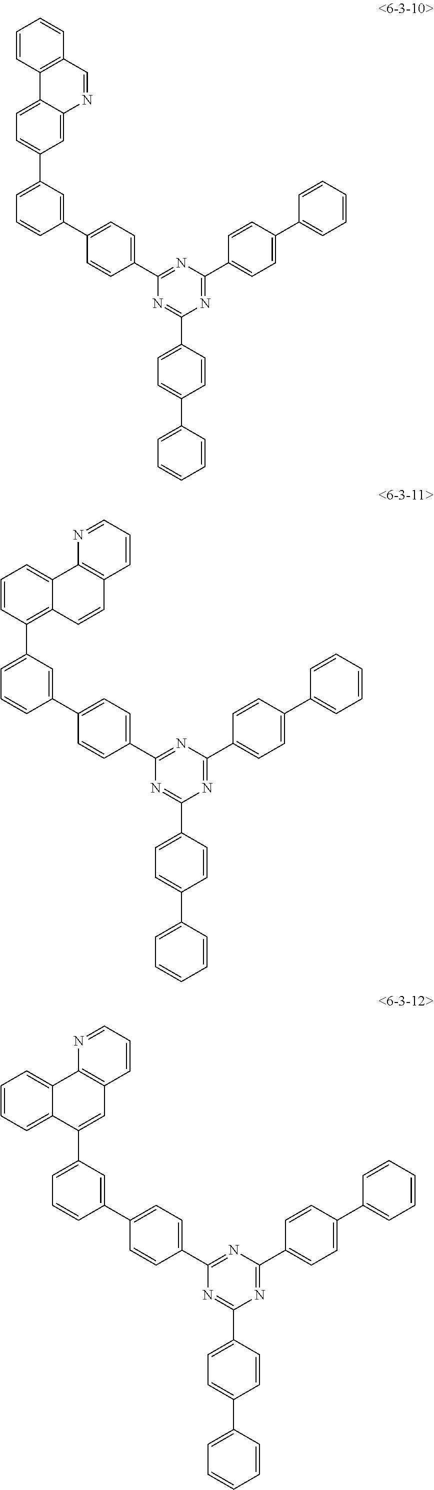 [SMILES: c1ccc(-c2ccc(-c3nc(-c4ccc(-c5ccccc5)cc4)nc(-c4ccc(-c5cccc(-c6ccc7c(c6)ncc6ccccc67)c5)cc4)n3)cc2)cc1 ; c1ccc(-c2ccc(-c3nc(-c4ccc(-c5ccccc5)cc4)nc(-c4ccc(-c5cccc(-c6cccc7c6ccc6cccnc67)c5)cc4)n3)cc2)cc1 ; c1ccc(-c2ccc(-c3nc(-c4ccc(-c5ccccc5)cc4)nc(-c4ccc(-c5cccc(-c6cc7cccnc7c7ccccc67)c5)cc4)n3)cc2)cc1]

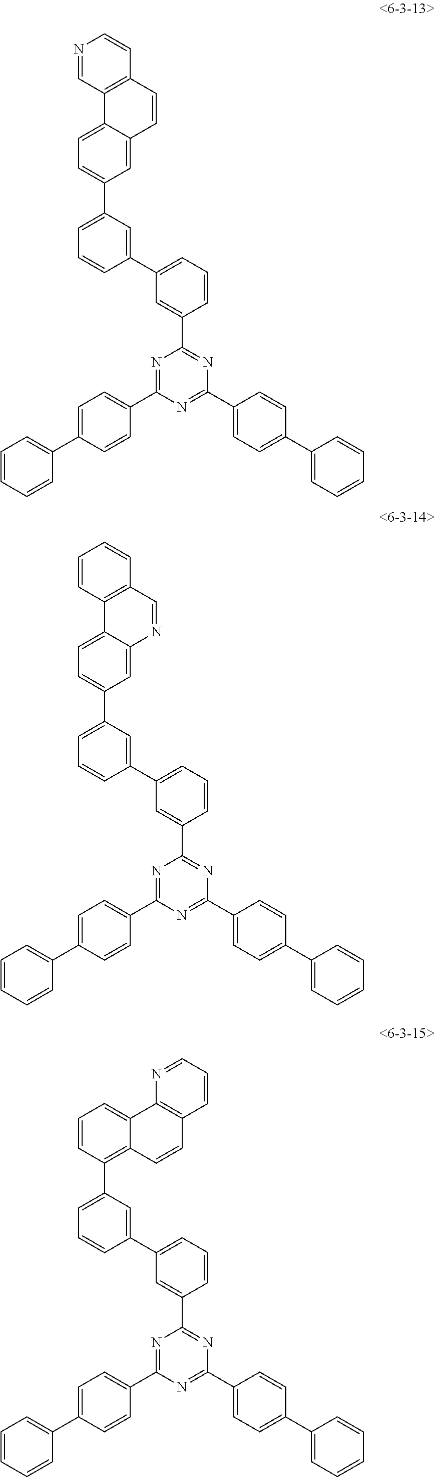 [SMILES: c1ccc(-c2ccc(-c3nc(-c4ccc(-c5ccccc5)cc4)nc(-c4cccc(-c5cccc(-c6ccc7c(ccc8ccncc87)c6)c5)c4)n3)cc2)cc1 ; c1ccc(-c2ccc(-c3nc(-c4ccc(-c5ccccc5)cc4)nc(-c4cccc(-c5cccc(-c6ccc7c(c6)ncc6ccccc67)c5)c4)n3)cc2)cc1 ; c1ccc(-c2ccc(-c3nc(-c4ccc(-c5ccccc5)cc4)nc(-c4cccc(-c5cccc(-c6cccc7c6ccc6cccnc67)c5)c4)n3)cc2)cc1]

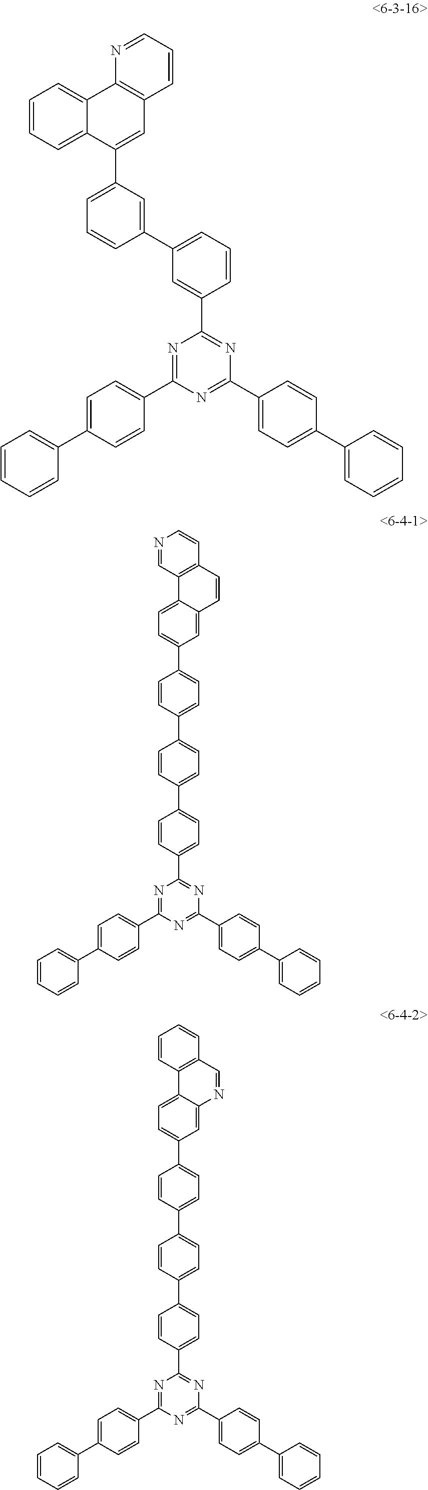 [SMILES: c1ccc(-c2ccc(-c3nc(-c4ccc(-c5ccccc5)cc4)nc(-c4cccc(-c5cccc(-c6cc7cccnc7c7ccccc67)c5)c4)n3)cc2)cc1 ; c1ccc(-c2ccc(-c3nc(-c4ccc(-c5ccccc5)cc4)nc(-c4ccc(-c5ccc(-c6ccc(-c7ccc8c(ccc9ccncc98)c7)cc6)cc5)cc4)n3)cc2)cc1 ; c1ccc(-c2ccc(-c3nc(-c4ccc(-c5ccccc5)cc4)nc(-c4ccc(-c5ccc(-c6ccc(-c7ccc8c(c7)ncc7ccccc78)cc6)cc5)cc4)n3)cc2)cc1]

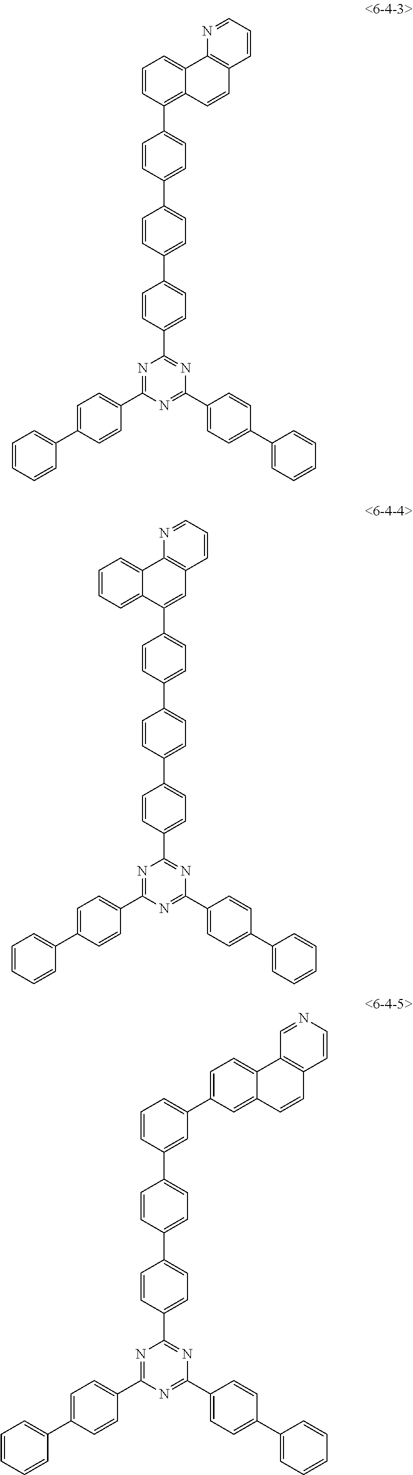 [SMILES: c1ccc(-c2ccc(-c3nc(-c4ccc(-c5ccccc5)cc4)nc(-c4ccc(-c5ccc(-c6ccc(-c7cccc8c7ccc7cccnc78)cc6)cc5)cc4)n3)cc2)cc1 ; c1ccc(-c2ccc(-c3nc(-c4ccc(-c5ccccc5)cc4)nc(-c4ccc(-c5ccc(-c6ccc(-c7cc8cccnc8c8ccccc78)cc6)cc5)cc4)n3)cc2)cc1 ; c1ccc(-c2ccc(-c3nc(-c4ccc(-c5ccccc5)cc4)nc(-c4ccc(-c5ccc(-c6cccc(-c7ccc8c(ccc9ccncc98)c7)c6)cc5)cc4)n3)cc2)cc1]

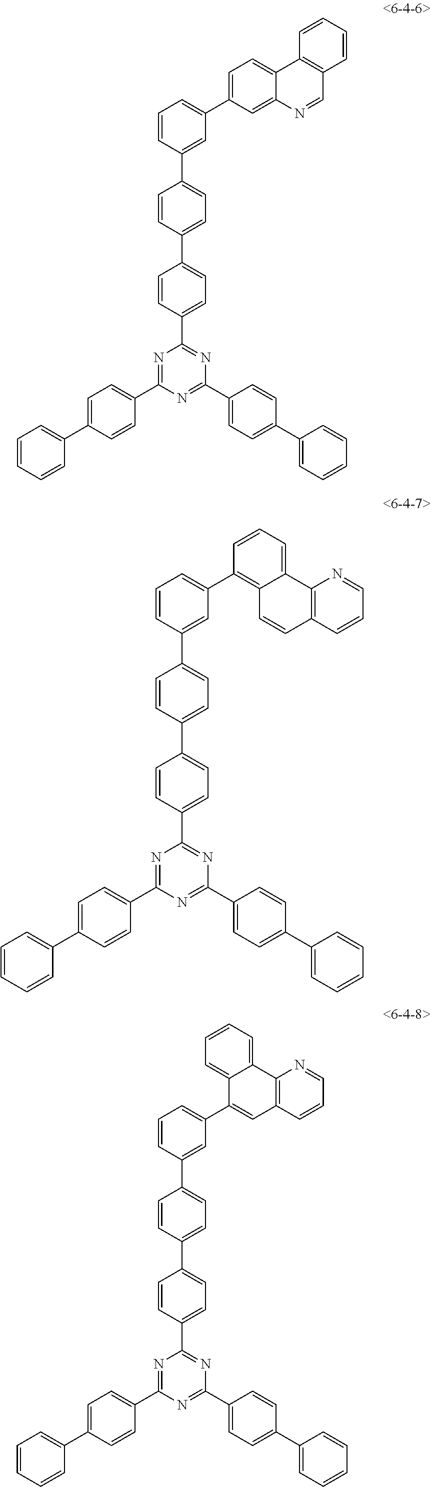 [SMILES: c1ccc(-c2ccc(-c3nc(-c4ccc(-c5ccccc5)cc4)nc(-c4ccc(-c5ccc(-c6cccc(-c7ccc8c(c7)ncc7ccccc78)c6)cc5)cc4)n3)cc2)cc1 ; c1ccc(-c2ccc(-c3nc(-c4ccc(-c5ccccc5)cc4)nc(-c4ccc(-c5ccc(-c6cccc(-c7cccc8c7ccc7cccnc78)c6)cc5)cc4)n3)cc2)cc1 ; c1ccc(-c2ccc(-c3nc(-c4ccc(-c5ccccc5)cc4)nc(-c4ccc(-c5ccc(-c6cccc(-c7cc8cccnc8c8ccccc78)c6)cc5)cc4)n3)cc2)cc1]

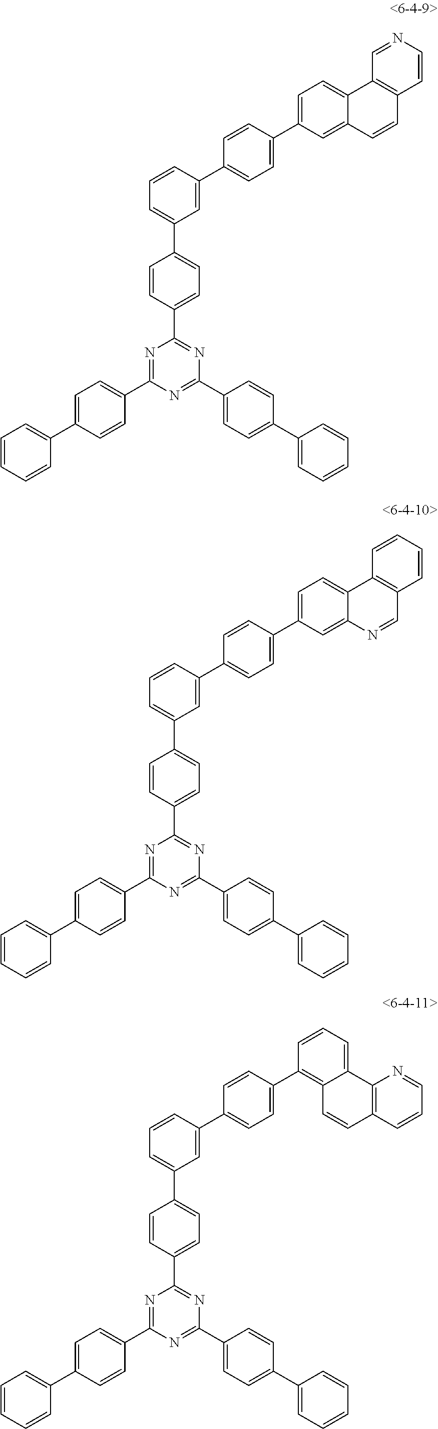 [SMILES: c1ccc(-c2ccc(-c3nc(-c4ccc(-c5ccccc5)cc4)nc(-c4ccc(-c5cccc(-c6ccc(-c7ccc8c(ccc9ccncc98)c7)cc6)c5)cc4)n3)cc2)cc1 ; c1ccc(-c2ccc(-c3nc(-c4ccc(-c5ccccc5)cc4)nc(-c4ccc(-c5cccc(-c6ccc(-c7ccc8c(c7)ncc7ccccc78)cc6)c5)cc4)n3)cc2)cc1 ; c1ccc(-c2ccc(-c3nc(-c4ccc(-c5ccccc5)cc4)nc(-c4ccc(-c5cccc(-c6ccc(-c7cccc8c7ccc7cccnc78)cc6)c5)cc4)n3)cc2)cc1]

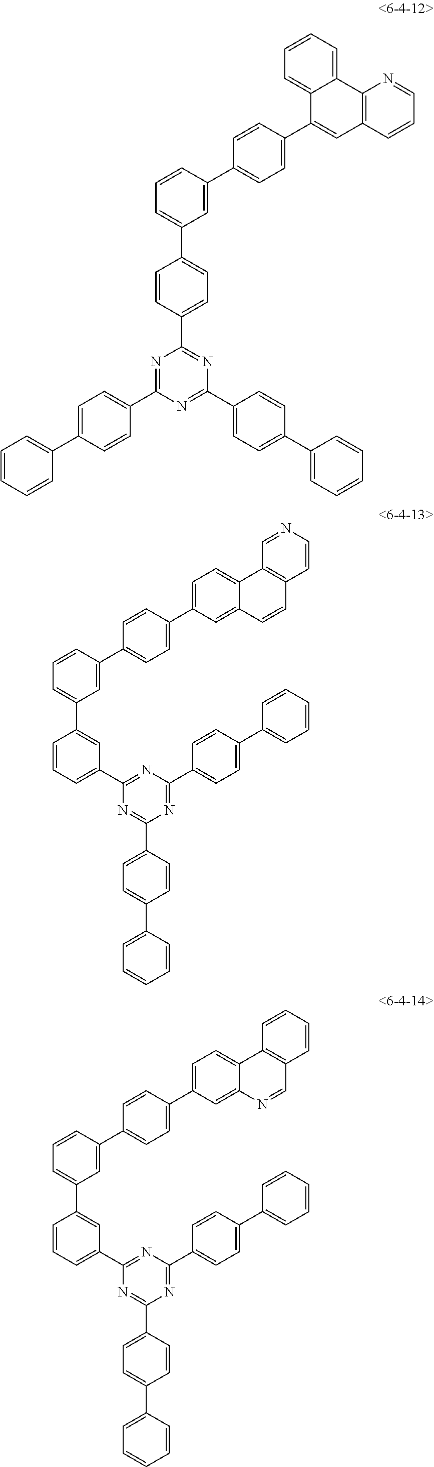 [SMILES: c1ccc(-c2ccc(-c3nc(-c4ccc(-c5ccccc5)cc4)nc(-c4ccc(-c5cccc(-c6ccc(-c7cc8cccnc8c8ccccc78)cc6)c5)cc4)n3)cc2)cc1 ; c1ccc(-c2ccc(-c3nc(-c4ccc(-c5ccccc5)cc4)nc(-c4cccc(-c5cccc(-c6ccc(-c7ccc8c(ccc9ccncc98)c7)cc6)c5)c4)n3)cc2)cc1 ; c1ccc(-c2ccc(-c3nc(-c4ccc(-c5ccccc5)cc4)nc(-c4cccc(-c5cccc(-c6ccc(-c7ccc8c(c7)ncc7ccccc78)cc6)c5)c4)n3)cc2)cc1]

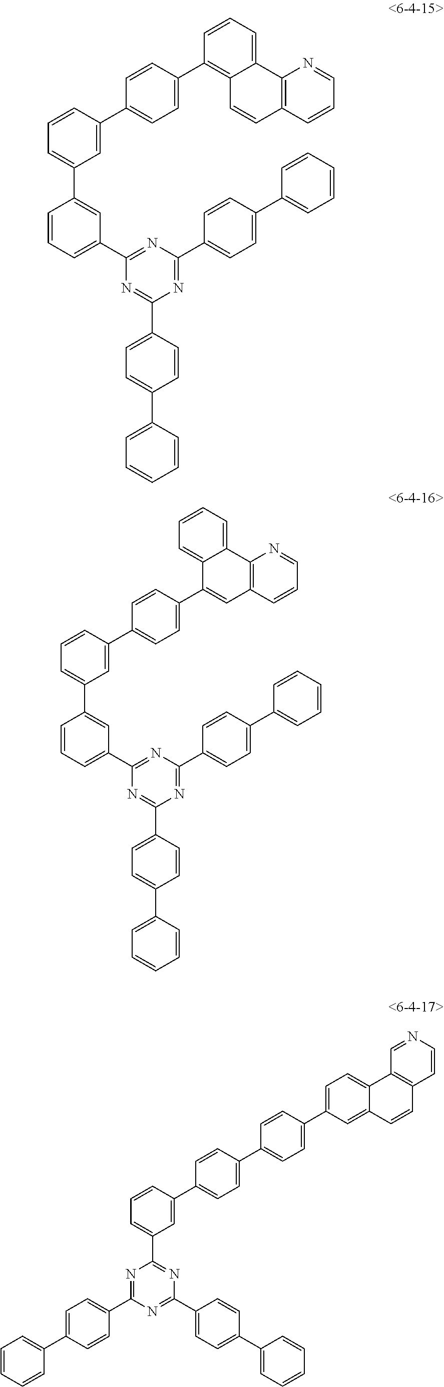 [SMILES: c1ccc(-c2ccc(-c3nc(-c4ccc(-c5ccccc5)cc4)nc(-c4cccc(-c5cccc(-c6ccc(-c7cccc8c7ccc7cccnc78)cc6)c5)c4)n3)cc2)cc1 ; c1ccc(-c2ccc(-c3nc(-c4ccc(-c5ccccc5)cc4)nc(-c4cccc(-c5cccc(-c6ccc(-c7cc8cccnc8c8ccccc78)cc6)c5)c4)n3)cc2)cc1 ; c1ccc(-c2ccc(-c3nc(-c4ccc(-c5ccccc5)cc4)nc(-c4cccc(-c5ccc(-c6ccc(-c7ccc8c(ccc9ccncc98)c7)cc6)cc5)c4)n3)cc2)cc1]

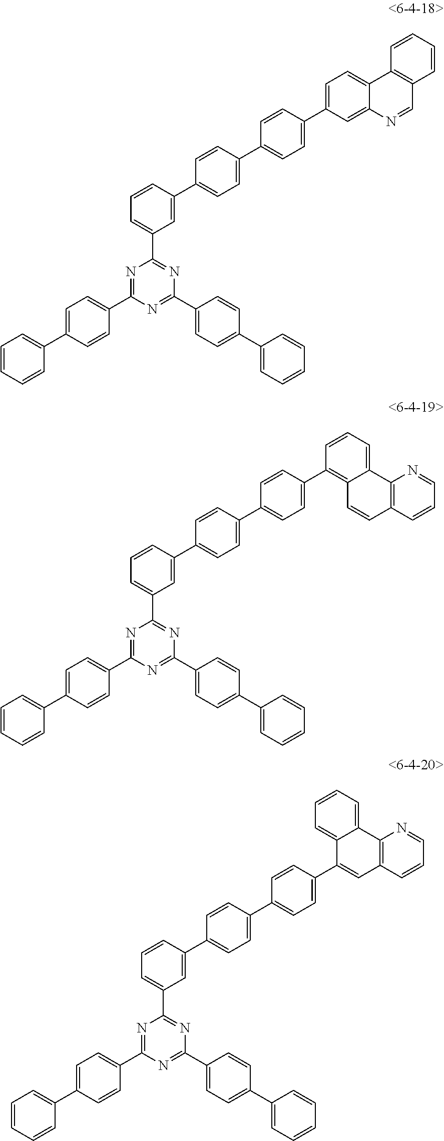 [SMILES: c1ccc(-c2ccc(-c3nc(-c4ccc(-c5ccccc5)cc4)nc(-c4cccc(-c5ccc(-c6ccc(-c7ccc8c(c7)ncc7ccccc78)cc6)cc5)c4)n3)cc2)cc1 ; c1ccc(-c2ccc(-c3nc(-c4ccc(-c5ccccc5)cc4)nc(-c4cccc(-c5ccc(-c6ccc(-c7cccc8c7ccc7cccnc78)cc6)cc5)c4)n3)cc2)cc1 ; c1ccc(-c2ccc(-c3nc(-c4ccc(-c5ccccc5)cc4)nc(-c4cccc(-c5ccc(-c6ccc(-c7cc8cccnc8c8ccccc78)cc6)cc5)c4)n3)cc2)cc1]

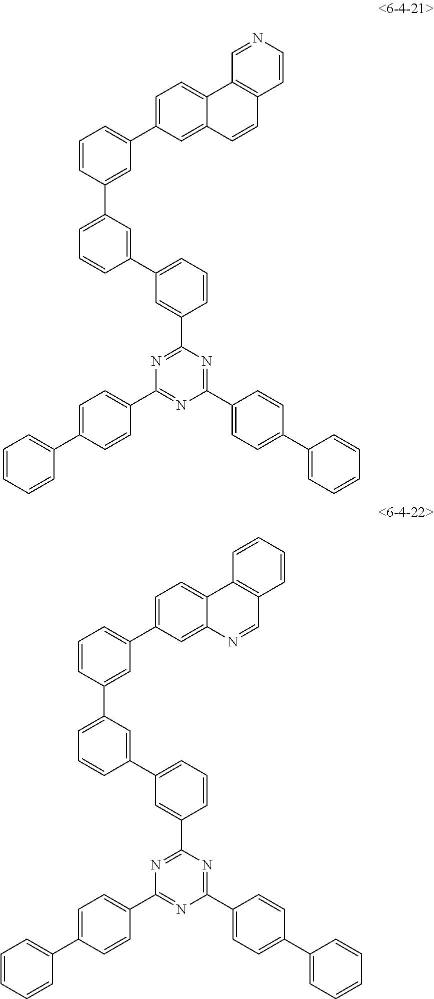 [SMILES: c1ccc(-c2ccc(-c3nc(-c4ccc(-c5ccccc5)cc4)nc(-c4cccc(-c5cccc(-c6cccc(-c7ccc8c(ccc9ccncc98)c7)c6)c5)c4)n3)cc2)cc1 ; c1ccc(-c2ccc(-c3nc(-c4ccc(-c5ccccc5)cc4)nc(-c4cccc(-c5cccc(-c6cccc(-c7ccc8c(c7)ncc7ccccc78)c6)c5)c4)n3)cc2)cc1]

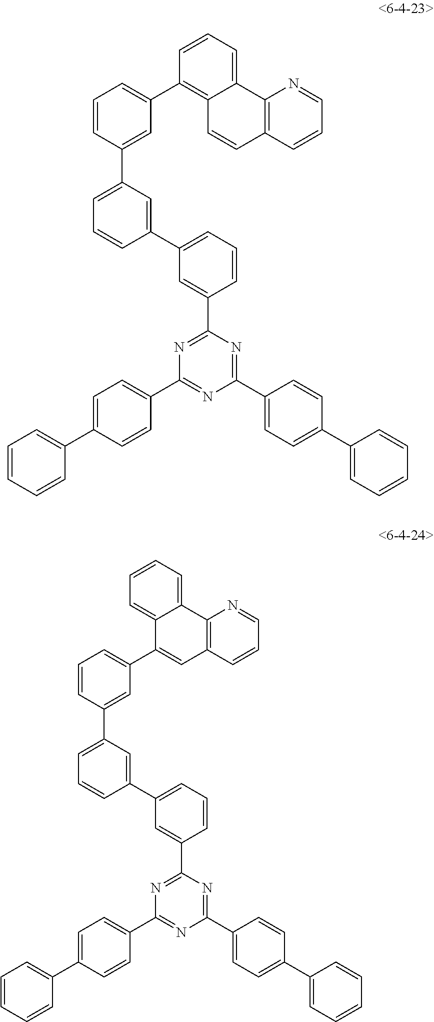 [SMILES: c1ccc(-c2ccc(-c3nc(-c4ccc(-c5ccccc5)cc4)nc(-c4cccc(-c5cccc(-c6cccc(-c7cccc8c7ccc7cccnc78)c6)c5)c4)n3)cc2)cc1 ; c1ccc(-c2ccc(-c3nc(-c4ccc(-c5ccccc5)cc4)nc(-c4cccc(-c5cccc(-c6cccc(-c7cc8cccnc8c8ccccc78)c6)c5)c4)n3)cc2)cc1]

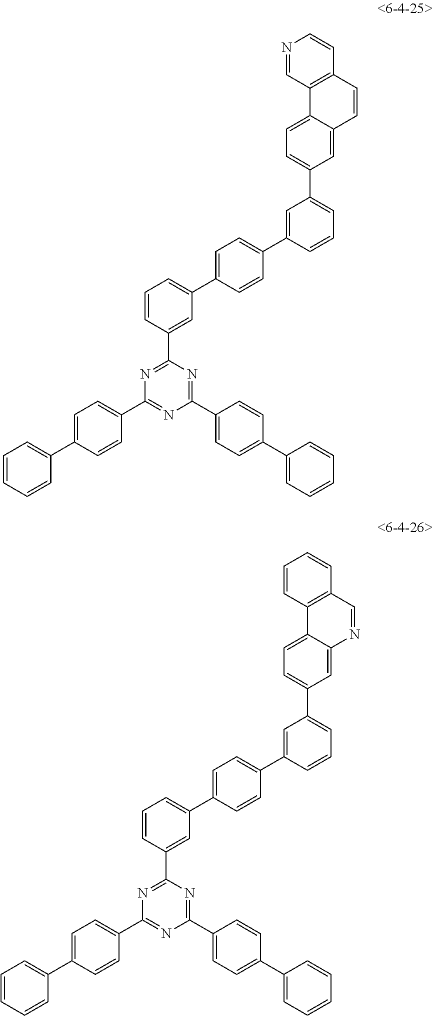 [SMILES: c1ccc(-c2ccc(-c3nc(-c4ccc(-c5ccccc5)cc4)nc(-c4cccc(-c5ccc(-c6cccc(-c7ccc8c(ccc9ccncc98)c7)c6)cc5)c4)n3)cc2)cc1 ; c1ccc(-c2ccc(-c3nc(-c4ccc(-c5ccccc5)cc4)nc(-c4cccc(-c5ccc(-c6cccc(-c7ccc8c(c7)ncc7ccccc78)c6)cc5)c4)n3)cc2)cc1]

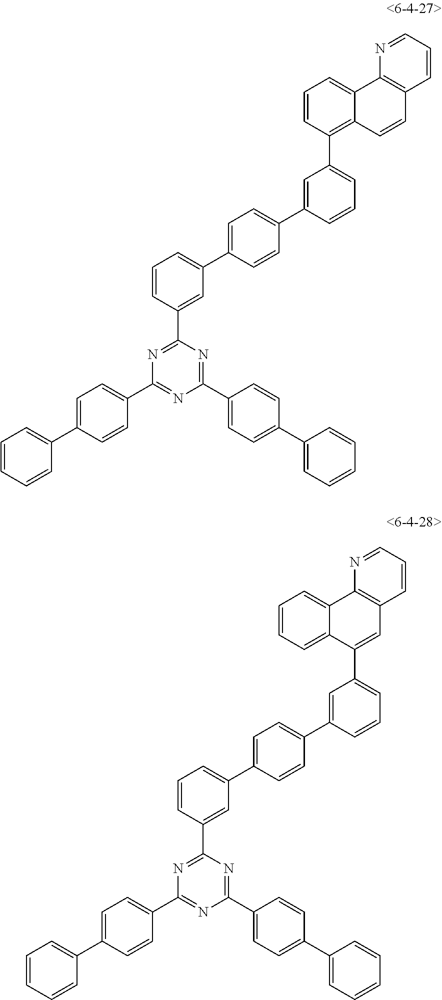 [SMILES: c1ccc(-c2ccc(-c3nc(-c4ccc(-c5ccccc5)cc4)nc(-c4cccc(-c5ccc(-c6cccc(-c7cccc8c7ccc7cccnc78)c6)cc5)c4)n3)cc2)cc1 ; c1ccc(-c2ccc(-c3nc(-c4ccc(-c5ccccc5)cc4)nc(-c4cccc(-c5ccc(-c6cccc(-c7cc8cccnc8c8ccccc78)c6)cc5)c4)n3)cc2)cc1]

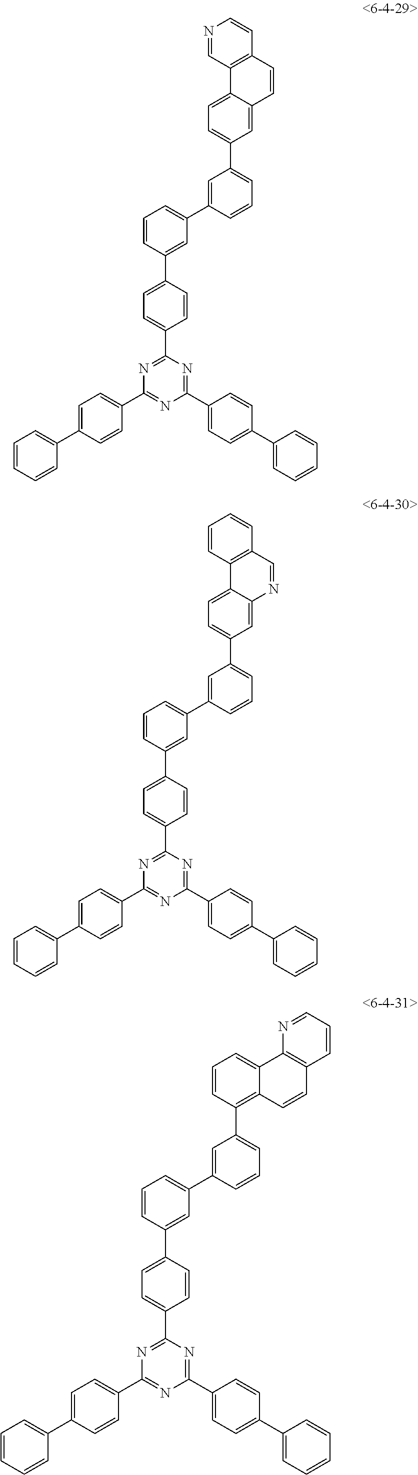 [SMILES: c1ccc(-c2ccc(-c3nc(-c4ccc(-c5ccccc5)cc4)nc(-c4ccc(-c5cccc(-c6cccc(-c7ccc8c(ccc9ccncc98)c7)c6)c5)cc4)n3)cc2)cc1 ; c1ccc(-c2ccc(-c3nc(-c4ccc(-c5ccccc5)cc4)nc(-c4ccc(-c5cccc(-c6cccc(-c7ccc8c(c7)ncc7ccccc78)c6)c5)cc4)n3)cc2)cc1 ; c1ccc(-c2ccc(-c3nc(-c4ccc(-c5ccccc5)cc4)nc(-c4ccc(-c5cccc(-c6cccc(-c7cccc8c7ccc7cccnc78)c6)c5)cc4)n3)cc2)cc1]

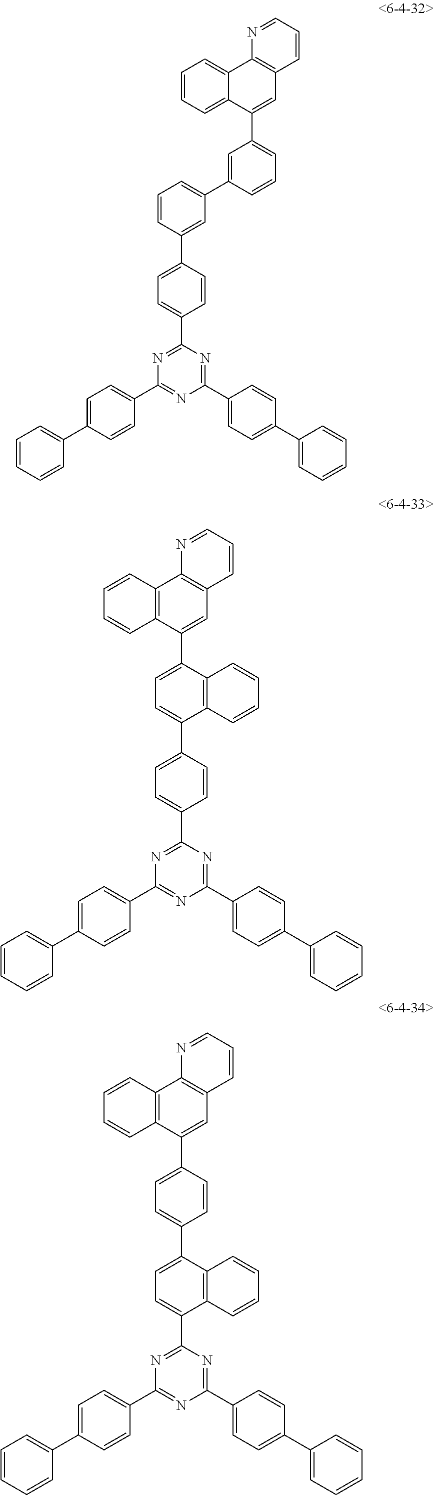 [SMILES: c1ccc(-c2ccc(-c3nc(-c4ccc(-c5ccccc5)cc4)nc(-c4ccc(-c5cccc(-c6cccc(-c7cc8cccnc8c8ccccc78)c6)c5)cc4)n3)cc2)cc1 ; c1ccc(-c2ccc(-c3nc(-c4ccc(-c5ccccc5)cc4)nc(-c4ccc(-c5ccc(-c6cc7cccnc7c7ccccc67)c6ccccc56)cc4)n3)cc2)cc1 ; c1ccc(-c2ccc(-c3nc(-c4ccc(-c5ccccc5)cc4)nc(-c4ccc(-c5ccc(-c6cc7cccnc7c7ccccc67)cc5)c5ccccc45)n3)cc2)cc1]

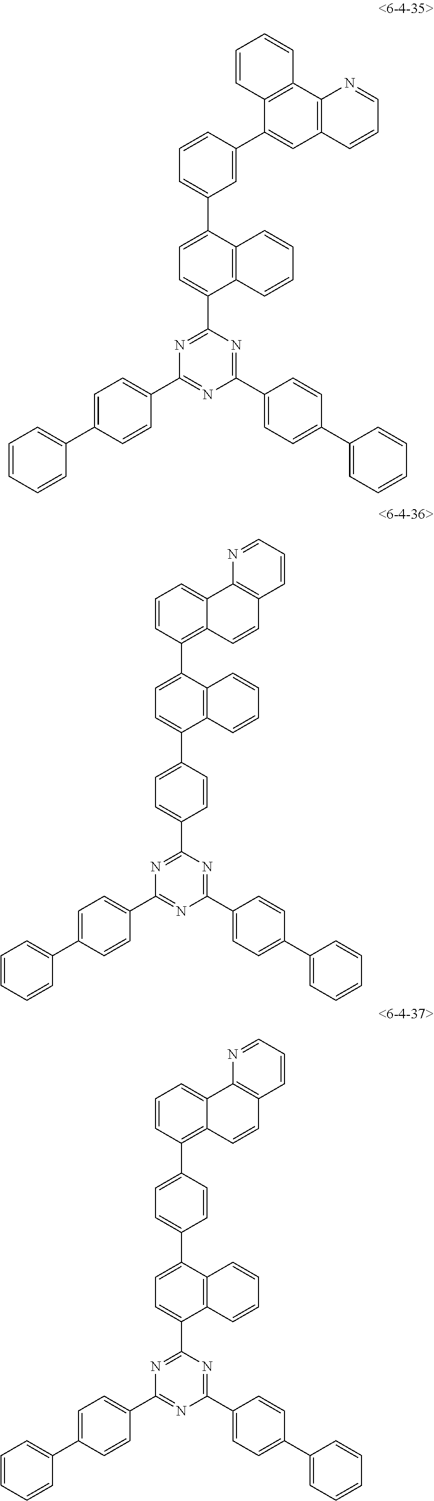 [SMILES: c1ccc(-c2ccc(-c3nc(-c4ccc(-c5ccccc5)cc4)nc(-c4ccc(-c5cccc(-c6cc7cccnc7c7ccccc67)c5)c5ccccc45)n3)cc2)cc1 ; c1ccc(-c2ccc(-c3nc(-c4ccc(-c5ccccc5)cc4)nc(-c4ccc(-c5ccc(-c6cccc7c6ccc6cccnc67)c6ccccc56)cc4)n3)cc2)cc1 ; c1ccc(-c2ccc(-c3nc(-c4ccc(-c5ccccc5)cc4)nc(-c4ccc(-c5ccc(-c6cccc7c6ccc6cccnc67)cc5)c5ccccc45)n3)cc2)cc1]

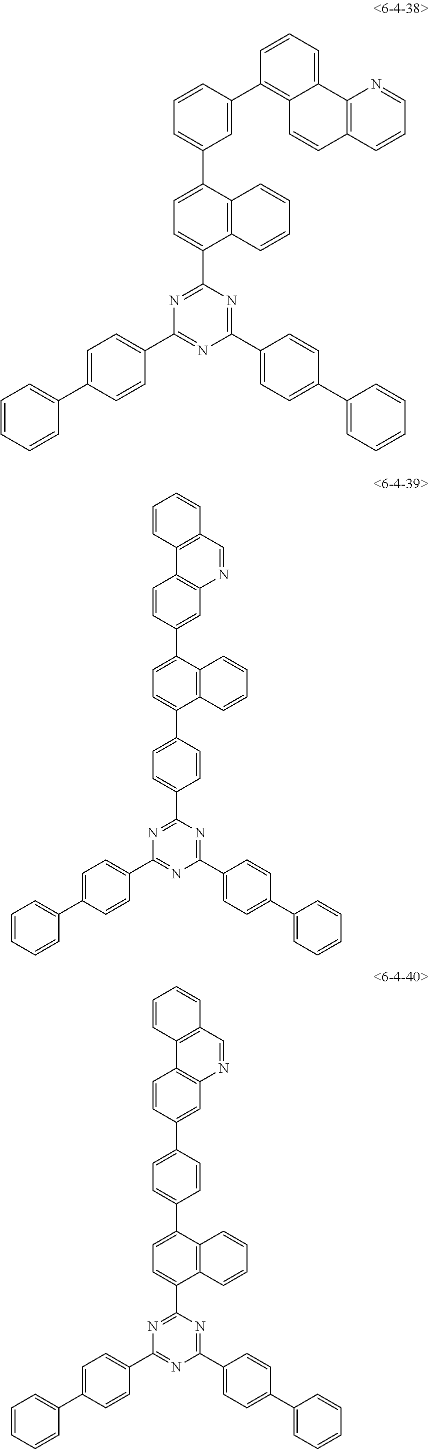 [SMILES: c1ccc(-c2ccc(-c3nc(-c4ccc(-c5ccccc5)cc4)nc(-c4ccc(-c5cccc(-c6cccc7c6ccc6cccnc67)c5)c5ccccc45)n3)cc2)cc1 ; c1ccc(-c2ccc(-c3nc(-c4ccc(-c5ccccc5)cc4)nc(-c4ccc(-c5ccc(-c6ccc7c(c6)ncc6ccccc67)c6ccccc56)cc4)n3)cc2)cc1 ; c1ccc(-c2ccc(-c3nc(-c4ccc(-c5ccccc5)cc4)nc(-c4ccc(-c5ccc(-c6ccc7c(c6)ncc6ccccc67)cc5)c5ccccc45)n3)cc2)cc1]

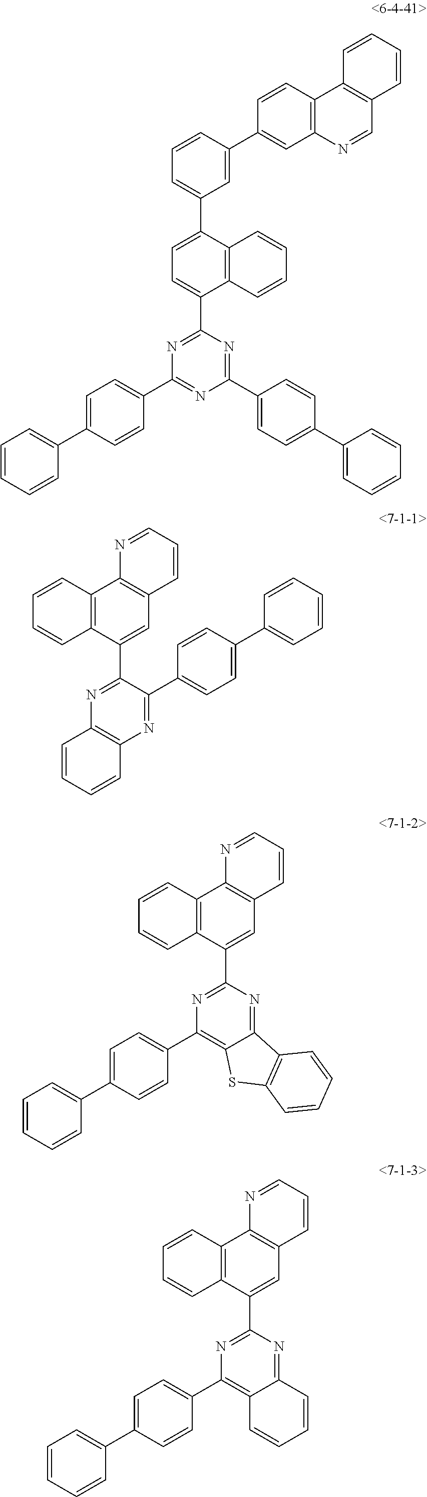 [SMILES: c1ccc(-c2ccc(-c3nc(-c4ccc(-c5ccccc5)cc4)nc(-c4ccc(-c5cccc(-c6ccc7c(c6)ncc6ccccc67)c5)c5ccccc45)n3)cc2)cc1 ; c1ccc(-c2ccc(-c3nc4ccccc4nc3-c3cc4cccnc4c4ccccc34)cc2)cc1 ; c1ccc(-c2ccc(-c3nc(-c4cc5cccnc5c5ccccc45)nc4c3sc3ccccc34)cc2)cc1 ; c1ccc(-c2ccc(-c3nc(-c4cc5cccnc5c5ccccc45)nc4ccccc34)cc2)cc1]

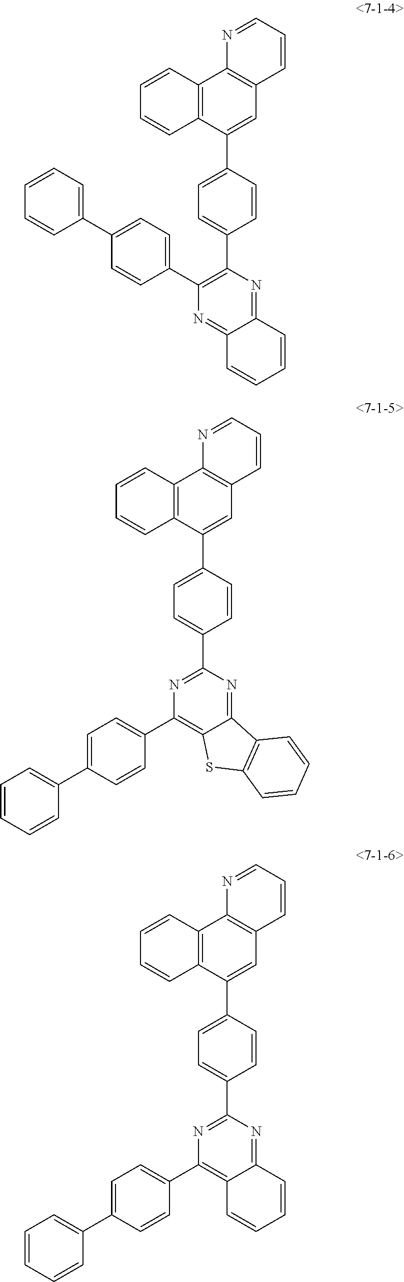

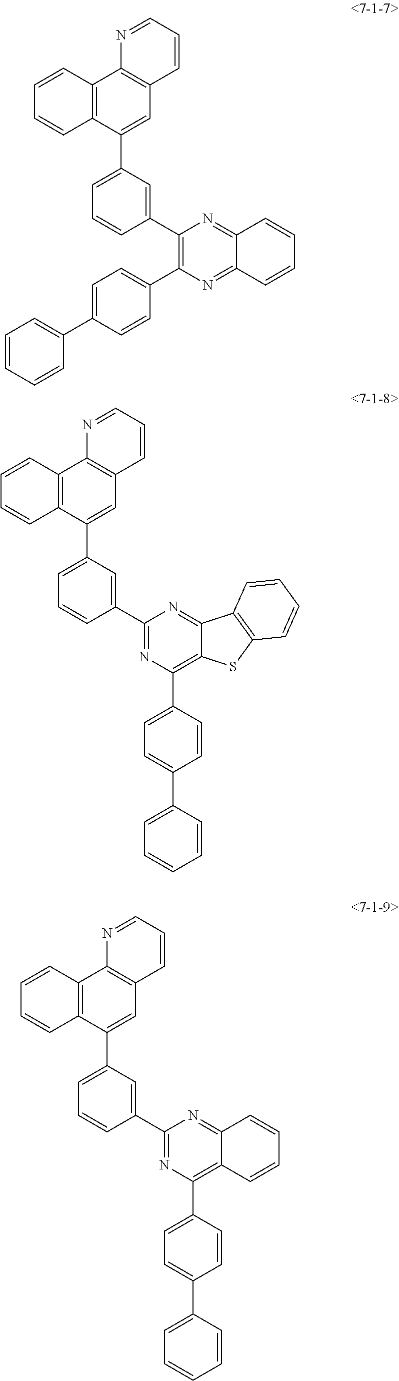 [SMILES: c1ccc(-c2ccc(-c3nc4ccccc4nc3-c3cccc(-c4cc5cccnc5c5ccccc45)c3)cc2)cc1 ; c1ccc(-c2ccc(-c3nc(-c4cccc(-c5cc6cccnc6c6ccccc56)c4)nc4c3sc3ccccc34)cc2)cc1 ; c1ccc(-c2ccc(-c3nc(-c4cccc(-c5cc6cccnc6c6ccccc56)c4)nc4ccccc34)cc2)cc1]

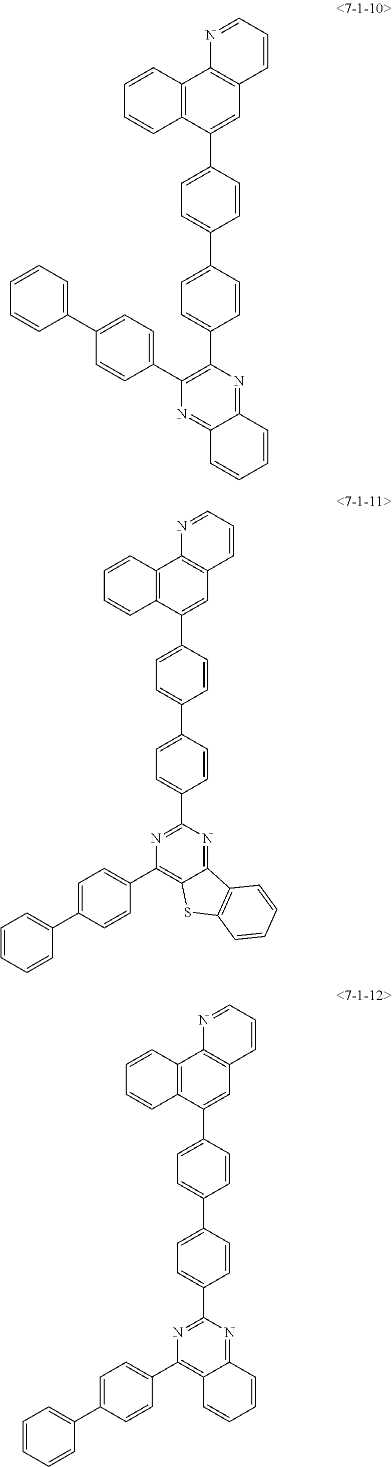 [SMILES: c1ccc(-c2ccc(-c3nc4ccccc4nc3-c3ccc(-c4ccc(-c5cc6cccnc6c6ccccc56)cc4)cc3)cc2)cc1 ; c1ccc(-c2ccc(-c3nc(-c4ccc(-c5ccc(-c6cc7cccnc7c7ccccc67)cc5)cc4)nc4c3sc3ccccc34)cc2)cc1 ; c1ccc(-c2ccc(-c3nc(-c4ccc(-c5ccc(-c6cc7cccnc7c7ccccc67)cc5)cc4)nc4ccccc34)cc2)cc1]

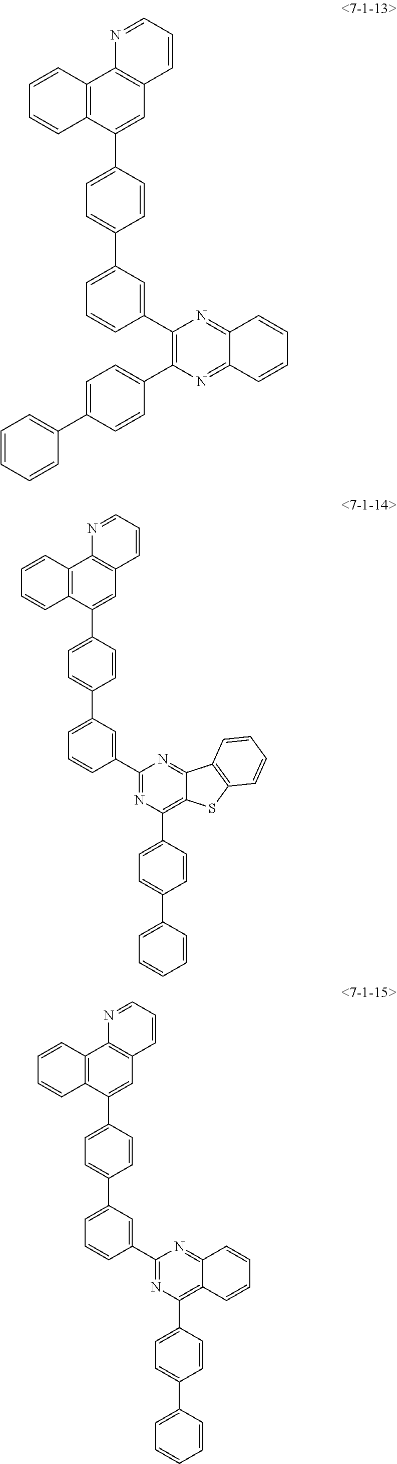 [SMILES: c1ccc(-c2ccc(-c3nc4ccccc4nc3-c3cccc(-c4ccc(-c5cc6cccnc6c6ccccc56)cc4)c3)cc2)cc1 ; c1ccc(-c2ccc(-c3nc(-c4cccc(-c5ccc(-c6cc7cccnc7c7ccccc67)cc5)c4)nc4c3sc3ccccc34)cc2)cc1 ; c1ccc(-c2ccc(-c3nc(-c4cccc(-c5ccc(-c6cc7cccnc7c7ccccc67)cc5)c4)nc4ccccc34)cc2)cc1]

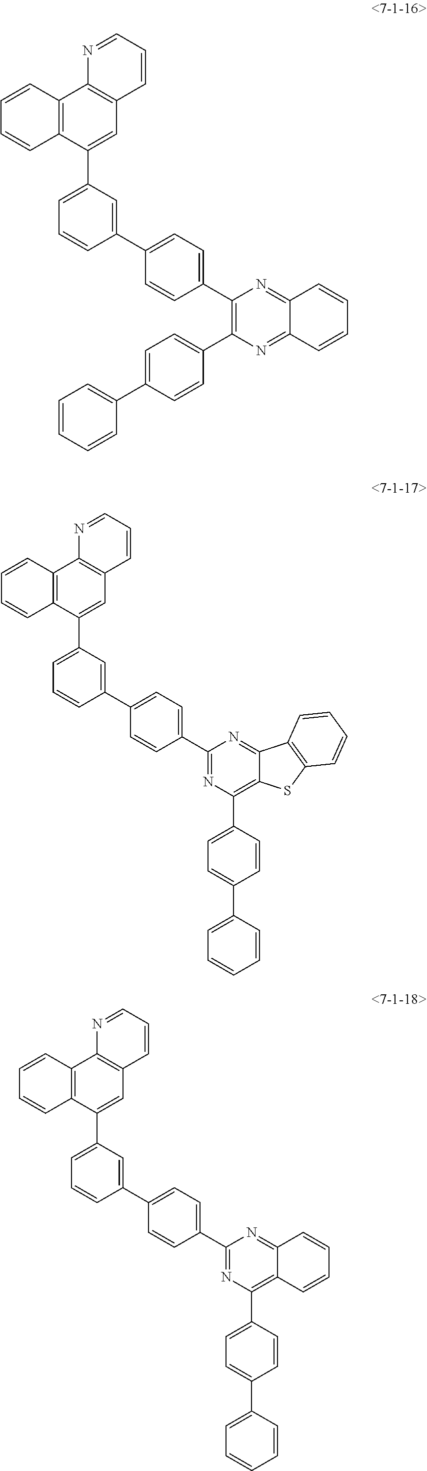 [SMILES: c1ccc(-c2ccc(-c3nc4ccccc4nc3-c3ccc(-c4cccc(-c5cc6cccnc6c6ccccc56)c4)cc3)cc2)cc1 ; c1ccc(-c2ccc(-c3nc(-c4ccc(-c5cccc(-c6cc7cccnc7c7ccccc67)c5)cc4)nc4c3sc3ccccc34)cc2)cc1 ; c1ccc(-c2ccc(-c3nc(-c4ccc(-c5cccc(-c6cc7cccnc7c7ccccc67)c5)cc4)nc4ccccc34)cc2)cc1]

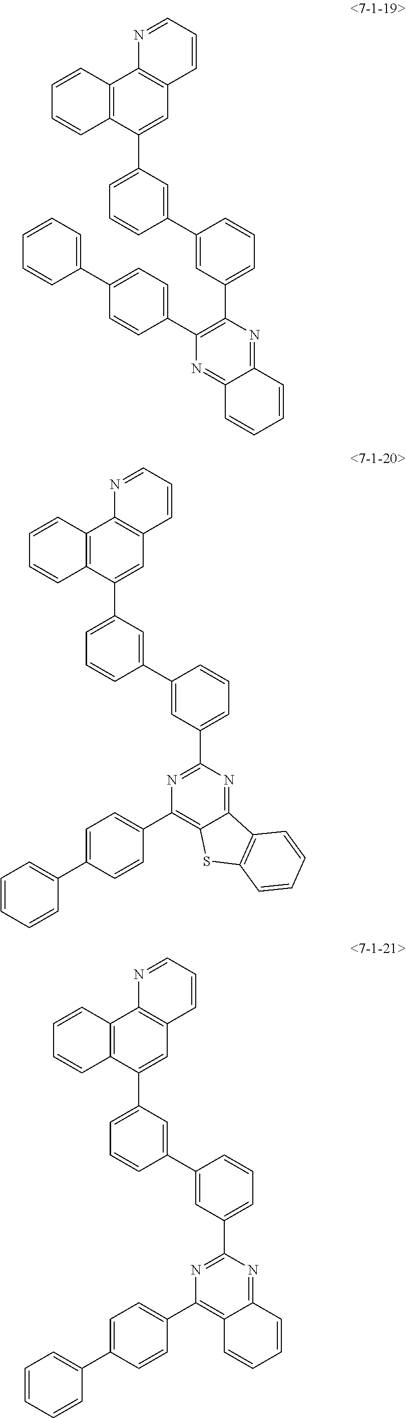 [SMILES: c1ccc(-c2ccc(-c3nc4ccccc4nc3-c3cccc(-c4cccc(-c5cc6cccnc6c6ccccc56)c4)c3)cc2)cc1 ; c1ccc(-c2ccc(-c3nc(-c4cccc(-c5cccc(-c6cc7cccnc7c7ccccc67)c5)c4)nc4c3sc3ccccc34)cc2)cc1 ; c1ccc(-c2ccc(-c3nc(-c4cccc(-c5cccc(-c6cc7cccnc7c7ccccc67)c5)c4)nc4ccccc34)cc2)cc1]

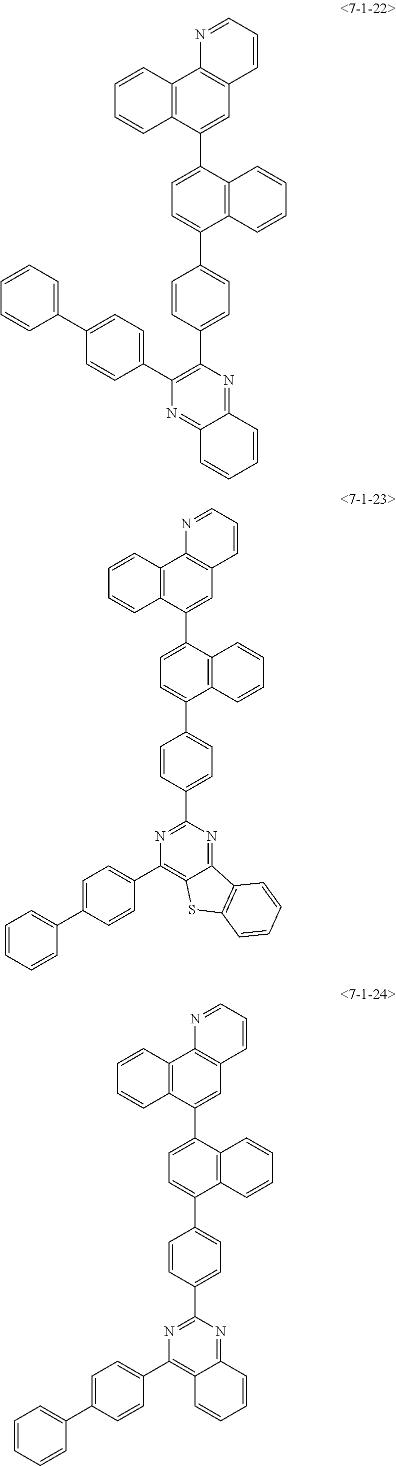

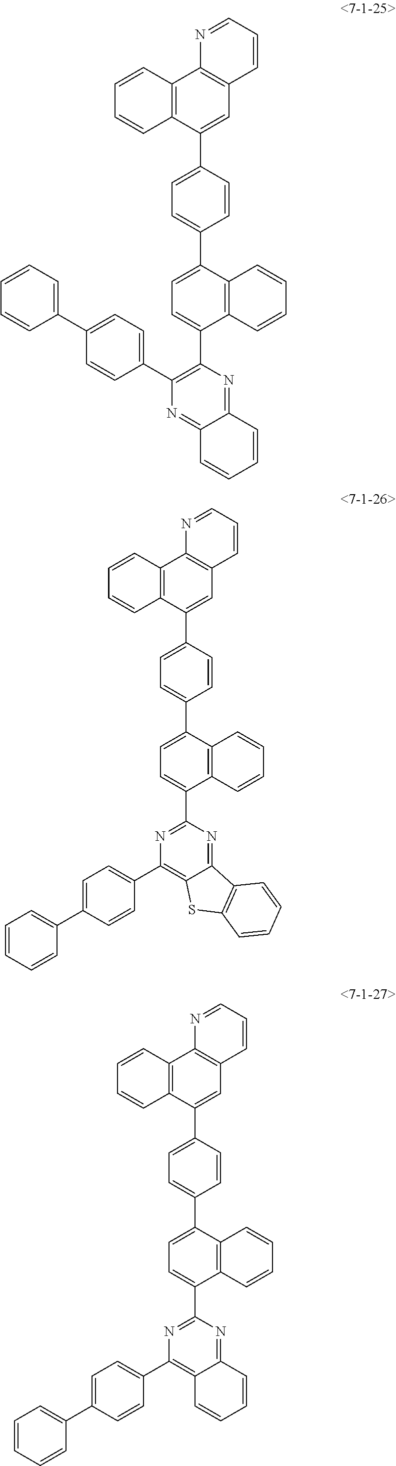

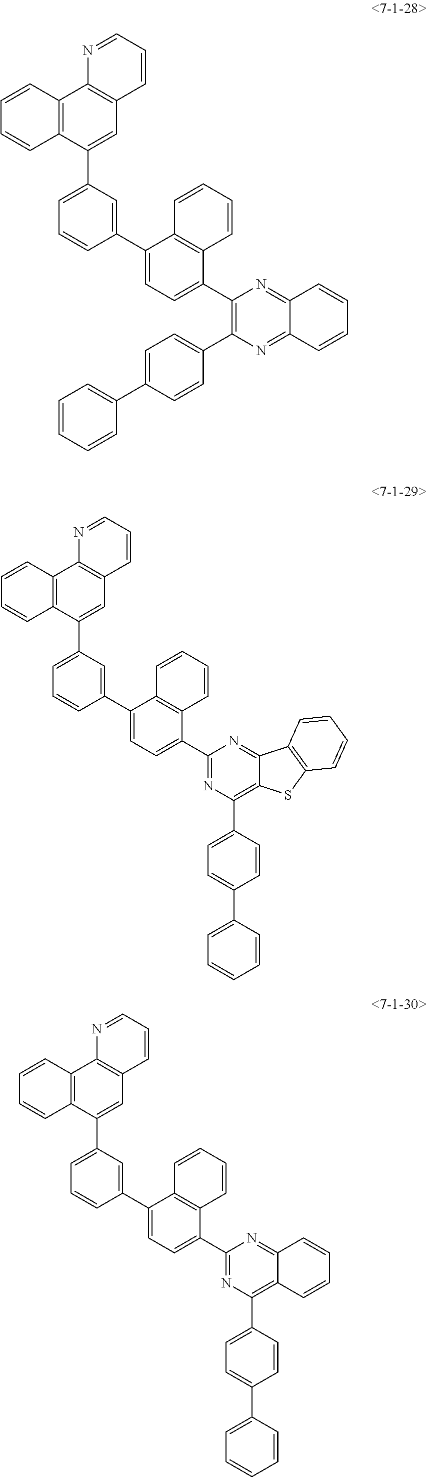 [SMILES: c1ccc(-c2ccc(-c3nc4ccccc4nc3-c3ccc(-c4cccc(-c5cc6cccnc6c6ccccc56)c4)c4ccccc34)cc2)cc1 ; c1ccc(-c2ccc(-c3nc(-c4ccc(-c5cccc(-c6cc7cccnc7c7ccccc67)c5)c5ccccc45)nc4c3sc3ccccc34)cc2)cc1 ; c1ccc(-c2ccc(-c3nc(-c4ccc(-c5cccc(-c6cc7cccnc7c7ccccc67)c5)c5ccccc45)nc4ccccc34)cc2)cc1]

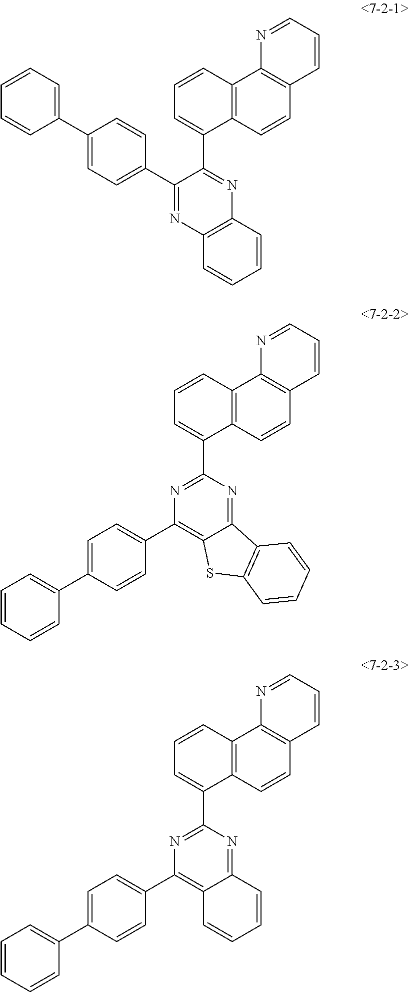 [SMILES: c1ccc(-c2ccc(-c3nc4ccccc4nc3-c3cccc4c3ccc3cccnc34)cc2)cc1 ; c1ccc(-c2ccc(-c3nc(-c4cccc5c4ccc4cccnc45)nc4c3sc3ccccc34)cc2)cc1 ; c1ccc(-c2ccc(-c3nc(-c4cccc5c4ccc4cccnc45)nc4ccccc34)cc2)cc1]

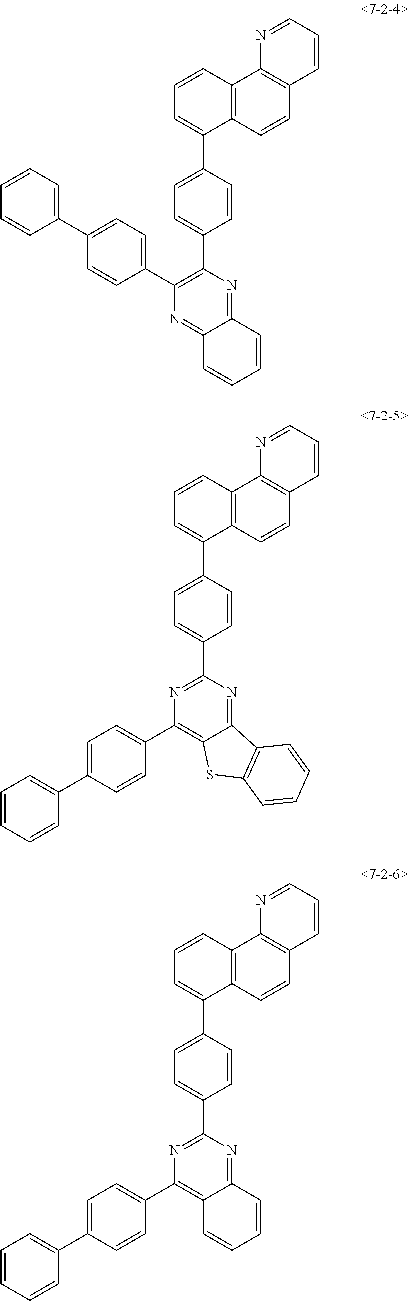 [SMILES: c1ccc(-c2ccc(-c3nc4ccccc4nc3-c3ccc(-c4cccc5c4ccc4cccnc45)cc3)cc2)cc1 ; c1ccc(-c2ccc(-c3nc(-c4ccc(-c5cccc6c5ccc5cccnc56)cc4)nc4c3sc3ccccc34)cc2)cc1 ; c1ccc(-c2ccc(-c3nc(-c4ccc(-c5cccc6c5ccc5cccnc56)cc4)nc4ccccc34)cc2)cc1]

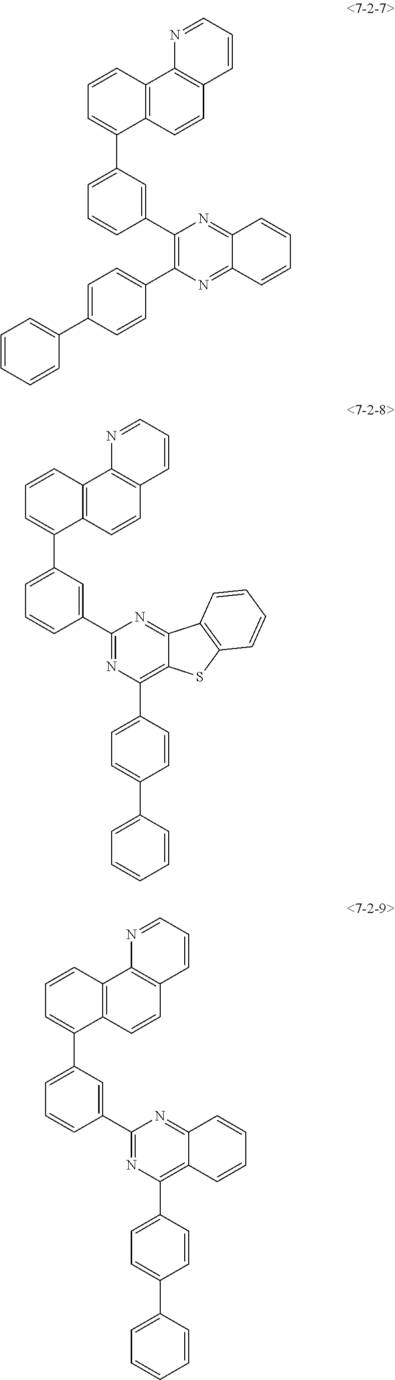

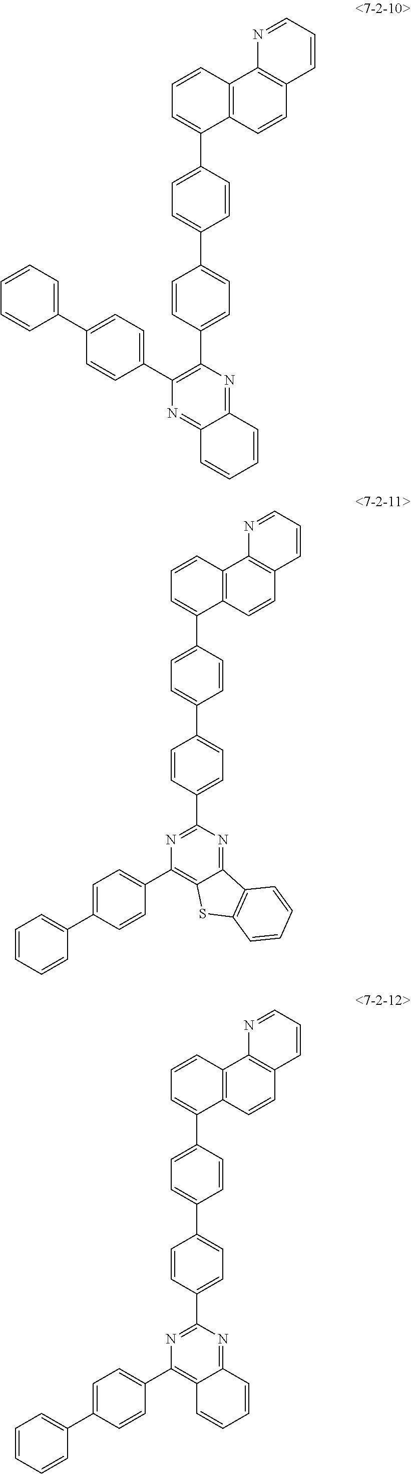 [SMILES: c1ccc(-c2ccc(-c3nc4ccccc4nc3-c3ccc(-c4ccc(-c5cccc6c5ccc5cccnc56)cc4)cc3)cc2)cc1 ; c1ccc(-c2ccc(-c3nc(-c4ccc(-c5ccc(-c6cccc7c6ccc6cccnc67)cc5)cc4)nc4c3sc3ccccc34)cc2)cc1 ; c1ccc(-c2ccc(-c3nc(-c4ccc(-c5ccc(-c6cccc7c6ccc6cccnc67)cc5)cc4)nc4ccccc34)cc2)cc1]

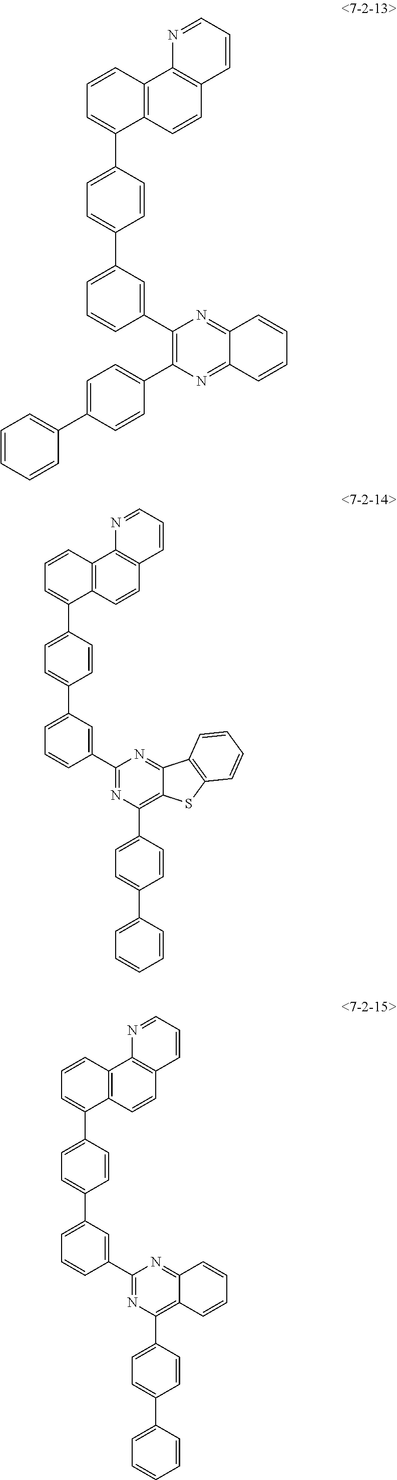 [SMILES: c1ccc(-c2ccc(-c3nc4ccccc4nc3-c3cccc(-c4ccc(-c5cccc6c5ccc5cccnc56)cc4)c3)cc2)cc1 ; c1ccc(-c2ccc(-c3nc(-c4cccc(-c5ccc(-c6cccc7c6ccc6cccnc67)cc5)c4)nc4c3sc3ccccc34)cc2)cc1 ; c1ccc(-c2ccc(-c3nc(-c4cccc(-c5ccc(-c6cccc7c6ccc6cccnc67)cc5)c4)nc4ccccc34)cc2)cc1]

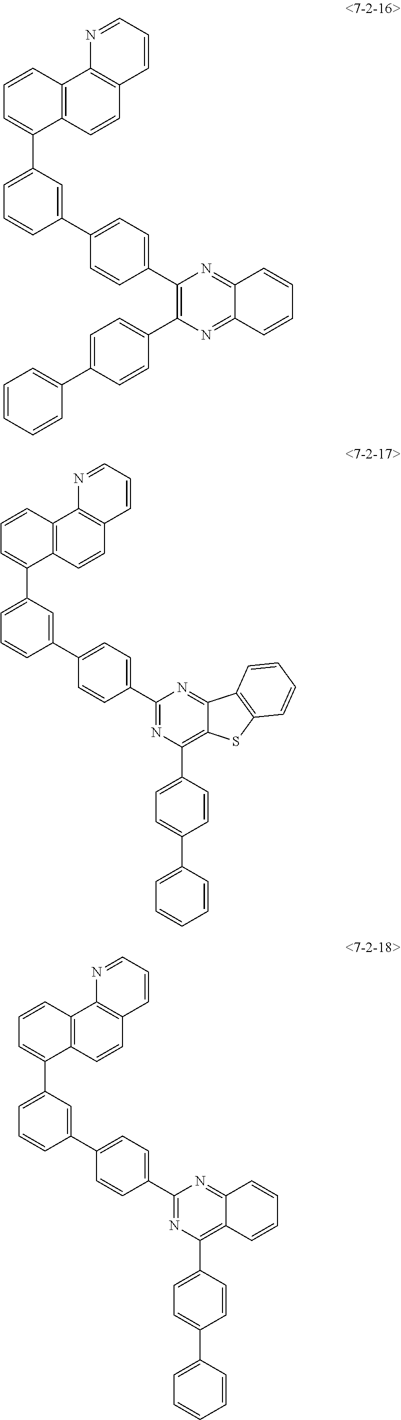 [SMILES: c1ccc(-c2ccc(-c3nc4ccccc4nc3-c3ccc(-c4cccc(-c5cccc6c5ccc5cccnc56)c4)cc3)cc2)cc1 ; c1ccc(-c2ccc(-c3nc(-c4ccc(-c5cccc(-c6cccc7c6ccc6cccnc67)c5)cc4)nc4c3sc3ccccc34)cc2)cc1 ; c1ccc(-c2ccc(-c3nc(-c4ccc(-c5cccc(-c6cccc7c6ccc6cccnc67)c5)cc4)nc4ccccc34)cc2)cc1]

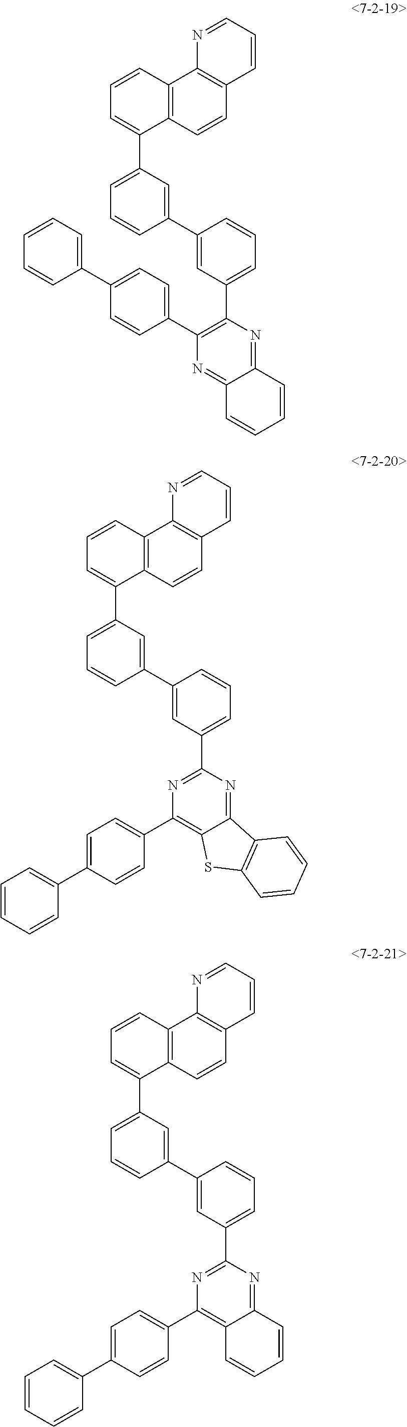 [SMILES: c1ccc(-c2ccc(-c3nc4ccccc4nc3-c3cccc(-c4cccc(-c5cccc6c5ccc5cccnc56)c4)c3)cc2)cc1 ; c1ccc(-c2ccc(-c3nc(-c4cccc(-c5cccc(-c6cccc7c6ccc6cccnc67)c5)c4)nc4c3sc3ccccc34)cc2)cc1 ; c1ccc(-c2ccc(-c3nc(-c4cccc(-c5cccc(-c6cccc7c6ccc6cccnc67)c5)c4)nc4ccccc34)cc2)cc1]

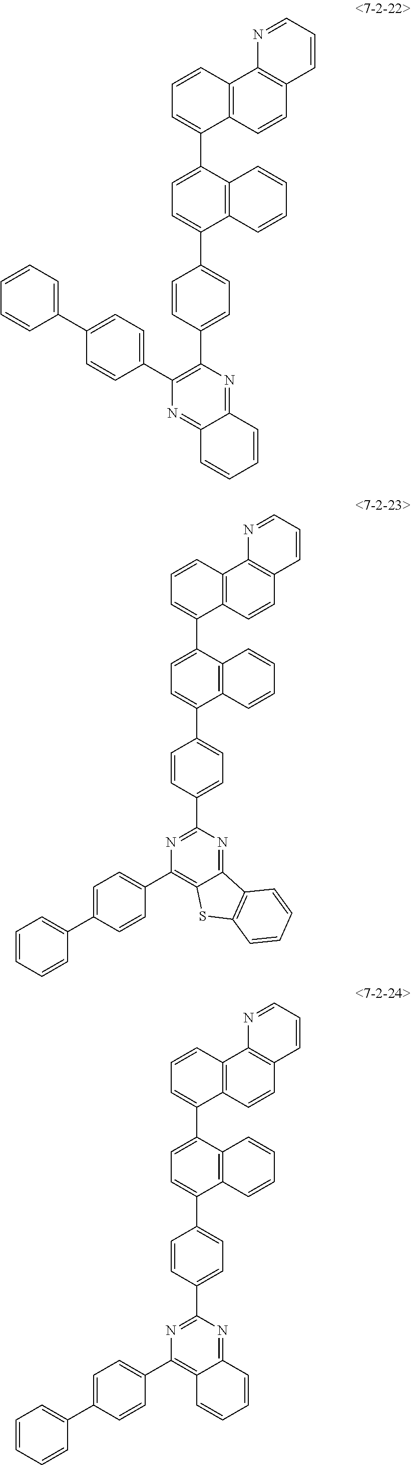 [SMILES: c1ccc(-c2ccc(-c3nc4ccccc4nc3-c3ccc(-c4ccc(-c5cccc6c5ccc5cccnc56)c5ccccc45)cc3)cc2)cc1 ; c1ccc(-c2ccc(-c3nc(-c4ccc(-c5ccc(-c6cccc7c6ccc6cccnc67)c6ccccc56)cc4)nc4c3sc3ccccc34)cc2)cc1 ; c1ccc(-c2ccc(-c3nc(-c4ccc(-c5ccc(-c6cccc7c6ccc6cccnc67)c6ccccc56)cc4)nc4ccccc34)cc2)cc1]

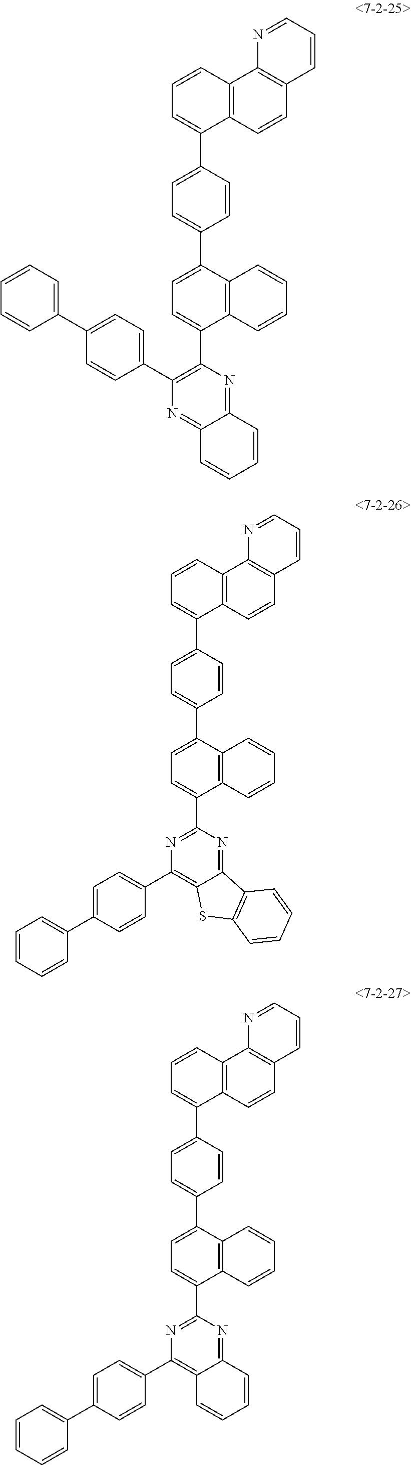 [SMILES: c1ccc(-c2ccc(-c3nc4ccccc4nc3-c3ccc(-c4ccc(-c5cccc6c5ccc5cccnc56)cc4)c4ccccc34)cc2)cc1 ; c1ccc(-c2ccc(-c3nc(-c4ccc(-c5ccc(-c6cccc7c6ccc6cccnc67)cc5)c5ccccc45)nc4c3sc3ccccc34)cc2)cc1 ; c1ccc(-c2ccc(-c3nc(-c4ccc(-c5ccc(-c6cccc7c6ccc6cccnc67)cc5)c5ccccc45)nc4ccccc34)cc2)cc1]

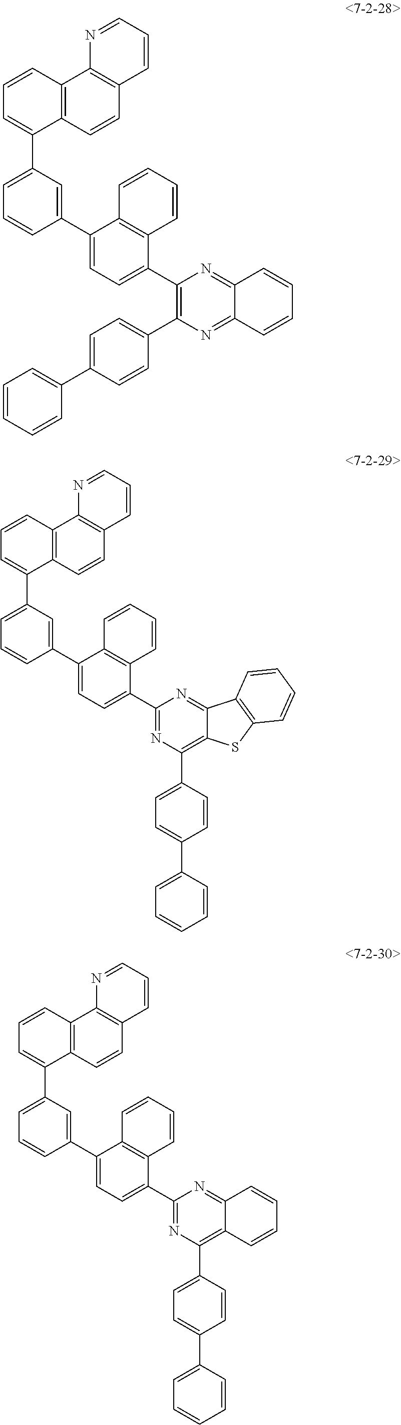 [SMILES: c1ccc(-c2ccc(-c3nc4ccccc4nc3-c3ccc(-c4cccc(-c5cccc6c5ccc5cccnc56)c4)c4ccccc34)cc2)cc1 ; c1ccc(-c2ccc(-c3nc(-c4ccc(-c5cccc(-c6cccc7c6ccc6cccnc67)c5)c5ccccc45)nc4c3sc3ccccc34)cc2)cc1 ; c1ccc(-c2ccc(-c3nc(-c4ccc(-c5cccc(-c6cccc7c6ccc6cccnc67)c5)c5ccccc45)nc4ccccc34)cc2)cc1]

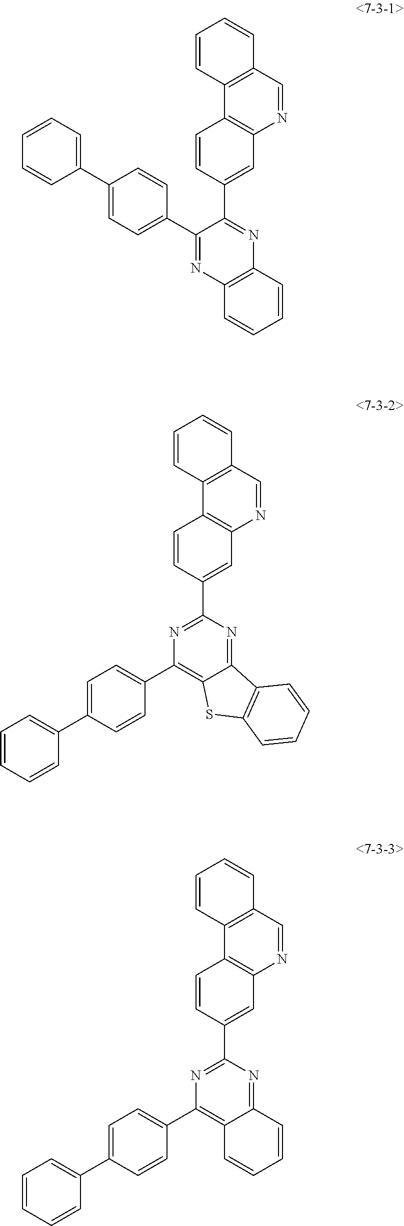 [SMILES: c1ccc(-c2ccc(-c3nc4ccccc4nc3-c3ccc4c(c3)ncc3ccccc34)cc2)cc1 ; c1ccc(-c2ccc(-c3nc(-c4ccc5c(c4)ncc4ccccc45)nc4c3sc3ccccc34)cc2)cc1 ; c1ccc(-c2ccc(-c3nc(-c4ccc5c(c4)ncc4ccccc45)nc4ccccc34)cc2)cc1]

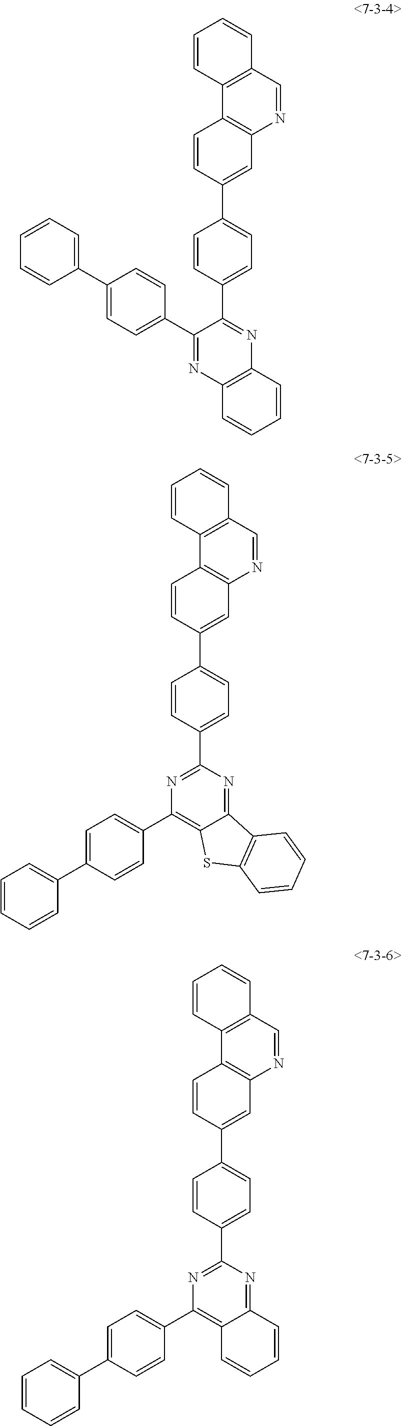 [SMILES: c1ccc(-c2ccc(-c3nc4ccccc4nc3-c3ccc(-c4ccc5c(c4)ncc4ccccc45)cc3)cc2)cc1 ; c1ccc(-c2ccc(-c3nc(-c4ccc(-c5ccc6c(c5)ncc5ccccc56)cc4)nc4c3sc3ccccc34)cc2)cc1 ; c1ccc(-c2ccc(-c3nc(-c4ccc(-c5ccc6c(c5)ncc5ccccc56)cc4)nc4ccccc34)cc2)cc1]

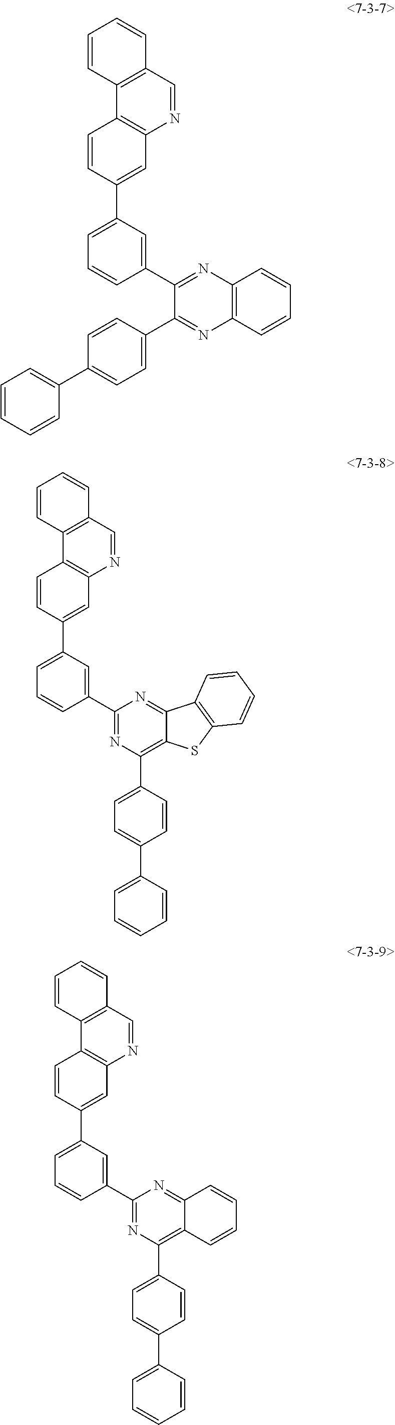 [SMILES: c1ccc(-c2ccc(-c3nc4ccccc4nc3-c3cccc(-c4ccc5c(c4)ncc4ccccc45)c3)cc2)cc1 ; c1ccc(-c2ccc(-c3nc(-c4cccc(-c5ccc6c(c5)ncc5ccccc56)c4)nc4c3sc3ccccc34)cc2)cc1 ; c1ccc(-c2ccc(-c3nc(-c4cccc(-c5ccc6c(c5)ncc5ccccc56)c4)nc4ccccc34)cc2)cc1]

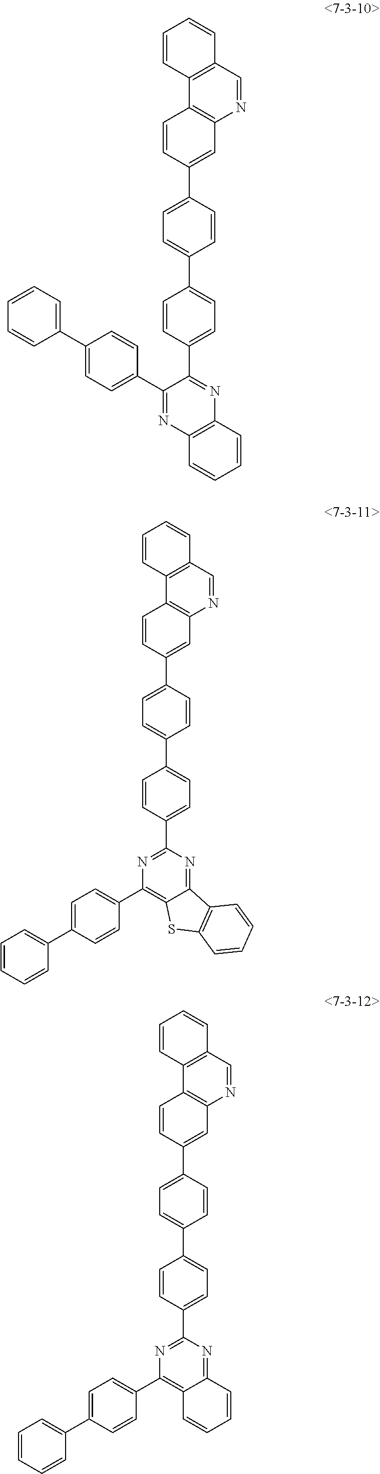 [SMILES: c1ccc(-c2ccc(-c3nc4ccccc4nc3-c3ccc(-c4ccc(-c5ccc6c(c5)ncc5ccccc56)cc4)cc3)cc2)cc1 ; c1ccc(-c2ccc(-c3nc(-c4ccc(-c5ccc(-c6ccc7c(c6)ncc6ccccc67)cc5)cc4)nc4c3sc3ccccc34)cc2)cc1 ; c1ccc(-c2ccc(-c3nc(-c4ccc(-c5ccc(-c6ccc7c(c6)ncc6ccccc67)cc5)cc4)nc4ccccc34)cc2)cc1]

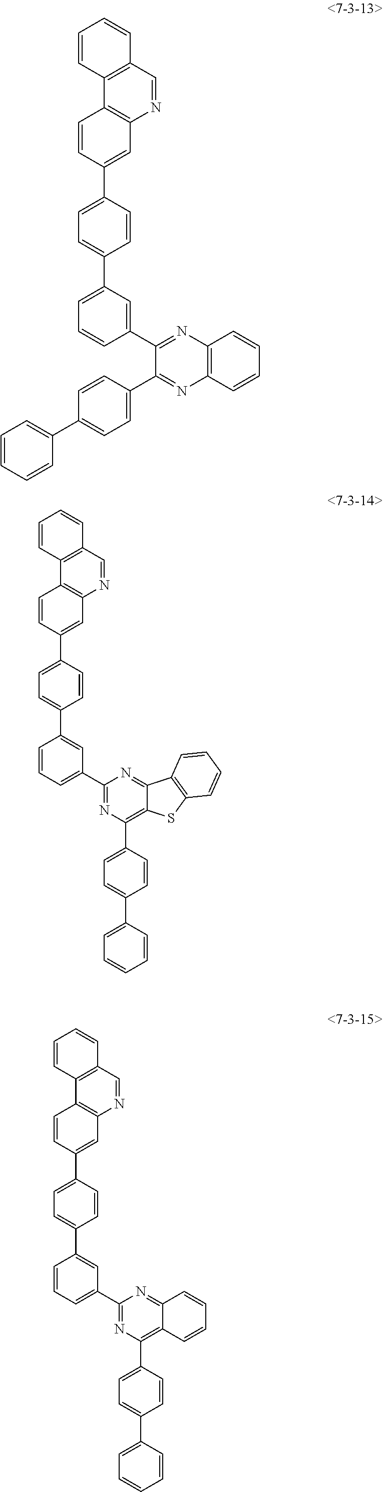 [SMILES: c1ccc(-c2ccc(-c3nc4ccccc4nc3-c3cccc(-c4ccc(-c5ccc6c(c5)ncc5ccccc56)cc4)c3)cc2)cc1 ; c1ccc(-c2ccc(-c3nc(-c4cccc(-c5ccc(-c6ccc7c(c6)ncc6ccccc67)cc5)c4)nc4c3sc3ccccc34)cc2)cc1 ; c1ccc(-c2ccc(-c3nc(-c4cccc(-c5ccc(-c6ccc7c(c6)ncc6ccccc67)cc5)c4)nc4ccccc34)cc2)cc1]

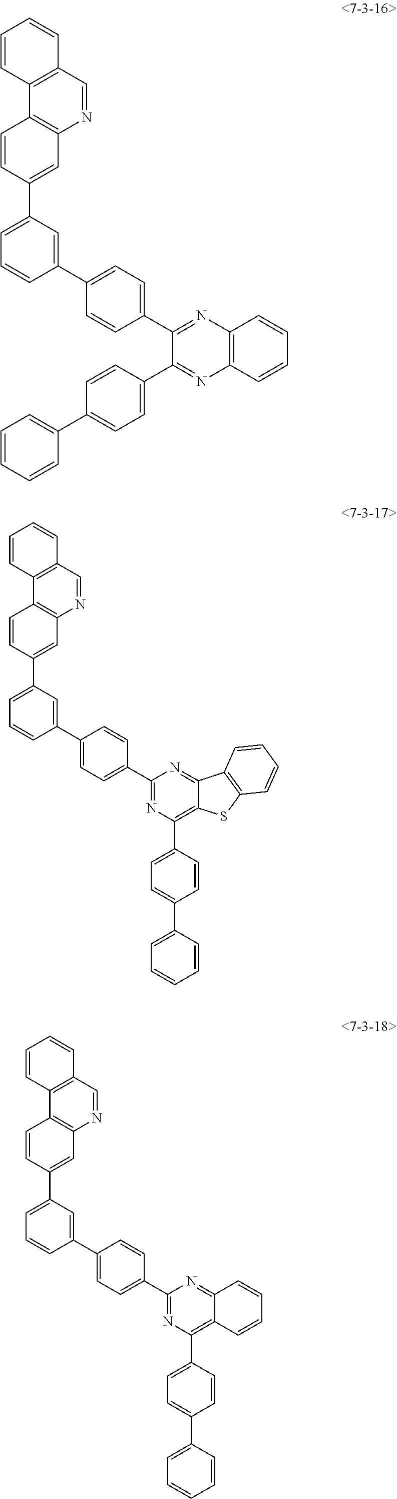 [SMILES: c1ccc(-c2ccc(-c3nc4ccccc4nc3-c3ccc(-c4cccc(-c5ccc6c(c5)ncc5ccccc56)c4)cc3)cc2)cc1 ; c1ccc(-c2ccc(-c3nc(-c4ccc(-c5cccc(-c6ccc7c(c6)ncc6ccccc67)c5)cc4)nc4c3sc3ccccc34)cc2)cc1 ; c1ccc(-c2ccc(-c3nc(-c4ccc(-c5cccc(-c6ccc7c(c6)ncc6ccccc67)c5)cc4)nc4ccccc34)cc2)cc1]

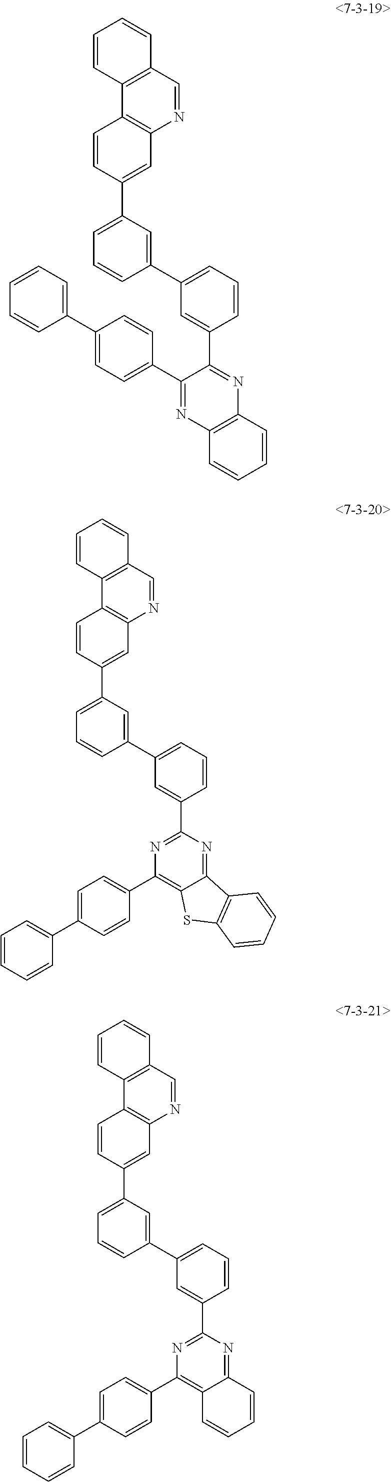 [SMILES: c1ccc(-c2ccc(-c3nc4ccccc4nc3-c3cccc(-c4cccc(-c5ccc6c(c5)ncc5ccccc56)c4)c3)cc2)cc1 ; c1ccc(-c2ccc(-c3nc(-c4cccc(-c5cccc(-c6ccc7c(c6)ncc6ccccc67)c5)c4)nc4c3sc3ccccc34)cc2)cc1 ; c1ccc(-c2ccc(-c3nc(-c4cccc(-c5cccc(-c6ccc7c(c6)ncc6ccccc67)c5)c4)nc4ccccc34)cc2)cc1]

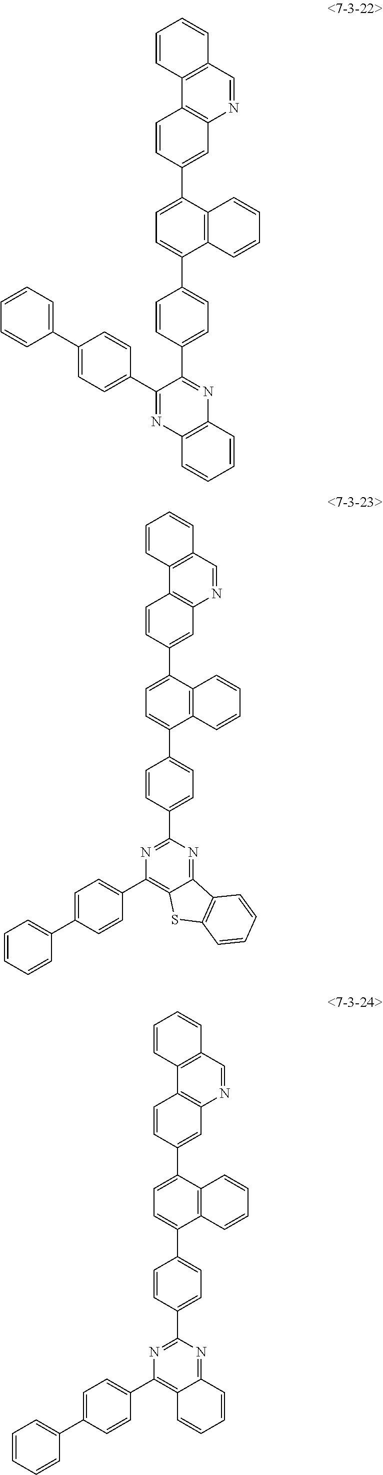 [SMILES: c1ccc(-c2ccc(-c3nc4ccccc4nc3-c3ccc(-c4ccc(-c5ccc6c(c5)ncc5ccccc56)c5ccccc45)cc3)cc2)cc1 ; c1ccc(-c2ccc(-c3nc(-c4ccc(-c5ccc(-c6ccc7c(c6)ncc6ccccc67)c6ccccc56)cc4)nc4c3sc3ccccc34)cc2)cc1 ; c1ccc(-c2ccc(-c3nc(-c4ccc(-c5ccc(-c6ccc7c(c6)ncc6ccccc67)c6ccccc56)cc4)nc4ccccc34)cc2)cc1]

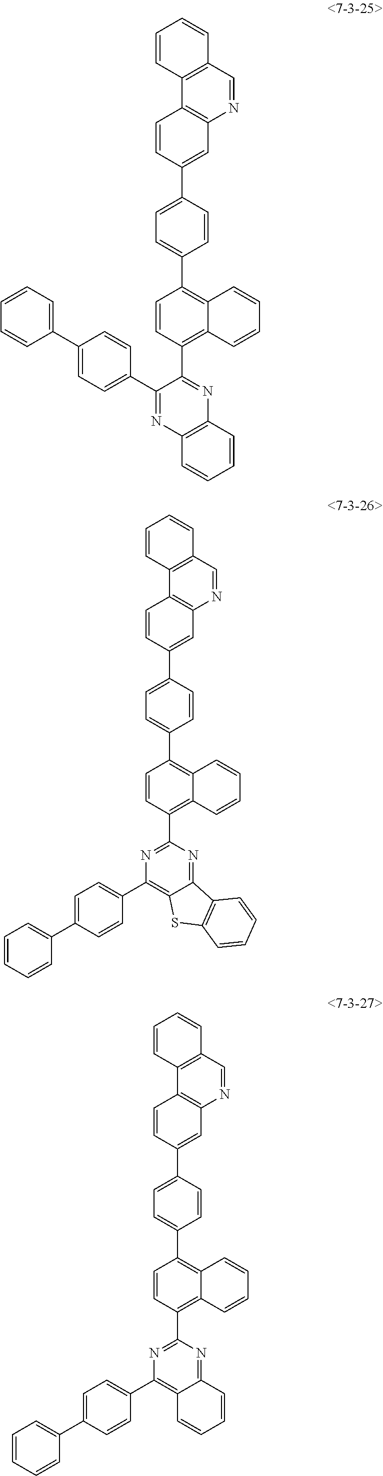 [SMILES: c1ccc(-c2ccc(-c3nc4ccccc4nc3-c3ccc(-c4ccc(-c5ccc6c(c5)ncc5ccccc56)cc4)c4ccccc34)cc2)cc1 ; c1ccc(-c2ccc(-c3nc(-c4ccc(-c5ccc(-c6ccc7c(c6)ncc6ccccc67)cc5)c5ccccc45)nc4c3sc3ccccc34)cc2)cc1 ; c1ccc(-c2ccc(-c3nc(-c4ccc(-c5ccc(-c6ccc7c(c6)ncc6ccccc67)cc5)c5ccccc45)nc4ccccc34)cc2)cc1]

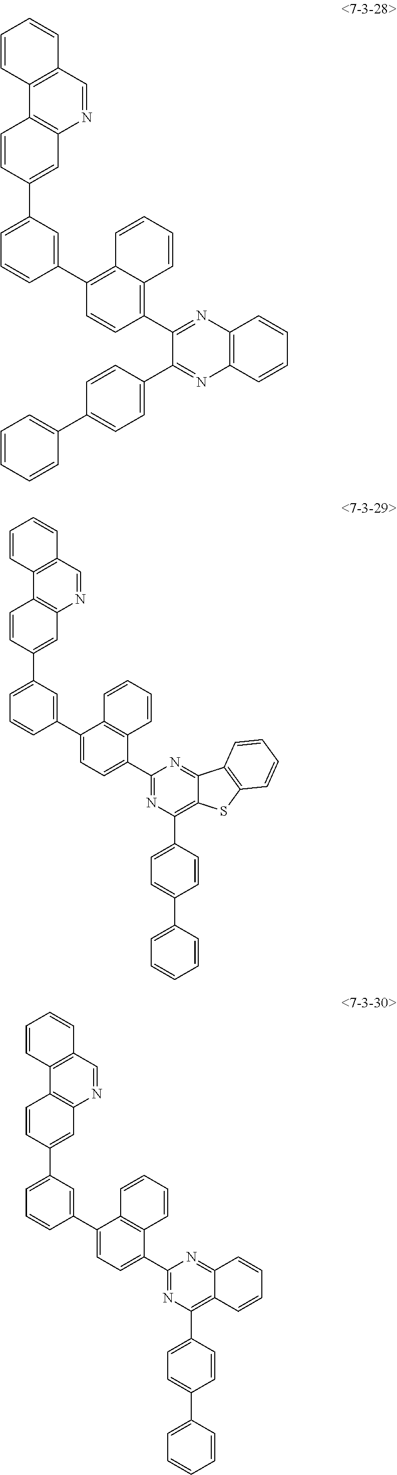

According to another aspect of the present invention, provided is an organic light emitting device comprising: a first electrode; a second electrode facing the first electrode; and an organic material layer interposed between the first electrode and the second electrode, wherein the organic material layer comprises the compound of chemical formula 1.

Furthermore, in the organic light emitting device, the first electrode is an anode, the second electrode is a cathode, the organic material layer comprises i) a light emitting layer, ii) a hole transport region interposed between the first electrode and the light emitting layer and including at least one of a hole injection layer, a hole transport layer, and an electron blocking layer, and iii) an electron transport region interposed between the light emitting layer and the second electrode and including at least one of a hole blocking layer, an electron transport layer and an electron injection layer, and, the electron transport region comprises the compound of chemical formula 1.

Furthermore, the electron transport layer of the organic light emitting device comprises the compound of chemical formula 1.

According to another aspect of the present invention, there is provided a display device including the organic light emitting device, wherein a first electrode of the organic light emitting device is electrically connected to a source electrode or a drain electrode of a thin film transistor.

Hereinafter, synthesis examples of compounds represented by chemical formula 1 and manufacturing examples of organic light emitting devices according to the present invention will be described in detail, but the present invention is not limited to the following examples.

### <Synthesis Method of Intermediate Products and FDMS Data>

### (1) Synthesis of Core 1-1 to Core 1-5

6-bromobenzo [j] phenanthridine (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 6-(4,5,5,5-tetramethyl-1,3,2-dioxaborolan-2-yl) benzo [j] phenanthridine.

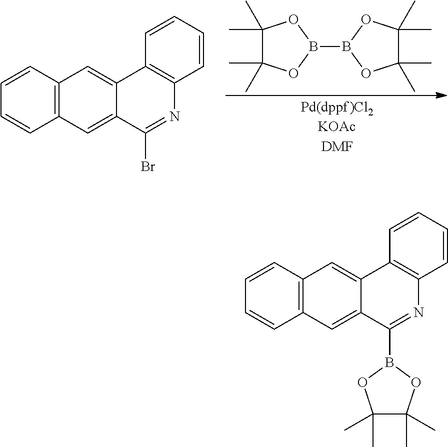

5-bromobenzo[b] phenanthridine (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 5-(4,4,5,5-tetramethyl-1,3,2-dioxaborolan-2-yl)benzo[b]phenanthridine.

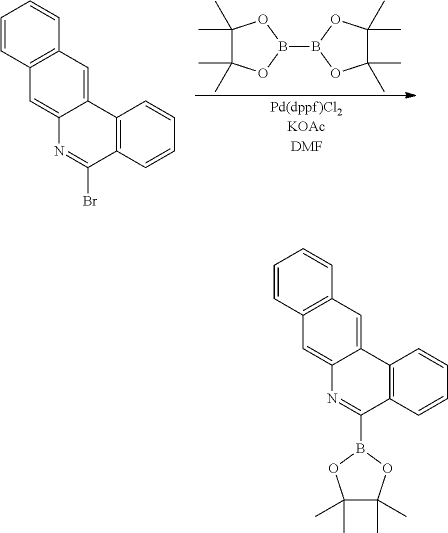

6-bromobenzo[c] phenanthridine (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 6-(4,4,5,5-tetramethyl-1,3,2-dioxaborolan-2-yl)benzo[c]phenanthridine.

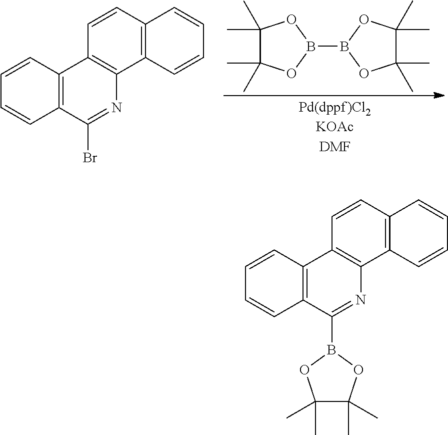

5-bromobenzo[i] phenanthridine (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 5-(4,4,5,5-tetramethyl-1,3,2-dioxaborolan-2-yl)benzo[i]phenanthridine.

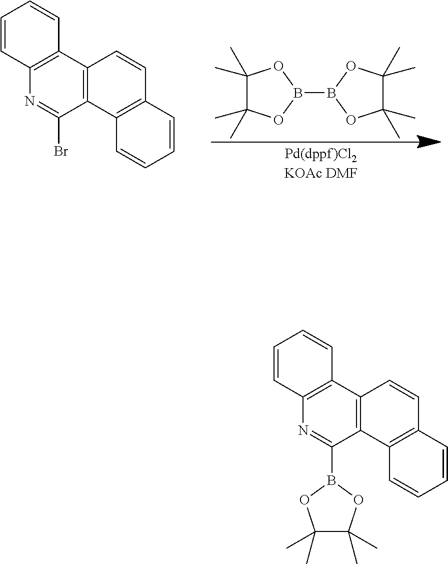

3-bromobenzo [1,2-h] quinoline (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 3-(4,4,5,5-tetramethyl-1,3,2-dioxaborolan-2-yl)naphtho[1,2-h]quinoline.

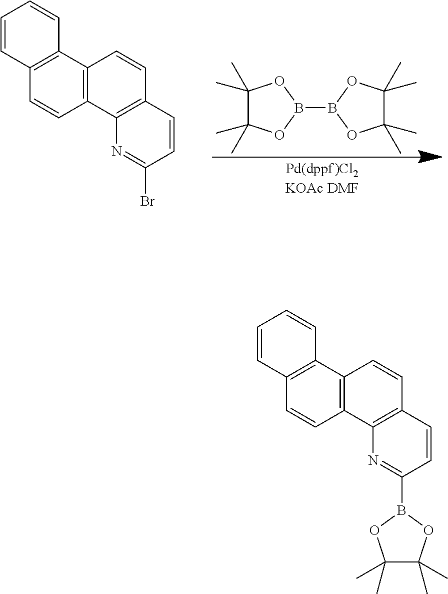

TABLE 1

| Compound | FD-MS |
|---|---|
| Core 1-1 | Chemical Formula: C₂₃H₂₂BNO₂ |
| | Molecular Weight: 355.24 |
| | m/z: 355.17 |
| Core 1-2 | Chemical Formula: C₂₃H₂₂BNO₂ |
| | Molecular Weight: 355.24 |
| | m/z: 355.17 |
| Core 1-3 | Chemical Formula: C₂₃H₂₂BNO₂ |
| | Molecular Weight: 355.24 |
| | m/z: 355.17 |
| Core 1-4 | Chemical Formula: C23H22BNO2 |
| | Molecular Weight: 355.24 |
| | m/z: 355.17 |
| Core 1-5 | Chemical Formula: C₂₃H₂₂BNO₂ |
| | Molecular Weight: 355.24 |
| | m/z: 355.17 |

### (2) Synthesis of Cores 2-1 to 2-4

8-bromobenzo [h] isoquinoline (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 8-(4,4,5,5-tetramethyl-1,3,2-dioxaborolan-2-yl)benzo[h]isoquinoline.

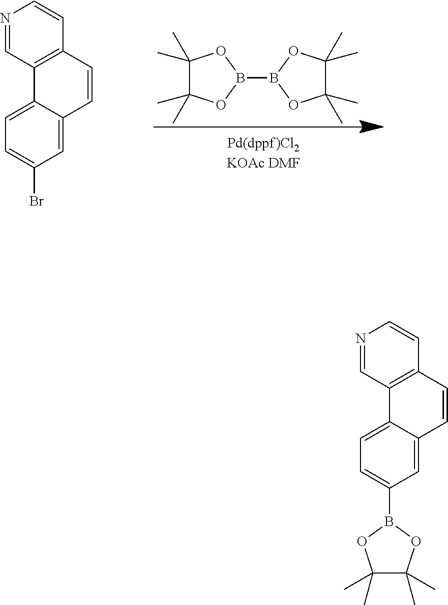

3-bromophenanthridine (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 3-(4,4,5,5-tetramethyl-1,3,2-dioxaborolan-2-yl)phenanthridine.

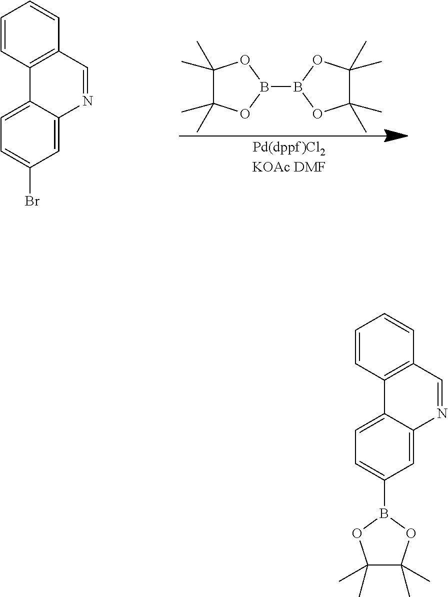

7-bromobenzo[h]quinoline (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 7-(4,4,5,5-tetramethyl-1,3,2-dioxaborolan-2-yl)benzo[h]quinoline.

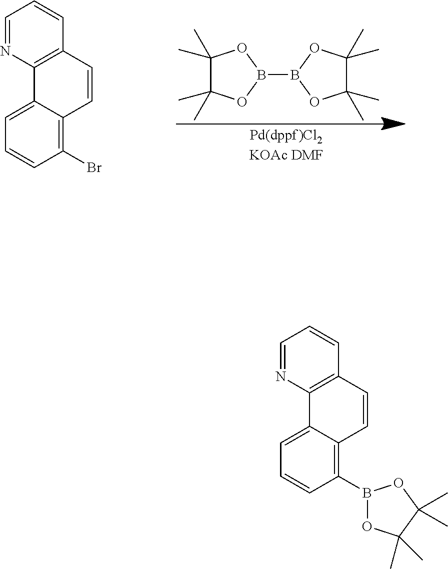

6-bromobenzo[h]quinoline (1 eq) was dissolved with DMF in a round bottom flask, and then bis(pinacolato)diboron (1.1 eq), Pd(dppf)Cl₂ (0.03 eq), and KOAc (3 eq) were added and stirred under reflux at 130° C. for 4 hours. Upon completion of the reaction, DMF was removed from the reaction products via distillation and the reaction product was extracted with CH₂Cl₂ and water. The resulting organic layer was dried using MgSO₄ and concentrated, and the resulting compound was subjected to silicagel column and recrystallization to obtain 6-(4,4,5,5-tetramethyl-1,3,2-dioxaborolan-2-yl)benzo[h]quinoline.

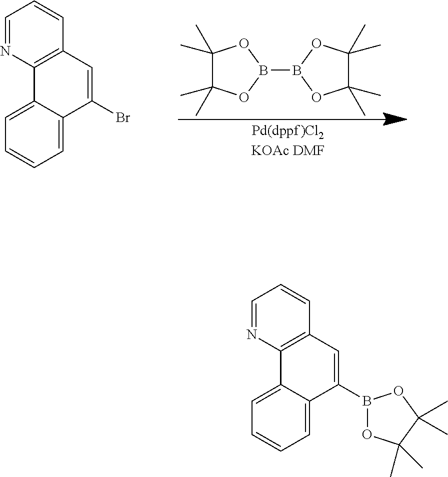

TABLE 2

| Compound | FD-MS |
|---|---|
| Core 2-1 | Chemical Formula: C₁₉H₂₀BNO₂ |
| | Molecular Weight: 305.18 |
| | m/z: 305.16 |
| Core 2-2 | Chemical Formula: C₁₉H₂₀BNO₂ |
| | Molecular Weight: 305.18 |
| | m/z: 305.16 |
| Core 2-3 | Chemical Formula: C₁₉H₂₀BNO₂ |
| | Molecular Weight: 305.18 |
| | m/z: 305.16 |
| Core 2-4 | Chemical Formula: C₁₉H₂₀BNO₂ |
| | Molecular Weight: 305.18 |
| | m/z: 305.16 |

### <Synthesis Example and FDMS Data of Final Products>

### Synthesis Example (Compounds 1-1-1 to 1-1-5)

6-(4,4,5,5-tetramethyl-1,3,2-dioxaborolan-2-yl)benzo[j]phenanthridine (20 g, 56.3 mmol) was dissolved with THE, and then 2-([1,1′-biphenyl]-4-yl)-4-chloro-6-phenyl-1,3,5-triazine (21.1 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 19 g (yield: 63%) of the final product.

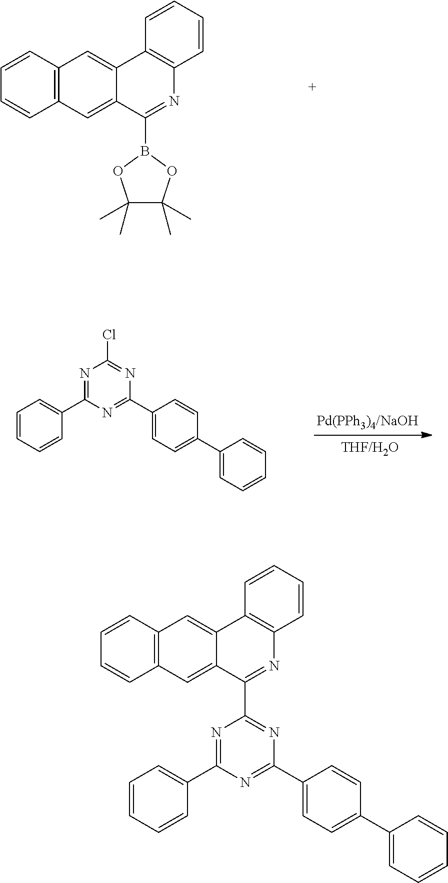 [SMILES: CC1(C)OB(c2nc3ccccc3c3cc4ccccc4cc23)OC1(C)C ; Clc1nc(-c2ccccc2)nc(-c2ccc(-c3ccccc3)cc2)n1 ; c1ccc(-c2ccc(-c3nc(-c4ccccc4)nc(-c4nc5ccccc5c5cc6ccccc6cc45)n3)cc2)cc1]

Compounds 1-1-2 to 1-1-5 are synthesized by the same method as Compound 1-1-1, using Cores 1-2 to 1-5.

### Synthesis Example (Compounds 1-2-1 to 1-2-5)

6-(4,4,5,5-tetramethyl-1,3,2-dioxaborolan-2-yl)benzo[j]phenanthridine (20 g, 56.3 mmol) was dissolved with THE, and then 2-([1,1′-biphenyl]-4-yl)-4-(4-bromophenyl)-6-phenyl-1,3,5-triazine (28.8 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 23 g (yield: 67%) of the final product.

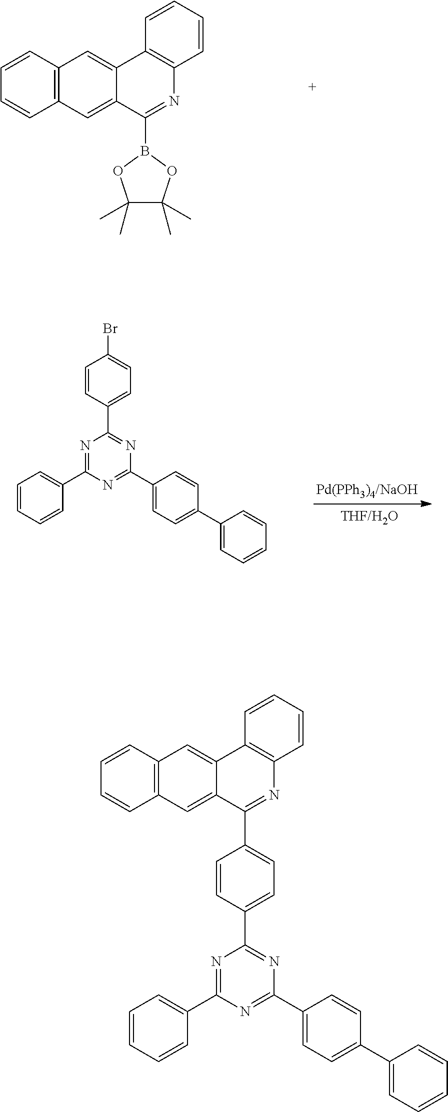 [SMILES: CC1(C)OB(c2nc3ccccc3c3cc4ccccc4cc23)OC1(C)C ; Brc1ccc(-c2nc(-c3ccccc3)nc(-c3ccc(-c4ccccc4)cc3)n2)cc1 ; c1ccc(-c2ccc(-c3nc(-c4ccccc4)nc(-c4ccc(-c5nc6ccccc6c6cc7ccccc7cc56)cc4)n3)cc2)cc1]

Compounds 1-2-2 to 1-2-5 are synthesized by the same method as Compound 1-2-1 using Cores 1-2 to 1-5.

### Synthesis Example (Compounds 1-2-6 to 1-2-10)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(3-bromophenyl)-6-phenyl-1,3,5-triazine (28.8 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 22 g (yield: 64%) of the final product.

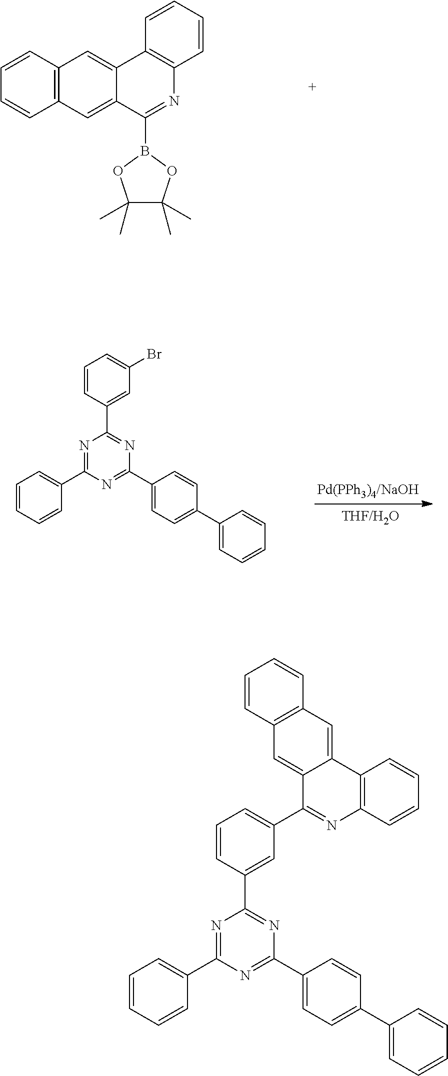 [SMILES: CC1(C)OB(c2nc3ccccc3c3cc4ccccc4cc23)OC1(C)C ; Brc1cccc(-c2nc(-c3ccccc3)nc(-c3ccc(-c4ccccc4)cc3)n2)c1 ; c1ccc(-c2ccc(-c3nc(-c4ccccc4)nc(-c4cccc(-c5nc6ccccc6c6cc7ccccc7cc56)c4)n3)cc2)cc1]

Compounds 1-2-7 to 1-2-10 are synthesized by the same method as Compound 1-2-6 using Cores 1-2 to 1-5.

### Synthesis Example (Compounds 1-3-1 to 1-3-5)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(4′-bromo-[1,1′-biphenyl]-4-yl)-6-phenyl-1,3,5-triazine (33.5 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 26 g (yield: 67%) of the final product.

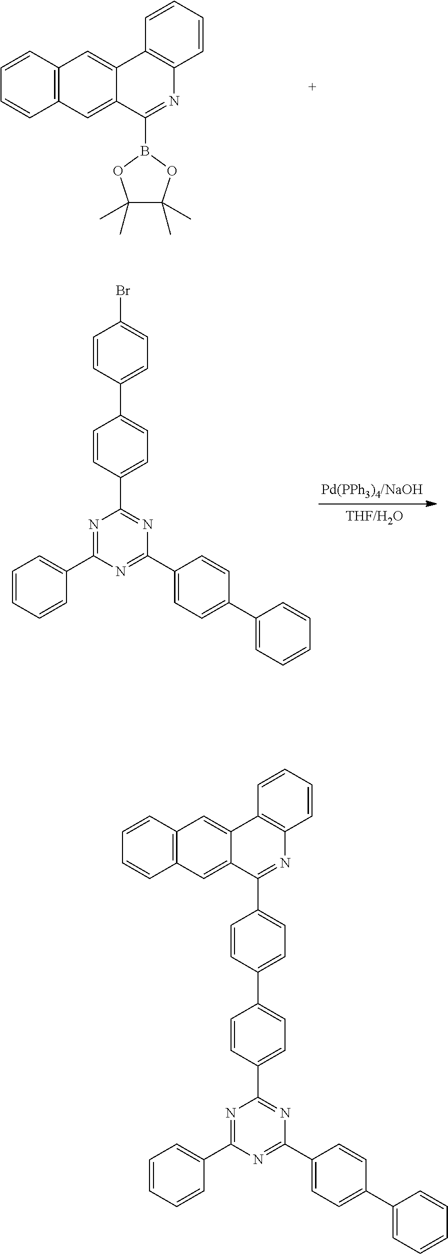 [SMILES: CC1(C)OB(c2nc3ccccc3c3cc4ccccc4cc23)OC1(C)C ; Brc1ccc(-c2ccc(-c3nc(-c4ccccc4)nc(-c4ccc(-c5ccccc5)cc4)n3)cc2)cc1 ; c1ccc(-c2ccc(-c3nc(-c4ccccc4)nc(-c4ccc(-c5ccc(-c6nc7ccccc7c7cc8ccccc8cc67)cc5)cc4)n3)cc2)cc1]

Compounds 1-3-2 to 1-3-5 are synthesized by the same method as Compound 1-3-1 using Cores 1-2 to 1-5.

### Synthesis Example (Compounds 1-3-6 to 1-3-10)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(4′-bromo-[1,1′-biphenyl]-3-yl)-6-phenyl-1,3,5-triazine (33.5 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 27 g (yield: 69%) of the final product.

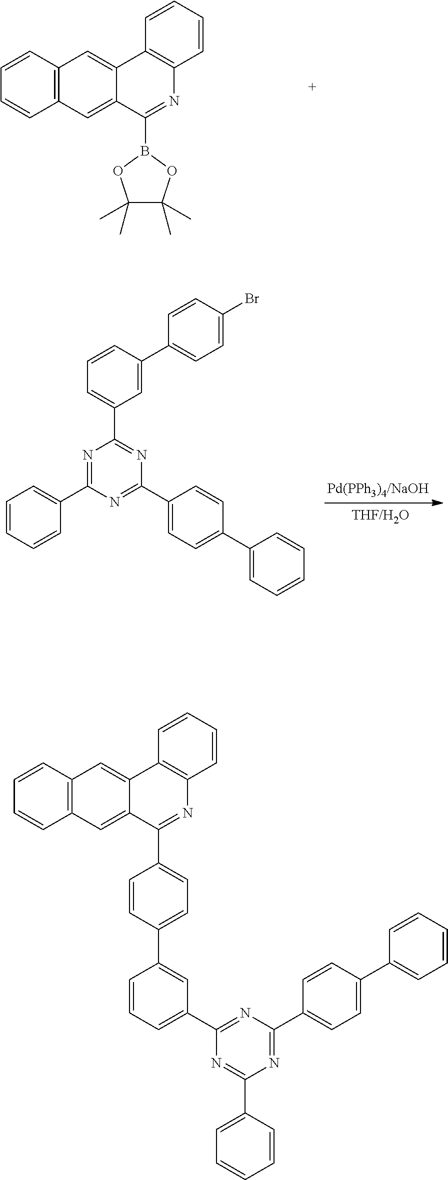 [SMILES: CC1(C)OB(c2nc3ccccc3c3cc4ccccc4cc23)OC1(C)C ; Brc1ccc(-c2cccc(-c3nc(-c4ccccc4)nc(-c4ccc(-c5ccccc5)cc4)n3)c2)cc1 ; c1ccc(-c2ccc(-c3nc(-c4ccccc4)nc(-c4cccc(-c5ccc(-c6nc7ccccc7c7cc8ccccc8cc67)cc5)c4)n3)cc2)cc1]

Compounds 1-3-7 to 1-3-10 are synthesized by the same method as Compound 1-3-6 using Cores 1-2 to 1-5.

### Synthesis Example (Compounds 1-3-11 to 1-3-15)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(3′-bromo-[1,1′-biphenyl]-4-yl)-6-phenyl-1,3,5-triazine (33.5 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 28 g (yield: 72%) of the final product.

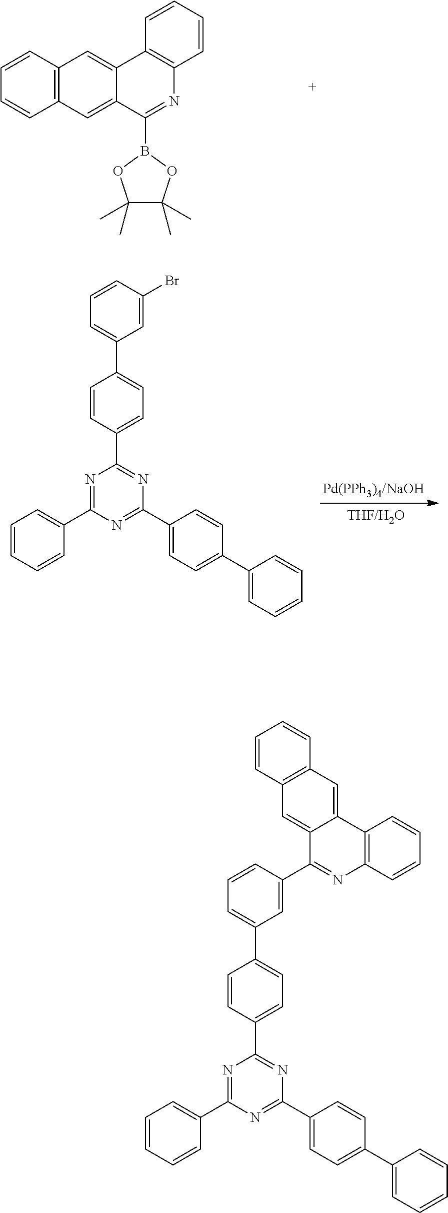 [SMILES: CC1(C)OB(c2nc3ccccc3c3cc4ccccc4cc23)OC1(C)C ; Brc1cccc(-c2ccc(-c3nc(-c4ccccc4)nc(-c4ccc(-c5ccccc5)cc4)n3)cc2)c1 ; c1ccc(-c2ccc(-c3nc(-c4ccccc4)nc(-c4ccc(-c5cccc(-c6nc7ccccc7c7cc8ccccc8cc67)c5)cc4)n3)cc2)cc1]

Compounds 1-3-12 to 1-3-15 are synthesized by the same method as Compound 1-3-11 using Cores 1-2 to 1-5.

### Synthesis Example (Compounds 1-3-16 to 1-3-20)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(3′-bromo-[1,1′-biphenyl]-3-yl)-6-phenyl-1,3,5-triazine (33.5 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 26 g (yield: 67%) of the final product.

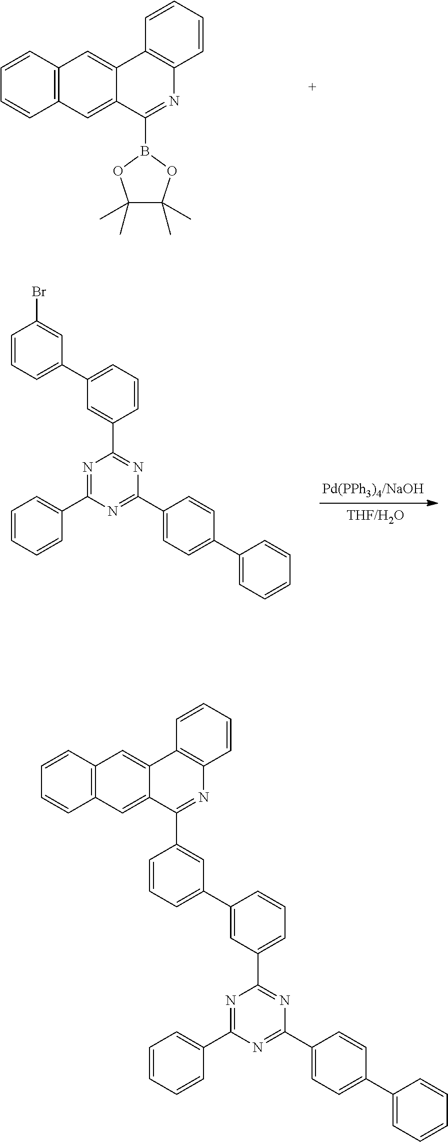 [SMILES: CC1(C)OB(c2nc3ccccc3c3cc4ccccc4cc23)OC1(C)C ; Brc1cccc(-c2cccc(-c3nc(-c4ccccc4)nc(-c4ccc(-c5ccccc5)cc4)n3)c2)c1 ; c1ccc(-c2ccc(-c3nc(-c4ccccc4)nc(-c4cccc(-c5cccc(-c6nc7ccccc7c7cc8ccccc8cc67)c5)c4)n3)cc2)cc1]

Compounds 1-3-17 to 1-3-20 are synthesized by the same method as Compound 1-3-16, using Cores 1-2 to 1-5.

### Synthesis Example (Compounds 1-4-1 to 1-4-5)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(4″-bromo-[1,1′: 4′,1″-terphenyl]-4-yl)-6-phenyl-1,3,5-triazine (38.2 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 30 g (yield: 70%) of the final product.

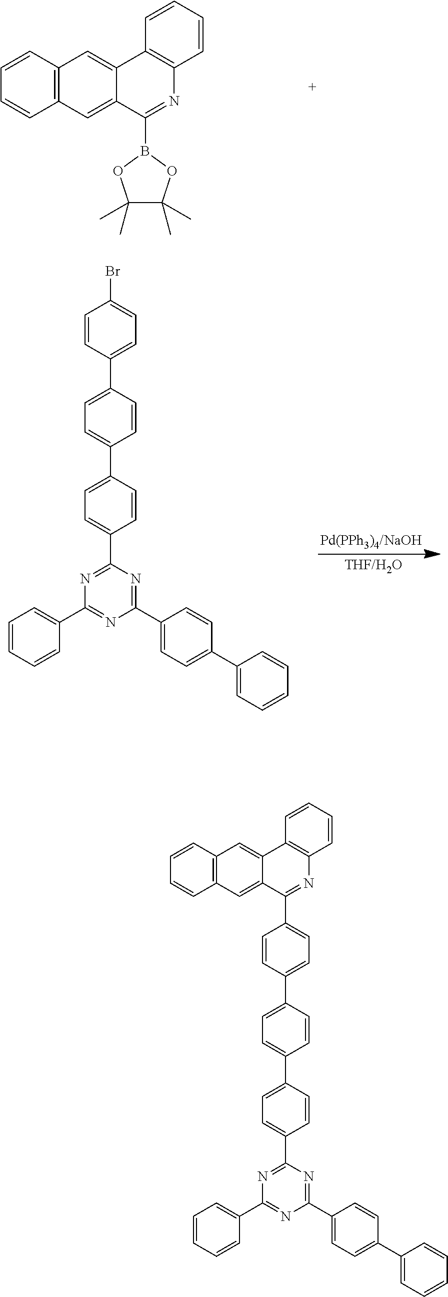

Compounds 1-4-2 to 1-4-5 are synthesized by the same method as Compound 1-4-1 using Cores 1-2 to 1-5.

### Synthesis Example (Compounds 1-4-6 to 1-4-10)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(3″-bromo-[1,1′: 4′,1″-terphenyl]-4-yl)-6-phenyl-1,3,5-triazine (38.2 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 28 g (yield: 65%) of the final product.

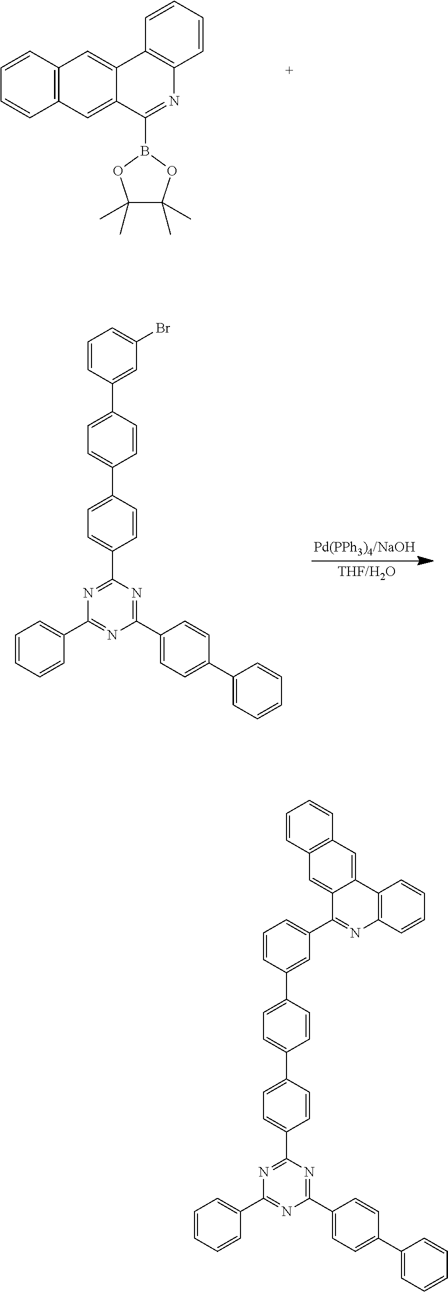 [SMILES: CC1(C)OB(c2nc3ccccc3c3cc4ccccc4cc23)OC1(C)C ; Brc1cccc(-c2ccc(-c3ccc(-c4nc(-c5ccccc5)nc(-c5ccc(-c6ccccc6)cc5)n4)cc3)cc2)c1 ; c1ccc(-c2ccc(-c3nc(-c4ccccc4)nc(-c4ccc(-c5ccc(-c6cccc(-c7nc8ccccc8c8cc9ccccc9cc78)c6)cc5)cc4)n3)cc2)cc1]

Compounds 1-4-7 to 1-4-10 are synthesized by the same method as Compound 1-4-6 using Cores 1-2 to 1-5.

### Synthesis Example (Compounds 1-4-11 to 1-4-15)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(4″-bromo-[1,1′: 3′,1″-terphenyl]-4-yl)-6-phenyl-1,3,5-triazine (38.2 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 29 g (yield: 67%) of the final product.

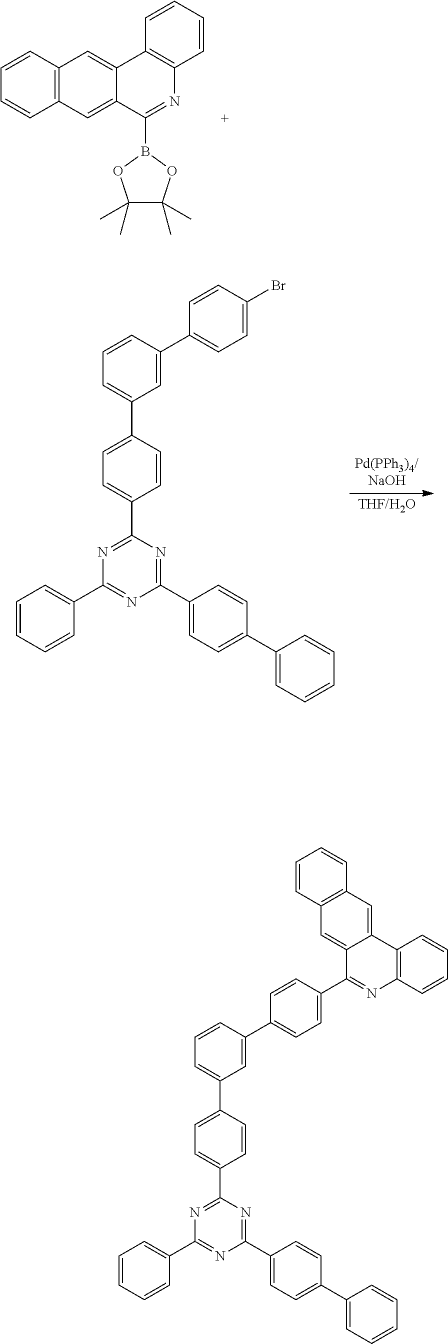 [SMILES: CC1(C)OB(c2nc3ccccc3c3cc4ccccc4cc23)OC1(C)C ; Brc1ccc(-c2cccc(-c3ccc(-c4nc(-c5ccccc5)nc(-c5ccc(-c6ccccc6)cc5)n4)cc3)c2)cc1 ; c1ccc(-c2ccc(-c3nc(-c4ccccc4)nc(-c4ccc(-c5cccc(-c6ccc(-c7nc8ccccc8c8cc9ccccc9cc78)cc6)c5)cc4)n3)cc2)cc1]

Compounds 1-4-12 to 1-4-15 are synthesized by the same method as Compound 1-4-11 using Cores 1-2 to 1-5.

### Synthesis Example (Compounds 1-4-16 to 1-4-20)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(4″-bromo-[1,1′: 3′,1″-terphenyl]-3-yl)-6-phenyl-1,3,5-triazine (38.2 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 27 g (yield: 63%) of the final product.

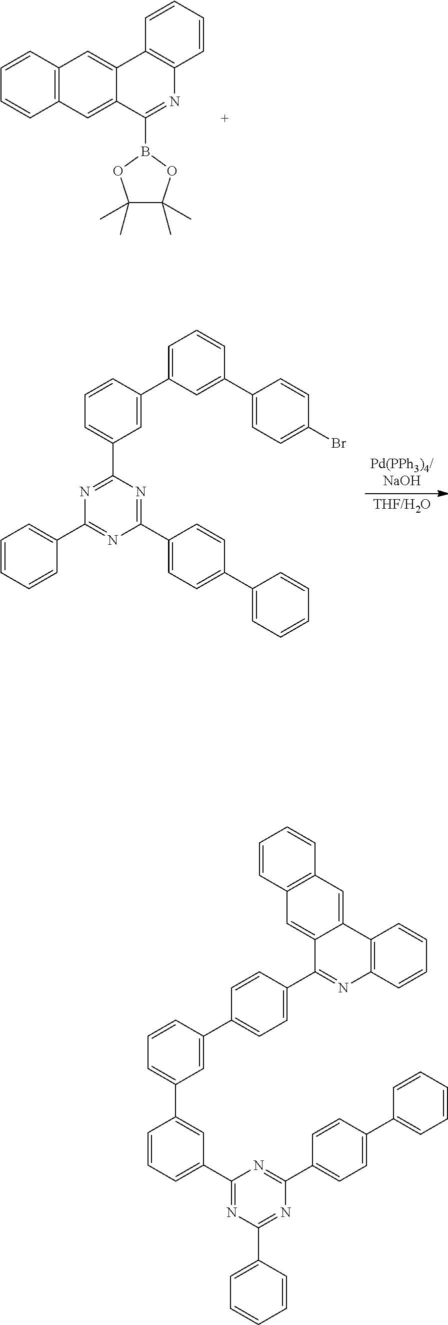 [SMILES: CC1(C)OB(c2nc3ccccc3c3cc4ccccc4cc23)OC1(C)C ; Brc1ccc(-c2cccc(-c3cccc(-c4nc(-c5ccccc5)nc(-c5ccc(-c6ccccc6)cc5)n4)c3)c2)cc1 ; c1ccc(-c2ccc(-c3nc(-c4ccccc4)nc(-c4cccc(-c5cccc(-c6ccc(-c7nc8ccccc8c8cc9ccccc9cc78)cc6)c5)c4)n3)cc2)cc1]

Compounds 1-4-17 to 1-4-20 are synthesized by the same method as Compound 1-4-16, using Cores 1-2 to 1-5.

### Synthesis Example (Compounds 1-4-21 to 1-4-25)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(4″-bromo-[1,1′: 4′,1″-terphenyl]-3-yl)-6-phenyl-1,3,5-triazine (38.2 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 26 g (yield: 60%) of the final product.

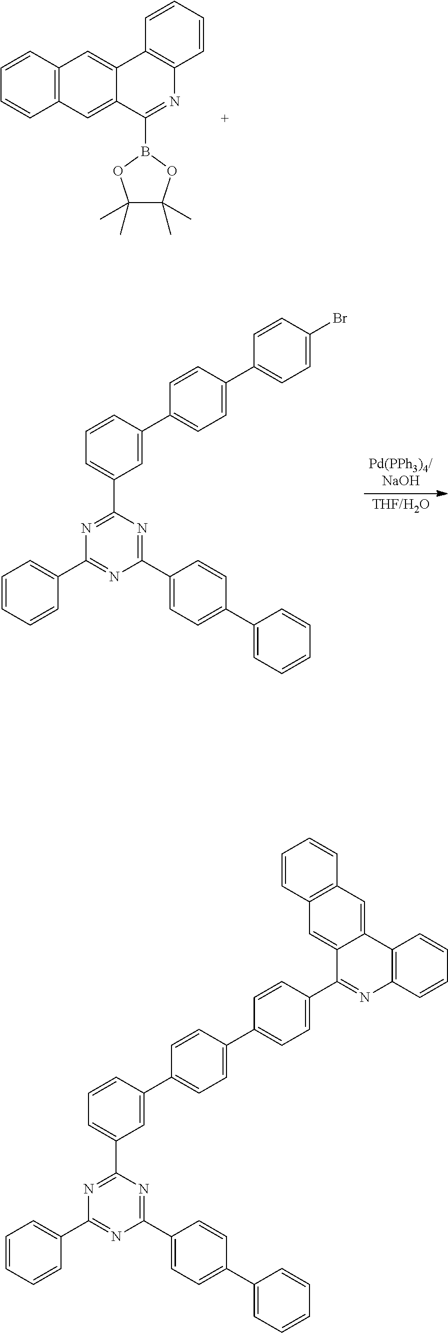 [SMILES: CC1(C)OB(c2nc3ccccc3c3cc4ccccc4cc23)OC1(C)C ; Brc1ccc(-c2ccc(-c3cccc(-c4nc(-c5ccccc5)nc(-c5ccc(-c6ccccc6)cc5)n4)c3)cc2)cc1 ; c1ccc(-c2ccc(-c3nc(-c4ccccc4)nc(-c4cccc(-c5ccc(-c6ccc(-c7nc8ccccc8c8cc9ccccc9cc78)cc6)cc5)c4)n3)cc2)cc1]

Compounds 1-4-22 to 1-4-25 are synthesized by the same method as Compound 1-4-21, using Cores 1-2 to 1-5.

### Synthesis Example (Compounds 1-4-26 to 1-4-30)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(3″-bromo-[1,1′: 3′,1″-terphenyl]-3-yl)-6-phenyl-1,3,5-triazine (38.2 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 27 g (yield: 63%) of the final product.

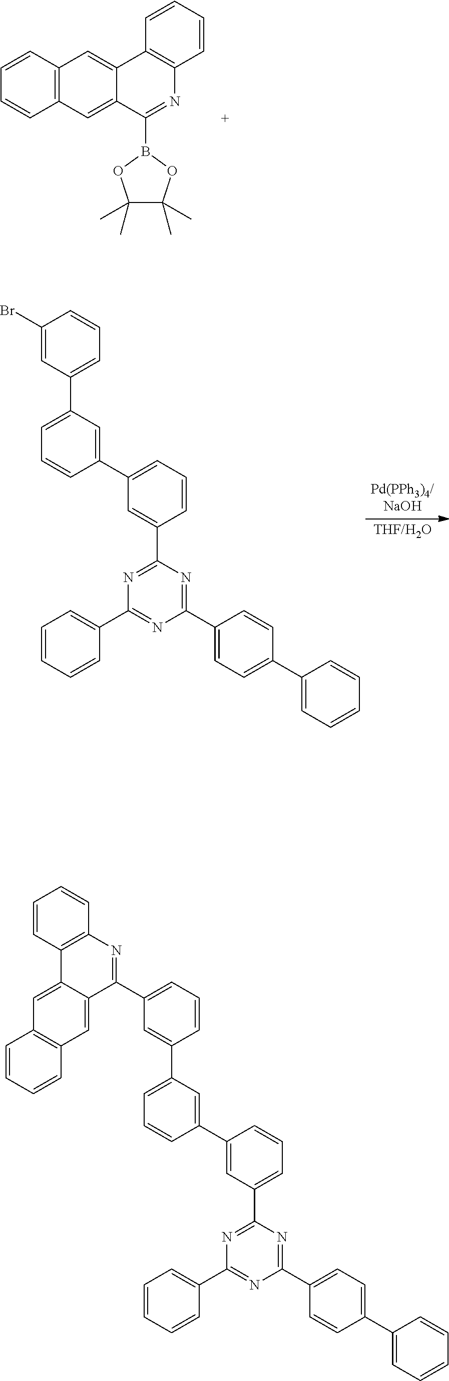 [SMILES: CC1(C)OB(c2nc3ccccc3c3cc4ccccc4cc23)OC1(C)C ; Brc1cccc(-c2cccc(-c3cccc(-c4nc(-c5ccccc5)nc(-c5ccc(-c6ccccc6)cc5)n4)c3)c2)c1 ; c1ccc(-c2ccc(-c3nc(-c4ccccc4)nc(-c4cccc(-c5cccc(-c6cccc(-c7nc8ccccc8c8cc9ccccc9cc78)c6)c5)c4)n3)cc2)cc1]

Compounds 1-4-27 to 1-4-30 are synthesized by the same method as Compound 1-4-26, using Cores 1-2 to 1-5.

### Synthesis Example (Compounds 1-4-31 to 1-4-35)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(3″-bromo-[1,1′: 4′,1″-terphenyl]-3-yl)-6-phenyl-1,3,5-triazine (38.2 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 28 g (yield: 65%) of the final product.

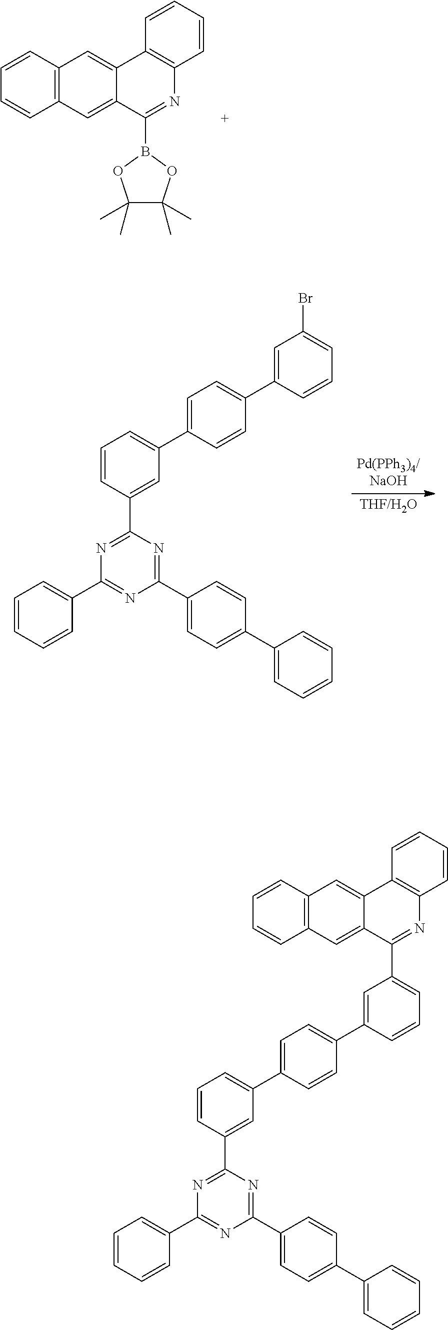 [SMILES: CC1(C)OB(c2nc3ccccc3c3cc4ccccc4cc23)OC1(C)C ; Brc1cccc(-c2ccc(-c3cccc(-c4nc(-c5ccccc5)nc(-c5ccc(-c6ccccc6)cc5)n4)c3)cc2)c1 ; c1ccc(-c2ccc(-c3nc(-c4ccccc4)nc(-c4cccc(-c5ccc(-c6cccc(-c7nc8ccccc8c8cc9ccccc9cc78)c6)cc5)c4)n3)cc2)cc1]

Compounds 1-4-32 to 1-4-35 are synthesized by the same method as Compound 1-4-31, using Cores 1-2 to 1-5.

### Synthesis Example (Compounds 1-4-36 to 1-4-40)

6-(4,4,5,5-tetramethyl-1,3,2-dioxaborolan-2-yl)benzo[j] phenanthridine (20 g, 56.3 mmol) was dissolved with THE, and then 2-([1,1′-biphenyl]-4-yl)-4-(3″-bromo-[1,1′: 3′,1″-terphenyl]-4-yl)-6-phenyl-1,3,5-triazine (38.2 g, 61.9 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 29 g (yield: 67%) of the final product.

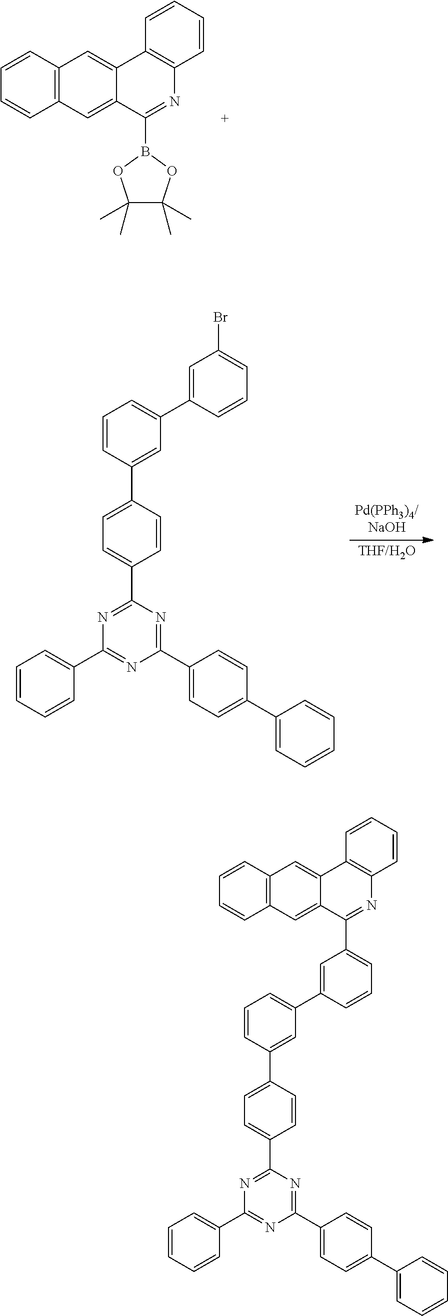 [SMILES: CC1(C)OB(c2nc3ccccc3c3cc4ccccc4cc23)OC1(C)C ; Brc1cccc(-c2cccc(-c3ccc(-c4nc(-c5ccccc5)nc(-c5ccc(-c6ccccc6)cc5)n4)cc3)c2)c1 ; c1ccc(-c2ccc(-c3nc(-c4ccccc4)nc(-c4ccc(-c5cccc(-c6cccc(-c7nc8ccccc8c8cc9ccccc9cc78)c6)c5)cc4)n3)cc2)cc1]

Compounds 1-4-37 to 1-4-40 are synthesized by the same method as Compound 1-4-36, using Cores 1-2 to 1-5.

TABLE 3

| Compound | FD-MS |
|---|---|
| 1-1-1~1-1-5 | Chemical Formula: C₃₈H₂₄N₄ |
| | Molecular Weight: 536.64 |
| | m/z: 536.20 |
| 1-2-1~1-2-5 | Chemical Formula: C₄₄H₂₈N₄ |
| | Molecular Weight: 612.74 |
| | m/z: 612.23 |
| 1-2-6~1-2-10 | Chemical Formula: C₄₄H₂₈N₄ |
| | Molecular Weight: 612.74 |
| | m/z: 612.23 |
| 1-3-1~1-3-5 | Chemical Formula: C₅₀H₃₂N₄ |
| | Molecular Weight: 688.83 |
| | m/z: 688.26 |
| 1-3-6~1-3-10 | Chemical Formula: C₅₀H₃₂N₄ |
| | Molecular Weight: 688.83 |
| | m/z: 688.26 |
| 1-3-11~1-3-15 | Chemical Formula: C₅₀H₃₂N₄ |
| | Molecular Weight: 688.83 |
| | m/z: 688.26 |
| 1-3-16~1-3-20 | Chemical Formula: C₅₀H₃₂N₄ |
| | Molecular Weight: 688.83 |
| | m/z: 688.26 |
| 1-4-1~1-4-5 | Chemical Formula: C₅₆H₃₆N₄ |
| | Molecular Weight: 764.93 |
| | m/z: 764.29 |
| 1-4-6~1-4-10 | Chemical Formula: C₅₆H₃₆N₄ |
| | Molecular Weight: 764.93 |
| | m/z: 764.29 |
| 1-4-11~1-4-15 | Chemical Formula: C₅₆H₃₆N₄ |
| | Molecular Weight: 764.93 |
| | m/z: 764.29 |
| 1-4-16~1-4-20 | Chemical Formula: C₅₆H₃₆N₄ |
| | Molecular Weight: 764.93 |
| | m/z: 764.29 |
| 1-4-21~1-4-25 | Chemical Formula: C₅₆H₃₆N₄ |
| | Molecular Weight: 764.93 |
| | m/z: 764.29 |
| 1-4-26~1-4-30 | Chemical Formula: C₅₆H₃₆N₄ |
| | Molecular Weight: 764.93 |
| | m/z: 764.29 |
| 1-4-31~1-4-35 | Chemical Formula: C₅₆H₃₆N₄ |
| | Molecular Weight: 764.93 |
| | m/z: 764.29 |
| 1-4-36~1-4-40 | Chemical Formula: C₅₆H₃₆N₄ |
| | Molecular Weight: 764.93 |
| | m/z: 764.29 |

### Synthesis Example (Compounds 2-1-1 to 2-1-4)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-chloro-6-phenyl-1,3,5-triazine (24.8 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 22 g (yield: 69%) of the final product.

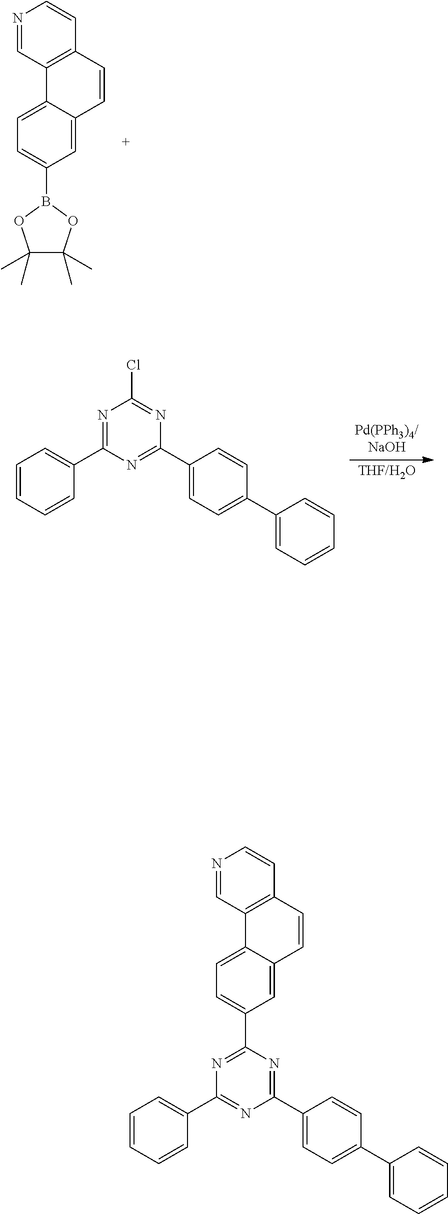 [SMILES: CC1(C)OB(c2ccc3c(ccc4ccncc43)c2)OC1(C)C ; Clc1nc(-c2ccccc2)nc(-c2ccc(-c3ccccc3)cc2)n1 ; c1ccc(-c2ccc(-c3nc(-c4ccccc4)nc(-c4ccc5c(ccc6ccncc65)c4)n3)cc2)cc1]

Compounds 2-1-2 to 2-1-4 can be synthesized by the same method as Compound 2-1-1, using Cores 2-2 to 2-4.

### Synthesis Example (Compounds 2-2-1 to 2-2-4)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(4-bromophenyl)-6-phenyl-1,3,5-triazine (33.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 24 g (yield: 65%) of the final product.

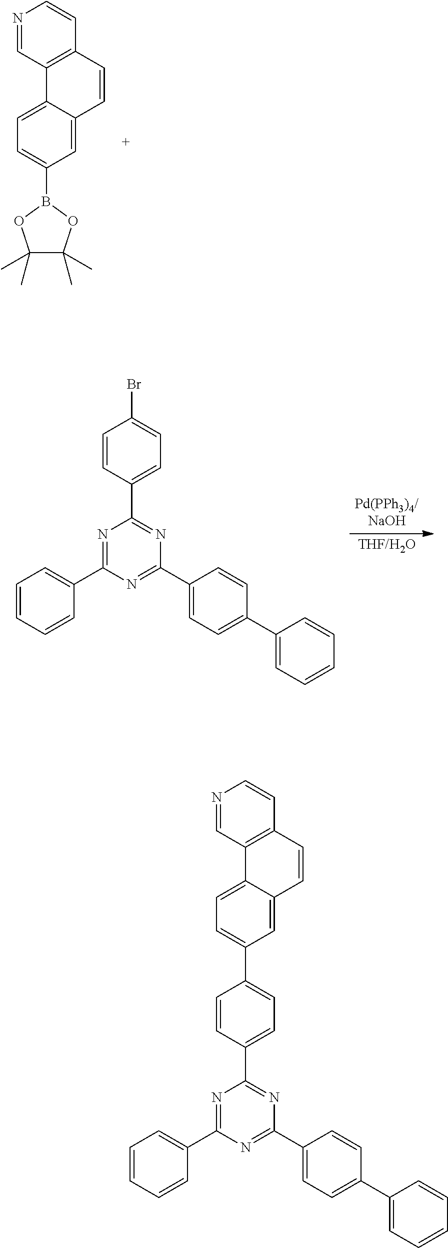 [SMILES: CC1(C)OB(c2ccc3c(ccc4ccncc43)c2)OC1(C)C ; Brc1ccc(-c2nc(-c3ccccc3)nc(-c3ccc(-c4ccccc4)cc3)n2)cc1 ; c1ccc(-c2ccc(-c3nc(-c4ccccc4)nc(-c4ccc(-c5ccc6c(ccc7ccncc76)c5)cc4)n3)cc2)cc1]

Compounds 2-2-2 to 2-2-4 can be synthesized by the same method as Compound 2-2-1 using Cores 2-2 to 2-4.

### Synthesis Example (Compounds 2-2-5 to 2-2-8)

8-(4,4,5,5-tetramethyl-1,3,2-dioxaborolan-2-yl)benzo[h]isoquinoline (20 g, 65.5 mmol) was dissolved with THF, and then 2-([1,1′-biphenyl]-4-yl)-4-(3-bromophenyl)-6-phenyl-1,3,5-triazine (33.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 24 g (yield: 65%) of the final product.

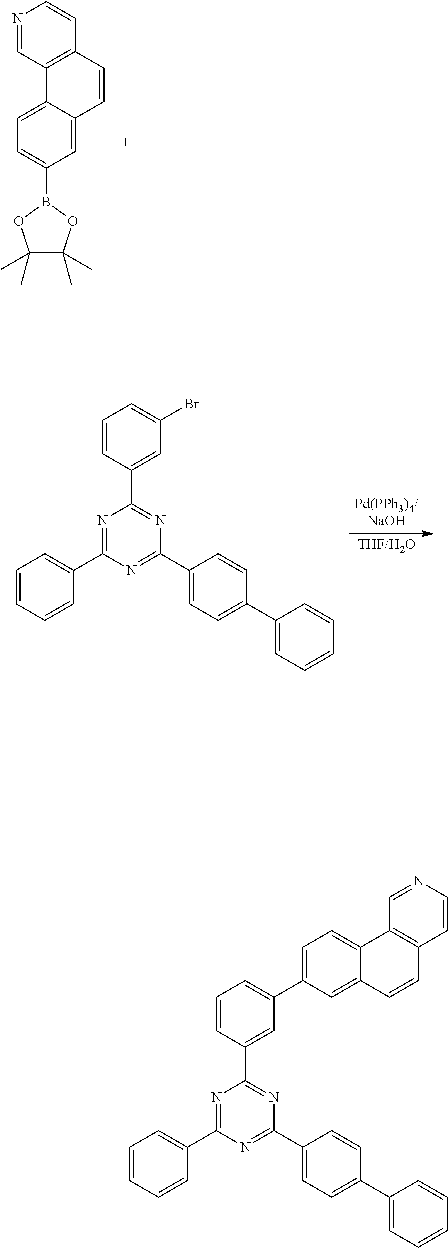 [SMILES: CC1(C)OB(c2ccc3c(ccc4ccncc43)c2)OC1(C)C ; Brc1cccc(-c2nc(-c3ccccc3)nc(-c3ccc(-c4ccccc4)cc3)n2)c1 ; c1ccc(-c2ccc(-c3nc(-c4ccccc4)nc(-c4cccc(-c5ccc6c(ccc7ccncc76)c5)c4)n3)cc2)cc1]

Compounds 2-2-6 to 2-2-8 can be synthesized by the same method as Compound 2-2-1 using Cores 2-2 to 2-4.

### Synthesis Example (Compounds 2-3-1 to 2-3-4)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(4′-bromo-[1,1′-biphenyl]-4-yl)-6-phenyl-1,3,5-triazine (38.9 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 27 g (yield: 64%) of the final product.

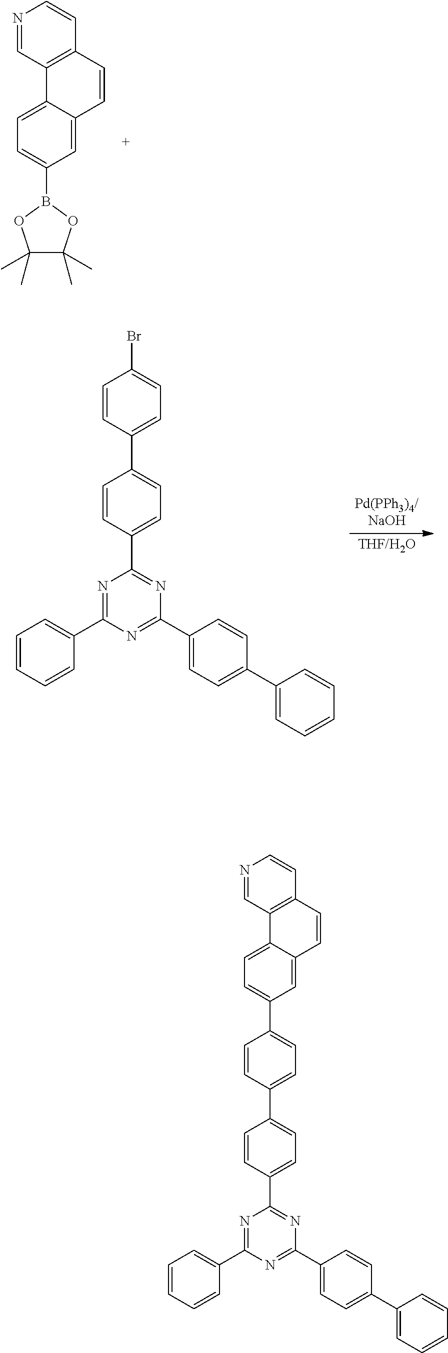 [SMILES: CC1(C)OB(c2ccc3c(ccc4ccncc43)c2)OC1(C)C ; Brc1ccc(-c2ccc(-c3nc(-c4ccccc4)nc(-c4ccc(-c5ccccc5)cc4)n3)cc2)cc1 ; c1ccc(-c2ccc(-c3nc(-c4ccccc4)nc(-c4ccc(-c5ccc(-c6ccc7c(ccc8ccncc87)c6)cc5)cc4)n3)cc2)cc1]

Compounds 2-3-2 to 2-3-4 can be synthesized by the same method as Compound 2-3-1 using Cores 2-2 to 2-4.

### Synthesis Example (Compounds 2-3-5 to 2-3-8)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(4′-bromo-[1,1′-biphenyl]-3-yl)-6-phenyl-1,3,5-triazine (38.9 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 28 g (yield: 67%) of the final product.

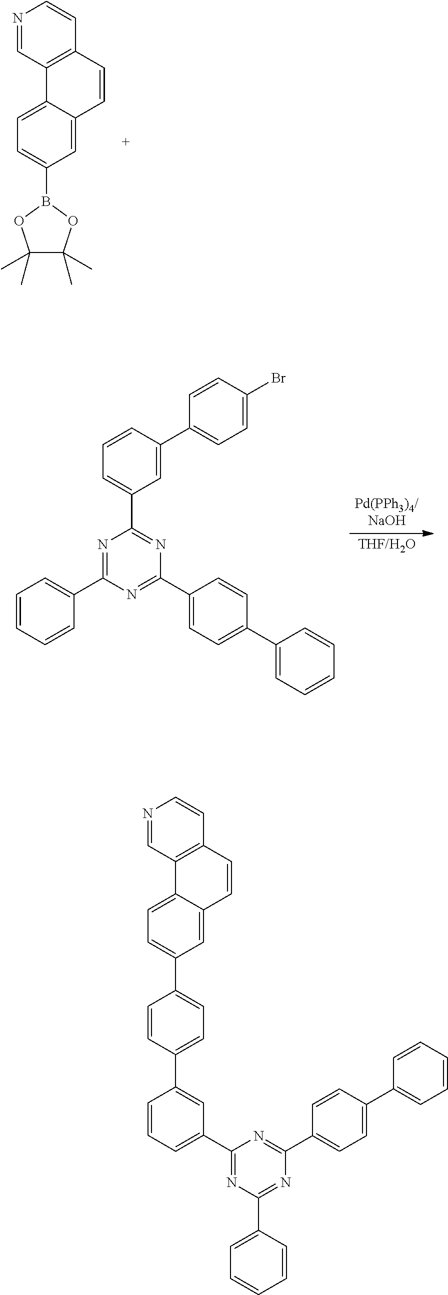 [SMILES: CC1(C)OB(c2ccc3c(ccc4ccncc43)c2)OC1(C)C ; Brc1ccc(-c2cccc(-c3nc(-c4ccccc4)nc(-c4ccc(-c5ccccc5)cc4)n3)c2)cc1 ; c1ccc(-c2ccc(-c3nc(-c4ccccc4)nc(-c4cccc(-c5ccc(-c6ccc7c(ccc8ccncc87)c6)cc5)c4)n3)cc2)cc1]

Compounds 2-3-6 to 2-3-8 can be synthesized by the same method as Compound 2-3-5, using Cores 2-2 to 2-4.

### Synthesis Example (Compounds 2-3-9 to 2-3-12)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(3′-bromo-[1,1′-biphenyl]-4-yl)-6-phenyl-1,3,5-triazine (39.0 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 28 g (yield: 67%) of the final product.

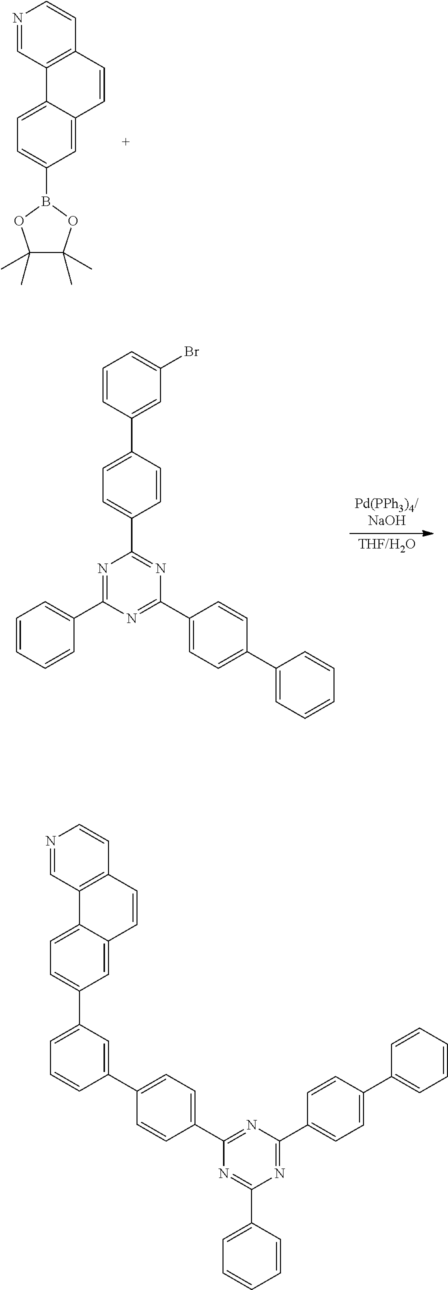 [SMILES: CC1(C)OB(c2ccc3c(ccc4ccncc43)c2)OC1(C)C ; Brc1cccc(-c2ccc(-c3nc(-c4ccccc4)nc(-c4ccc(-c5ccccc5)cc4)n3)cc2)c1 ; c1ccc(-c2ccc(-c3nc(-c4ccccc4)nc(-c4ccc(-c5cccc(-c6ccc7c(ccc8ccncc87)c6)c5)cc4)n3)cc2)cc1]

Compounds 2-3-10 to 2-3-12 can be synthesized by the same method as Compound 2-3-9, using Cores 2-2 to 2-4.

### Synthesis Example (Compounds 2-3-13 to 2-3-16)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(3′-bromo-[1,1′-biphenyl]-3-yl)-6-phenyl-1,3,5-triazine (39.0 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 27 g (yield: 65%) of the final product.

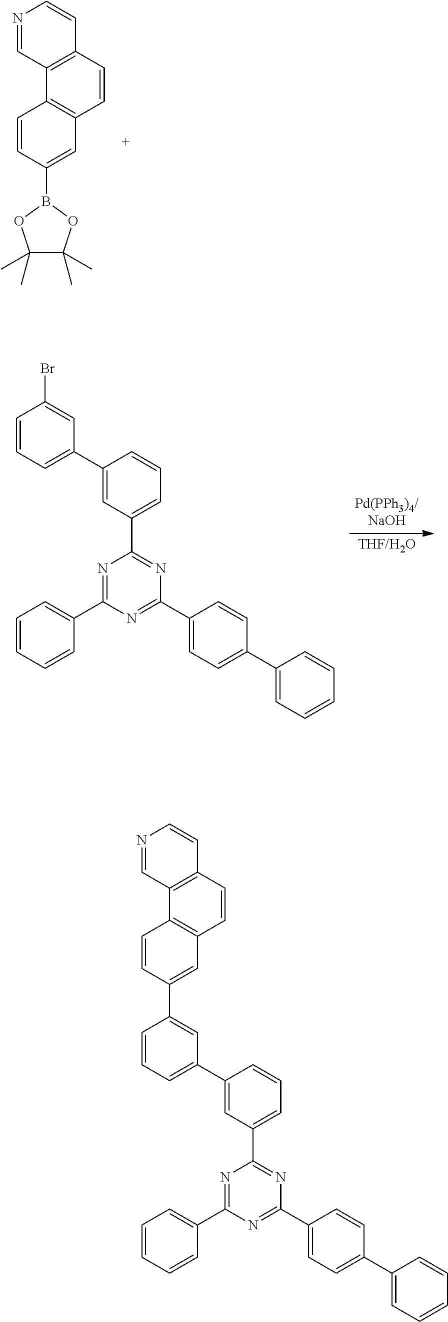 [SMILES: CC1(C)OB(c2ccc3c(ccc4ccncc43)c2)OC1(C)C ; Brc1cccc(-c2cccc(-c3nc(-c4ccccc4)nc(-c4ccc(-c5ccccc5)cc4)n3)c2)c1 ; c1ccc(-c2ccc(-c3nc(-c4ccccc4)nc(-c4cccc(-c5cccc(-c6ccc7c(ccc8ccncc87)c6)c5)c4)n3)cc2)cc1]

Compounds 2-3-14 to 2-3-16 can be synthesized by the same method as Compound 2-3-13, using Cores 2-2 to 2-4.

### Synthesis Example (Compounds 2-4-1 to 2-4-4)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(4″-bromo-[1,1′: 4′,1″-terphenyl]-4-yl)-6-phenyl-1,3,5-triazine (44.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 29 g (yield: 62%) of the final product.

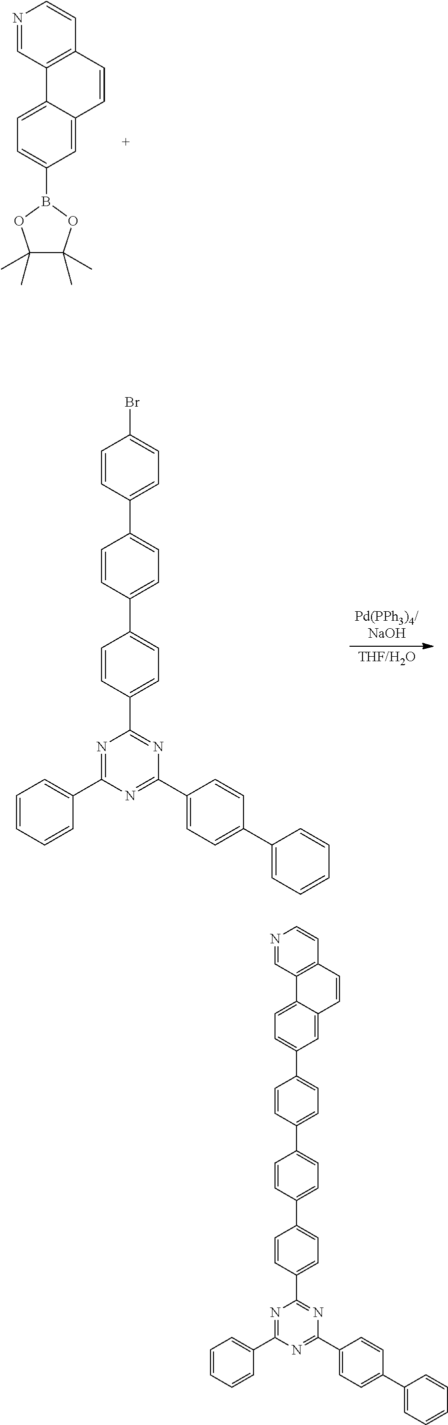

Compounds 2-4-2 to 2-4-4 can be synthesized by the same method as Compound 2-4-1 using Cores 2-2 to 2-4.

### Synthesis Example (Compounds 2-4-5 to 2-4-8)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(3″-bromo-[1,1′: 4′,1″-terphenyl]-4-yl)-6-phenyl-1,3,5-triazine (44.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 30 g (yield: 64%) of the final product.

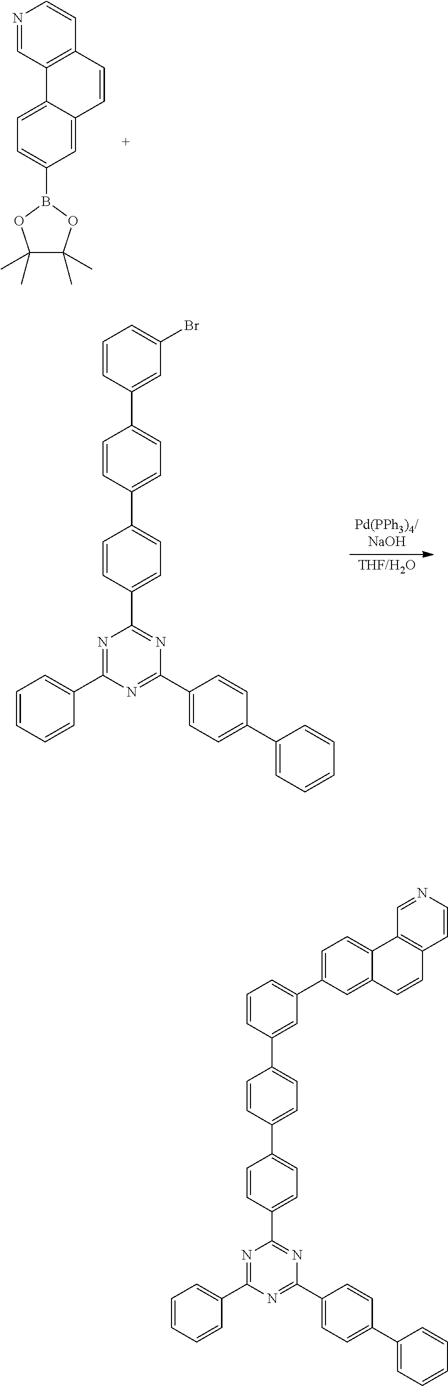 [SMILES: CC1(C)OB(c2ccc3c(ccc4ccncc43)c2)OC1(C)C ; Brc1cccc(-c2ccc(-c3ccc(-c4nc(-c5ccccc5)nc(-c5ccc(-c6ccccc6)cc5)n4)cc3)cc2)c1 ; c1ccc(-c2ccc(-c3nc(-c4ccccc4)nc(-c4ccc(-c5ccc(-c6cccc(-c7ccc8c(ccc9ccncc98)c7)c6)cc5)cc4)n3)cc2)cc1]

Compounds 2-4-6 to 2-4-8 can be synthesized by the same method as Compound 2-4-5, using cores 2-2 to 2-4.

### Synthesis Example (Compounds 2-4-9 to 2-4-12)

8-(4,4,5,5-tetramethyl-1,3,2-dioxaborolan-2-yl)benzo[h]isoquinoline (20 g, 65.5 mmol) was dissolved with THF, and then 2-([1,1′-biphenyl]-4-yl)-4-(4″-bromo-[1,1′: 3′,1″-terphenyl]-4-yl)-6-phenyl-1,3,5-triazine (44.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 32 g (yield: 68%) of the final product.

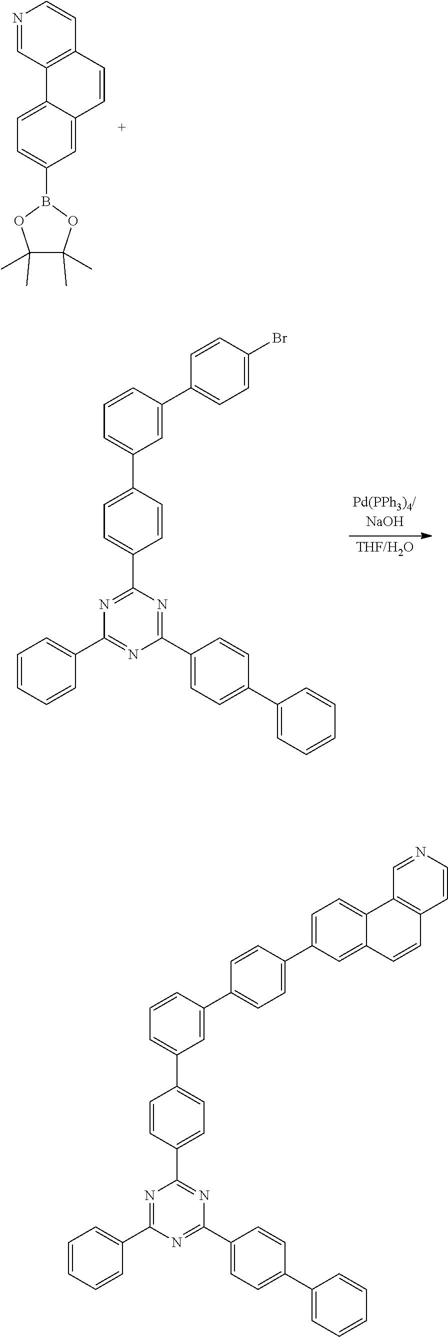 [SMILES: CC1(C)OB(c2ccc3c(ccc4ccncc43)c2)OC1(C)C ; Brc1ccc(-c2cccc(-c3ccc(-c4nc(-c5ccccc5)nc(-c5ccc(-c6ccccc6)cc5)n4)cc3)c2)cc1 ; c1ccc(-c2ccc(-c3nc(-c4ccccc4)nc(-c4ccc(-c5cccc(-c6ccc(-c7ccc8c(ccc9ccncc98)c7)cc6)c5)cc4)n3)cc2)cc1]

Compounds 2-4-10 to 2-4-12 can be synthesized by the same method as Compound 2-4-9, using Cores 2-2 to 2-4.

### Synthesis Example (Compounds 2-4-13 to 2-4-16)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(4″-bromo-[1,1′: 3′,1″-terphenyl]-3-yl)-6-phenyl-1,3,5-triazine (44.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 30 g (yield: 64%) of the final product.

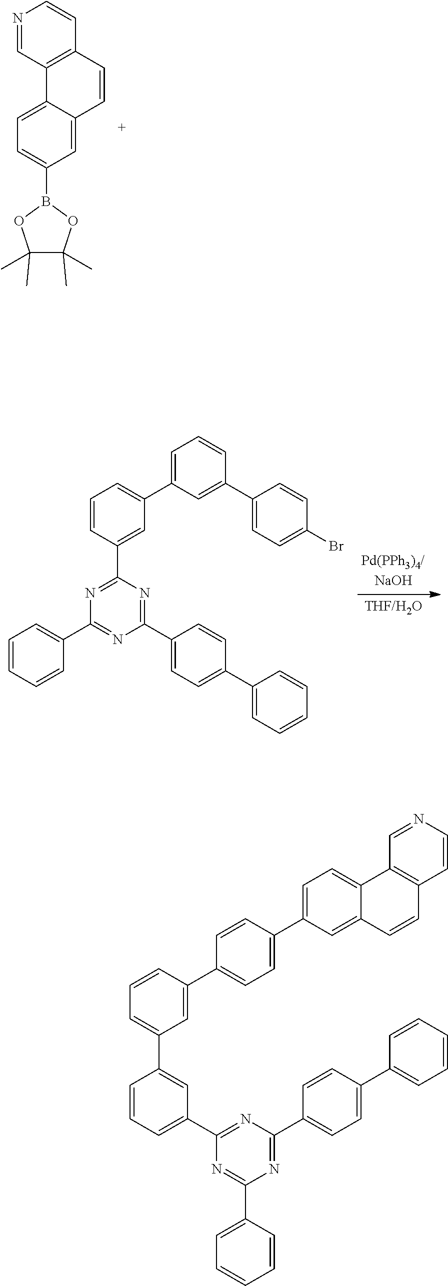 [SMILES: CC1(C)OB(c2ccc3c(ccc4ccncc43)c2)OC1(C)C ; Brc1ccc(-c2cccc(-c3cccc(-c4nc(-c5ccccc5)nc(-c5ccc(-c6ccccc6)cc5)n4)c3)c2)cc1 ; c1ccc(-c2ccc(-c3nc(-c4ccccc4)nc(-c4cccc(-c5cccc(-c6ccc(-c7ccc8c(ccc9ccncc98)c7)cc6)c5)c4)n3)cc2)cc1]

Compounds 2-4-14 to 2-4-16 can be synthesized by the same method as Compound 2-4-13, using Cores 2-2 to 2-4.

### Synthesis Example (Compounds 2-4-17 to 2-4-20)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(4″-bromo-[1,1′:4′,1″-terphenyl]-3-yl)-6-phenyl-1,3,5-triazine (44.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 31 g (yield: 67%) of the final product.

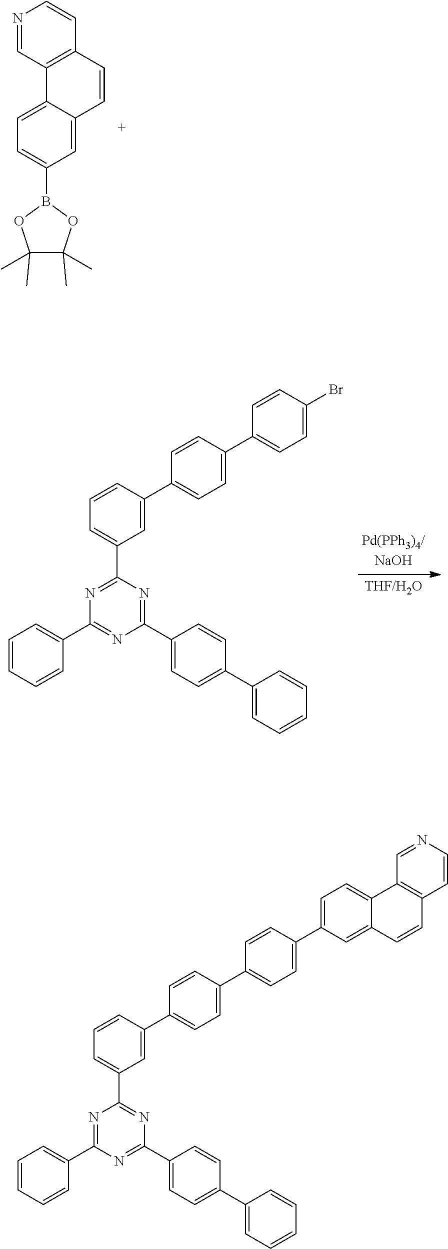 [SMILES: CC1(C)OB(c2ccc3c(ccc4ccncc43)c2)OC1(C)C ; Brc1ccc(-c2ccc(-c3cccc(-c4nc(-c5ccccc5)nc(-c5ccc(-c6ccccc6)cc5)n4)c3)cc2)cc1 ; c1ccc(-c2ccc(-c3nc(-c4ccccc4)nc(-c4cccc(-c5ccc(-c6ccc(-c7ccc8c(ccc9ccncc98)c7)cc6)cc5)c4)n3)cc2)cc1]

Compounds 2-4-18 to 2-4-20 can be synthesized by the same method as Compound 2-4-17, using Cores 2-2 to 2-4.

### Synthesis Example (Compounds 2-4-21 to 2-4-24)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(3″-bromo-[1,1′: 3′,1″-terphenyl]-3-yl)-6-phenyl-1,3,5-triazine (44.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 30 g (yield: 64%) of the final product.

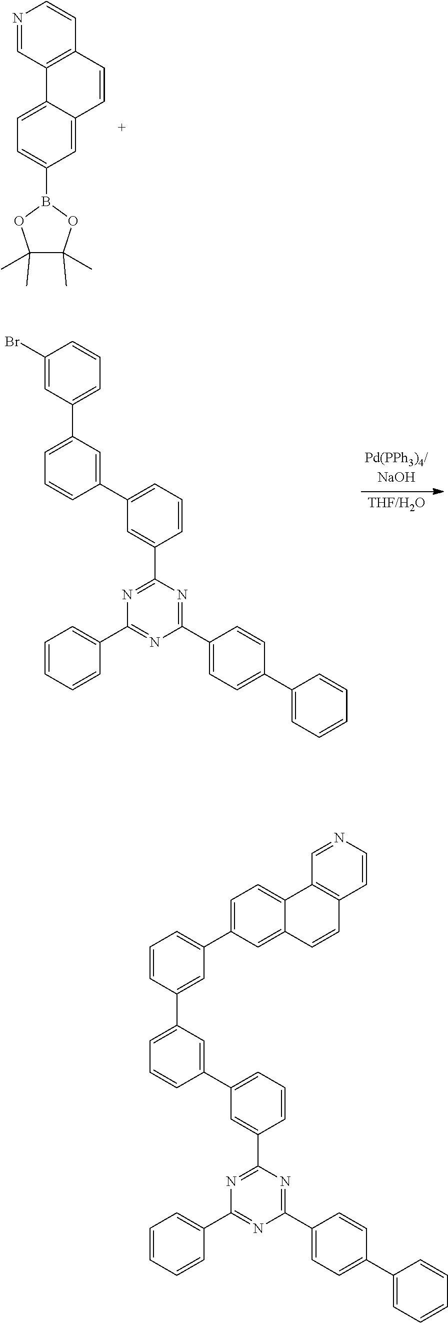 [SMILES: CC1(C)OB(c2ccc3c(ccc4ccncc43)c2)OC1(C)C ; Brc1cccc(-c2cccc(-c3cccc(-c4nc(-c5ccccc5)nc(-c5ccc(-c6ccccc6)cc5)n4)c3)c2)c1 ; c1ccc(-c2ccc(-c3nc(-c4ccccc4)nc(-c4cccc(-c5cccc(-c6cccc(-c7ccc8c(ccc9ccncc98)c7)c6)c5)c4)n3)cc2)cc1]

Compounds 2-4-22 to 2-4-24 can be synthesized by the same method as Compound 2-4-21, using Cores 2-2 to 2-4.

### Synthesis Example (Compounds 2-4-25 to 2-4-28)

8-(4,4,5,5-tetramethyl-1,3,2-dioxaborolan-2-yl)benzo[h]isoquinoline (20 g, 65.5 mmol) was dissolved with THF, and then 2-([1,1′-biphenyl]-4-yl)-4-(3″-bromo-[1,1′: 4′,1″-terphenyl]-3-yl)-6-phenyl-1,3,5-triazine (44.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 33 g (yield: 70%) of the final product.

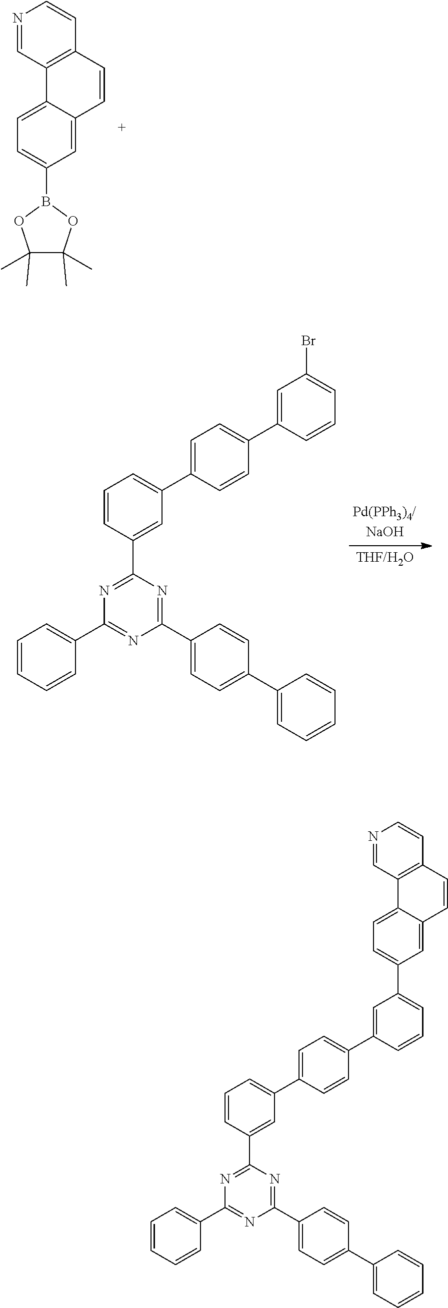 [SMILES: CC1(C)OB(c2ccc3c(ccc4ccncc43)c2)OC1(C)C ; Brc1cccc(-c2ccc(-c3cccc(-c4nc(-c5ccccc5)nc(-c5ccc(-c6ccccc6)cc5)n4)c3)cc2)c1 ; c1ccc(-c2ccc(-c3nc(-c4ccccc4)nc(-c4cccc(-c5ccc(-c6cccc(-c7ccc8c(ccc9ccncc98)c7)c6)cc5)c4)n3)cc2)cc1]

Compounds 2-4-26 to 2-4-28 can be synthesized by the same method as Compound 2-4-25, using Cores 2-2 to 2-4.

### Synthesis Example (Compounds 2-4-29 to 2-4-32)

8-(4,4,5,5-tetramethyl-1,3,2-dioxaborolan-2-yl)benzo[h]isoquinoline (20 g, 65.5 mmol) was dissolved with THE, and then 2-([1,1′-biphenyl]-4-yl)-4-(3″-bromo-[1,1′: 3′,1″-terphenyl]-4-yl)-6-phenyl-1,3,5-triazine (44.5 g, 72.1 mmol), Pd(PPh₃)₄ (2.0 g, 1.7 mmol), NaOH (6.8 g, 168.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 32 g (yield: 68%) of the final product.

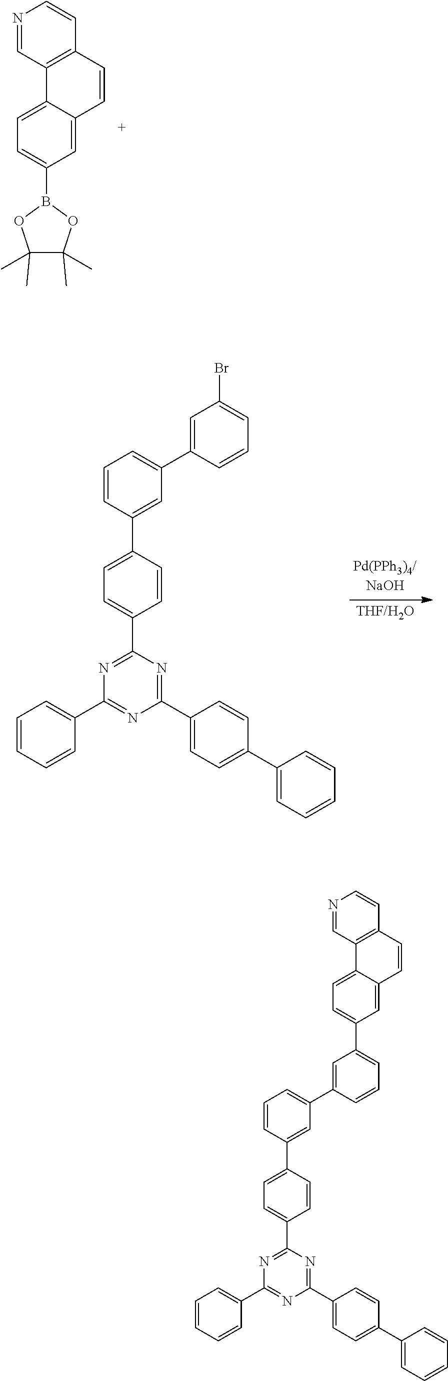 [SMILES: CC1(C)OB(c2ccc3c(ccc4ccncc43)c2)OC1(C)C ; Brc1cccc(-c2cccc(-c3ccc(-c4nc(-c5ccccc5)nc(-c5ccc(-c6ccccc6)cc5)n4)cc3)c2)c1 ; c1ccc(-c2ccc(-c3nc(-c4ccccc4)nc(-c4ccc(-c5cccc(-c6cccc(-c7ccc8c(ccc9ccncc98)c7)c6)c5)cc4)n3)cc2)cc1]

Compounds 2-4-30 to 2-4-32 can be synthesized by the same method as Compound 2-4-29, using Cores 2-2 to 2-4.

TABLE 4

| Compound | FD-MS |
|---|---|
| 2-1-1~2-1-4 | Chemical Formula: C₃₄H₂₂N₄ |
| | Molecular Weight: 486.58 |
| | m/z: 486.18 |
| 2-2-1~2-2-4 | Chemical Formula: C₄₀H₂₆N₄ |
| | Molecular Weight: 562.68 |
| | m/z: 562.22 |
| 2-2-5~2-2-8 | Chemical Formula: C₄₀H₂₆N₄ |
| | Molecular Weight: 562.68 |
| | m/z: 562.22 |
| 2-2-9~2-2-12 | Chemical Formula: C₄₀H₂₆N₄ |
| | Molecular Weight: 562.68 |
| | m/z: 562.22 |
| 2-3-1~2-3-4 | Chemical Formula: C₄₆H₃₀N₄ |
| | Molecular Weight: 638.77 |
| | m/z: 638.25 |
| 2-3-5~2-3-8 | Chemical Formula: C₄₆H₃₀N₄ |
| | Molecular Weight: 638.77 |
| | m/z: 638.25 |
| 2-3-9~2-3-12 | Chemical Formula: C₄₆H₃₀N₄ |
| | Molecular Weight: 638.77 |
| | m/z: 638.25 |
| 2-3-13~2-3-16 | Chemical Formula: C₄₆H₃₀N₄ |
| | Molecular Weight: 638.77 |
| | m/z: 638.25 |
| 2-4-1~2-4-4 | Chemical Formula: C₅₂H₃₄N₄ |
| | Molecular Weight: 714.87 |
| | m/z: 714.28 |
| 2-4-5~2-4-8 | Chemical Formula: C₅₂H₃₄N₄ |
| | Molecular Weight: 714.87 |
| | m/z: 714.28 |
| 2-4-9~2-4-12 | Chemical Formula: C₅₂H₃₄N₄ |
| | Molecular Weight: 714.87 |
| | m/z: 714.28 |
| 2-4-13~2-4-16 | Chemical Formula: C₅₂H₃₄N₄ |
| | Molecular Weight: 714.87 |
| | m/z: 714.28 |
| 2-4-17~2-4-20 | Chemical Formula: C₅₂H₃₄N₄ |
| | Molecular Weight: 714.87 |
| | m/z: 714.28 |
| 2-4-21~2-4-24 | Chemical Formula: C₅₂H₃₄N₄ |
| | Molecular Weight: 714.87 |
| | m/z: 714.28 |
| 2-4-25~2-4-28 | Chemical Formula: C₅₂H₃₄N₄ |
| | Molecular Weight: 714.87 |
| | m/z: 714.28 |
| 2-4-29~2-4-32 | Chemical Formula: C₅₂H₃₄N₄ |
| | Molecular Weight: 714.87 |
| | m/z: 714.28 |

### Synthesis Example (Compound 3-1-1)

6-(4,4,5,5-tetramethyl-1,3,2-dioxaborolan-2-yl)benzo[h]quinoline (20 g, 64.9 mmol) was dissolved with THF, and then 2-bromo-4,6-diphenyl-1,3,5-triazine (22.3 g, 71.4 mmol), Pd(PPh₃)₄ (2.3 g, 2 mmol), NaOH (7.8 g, 194.7 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 17.8 g (yield: 67%) of the final product.

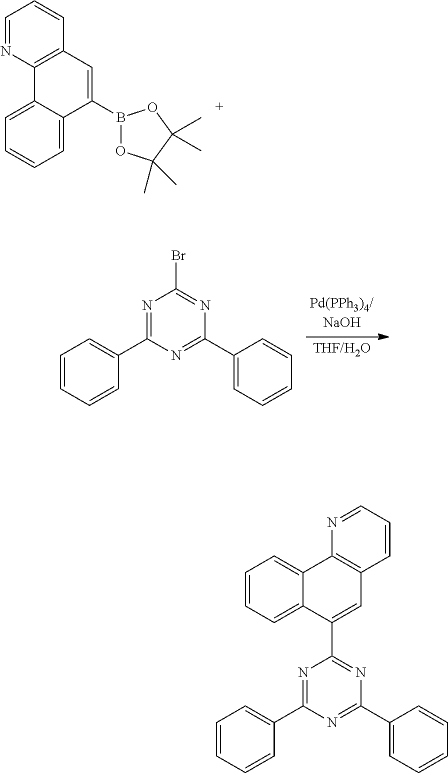 [SMILES: CC1(C)OB(c2cc3cccnc3c3ccccc23)OC1(C)C ; Brc1nc(-c2ccccc2)nc(-c2ccccc2)n1 ; c1ccc(-c2nc(-c3ccccc3)nc(-c3cc4cccnc4c4ccccc34)n2)cc1]

### Synthesis Example (Compound 3-1-2)

6-(4,4,5,5-tetramethyl-1,3,2-dioxaborolan-2-yl)benzo[h]quinoline (20 g, 64.9 mmol) was dissolved with THF, and then 2-(4-bromophenyl)-4,6-diphenyl-1,3,5-triazine (27.7 g, 71.4 mmol), Pd(PPh₃)₄ (2.3 g, 2 mmol), NaOH (7.8 g, 194.7 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 21.2 g (yield: 67%) of the final product.

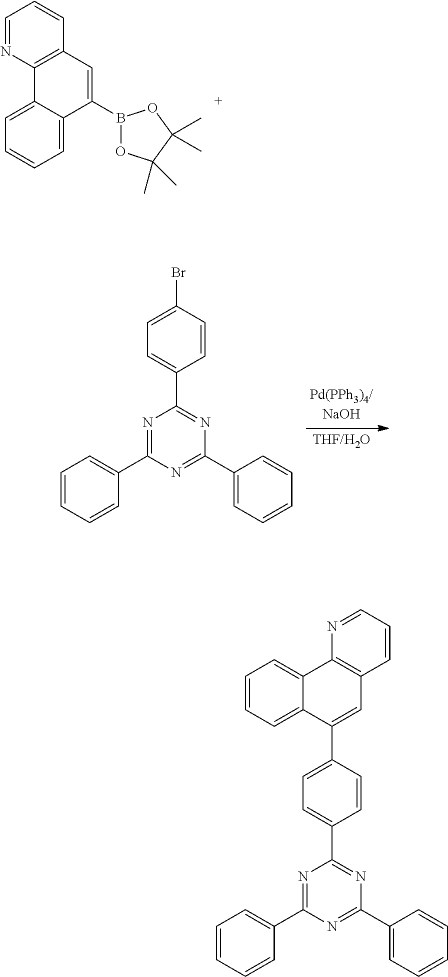 [SMILES: CC1(C)OB(c2cc3cccnc3c3ccccc23)OC1(C)C ; Brc1ccc(-c2nc(-c3ccccc3)nc(-c3ccccc3)n2)cc1 ; c1ccc(-c2nc(-c3ccccc3)nc(-c3ccc(-c4cc5cccnc5c5ccccc45)cc3)n2)cc1]

### Synthesis Example (Compound 3-2-1)

7-chlorobenzo[h]quinoline (20 g, 93.6 mmol) was dissolved with DMF in a round bottom flask, and then bis(pinacolato)diboron (26.1 g, 103 mmol), Pd(dppf)Cl₂ (2.1 g, 2.8 mmol) and KOAc (38.8 g, 280.8 mmol) were added and stirred under reflux at 130° C. for 4 hours. Upon completion of the reaction, DMF was removed from the reaction product via distillation and the reaction product was extracted with CH₂Cl₂ and water. The resulting organic layer was dried using MgSO₄ and concentrated, and the resulting compound was subjected to silicagel column and recrystallization to obtain 7-(4,4,5,5-tetramethyl-1,3,2-dioxaborolan-2-yl)benzo[h]quinoline.

The resulting 7-(4,4,5,5-tetramethyl-1,3,2-dioxaborolan-2-yl)benzo[h]quinoline (20 g, 65.5 mmol) was dissolved with THF, and then 2-(4-bromophenyl)-4,6-diphenyl-1,3,5-triazine (27.7 g, 71.4 mmol), Pd(PPh₃)₄ (2.3 g, 2 mmol), NaOH (7.8 g, 194.7 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 23 g (yield: 51%) of the final product.

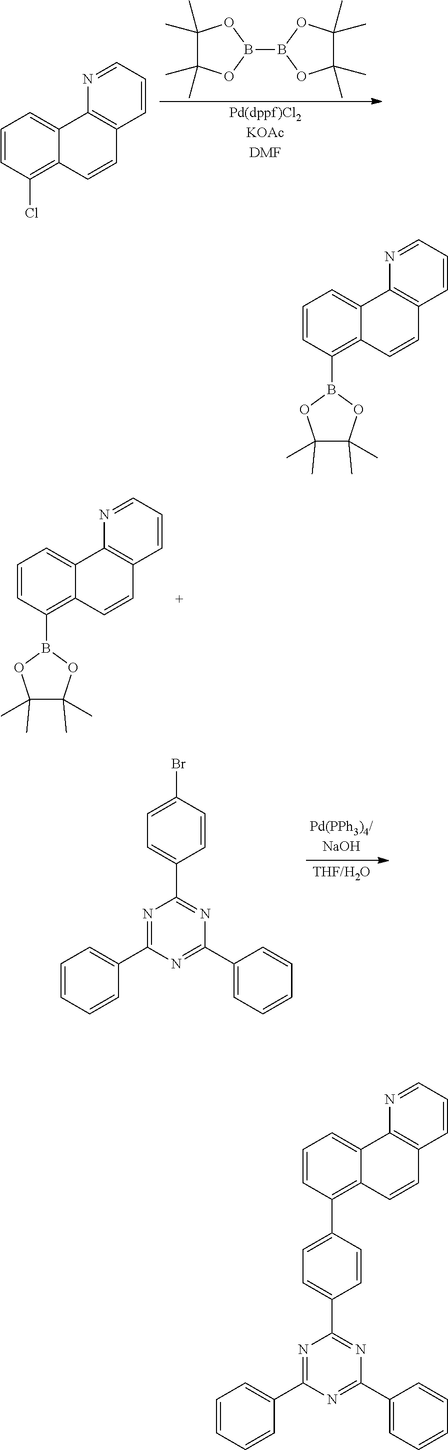

### Synthesis Example (Compound 3-3-1)

3-(4,4,5,5-tetramethyl-1,3,2-dioxaborolan-2-yl)phenanthridine (20 g, 65.5 mmol) was dissolved with THE, and then 2-(4-bromophenyl)-4,6-diphenyl-1,3,5-triazine (28 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 21 g (yield: 66%) of the final product.

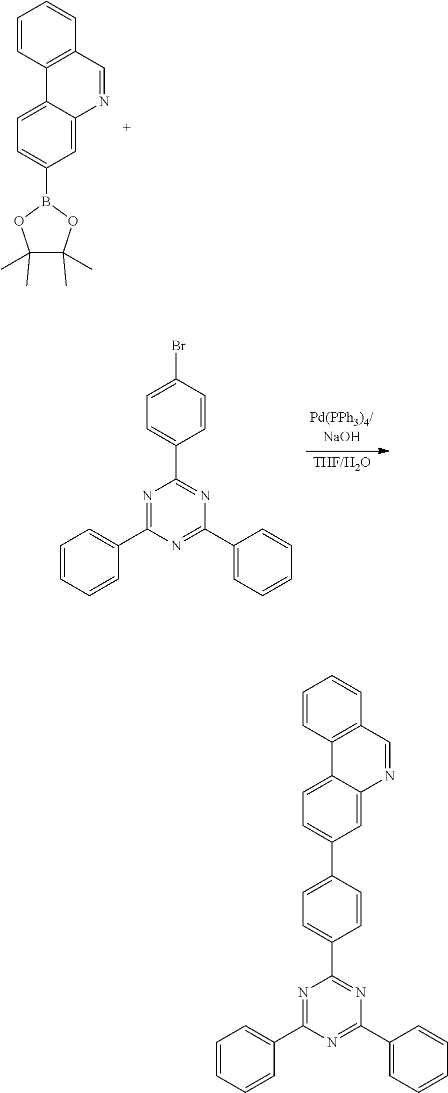 [SMILES: CC1(C)OB(c2ccc3c(c2)ncc2ccccc23)OC1(C)C ; Brc1ccc(-c2nc(-c3ccccc3)nc(-c3ccccc3)n2)cc1 ; c1ccc(-c2nc(-c3ccccc3)nc(-c3ccc(-c4ccc5c(c4)ncc4ccccc45)cc3)n2)cc1]

### Synthesis Example (Compound 4-1-3)

6-(4,4,5,5-tetramethyl-1,3,2-dioxaborolan-2-yl)benzo[h]quinoline (20 g, 65.5 mmol) was dissolved with THF, and then 2-bromo-1-phenyl-1H-benzo[d]imidazole (19.7 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 16.3 g (yield: 67%) of the final product.

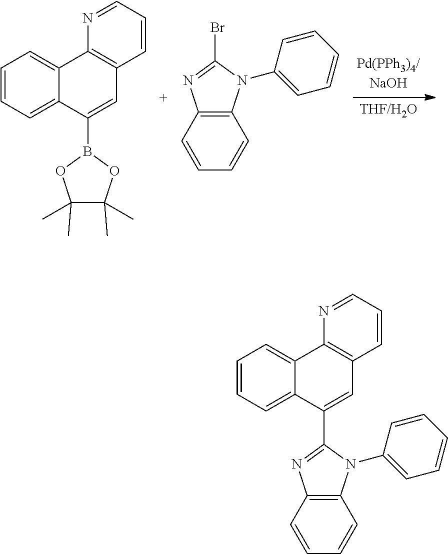 [SMILES: CC1(C)OB(c2cc3cccnc3c3ccccc23)OC1(C)C ; Brc1nc2ccccc2n1-c1ccccc1 ; c1ccc(-n2c(-c3cc4cccnc4c4ccccc34)nc3ccccc32)cc1]

### Synthesis Example (Compound 4-1-8)

6-(4,4,5,5-tetramethyl-1,3,2-dioxaborolan-2-yl)benzo[h]quinoline (20 g, 65.5 mmol) was dissolved with THF, and then 2-(4-bromophenyl)-1-phenyl-1H-benzo[d]imidazole (25.2 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 19 g (yield: 65%) of the final product.

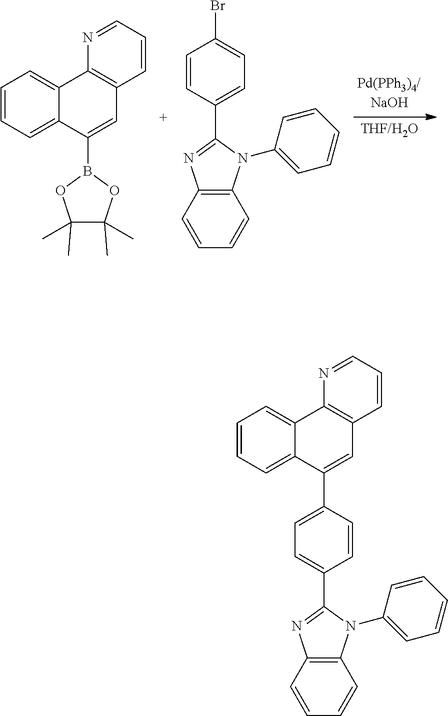

### Synthesis Example (Compound 4-2-3)

7-(4,4,5,5-tetramethyl-1,3,2-dioxaborolan-2-yl)benzo[h] quinoline (20 g, 65.5 mmol) was dissolved with THF, and then 2-bromo-1-phenyl-1H-benzo[d]imidazole (19.7 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 16.3 g (yield: 67%) of the final product.

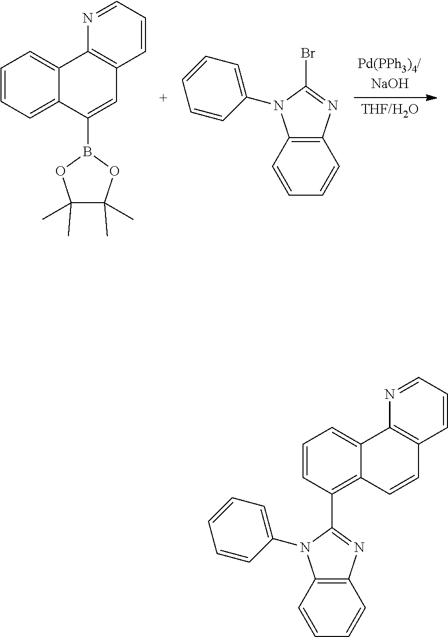

### Synthesis Example (Compound 4-2-8)

7-(4,4,5,5-tetramethyl-1,3,2-dioxaborolan-2-yl)benzo[h]quinoline (20 g, 65.5 mmol) was dissolved with THE, and then 2-(4-bromophenyl)-1-phenyl-1H-benzo[d]imidazole (25.2 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 19.1 g (yield: 65%) of the final product.

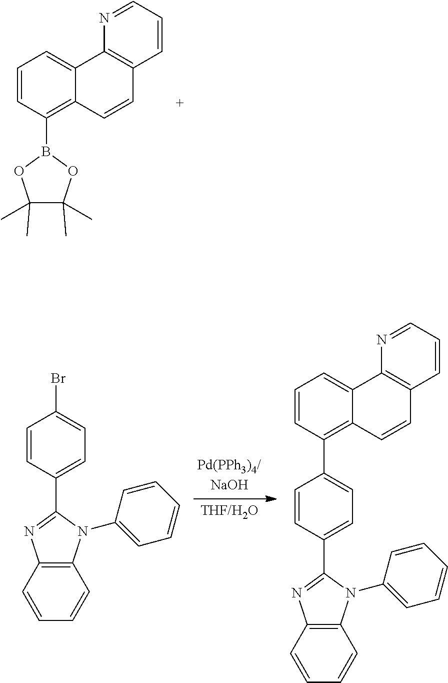 [SMILES: CC1(C)OB(c2cccc3c2ccc2cccnc23)OC1(C)C ; Brc1ccc(-c2nc3ccccc3n2-c2ccccc2)cc1 ; c1ccc(-n2c(-c3ccc(-c4cccc5c4ccc4cccnc45)cc3)nc3ccccc32)cc1]

### Synthesis Example (Compound Apr. 2, 2010)

7-(4,4,5,5-tetramethyl-1,3,2-dioxaborolan-2-yl)benzo[h]quinoline (20 g, 65.5 mmol) was dissolved with THE, and then 2-(4-bromophenyl)-4-phenylquinazoline (26 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 19.3 g (yield: 64%) of the final product.

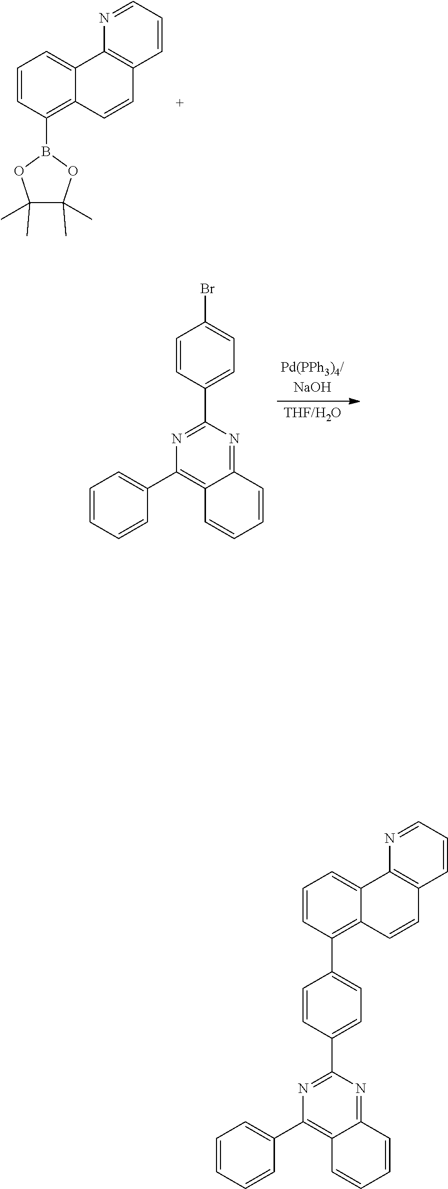 [SMILES: CC1(C)OB(c2cccc3c2ccc2cccnc23)OC1(C)C ; Brc1ccc(-c2nc(-c3ccccc3)c3ccccc3n2)cc1 ; c1ccc(-c2nc(-c3ccc(-c4cccc5c4ccc4cccnc45)cc3)nc3ccccc23)cc1]

### Synthesis Example (Compound 4-3-1)

3-(4,4,5,5-tetramethyl-1,3,2-dioxaborolan-2-yl)phenanthridine (20 g, 65.5 mmol) was dissolved with THE, and then 2-chloro-3-phenylquinoxaline (17.4 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 16.8 g (yield: 67%) of the final product.

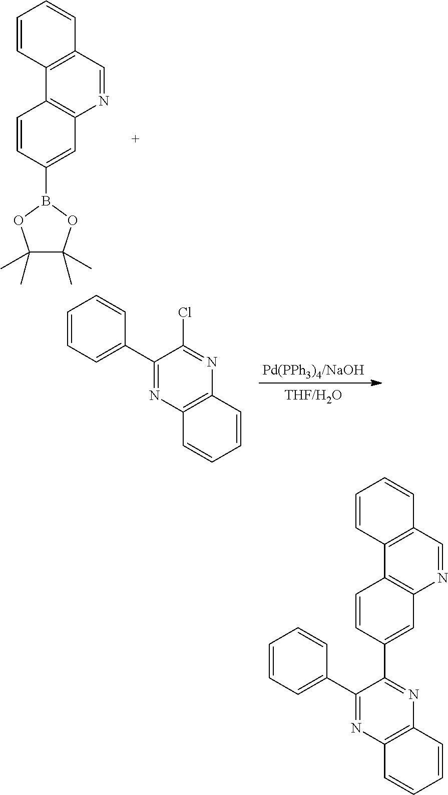

### Synthesis Example (Compound 4-3-7)

3-(4,4,5,5-tetramethyl-1,3,2-dioxaborolan-2-yl)phenanthridine (20 g, 65.5 mmol) was dissolved with THE, and then 1-bromo-4-iodobenzene (20.4 g, 72 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 3-(4-bromophenyl)phenanthridine as an intermediate product.

The intermediate product 3-(4-bromophenyl)phenanthridine (14.1 g, 42.2 mmol) was dissolved with DMF in a round bottom flask, and then bis(pinacolato)diboron (11.8 g, 46.4 mmol), Pd(dppf)Cl₂ (0.9 g, 1.3 mmol) and KOAc (17.5 g, 126.6 mmol) were added and stirred under reflux at 130° C. for 4 hours. Upon completion of the reaction, DMF was removed from the reaction product via distillation and the reaction product was extracted with CH₂Cl₂ and water. The resulting organic layer was dried using MgSO₄ and concentrated, and the resulting compound was subjected to silicagel column and recrystallization to obtain 3-(4-(4,4,5,5-tetramethyl-1,3,2-dioxaborolan-2-yl)phenyl)phenanthridine.

The resulting 3-(4-(4,4,5,5-tetramethyl-1,3,2-dioxaborolan-2-yl)phenyl) phenanthridine (16.1 g, 42 mmol) was dissolved with THE, and then 4′-chloro-4,2′: 6′,4″-terpyridine (12.4 g, 46.2 mmol), Pd(PPh₃)₄ (1.5 g, 1.3 mmol), NaOH (5 g, 126 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 13.6 g (yield: 42.7%) of the final product, 3-(4-([4,2′: 6′,4″-terpyridin]-4′-yl)phenyl) phenanthridine.

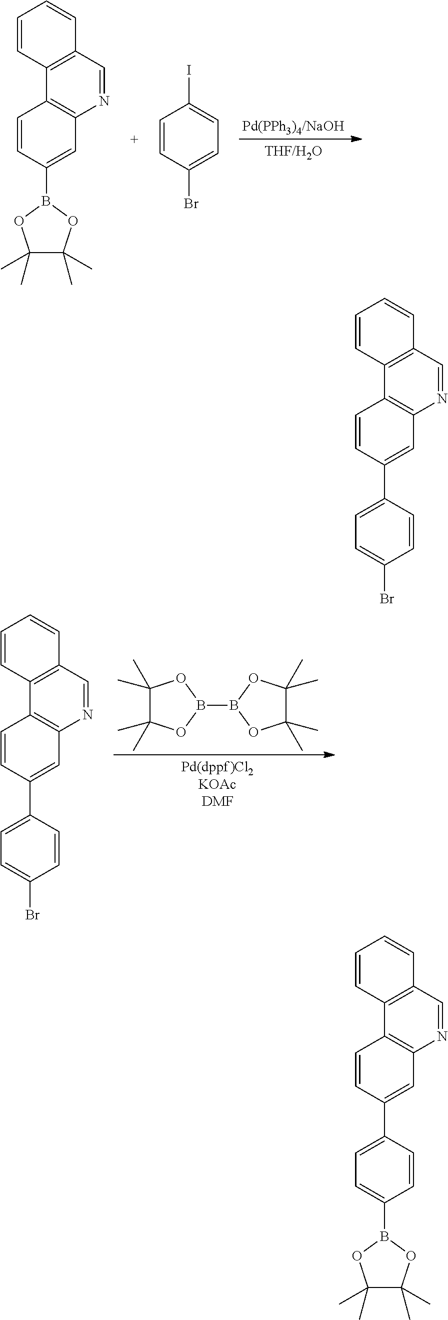

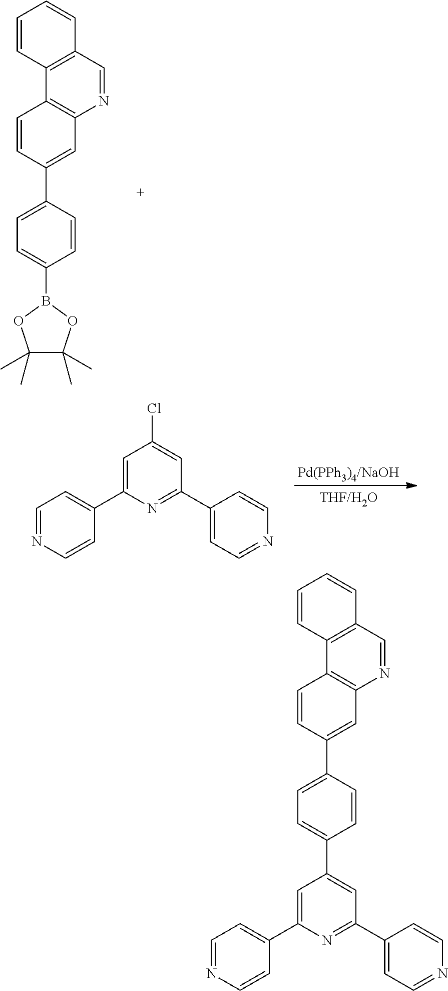 [SMILES: CC1(C)OB(c2ccc(-c3ccc4c(c3)ncc3ccccc34)cc2)OC1(C)C ; Clc1cc(-c2ccncc2)nc(-c2ccncc2)c1 ; c1ccc2c(c1)cnc1cc(-c3ccc(-c4cc(-c5ccncc5)nc(-c5ccncc5)c4)cc3)ccc12]

### Synthesis Example (Compound 4-3-9)

3-(4-(4,4,5,5-tetramethyl-1,3,2-dioxaborolan-2-yl)phenyl) phenanthridine (20 g, 52.3 mmol) was dissolved with THE, and then 2-chloro-4-phenylbenzo[4,5]thieno[3,2-d]pyrimidine (17.1 g, 57.6 mmol), Pd(PPh₃)₄ (1.8 g, 1.6 mmol), NaOH (6.3 g, 157 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 18.1 g (yield: 67%) of the final product.

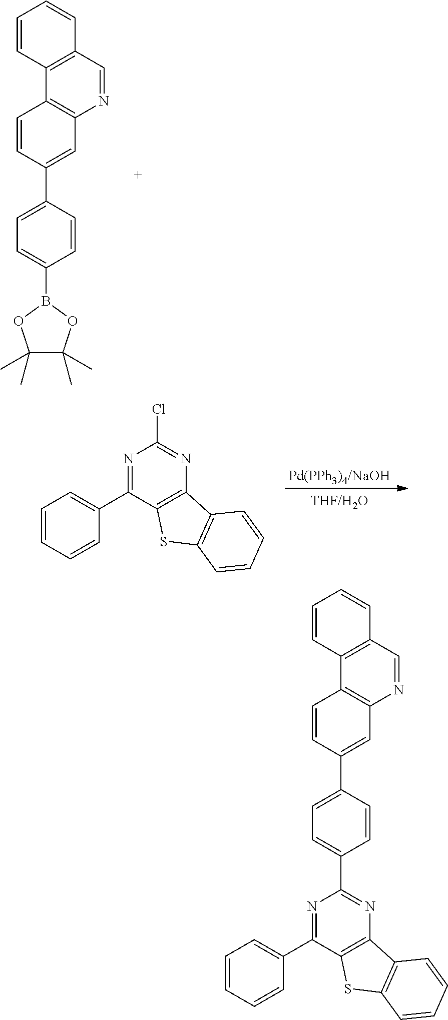 [SMILES: CC1(C)OB(c2ccc(-c3ccc4c(c3)ncc3ccccc34)cc2)OC1(C)C ; Clc1nc(-c2ccccc2)c2sc3ccccc3c2n1 ; c1ccc(-c2nc(-c3ccc(-c4ccc5c(c4)ncc4ccccc45)cc3)nc3c2sc2ccccc23)cc1]

### Synthesis Example (Compound 5-1-1)

6-(4,4,5,5-tetramethyl-1,3,2-dioxaborolan-2-yl)benzo[j] phenanthridine (20 g, 59.7 mmol) was dissolved with THF, and then 2,4-di([1,1′-biphenyl]-4-yl)-6-chloro-1,3,5-triazine (27.6 g, 65.6 mmol), Pd(PPh₃)₄ (2.0 g, 1.7 mmol), NaOH (7.2 g, 179 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 17.8 g (yield: 67%) of the final 10 product.

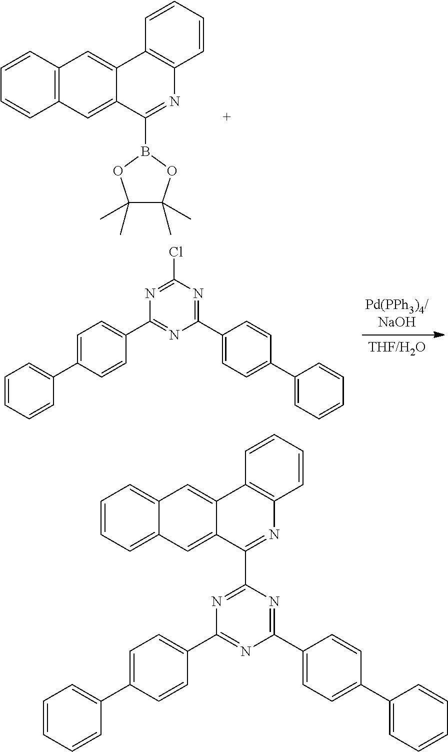

### Synthesis Example (Compound 5-2-5)

3-(4,4,5,5-tetramethyl-1,3,2-dioxaborolan-2-yl)naphtho[1,2-h] quinolin (20 g, 56.3 mmol) was dissolved with THE, and then 2,4-di([1,1′-biphenyl]-4-yl)-6-chloro-1,3,5-triazine (33.5 g, 61.9 mmol), Pd(PPh₃)₄ (2.0 g, 1.7 mmol), NaOH (7.2 g, 179 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 25 g (yield: 69%) of the final product.

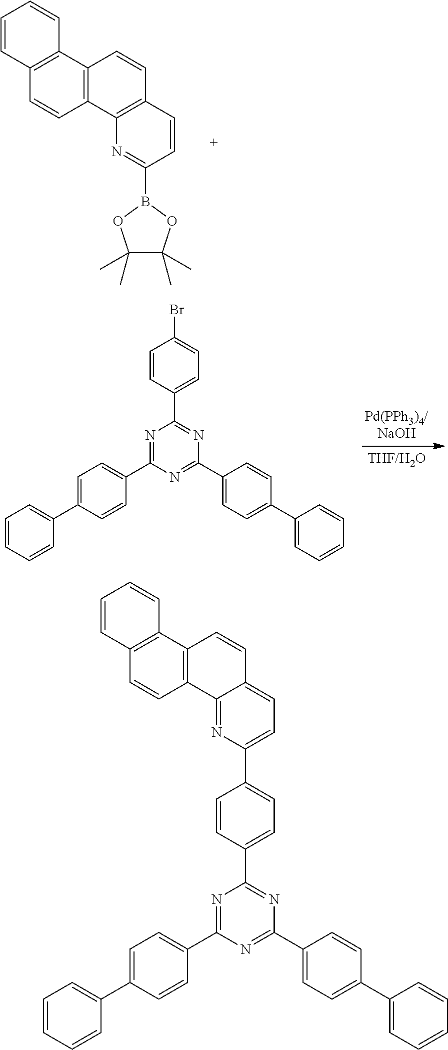

### Synthesis Example (Compound 5-3-11)

6-(4,4,5,5-tetramethyl-1,3,2-dioxaborolan-2-yl)benzo[j] phenanthridine (20 g, 56.3 mmol) was dissolved with THF, and then, 4-di([1,1′-biphenyl]-4-yl)-6-(3′-bromo-[1,1′-biphenyl]-4-yl)-1,3,5-triazine (38.2 g, 61.9 mmol), Pd(PPh₃)₄ (2.0 g, 1.7 mmol), NaOH (7.2 g, 179 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 25 g (yield: 65%) of the final product.

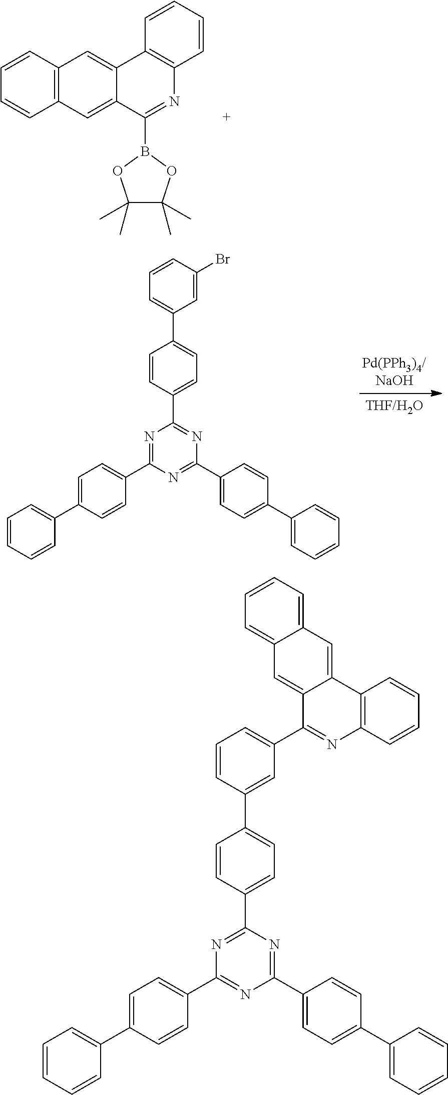

### Synthesis Example (Compound 5-4-11)

6-(4,4,5,5-tetramethyl-1,3,2-dioxaborolan-2-yl)benzo[j] phenanthridine (20 g, 56.3 mmol) was dissolved with THE, and then 2,4-di([1,1′-biphenyl]-4-yl)-6-(4″-bromo-[1,1′: 3′,1″-terphenyl]-4-yl)-1,3,5-triazine (42.9 g, 61.9 mmol), Pd(PPh₃)₄ (2.0 g, 1.7 mmol), NaOH (7.2 g, 179 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 30 g (yield: 68%) of the final product.

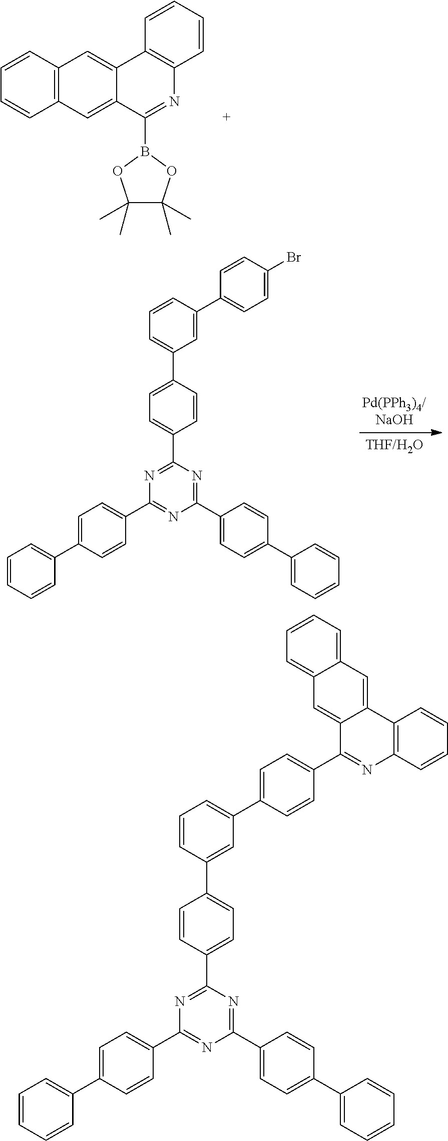

### Synthesis Example (Compound 6-1-1)

8-(4,4,5,5-tetramethyl-1,3,2-dioxaborolan-2-yl)benzo[h]isoquinoline (20 g, 65.5 mmol) was dissolved with THE, and then 2,4-di([1,1′-biphenyl]-4-yl)-6-chloro-1,3,5-triazine (30.3 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 20 g (yield: 68%) of the final product.

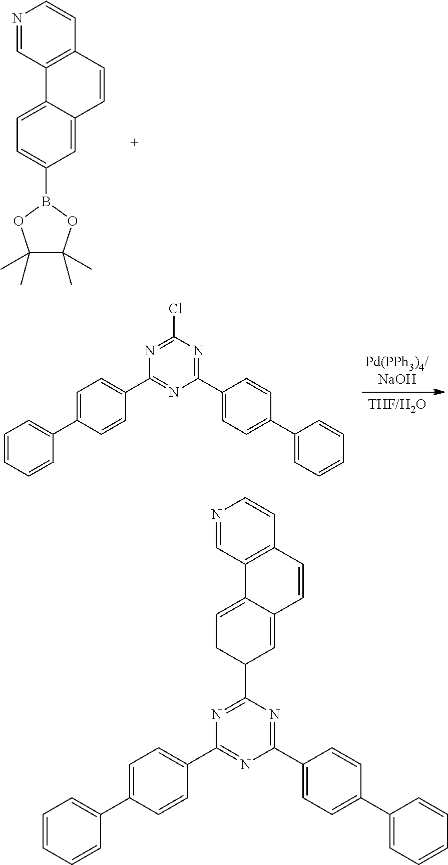 [SMILES: CC1(C)OB(c2ccc3c(ccc4ccncc43)c2)OC1(C)C ; Clc1nc(-c2ccc(-c3ccccc3)cc2)nc(-c2ccc(-c3ccccc3)cc2)n1 ; C1=c2ccc3ccncc3c2=CCC1c1nc(-c2ccc(-c3ccccc3)cc2)nc(-c2ccc(-c3ccccc3)cc2)n1]

### Synthesis Example (Compound 6-3-13)

8-(4,4,5,5-tetramethyl-1,3,2-dioxaborolan-2-yl)benzo[h]isoquinoline (20 g, 65.5 mmol) was dissolved with THE, and then 2,4-di([1,1′-biphenyl]-4-yl)-6-(3′-bromo-[1,1′-biphenyl]-3-yl)-1,3,5-triazine (22 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 25 g (yield: 67%) of the final product.

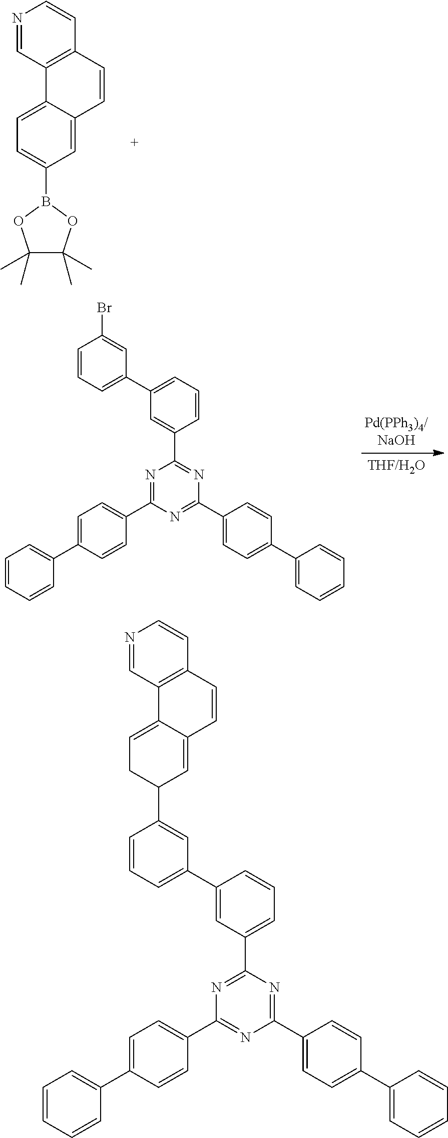

### Synthesis Example (Compound 6-4-22)

3-(4,4,5,5-tetramethyl-1,3,2-dioxaborolan-2-yl)phenanthridine (20 g, 65.5 mmol) was dissolved with THE, and then 2,4-di([1,1′-biphenyl]-4-yl)-6-(3″-bromo-[1,1′: 3′,1″-terphenyl]-3-yl)-1,3,5-triazine (49.9 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 28 g (yield: 67%) of the final product.

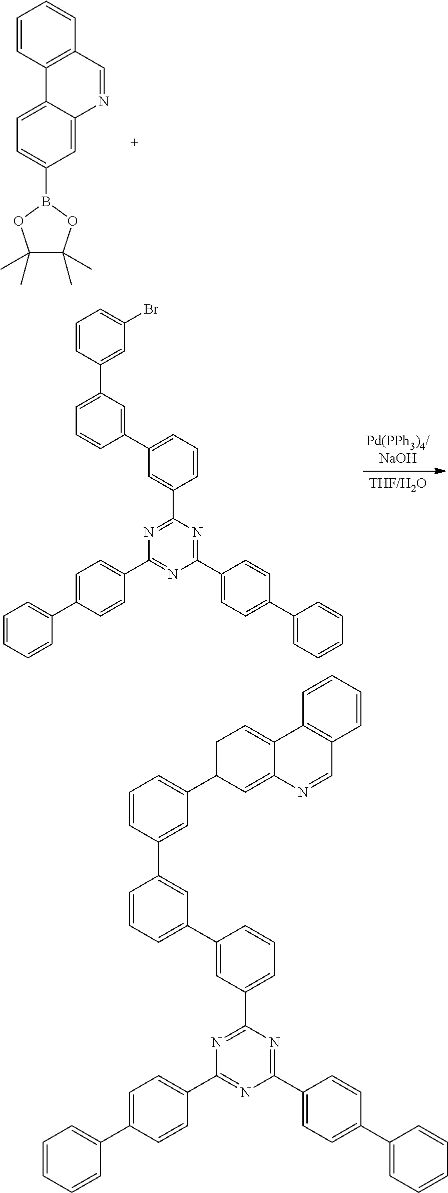

### Synthesis Example (Compound 7-1-2)

6-(4,4,5,5-tetramethyl-1,3,2-dioxaborolan-2-yl)benzo[h]quinoline (20 g, 65.5 mmol) was dissolved with THF, and then 4-([1,1′-biphenyl]-4-yl)-2-chlorobenzo[4,5] thieno[3,2-d]pyrimidine (26.9 g, 72.1 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 18 g (yield: 67%) of the final product.

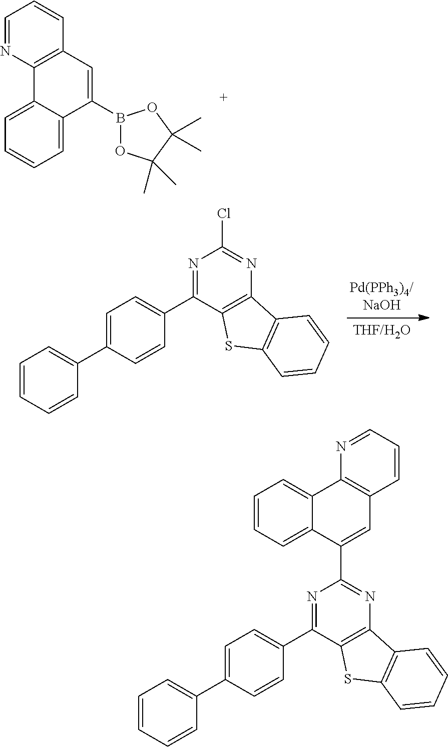 [SMILES: CC1(C)OB(c2cc3cccnc3c3ccccc23)OC1(C)C ; Clc1nc(-c2ccc(-c3ccccc3)cc2)c2sc3ccccc3c2n1 ; c1ccc(-c2ccc(-c3nc(-c4cc5cccnc5c5ccccc45)nc4c3sc3ccccc34)cc2)cc1]

### Synthesis Example (Compound 7-1-6)

6-(4,4,5,5-tetramethyl-1,3,2-dioxaborolan-2-yl)benzo[h]quinoline (20 g, 65.54 mmol) was dissolved with THE, and then 1-bromo-4-iodobenzene (20.4 g, 72 mmol), Pd(PPh₃)₄ (2.3 g, 2 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 14.2 g of 6-(4-bromophenyl) benzo[h]quinoline as an intermediate product.

The intermediate product 6-(4-bromophenyl)benzo[h]quinoline (14.2 g, 42.6 mmol) was dissolved with DMF in a round bottom flask, and then bis(pinacolato)diboron (11.9 g, 46.7 mmol), Pd(dppf)Cl₂ (0.9 g, 1.3 mmol) and KOAc (17.6 g, 127.5 mmol) were added and stirred under reflux at 130° C. for 4 hours. Upon completion of the reaction, DMF was removed from the reaction product via distillation and the reaction product was extracted with CH₂Cl₂ and water. The resulting organic layer was dried using MgSO₄ and concentrated, and the resulting compound was subjected to silicagel column and recrystallization to obtain 6-(4-(4,4,5,5-tetramethyl-1,3,2-dioxaborolan-2-yl)phenyl)benzo[h]quinoline.

The resulting 6-(4-(4,4,5,5-tetramethyl-1,3,2-dioxaborolan-2-yl)phenyl) benzo[h]quinoline (16 g, 42 mmol) was dissolved with THE, and then, 4-([1,1′-biphenyl]-4-yl)-2-chloroquinazoline (14.6 g, 46 mmol), Pd(PPh₃)₄ (2.3 g, 1.3 mmol), NaOH (7.9 g, 125.9 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 14.6 g (yield: 42%) of the final product, 6-(4-(4-([1,1′-biphenyl]-4-yl)quinazolin-2-yl)phenyl) benzo[h]quinoline.

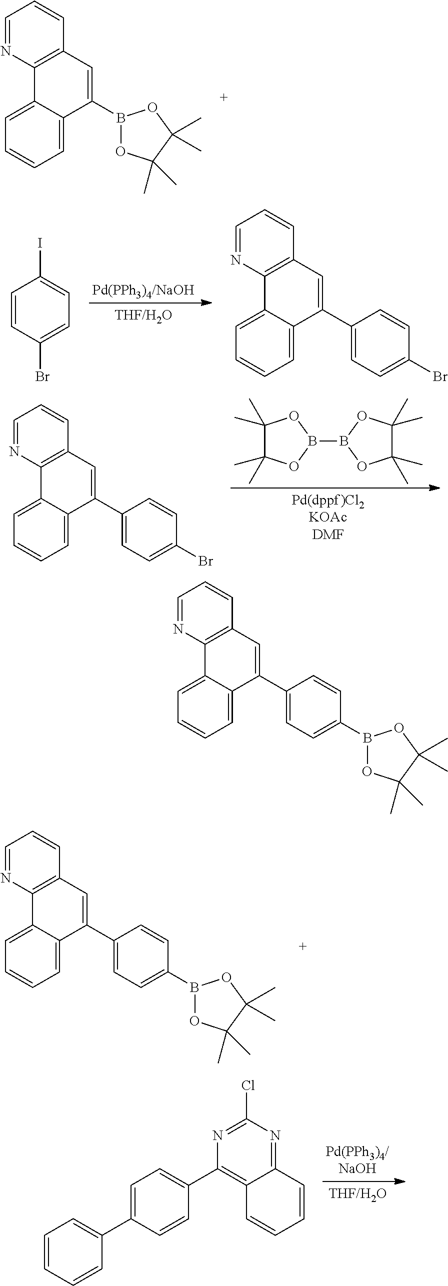

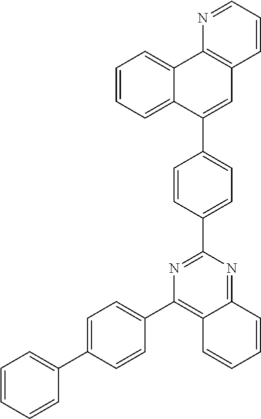 [SMILES: c1ccc(-c2ccc(-c3nc(-c4ccc(-c5cc6cccnc6c6ccccc56)cc4)nc4ccccc34)cc2)cc1]

### Synthesis Example (Compound 7-1-10)

6-(4,4,5,5-tetramethyl-1,3,2-dioxaborolan-2-yl)benzo[h]quinoline (20 g, 65.54 mmol) was dissolved with THE, and then 4-bromo-4′-iodo-1,1′-biphenyl (25.9 g, 72.1 mmol), Pd(PPh₃)₄ (2.3 g, 2.0 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 17.9 g of 6-(4′-bromo-[1,1′-biphenyl]-4-yl)benzo[h]quinoline as an intermediate product.

17.9 g of the intermediate product, 6-(4-bromophenyl)benzo[h]quinoline was dissolved with DMF in a round bottom flask, and then bis(pinacolato)diboron (12.2 g 48 mmol), Pd(dppf)Cl₂ (1 g, 1.3 mmol) and KOAc (18.1 g, 130.9 mmol) were added and stirred under reflux at 130° C. for 4 hours. Upon completion of the reaction, DMF was removed from the reaction product via distillation and the reaction product was extracted with CH₂Cl₂ and water. The resulting organic layer was dried using MgSO₄ and concentrated, and the resulting compound was subjected to silicagel column and recrystallization to obtain 6-(4′-(4,4,5,5-tetramethyl-1,3,2-dioxaborolan-2-yl)-[1,1′-biphenyl]-4-yl)benzo[h]quinoline.

The resulting 6-(4′-(4,4,5,5-tetramethyl-1,3,2-dioxaborolan-2-yl)-[1,1′-biphenyl]-4-yl) benzo[h]quinoline (20 g, 42 mmol) was dissolved with THE, and then, 4-([1,1′-biphenyl]-4-yl)-2-chloroquinazoline (14.6 g, 46 mmol), Pd(PPh₃)₄ (1.5 g, 1.3 mmol), NaOH (5 g, 126 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 17.5 g (yield: 43.6%) of the final product, 6-(4-(4-([1,1′-biphenyl]-4-yl)quinazolin-2-yl)phenyl)benzo[h]quinoline.

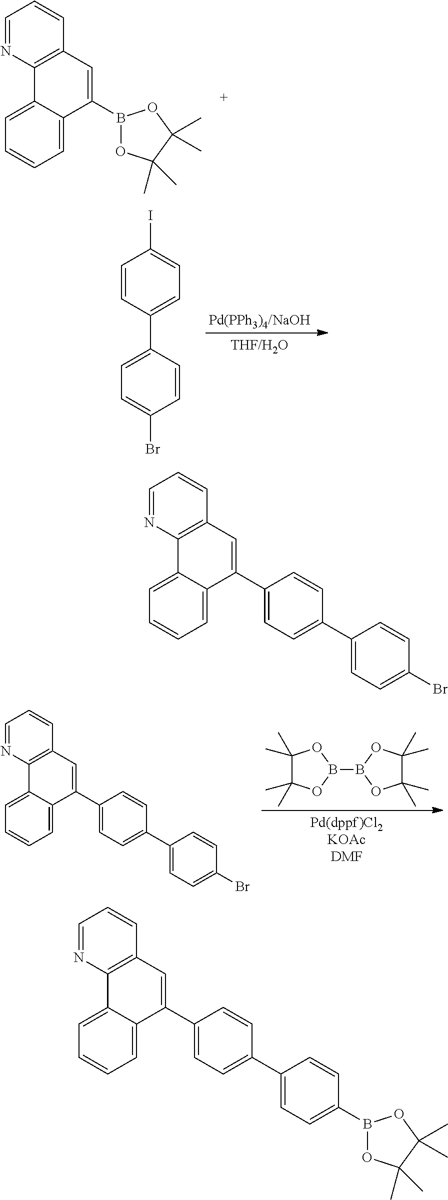

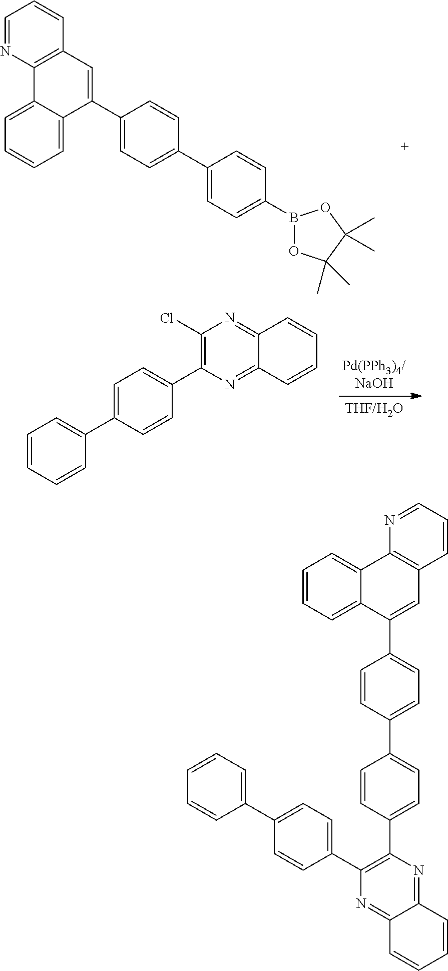

### Synthesis Example (Compound 7-2-19)

7-(4,4,5,5-tetramethyl-1,3,2-dioxaborolan-2-yl)benzo[h]quinoline (20 g, 65.54 mmol) was dissolved with THE, and then 1-bromo-3-iodobenzene (20.4 g, 72.1 mmol), Pd(PPh₃)₄ (2.3 g, 2.0 mmol), NaOH (7.9 g, 196.6 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 17.9 g of 7-(3-bromophenyl)benzo[h]quinoline as an intermediate product.

The intermediate product, 7-(3-bromophenyl)benzo[h]quinoline (17.9 g, 53.6 mmol) was dissolved with DMF in a round bottom flask, and then bis(pinacolato)diboron (15 g 58.9 mmol), Pd(dppf)Cl₂ (1.2 g, 1.6 mmol) and KOAc (22.2 g, 138.2 mmol) were added and stirred under reflux at 130° C. for 4 hours. Upon completion of the reaction, DMF was removed from the reaction product via distillation and the reaction product was extracted with CH₂Cl₂ and water. The resulting organic layer was dried using MgSO₄ and concentrated, and the resulting compound was subjected to silicagel column and recrystallization to obtain 7-(3-(4,4,5,5-tetramethyl-1,3,2-dioxaborolan-2-yl)phenyl)benzo[h]quinoline.

The resulting 7-(3-(4,4,5,5-tetramethyl-1,3,2-dioxaborolan-2-yl)phenyl)benzo[h] quinoline (14.3 g, 37.5 mmol) was dissolved with THF, and then 1-bromo-3-iodobenzene (11.7 g, 41.3 mmol), Pd(PPh₃)₄ (1.3 g, 1.1 mmol), NaOH (4.5 g, 112.5 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 7-(3′-bromo-[1,1′-biphenyl]-3-yl)benzo[h]quinoline (10.7 g, 26 mmol).

Repeatedly, 7-(3′-bromo-[1,1′-biphenyl]-3-yl)benzo[h]quinoline (10.7 g, 26 mmol) was dissolved with DMF in a round bottom flask, and then bis(pinacolato)diboron (7.3 g, 28.7 mmol), Pd(dppf)Cl₂ (0.6 g, 0.8 mmol) and KOAc (10.8 g, 78.2 mmol) were added and stirred under reflux at 130° C. for 4 hours. Upon completion of the reaction, DMF was removed from the reaction product via distillation and the reaction product was extracted with CH₂Cl₂ and water. The resulting organic layer was dried using MgSO₄ and concentrated, and the resulting compound was subjected to silicagel column and recrystallization to obtain 7-(3′-(4,4,5,5-tetramethyl-1,3,2-dioxaborolan-2-yl)-[1,1′-biphenyl]-3-yl)benzo[h]quinoline (8.3 g, 18.1 mmol).

Finally, 7-(3′-(4,4,5,5-tetramethyl-1,3,2-dioxaborolan-2-yl)-[1,1′-biphenyl]-3-yl) benzo[h]quinoline (8.3, 18.1 mmol) was dissolved with THE, and then 2-([1,1′-biphenyl]-4-yl)-3-chloroquinoxaline (6.3 g, 20 mmol), Pd(PPh₃)₄ (0.6 g, 0.5 mmol), NaOH (2.2 g, 54.4 mmol) and water were added and stirred under reflux at 100° C. for 3 hours. Upon completion of the reaction, the organic layer was extracted with E.A. and water, and was dried using MgSO₄ and concentrated. The resulting organic material was subjected to silicagel column and recrystallization to obtain 7.6 g (yield: 19%) of 7-(3′-(3-([1,1′-biphenyl]-4-yl) quinoxalin-2-yl)-[1,1′-biphenyl]-3-yl)benzo[h]quinoline.

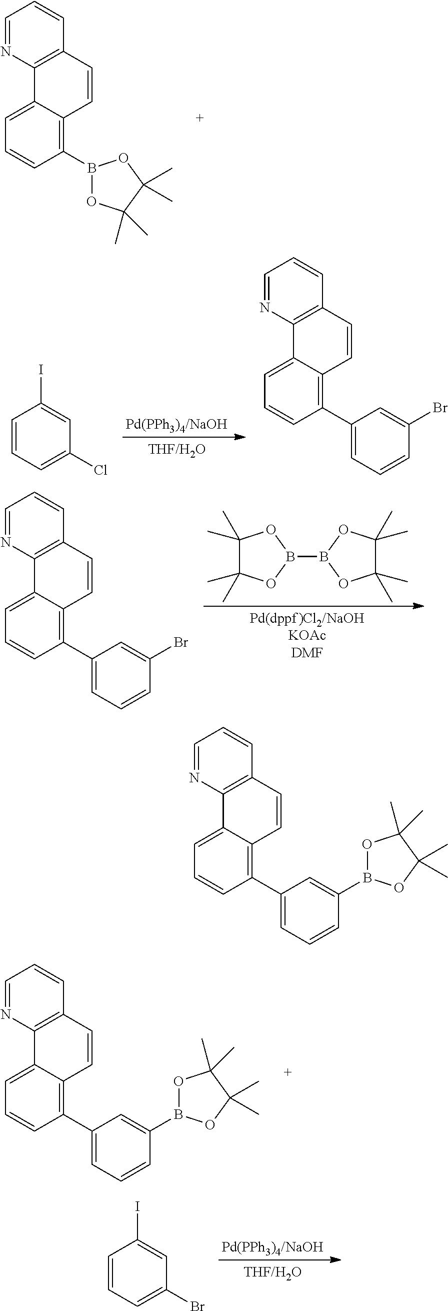

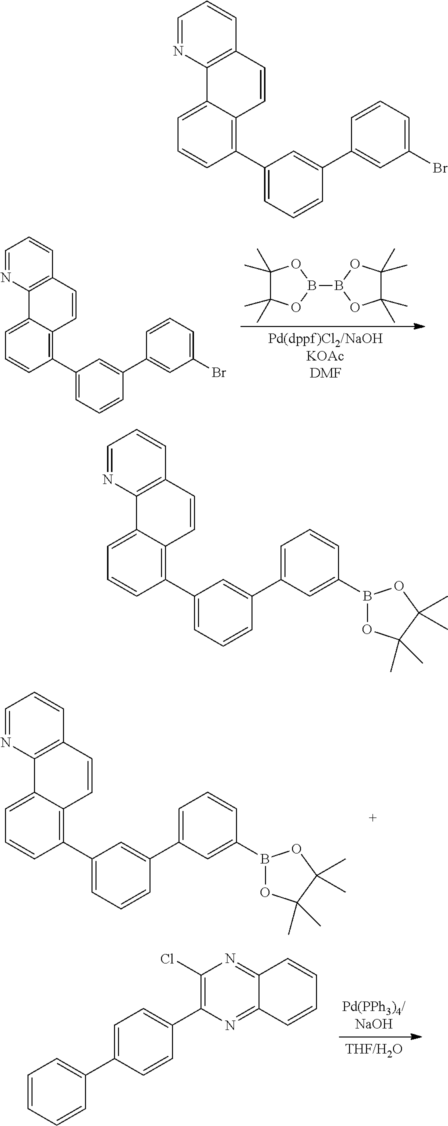

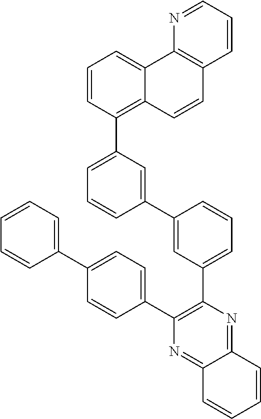 [SMILES: c1ccc(-c2ccc(-c3nc4ccccc4nc3-c3cccc(-c4cccc(-c5cccc6c5ccc5cccnc56)c4)c3)cc2)cc1]

The remaining compounds can be synthesized in similar ways.

TABLE 5

| Compound | FD-MS |
|---|---|
| 3-1-1, 4-2-2, 4-3-2 | Chemical Formula: C₂₈H₁₈N₄ |
| | Molecular Weight: 410.48 |
| | m/z: 410.15 |
| 3-1-2, 3-1-3, 3-2-1, 3-2-2, 3-3-1, 3-3-2, 4-1- | Chemical Formula: C₃₄H₂₂N₄ |
| 7, 4-1-12, 4-2-7, 4-2-12, 4-3-7, 4-3-12 | Molecular Weight: 486.58 |
| | m/z: 486.18 |
| 3-1-4, 3-1-6, 3-1-8, 3-2-3, 3-2-5, 3-2-7, 3-3- | Chemical Formula: C₄₄H₂₈N₄ |
| 3, 3-3-5, 3-3-7, 4-1-37, 4-1-42, 4-1-47, 4-2- | Molecular Weight: 612.74 |
| 37, 4-2-42, 4-2-47, | m/z: 612.23 |
| 4-3-37, 4-3-42, 4-3-47, 5-1-1, 5-1-2, 5-1-3, 5- | |
| 1-4, 5-1-5 | |
| 3-1-5, 3-1-7, 3-1-9, 3-2-4, 3-2-6, 3-2-8, 3-3- | Chemical Formula: C₅₀H₃₂N₄ |
| 4, 3-3-6, 3-3-8 | Molecular Weight: 688.83 |
| | m/z: 688.26 |
| 4-1-1, 4-1-5, 4-2-1, | Chemical Formula: C₂₇H₁₇N₃ |
| 4-2-5, 4-3-1, 4-3-5 | Molecular Weight: 383.45 |
| | m/z: 383.14 |
| 4-1-2 | Chemical Formula: C₂₈H₁₈N₄ |
| | Molecular Weight: 410.48 |
| | m/z: 410.15 |
| 4-1-3, 4-2-3, 4-3-3 | emical Formula: C₂₆H₁₇N₃ |
| | Molecular Weight: 371.44 |
| | m/z: 371.14 |
| 4-1-4, 4-2-4, 4-3-4 | Chemical Formula: C₂₉H₁₇N₃S |
| | Molecular Weight: 439.54 |
| | m/z: 439.11 |
| 4-1-6, 4-1-10, 4-1-11, 4-1-15, 4-2-6, 4-2-10 | Chemical Formula: C₃₃H₂₁N₃ |
| 4-2-11, 4-2-15, 4-3-6, 4-3-10, 4-3-11, 4-3-15, | Molecular Weight: 459.55 |
| 7-1-1, 7-1-3, 7-2-1, 7-2-3, 7-3-1, 7-3-3 | m/z: 459.17 |
| 4-1-8, 4-1-13, 4-2-8, 4-2-13, 4-3-8, 4-3-13 | Chemical Formula: C₃₂H₂₁N₃ |
| | Molecular Weight: 447.54 |
| | m/z: 447.17 |
| 4-1-9, 4-1-14, 4-2-9, 4-2-14, 4-3-9, 4-3- | Chemical Formula: C₃₅H₂₁N₃S |
| 14, 7-1-2, 7-2-2, 7-3-2 | Molecular Weight: 515.63 |
| | m/z: 515.15 |
| 4-1-16, 4-1-20, 4-1-21, 4-1-25, 4-1-26, 4- | Chemical Formula: C₃₉H₂₅N₃ |
| 1-30, 4-1-31, 4-1-35, 4-2-16, 4-2-20, 4-2- | Molecular Weight: 535.65 |
| 21, 4-2-25. 4-2-26, 4-2-30, 4-2-31, 4-2-35, | m/z: 535.20 |
| 4-3-16, 4-3-20, 4-3-21, 4-3-25, 4-3-26, 4-3- | |
| 30, 4-3-31, 4-3-35, 7-1-4, 7-1-6, 7-1-7, 7-1- | |
| 9, 7-2-4, 7-2-6, 7-2-7, 7-2-9, 7-3-4, 7-3-6, 7- | |
| 3-7, 7-3-9 | |
| 4-1-17, 4-1-22, 4-1-27, 4-1-32, 4-2-17, 4-2- | Chemical Formula: C₄₀H₂₆N₄ |
| 22, 4-2-27, 4-2-32, 4-3-17, 4-3-22, 4-3-27, 4- | Molecular Weight: 562.68 |
| 3-32, 6-1-1~6-1-4 | m/z: 562.22 |
| 4-1-18, 4-1-23, 4-1-28, 4-1-33, 4-2-18, 4-2- | Chemical Formula: C₃₈H₂₅N₃ |
| 23. 4-2-28, 4-2-33, 4-3-18, 4-3-23, 4-3-28, 4- | Molecular Weight: 523.64 |
| 3-33 | m/z: 523.20 |
| 4-1-19, 4-1-24, 4-1-29, 4-1-34, 4-2-19, 4-2- | Chemical Formula: C₄₁H₂₅N₃S |
| 24, 4-2-29, 4-2-34, 4-3-19, 4-3-24, 4-3-29, 4- | Molecular Weight: 591.73 |
| 3-34, 7-1-5, 7-1-8, 7-2-5, 7-2-8, 7-3-5, 7-3-8 | m/z: 591.18 |
| 4-1-36, 4-1-40, 4-1-41, 4-1-45, 4-1-46, 4-1- | Chemical Formula: C₄₃H₂₇N₃ |
| 50, 4-2-36, 4-2-40, 4-2-41, 4-2-45, | Molecular Weight: 585.71 |
| 4-2-46, 4-2-50, 4-3-36, 4-3-40, 4-3-41, 4-3- | m/z: 585.22 |
| 45. 4-3-46, 4-3-50 | |
| 4-1-38, 4-1-43, 4-1-48, 4-2-38, 4-2-43, 4-2- | Chemical Formula: C₄₂H₂₇N₃ |
| 48, 4-3-38, 4-3-43, 4-3-48 | Molecular Weight: 573.70 |
| | m/z: 573.22 |
| 4-1-39, 4-1-44, 4-1-49, 4-2-39, 4-2-44, 4-2- | Chemical Formula: C₄₅H₂₇N₃S |
| 49, 4-3-39, 4-3-44, 4-3-49 | Molecular Weight: 641.79 |
| | m/z: 641.19 |
| 5-2-1~5-2-10 | Chemical Formula: C₅₀H₃₂N₄ |
| | Molecular Weight: 688.83 |
| | m/z: 688.26 |
| 5-3-1~5-3-20, 6-4-33~6-4-41 | Chemical Formula: C₅₆H₃₆N₄ |
| | Molecular Weight: 764.93 |
| | m/z: 764.29 |
| 5-4-1~5-4-40 | Chemical Formula: C₆₂H₄₀N₄ |
| | Molecular Weight: 841.03 |
| | m/z: 840.33 |
| 6-2-1~2-8 | Chemical Formula: C₄₆H₃₀N₄ |
| | Molecular Weight: 638.77 |
| | m/z: 638.25 |
| 6-3-1~6-3-16 | Chemical Formula: C₅₂H₃₄N₄ |
| | Molecular Weight: 714.87 |
| | m/z: 714.28 |
| 6-4-1~4-32 | Chemical Formula: C₅₈H₃₈N₄ |
| | Molecular Weight: 790.97 |
| | m/z: 790.31 |
| 7-1-10, 7-1-12, 7-1-13, 7-1-15, 7-1-16, 7-1- | Chemical Formula: C₄₅H₂₉N₃ |
| 18, 7-1-19, 7-1-21, 7-2-10, 7-2-12, 7-2-13, 7- | Molecular Weight: 611.75 |
| 2-15, 7-2-16, 7-2-18, | m/z: 611.24 |
| 7-2-19, 7-2-21, 7-3-10, 7-3-12, 7-3-13, 7-3- | |
| 15, 7-3-16, 7-3-18, 7-3-19, 7-3-21 | |
| 7-1-11, 7-1-14, 7-1-17, 7-1-20, 7-2-11, 7-2- | Chemical Formula: C₄₇H₂₉N₃S |
| 14, 7-2-17, 7-2-20, 7-3-11, 7-3-14, 7-3-17, 7- | Molecular Weight: 667.83 |
| 3-20 | m/z: 667.21 |
| 7-1-22, 7-1-24, 7-1-25, 7-1-27, 7-1-28, 7-1- | Chemical Formula: C₄₉H₃₁N₃ |
| 30, 7-2-22, 7-2-24, 7-2-25, 7-2-27, 7-2-28, 7- | Molecular Weight: 661.81 |
| 2-30, 7-3-22, 7-3-24, 7-3-25, 7-3-27, 7-3-28, | m/z: 661.25 |
| 7-3-30 | |
| 7-1-23, 7-1-26, 7-1-29, 7-2-23, 7-2-26, 7-2- | Chemical Formula: C₅₁H₃₁N₃S |
| 29, 7-3-23, 7-3-26, 7-3-29 | Molecular Weight: 717.89 |
| | m/z: 717.22 |

### <Examples of Manufacturing Organic Light Emitting Device>

### Examples 1-12 (Application Examples to Electron Transport Layer of Green Organic Light Emitting Device)

A glass substrate (Corning 7059 Glass) on which an ITO (Indium Tin Oxide) thin film of a thickness of 1000 Å was deposited, was immersed in the D.I. water in which detergent was dissolved, and then cleaned while applying ultrasonic wave. The detergent used was a product of Fischer Co. and the D.I. water secondarily filtered through a filter of Millipore Co. Ltd was used. After the ITO film was cleaned for 30 minutes, the ultrasonic cleanings were performed two times with the D.I. water per 10 minutes. After the D.I. water cleanings, ultrasonic cleanings with isopropyl alcohol, acetone, and methanol solvent were performed in the order, and then the glass substrate was dried.

A film of 4,4′,4″-Tris[2-naphthyl(phenyl)amino] triphenylamine (hereinafter, referred to as 2-TNATA) with a thickness of 60 nm was deposited on the ITO film (anode) as a hole injection layer by vacuum-evaporation, and then a film of 4,4-bis[N-(1-naphthyl)-N-phenylamino]biphenyl (hereinafter referred to NPB) with a thickness of 60 nm was deposited on the hole injection layer by vacuum evaporation.

Subsequently, a light emitting layer having a thickness of 30 nm was deposited on the hole transport layer by vacuum-evaporating a mixture of 4,4′-N,N′-dicarbazole-biphenyl (hereinafter referred to as CBP) as a host and tris(2-phenylpyridine)-iridium (hereinafter referred to as Ir(ppy)₃) as a dopant at a weight ratio of 95:5.

Then, a hole blocking layer having a thickness of 10 nm was deposited on the light emitting layer by vacuum-evaporating (1,1′-bisphenyl)-4-olato)bis(2-methyl-8-quinolinolato) aluminum (abbreviated as BAlq), and then one of the compounds represented by Chemical Formula 1 of the present invention was deposited on the hole blocking layer to a thickness of 40 nm by vacuum-evaporation to form an electron transport layer. Then, LiF which is a halogenated alkali metal, was deposited to a thickness of 0.2 nm on the electron transport layer to form an electron injection layer, and then Al was deposited to a thickness of 150 nm to form a cathode, thereby manufacturing an organic light emitting device.

### Comparative Example 1

An organic light emitting device was prepared in the same manner as in the manufacturing example except that ET1 was used instead of the compound represented by Chemical Formula 1 of the present invention as an electron transporting layer material.

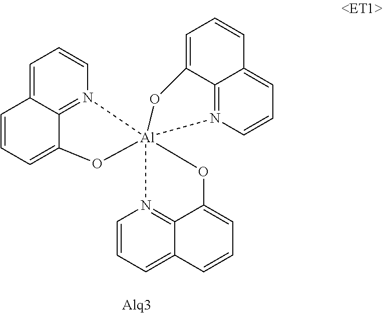

### Comparative Example 2

An organic light emitting device was prepared in the same manner as in the manufacturing example except that ET2 was used instead of the compound represented by Chemical Formula 1 of the present invention as an electron transport layer material.

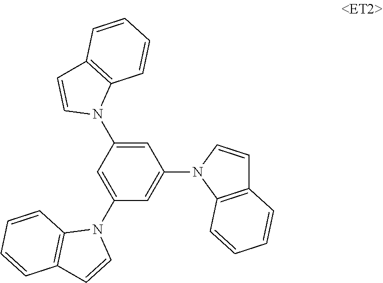 [SMILES: c1ccc2c(c1)ccn2-c1cc(-n2ccc3ccccc32)cc(-n2ccc3ccccc32)c1]

TABLE 6

| | Electron | Driving | Current | Light |
|---|---|---|---|---|
| | Transport | voltage | Efficiency | Emitting |
| | Layer | (V) | (cd/A) | Color |
| Example 1 | Compound 1-1-1 | 5.1 | 40.1 | Green |
| Example 2 | Compound 1-1-2 | 5.2 | 39.5 | Green |
| Example 3 | Compound 1-1-3 | 5.1 | 40.0 | Green |
| Example 4 | Compound 1-1-4 | 5.3 | 39.2 | Green |
| Example 5 | Compound 1-1-5 | 5.2 | 39.9 | Green |
| Example 6 | Compound 1-2-2 | 5.3 | 38.9 | Green |
| Example 7 | Compound 1-3-3 | 5.2 | 39.3 | Green |
| Example 8 | Compound 1-4-4 | 5.2 | 39.7 | Green |
| Example 9 | Compound 2-1-1 | 5.2 | 40.0 | Green |
| Example 10 | Compound 2-2-2 | 5.4 | 39.5 | Green |
| Example 11 | Compound 2-3-3 | 5.5 | 39.2 | Green |
| Example 12 | Compound 2-4-4 | 5.3 | 39.6 | Green |
| Comparative | ET1 | 6.2 | 23.7 | Green |
| Example 1 | | | | |
| Comparative | ET2 | 5.9 | 28.3 | Green |
| Example 2 | | | | |

As can be seen from the results of Table 6, a green organic light emitting device (OLED) employing the compounds of the present invention has a lower driving voltage and a higher efficiency than the devices employing ET1, Alq3 which is widely used as an electron transport layer material or ET2.

### Examples 13-24 (Application Examples to Electron Transport Layer of Blue Organic Light Emitting Device)

A glass substrate (Corning 7059 Glass) on which an ITO (Indium Tin Oxide) thin film of a thickness of 1000 Å was deposited, was immersed in the D.I. water in which detergent was dissolved, and then cleaned while applying ultrasonic wave. The detergent used was a product of Fischer Co. and the D.I. water secondarily filtered through a filter of Millipore Co. Ltd was used. After the ITO film was cleaned for 30 minutes, the ultrasonic cleanings were performed two times with the D.I. water per 10 minutes. After the D.I. water cleanings, ultrasonic cleanings with isopropyl alcohol, acetone, and methanol solvent were performed in the order, and then the glass substrate was dried.

A hole injection layer having a thickness of 60 nm was deposited by vacuum-evaporating 2-TNATA on the ITO anode layer, and a hole transport layer having a thickness of 30 nm was deposited by vacuum evaporating 4,4′-bis [N-(1-naphthyl)-N-phenylamino]biphenyl (hereinafter, NPB) on the hole injection layer.

A light emitting layer having a thickness of 30 nm was deposited on the hole transport layer by co-evaporating ADN as a host and 4,4′-bis[2-(4-(N,N-diphenylamino)phenyl) vinyl]biphenyl (hereinafter, DPAVBi) as a dopant at a weight ratio of 98:2.

An electron injection layer having a thickness of 30 nm was deposited on the light emitting layer by vacuum-evaporating one of the compounds represented by Chemical Formula 1 of the present invention, and LiF having a thickness of 1 nm was deposited on the electron transport layer by vacuum evaporation to form an electron injection layer, and Al was vacuum-deposited on the electron injection layer to form a cathode having a thickness of 300 nm, thereby manufacturing an organic light emitting device.

### Comparative Example 3

An organic light emitting device was prepared in the same manner as in the above manufacturing example except that ET1 was used instead of the compound represented by Chemical Formula 1 of the present invention as an electron transporting layer material.

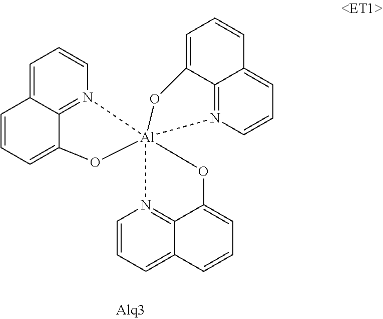

### Comparative Example 4

An organic light emitting device was prepared in the same manner as in the above manufacturing example except that ET3 was used instead of the compound represented by Chemical Formula 1 of the present invention as an electron transporting layer material.

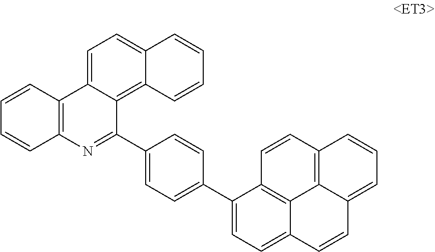 [SMILES: c1ccc2c(c1)ccc1c3ccccc3nc(-c3ccc(-c4ccc5ccc6cccc7ccc4c5c67)cc3)c21]

TABLE 7

| | Electron | Driving | Current | Light- |
|---|---|---|---|---|
| | Transport | voltage | Efficiency | Emitting |
| | Layer | (V) | (cd/A) | Color |
| Example 13 | Compound 1-1-1 | 5.4 | 6.9 | Blue |
| Example 14 | Compound 1-2-1 | 5.6 | 6.5 | Blue |
| Example 15 | Compound 1-2-3 | 5.8 | 6.4 | Blue |
| Example 16 | Compound 1-3-1 | 5.6 | 6.7 | Blue |
| Example 17 | Compound 1-3-5 | 5.7 | 6.6 | Blue |
| Example 18 | Compound 1-4-2 | 5.9 | 6.5 | Blue |
| Example 19 | Compound 1-4-5 | 5.8 | 6.8 | Blue |
| Example 20 | Compound 1-4-8 | 5.7 | 6.7 | Blue |
| Example 21 | Compound 2-1-2 | 5.5 | 6.8 | Blue |
| Example 22 | Compound 2-2-3 | 5.8 | 6.6 | Blue |
| Example 23 | Compound 2-3-5 | 6.0 | 6.4 | Blue |
| Example 24 | Compound 2-4-9 | 5.9 | 6.5 | Blue |
| Comparative | ET1 | 7.4 | 4.1 | Blue |
| Example 3 | | | | |
| Comparative | ET3 | 6.7 | 5.7 | Blue |
| Example 4 | | | | |

As can be seen from the results of Table 7, a blue organic light emitting device (OLED) employing the compounds of the present invention has a lower driving voltage and a higher efficiency than the devices employing ET1, Alq3 which is widely used as an electron transport layer material or ET3.

### Examples 25-28 (Application Examples to Electron Transport Layer of Blue Organic Light Emitting Device)

A glass substrate on which an ITO (Indium Tin Oxide) thin film of a thickness of 1500 Å was deposited, was immersed in the D.I. water in which detergent was dissolved, and then cleaned while applying ultrasonic wave. The detergent used was a product of Fischer Co. and the D.I. water secondarily filtered through a filter of Millipore Co. Ltd was used. After the ITO film was cleaned for 30 minutes, the ultrasonic cleanings were performed two times with the D.I. water per 10 minutes. After the D.I. water cleanings, ultrasonic cleanings with isopropyl alcohol, acetone, and methanol solvent were performed in the order, and then the glass substrate was dried.

A compound represented by the following HIL1 was deposited to a thickness of 250 Å on the ITO anode layer by vacuum-evaporation, and a compound represented by the following HIL2 was deposited to a thickness of 60 Å thereon by vacuum-evaporation to obtain a hole injection layer.

A hole transport layer having a thickness of 500 Å was deposited on the hole injection layer by vacuum evaporating a compound represented by the following HTL.

A light-emitting layer having a thickness of 200 Å was deposited on the hole transport layer by co-evaporating ADN, a host, and a compound represented by the following BD, a dopant, at a weight ratio of 4%.

An electron transport layer and electron injection layer having a thickness of 300 Å was deposited on the light-emitting layer by co-evaporating one of the compounds of Chemical Formula 1 of the present invention and Liq of a weight ratio of 50%.

A cathode having a thickness of 1500 Å was deposited on the electron transport layer and electron injection layer by vacuum evaporating Al to obtain an organic light emitting device.

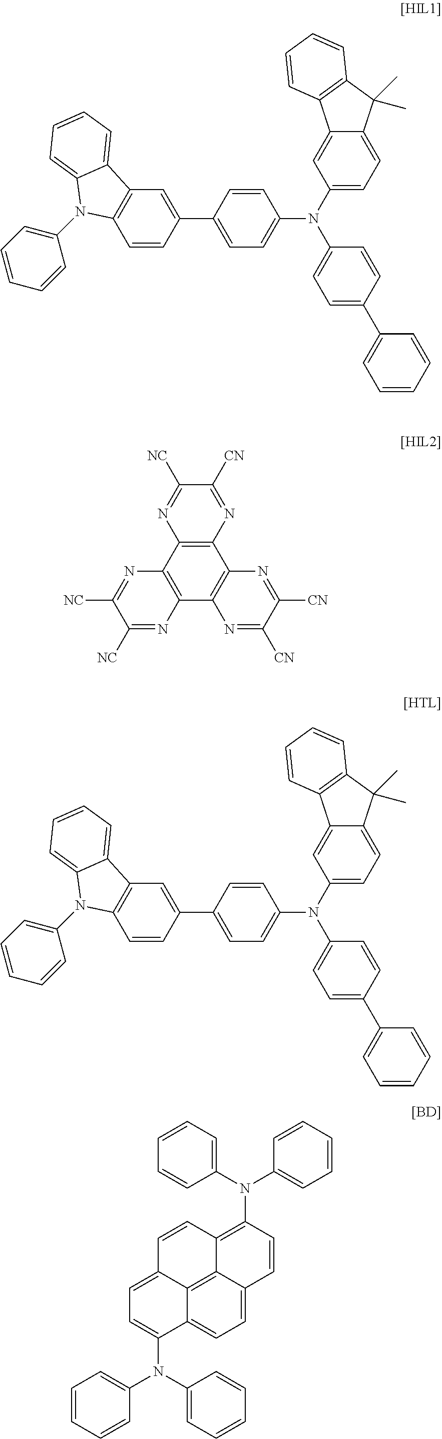 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21 ; N#Cc1nc2c3nc(C#N)c(C#N)nc3c3nc(C#N)c(C#N)nc3c2nc1C#N ; CC1(C)c2ccccc2-c2cc(N(c3ccc(-c4ccccc4)cc3)c3ccc(-c4ccc5c(c4)c4ccccc4n5-c4ccccc4)cc3)ccc21 ; c1ccc(N(c2ccccc2)c2ccc3ccc4c(N(c5ccccc5)c5ccccc5)ccc5ccc2c3c54)cc1]

### Comparative Example 5

An organic light-emitting device was prepared in the same manner as in the above example except that the following ET4 was used instead of the compound represented by Chemical Formula 1 of the present invention as an electron transport layer material.

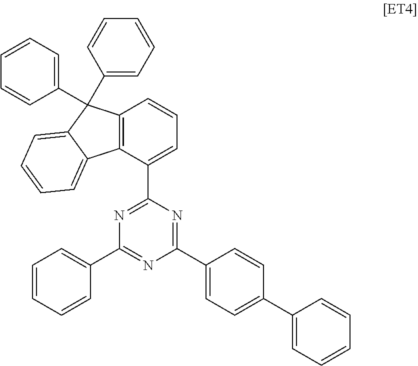 [SMILES: c1ccc(-c2ccc(-c3nc(-c4ccccc4)nc(-c4cccc5c4-c4ccccc4C5(c4ccccc4)c4ccccc4)n3)cc2)cc1]

TABLE 8

| | Electron | Driving | Current | Light- |
|---|---|---|---|---|
| | Transport | voltage | Efficiency | Emitting |
| | Layer | (V) | (cd/A ) | Color |
| Example 25 | Compound 2-1-2 | 4.4 | 8.8 | Blue |
| Example 26 | Compound 2-1-3 | 4.6 | 8.7 | Blue |
| Example 27 | Compound 2-1-4 | 4.7 | 7.1 | Blue |
| Example 28 | Compound 2-2-3 | 4.2 | 9.4 | Blue |
| Comparative | ET4 | 5.3 | 8.3 | Blue |
| Example 5 | | | | |

As can be seen from the results of Table 8, the blue organic light emitting device (OLED) employing the compounds of the present invention has a lower driving voltage and a higher efficiency than ET4 of Comparative Example 5.

### Example 29-40 (Application Examples to Electron Transport Layer of Blue Organic Light Emitting Device)

A glass substrate on which an ITO (Indium Tin Oxide) thin film of a thickness of 1500 Å was deposited, was immersed in the D.I. water in which detergent was dissolved, and then cleaned while applying ultrasonic wave. The detergent used was a product of Fischer Co. and the D.I. water secondarily filtered through a filter of Millipore Co. Ltd was used. After the ITO film was cleaned for 30 minutes, the ultrasonic cleanings were performed two times with the D.I. water per 10 minutes. After the D.I. water cleanings, ultrasonic cleanings with isopropyl alcohol, acetone, and methanol solvent were performed in the order, and then the glass substrate was dried.

A compound represented by the above HIL1 was deposited to a thickness of 250 Å on the ITO anode layer by vacuum-evaporation, and a compound represented by the above HIL2 was deposited to a thickness of 60 Å thereon by vacuum-evaporation to obtain a hole injection layer.

A hole transport layer having a thickness of 500 Å was deposited on the hole injection layer by vacuum evaporating a compound represented by the above HTL.

A light-emitting layer having a thickness of 200 Å was deposited on the hole transport layer by co-evaporating ADN, a host, and a compound represented by the above BD, a dopant, at a weight ratio of 4%.

An electron transport layer and electron injection layer having a thickness of 300 Å was deposited on the light-emitting layer by co-evaporating one of the compounds of Chemical Formula 1 of the present invention and Liq of a weight ratio of 50%.

A cathode having a thickness of 1500 Å was deposited on the electron transport layer and electron injection layer by vacuum evaporating Al to obtain an organic light emitting device.

TABLE 9

| | Electron | Driving | Current | Light- |
|---|---|---|---|---|
| | Transport | voltage | Efficiency | Emitting |
| | Layer | (V) | (cd/A) | Color |
| Example 29 | Compound 3-1-1 | 4.8 | 8.0 | Blue |
| Example 30 | Compound 3-1-2 | 4.7 | 8.3 | Blue |
| Example 31 | Compound 3-2-1 | 4.6 | 8.4 | Blue |
| Example 32 | Compound 3-3-1 | 4.8 | 8.2 | Blue |
| Example 33 | Compound 4-1-3 | 4.6 | 8.3 | Blue |
| Example 34 | Compound 4-1-8 | 4.5 | 8.5 | Blue |
| Example 35 | Compound 4-2-3 | 4.6 | 8.4 | Blue |
| Example 36 | Compound 4-2-6 | 4.7 | 8.4 | Blue |
| Example 37 | Compound 4-2-8 | 4.8 | 8.2 | Blue |
| Example 38 | Compound 4-3-1 | 4.7 | 8.3 | Blue |
| Example 39 | Compound 4-3-7 | 4.6 | 8.5 | Blue |
| Example 40 | Compound 4-3-9 | 4.6 | 8.4 | Blue |
| Example 41 | Compound 5-1-1 | 4.8 | 8.5 | Blue |
| Example 42 | Compound 5-2-5 | 4.7 | 8.6 | Blue |
| Example 43 | Compound 5-3-11 | 4.7 | 8.8 | Blue |
| Example 44 | Compound 5-4-11 | 4.5 | 8.9 | Blue |
| Example 45 | Compound 6-1-1 | 4.9 | 8.4 | Blue |
| Example 46 | Compound 6-3-13 | 4.7 | 8.6 | Blue |
| Example 47 | Compound 6-4-22 | 4.6 | 8.8 | Blue |
| Example 48 | Compound 7-1-2 | 4.7 | 8.5 | Blue |
| Example 49 | Compound 7-1-6 | 4.5 | 8.7 | Blue |
| Example 50 | Compound 7-1-10 | 4.4 | 8.9 | Blue |
| Example 51 | Compound 7-2-19 | 4.5 | 8.8 | Blue |
| Comparative | ET4 | 5.3 | 8.3 | Blue |
| Example 5 | | | | |

As can be seen from the results of Table 9, the blue organic light emitting device (OLED) employing the compounds of the present invention has a lower driving voltage and a higher efficiency than ET4 of Comparative Example 5.

While the present invention has been particularly shown and described with reference to exemplary embodiments thereof, it will be understood by those skilled in the art that various changes in form and details may be made therein without departing from the essential characteristics thereof. Thus, the embodiments disclosed herein are not intended to limit the invention but are to be considered in all respects as illustrative and not restrictive. The scope of protection of the present invention should be construed by the following claims, all of which are within the scope of the present invention.

The compound of the present invention can be used in an organic light emitting device and an organic EL display device including the same.

1. A compound represented by chemical formula 1,
A₁ [symbol] L [symbol] A₂ <Chemical Formula 1>,
wherein A₁ is a group represented by any one of the following structures: 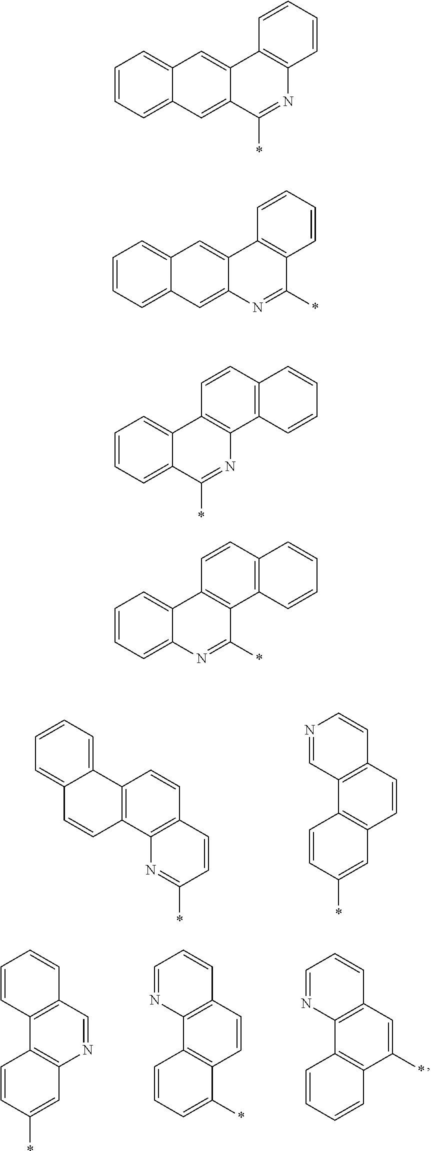
L is a group having the structure below, l, m, and n are each independently 0 or 1, and l, m, and n are not all 0 at the same time, 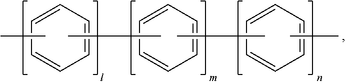
A₂ is any one of the following structures, 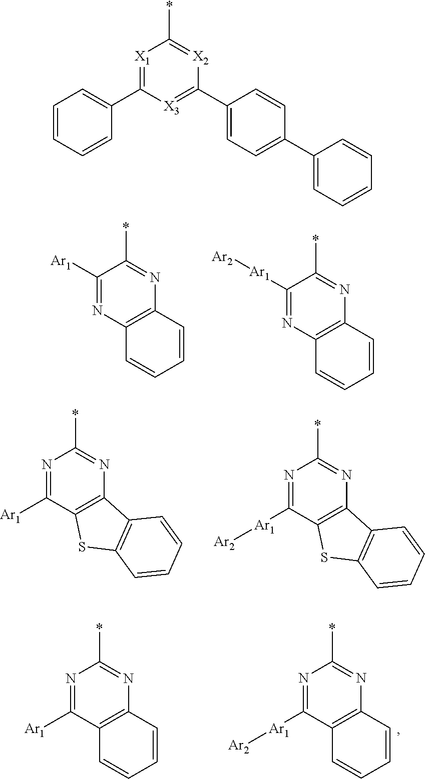
wherein X₁ to X₃ are each independently C or N, at least one of X₁ to X₃ is N, and Ar₁ and Ar₂ are each independently hydrogen, deuterium, a halogen group, a cyano group, a substituted or unsubstituted C₁ to C₆₀ alkyl group, a substituted or unsubstituted C₃ to C₁₀ cycloalkyl group, a substituted or unsubstituted C₆ to C₆₀ aryl group, a substituted or unsubstituted C₁ to C₆₀ heteroaryl group.

2. The compound of claim 1, wherein A₂ is represented by the following structural formula:

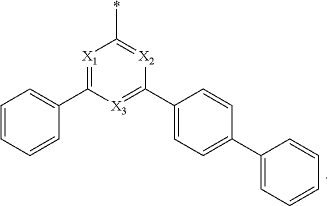

3. The compound of claim 1, wherein the compound of chemical formula 1 is any one of the following compounds:

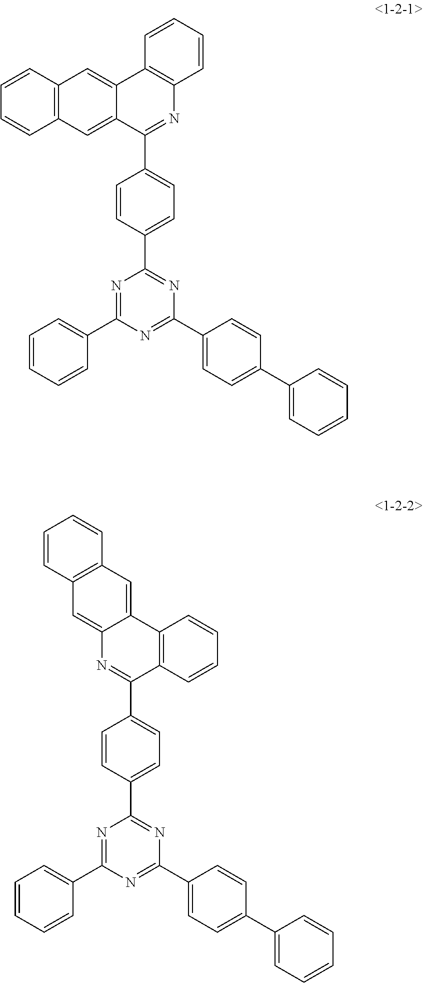 [SMILES: c1ccc(-c2ccc(-c3nc(-c4ccccc4)nc(-c4ccc(-c5nc6ccccc6c6cc7ccccc7cc56)cc4)n3)cc2)cc1 ; c1ccc(-c2ccc(-c3nc(-c4ccccc4)nc(-c4ccc(-c5nc6cc7ccccc7cc6c6ccccc56)cc4)n3)cc2)cc1]

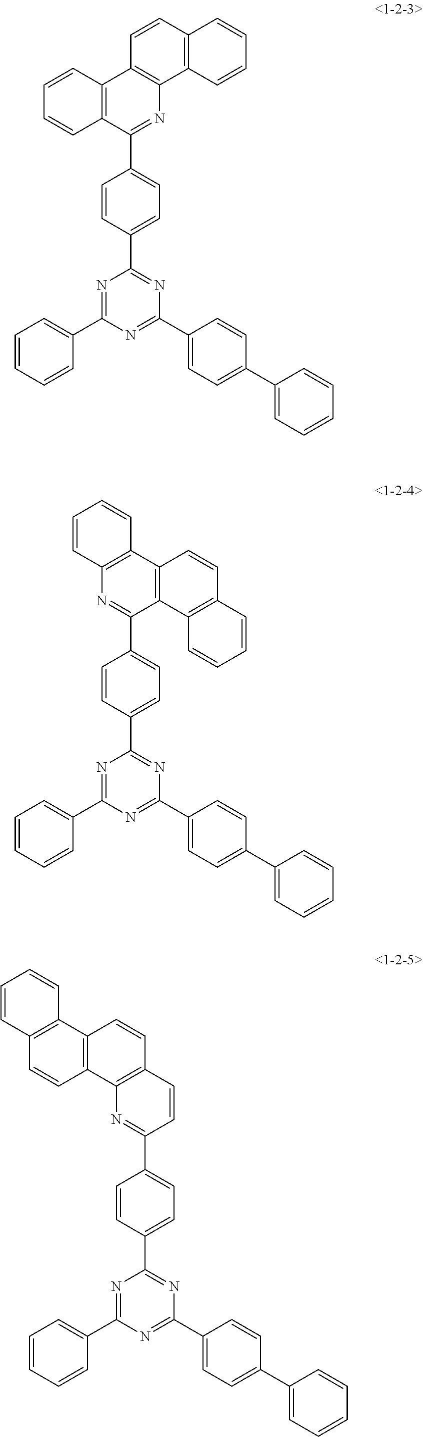 [SMILES: c1ccc(-c2ccc(-c3nc(-c4ccccc4)nc(-c4ccc(-c5nc6c7ccccc7ccc6c6ccccc56)cc4)n3)cc2)cc1 ; c1ccc(-c2ccc(-c3nc(-c4ccccc4)nc(-c4ccc(-c5nc6ccccc6c6ccc7ccccc7c56)cc4)n3)cc2)cc1 ; c1ccc(-c2ccc(-c3nc(-c4ccccc4)nc(-c4ccc(-c5ccc6ccc7c8ccccc8ccc7c6n5)cc4)n3)cc2)cc1]

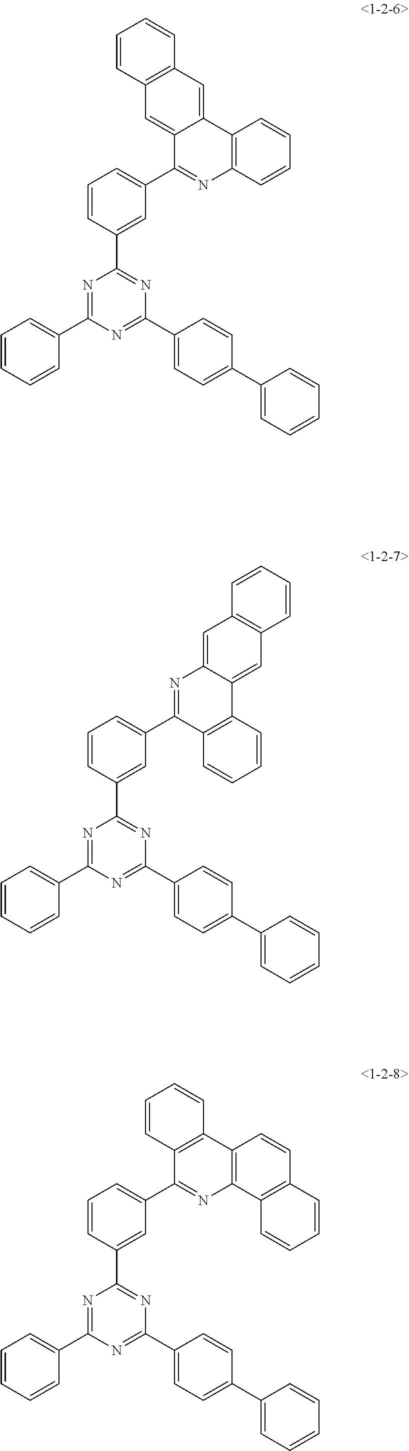 [SMILES: c1ccc(-c2ccc(-c3nc(-c4ccccc4)nc(-c4cccc(-c5nc6ccccc6c6cc7ccccc7cc56)c4)n3)cc2)cc1 ; c1ccc(-c2ccc(-c3nc(-c4ccccc4)nc(-c4cccc(-c5nc6cc7ccccc7cc6c6ccccc56)c4)n3)cc2)cc1 ; c1ccc(-c2ccc(-c3nc(-c4ccccc4)nc(-c4cccc(-c5nc6c7ccccc7ccc6c6ccccc56)c4)n3)cc2)cc1]

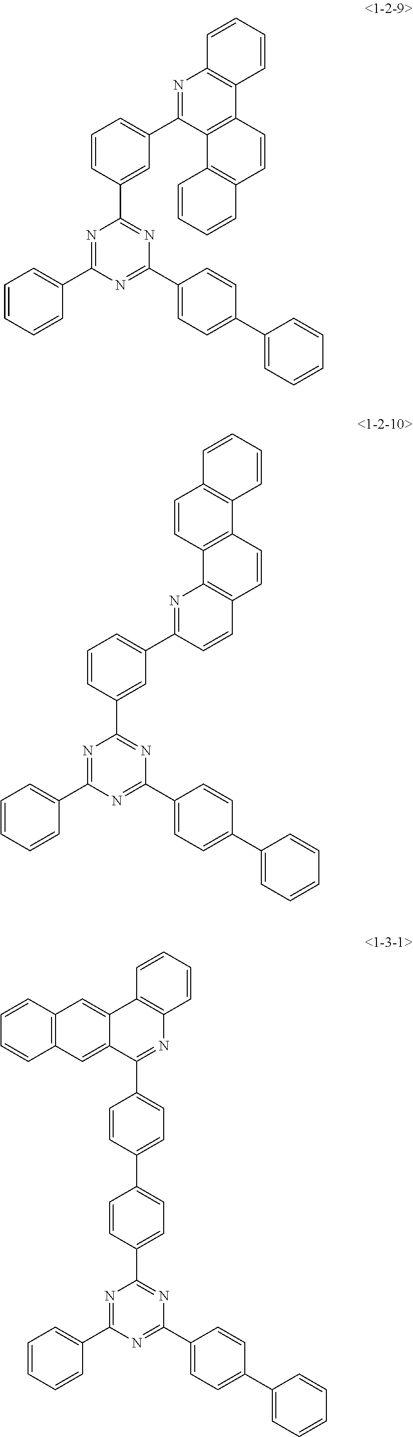 [SMILES: c1ccc(-c2ccc(-c3nc(-c4ccccc4)nc(-c4cccc(-c5nc6ccccc6c6ccc7ccccc7c56)c4)n3)cc2)cc1 ; c1ccc(-c2ccc(-c3nc(-c4ccccc4)nc(-c4cccc(-c5ccc6ccc7c8ccccc8ccc7c6n5)c4)n3)cc2)cc1 ; c1ccc(-c2ccc(-c3nc(-c4ccccc4)nc(-c4ccc(-c5ccc(-c6nc7ccccc7c7cc8ccccc8cc67)cc5)cc4)n3)cc2)cc1]

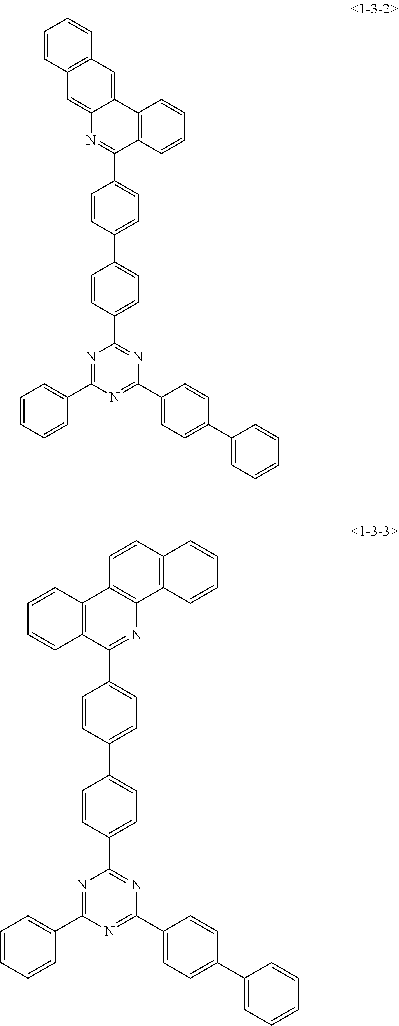 [SMILES: c1ccc(-c2ccc(-c3nc(-c4ccccc4)nc(-c4ccc(-c5ccc(-c6nc7cc8ccccc8cc7c7ccccc67)cc5)cc4)n3)cc2)cc1 ; c1ccc(-c2ccc(-c3nc(-c4ccccc4)nc(-c4ccc(-c5ccc(-c6nc7c8ccccc8ccc7c7ccccc67)cc5)cc4)n3)cc2)cc1]

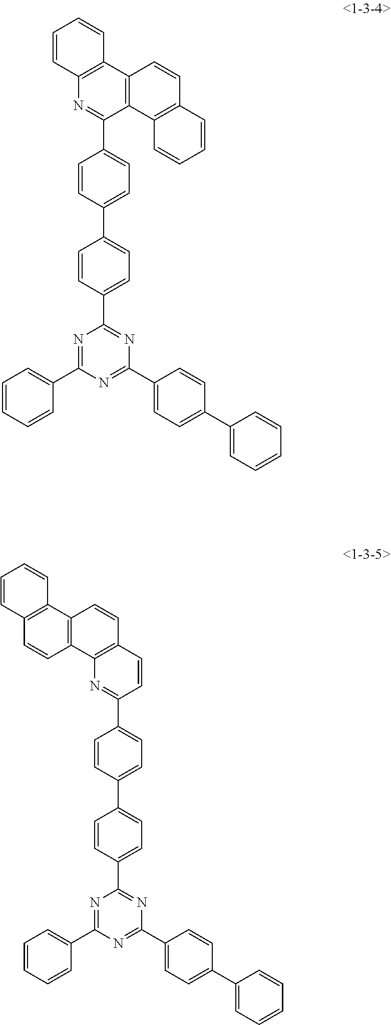 [SMILES: c1ccc(-c2ccc(-c3nc(-c4ccccc4)nc(-c4ccc(-c5ccc(-c6nc7ccccc7c7ccc8ccccc8c67)cc5)cc4)n3)cc2)cc1 ; c1ccc(-c2ccc(-c3nc(-c4ccccc4)nc(-c4ccc(-c5ccc(-c6ccc7ccc8c9ccccc9ccc8c7n6)cc5)cc4)n3)cc2)cc1]

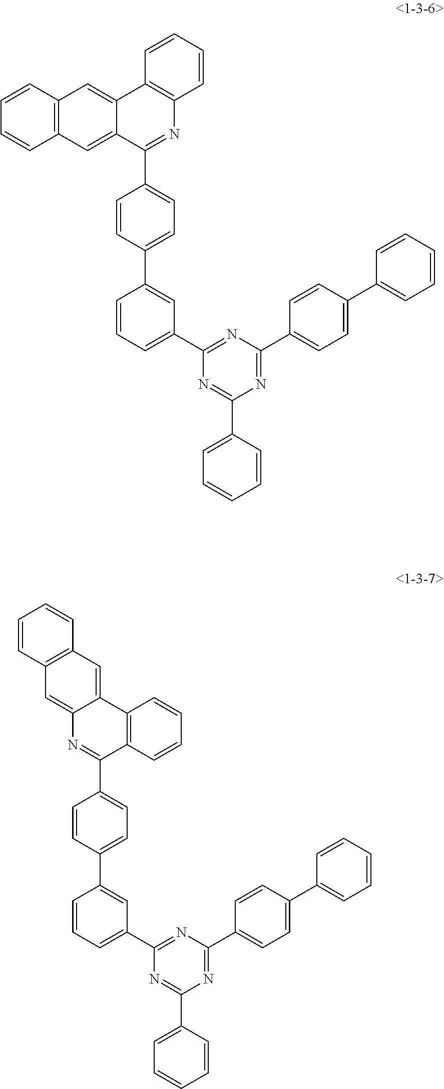 [SMILES: c1ccc(-c2ccc(-c3nc(-c4ccccc4)nc(-c4cccc(-c5ccc(-c6nc7ccccc7c7cc8ccccc8cc67)cc5)c4)n3)cc2)cc1 ; c1ccc(-c2ccc(-c3nc(-c4ccccc4)nc(-c4cccc(-c5ccc(-c6nc7cc8ccccc8cc7c7ccccc67)cc5)c4)n3)cc2)cc1]

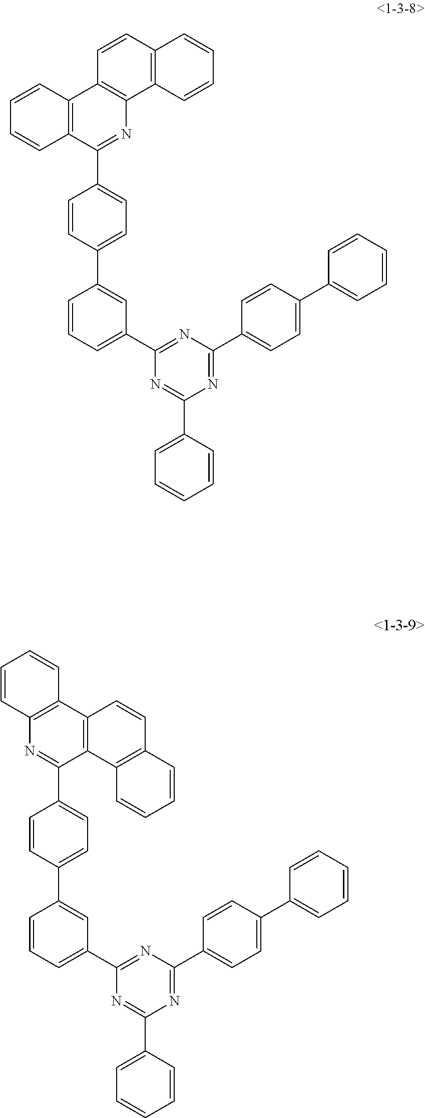 [SMILES: c1ccc(-c2ccc(-c3nc(-c4ccccc4)nc(-c4cccc(-c5ccc(-c6nc7c8ccccc8ccc7c7ccccc67)cc5)c4)n3)cc2)cc1 ; c1ccc(-c2ccc(-c3nc(-c4ccccc4)nc(-c4cccc(-c5ccc(-c6nc7ccccc7c7ccc8ccccc8c67)cc5)c4)n3)cc2)cc1]

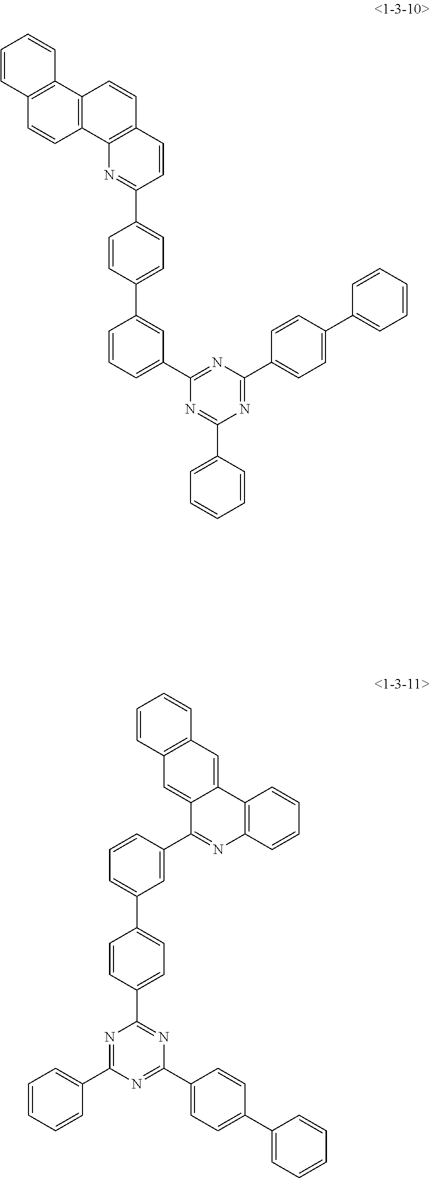 [SMILES: c1ccc(-c2ccc(-c3nc(-c4ccccc4)nc(-c4cccc(-c5ccc(-c6ccc7ccc8c9ccccc9ccc8c7n6)cc5)c4)n3)cc2)cc1 ; c1ccc(-c2ccc(-c3nc(-c4ccccc4)nc(-c4ccc(-c5cccc(-c6nc7ccccc7c7cc8ccccc8cc67)c5)cc4)n3)cc2)cc1]

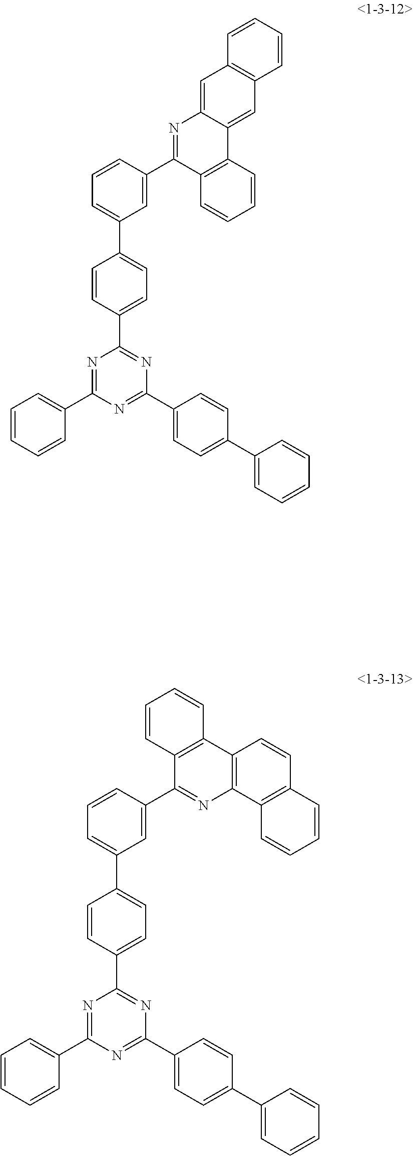 [SMILES: c1ccc(-c2ccc(-c3nc(-c4ccccc4)nc(-c4ccc(-c5cccc(-c6nc7cc8ccccc8cc7c7ccccc67)c5)cc4)n3)cc2)cc1 ; c1ccc(-c2ccc(-c3nc(-c4ccccc4)nc(-c4ccc(-c5cccc(-c6nc7c8ccccc8ccc7c7ccccc67)c5)cc4)n3)cc2)cc1]

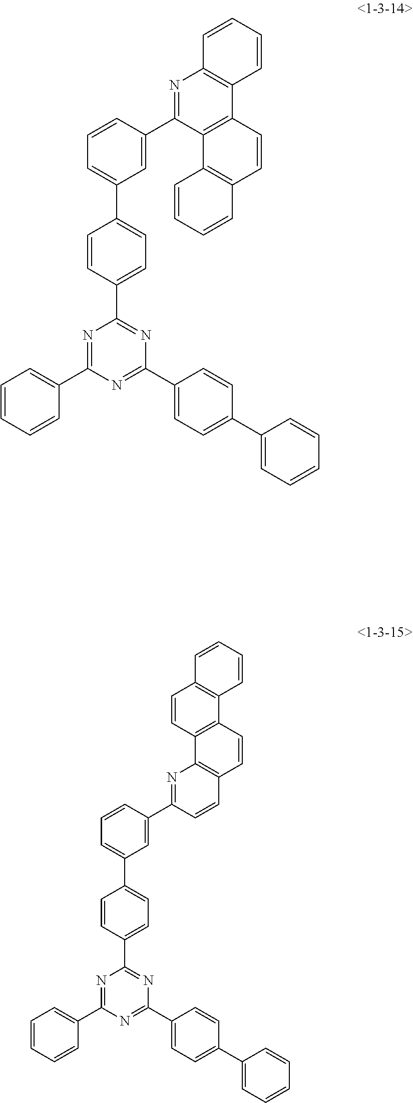 [SMILES: c1ccc(-c2ccc(-c3nc(-c4ccccc4)nc(-c4ccc(-c5cccc(-c6nc7ccccc7c7ccc8ccccc8c67)c5)cc4)n3)cc2)cc1 ; c1ccc(-c2ccc(-c3nc(-c4ccccc4)nc(-c4ccc(-c5cccc(-c6ccc7ccc8c9ccccc9ccc8c7n6)c5)cc4)n3)cc2)cc1]

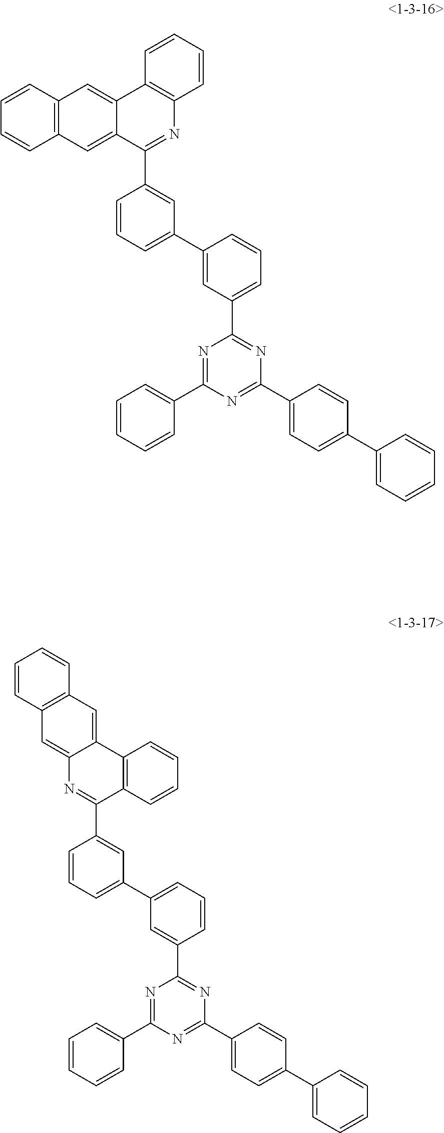 [SMILES: c1ccc(-c2ccc(-c3nc(-c4ccccc4)nc(-c4cccc(-c5cccc(-c6nc7ccccc7c7cc8ccccc8cc67)c5)c4)n3)cc2)cc1 ; c1ccc(-c2ccc(-c3nc(-c4ccccc4)nc(-c4cccc(-c5cccc(-c6nc7cc8ccccc8cc7c7ccccc67)c5)c4)n3)cc2)cc1]

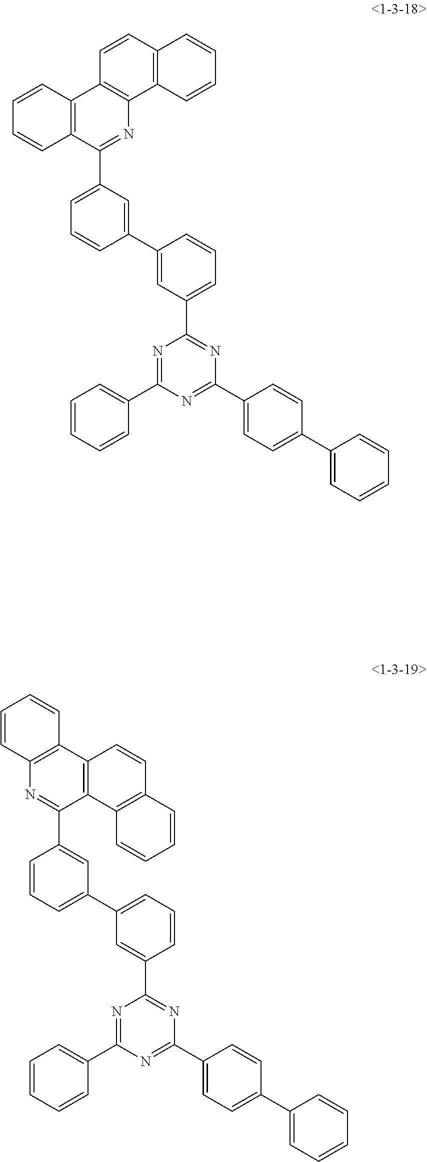 [SMILES: c1ccc(-c2ccc(-c3nc(-c4ccccc4)nc(-c4cccc(-c5cccc(-c6nc7c8ccccc8ccc7c7ccccc67)c5)c4)n3)cc2)cc1 ; c1ccc(-c2ccc(-c3nc(-c4ccccc4)nc(-c4cccc(-c5cccc(-c6nc7ccccc7c7ccc8ccccc8c67)c5)c4)n3)cc2)cc1]

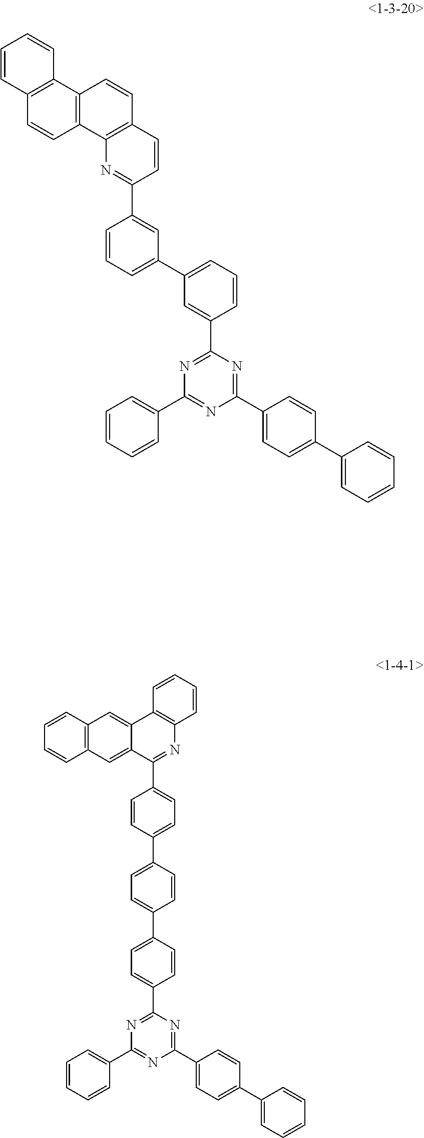 [SMILES: c1ccc(-c2ccc(-c3nc(-c4ccccc4)nc(-c4cccc(-c5cccc(-c6ccc7ccc8c9ccccc9ccc8c7n6)c5)c4)n3)cc2)cc1 ; c1ccc(-c2ccc(-c3nc(-c4ccccc4)nc(-c4ccc(-c5ccc(-c6ccc(-c7nc8ccccc8c8cc9ccccc9cc78)cc6)cc5)cc4)n3)cc2)cc1]

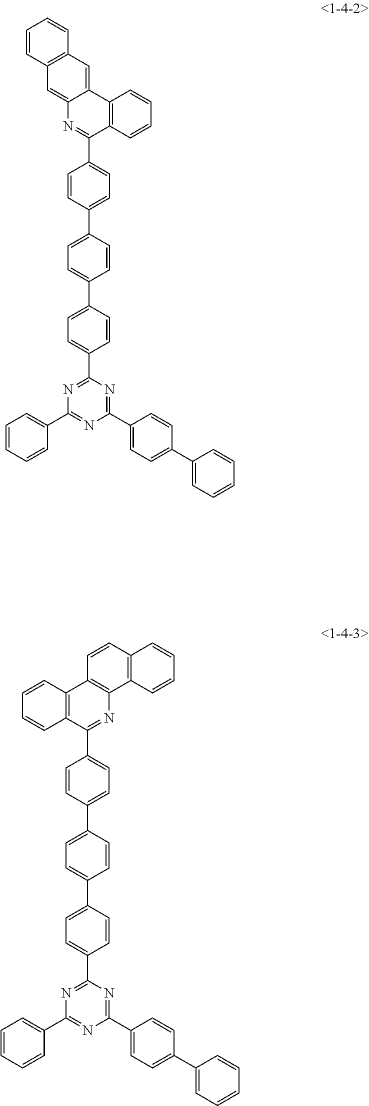 [SMILES: c1ccc(-c2ccc(-c3nc(-c4ccccc4)nc(-c4ccc(-c5ccc(-c6ccc(-c7nc8cc9ccccc9cc8c8ccccc78)cc6)cc5)cc4)n3)cc2)cc1 ; c1ccc(-c2ccc(-c3nc(-c4ccccc4)nc(-c4ccc(-c5ccc(-c6ccc(-c7nc8c9ccccc9ccc8c8ccccc78)cc6)cc5)cc4)n3)cc2)cc1]

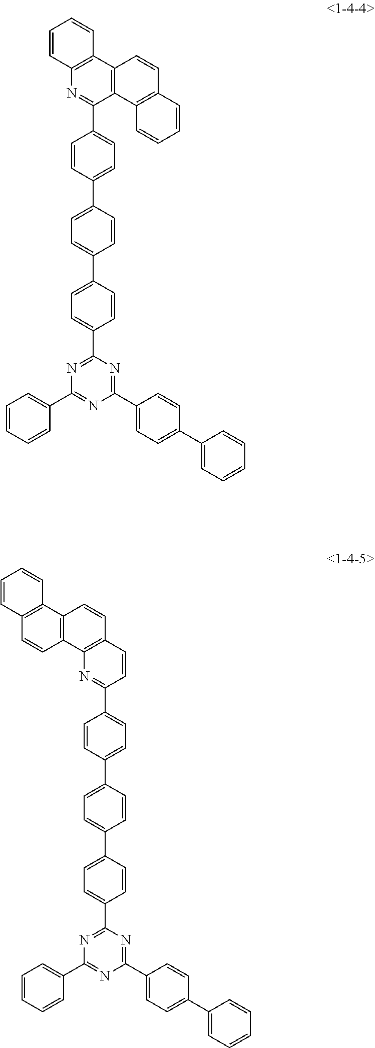 [SMILES: c1ccc(-c2ccc(-c3nc(-c4ccccc4)nc(-c4ccc(-c5ccc(-c6ccc(-c7nc8ccccc8c8ccc9ccccc9c78)cc6)cc5)cc4)n3)cc2)cc1 ; c1ccc(-c2ccc(-c3nc(-c4ccccc4)nc(-c4ccc(-c5ccc(-c6ccc(-c7ccc8ccc9c%10ccccc%10ccc9c8n7)cc6)cc5)cc4)n3)cc2)cc1]

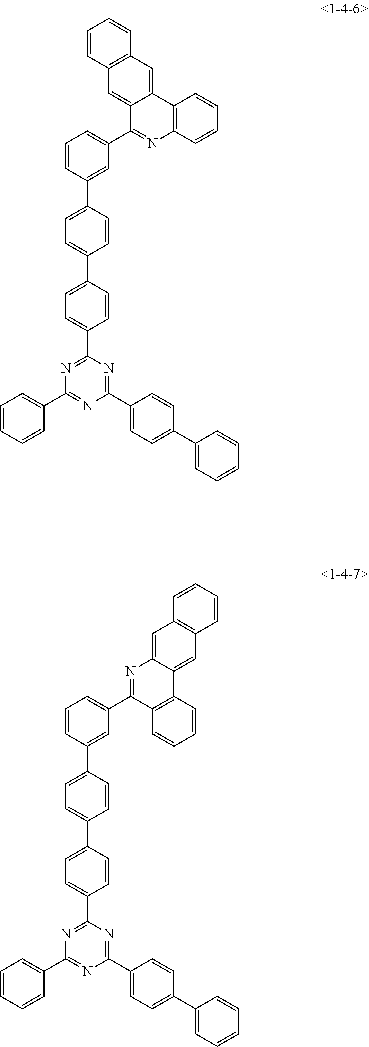 [SMILES: c1ccc(-c2ccc(-c3nc(-c4ccccc4)nc(-c4ccc(-c5ccc(-c6cccc(-c7nc8ccccc8c8cc9ccccc9cc78)c6)cc5)cc4)n3)cc2)cc1 ; c1ccc(-c2ccc(-c3nc(-c4ccccc4)nc(-c4ccc(-c5ccc(-c6cccc(-c7nc8cc9ccccc9cc8c8ccccc78)c6)cc5)cc4)n3)cc2)cc1]

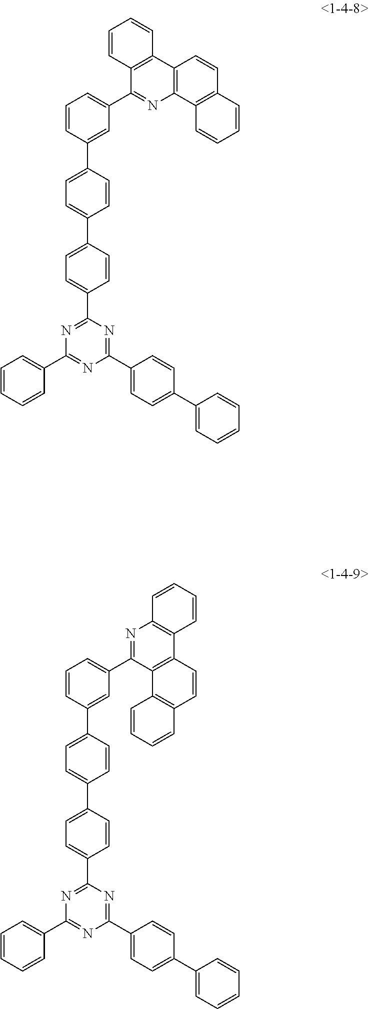 [SMILES: c1ccc(-c2ccc(-c3nc(-c4ccccc4)nc(-c4ccc(-c5ccc(-c6cccc(-c7nc8c9ccccc9ccc8c8ccccc78)c6)cc5)cc4)n3)cc2)cc1 ; c1ccc(-c2ccc(-c3nc(-c4ccccc4)nc(-c4ccc(-c5ccc(-c6cccc(-c7nc8ccccc8c8ccc9ccccc9c78)c6)cc5)cc4)n3)cc2)cc1]

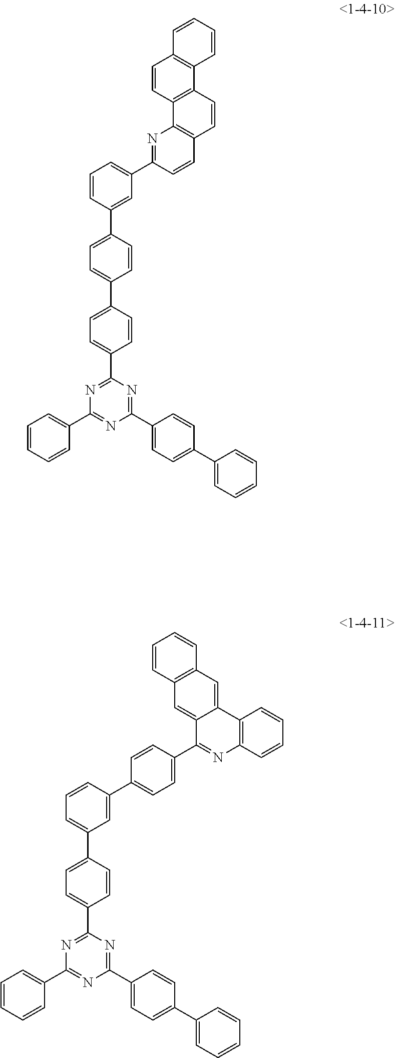 [SMILES: c1ccc(-c2ccc(-c3nc(-c4ccccc4)nc(-c4ccc(-c5ccc(-c6cccc(-c7ccc8ccc9c%10ccccc%10ccc9c8n7)c6)cc5)cc4)n3)cc2)cc1 ; c1ccc(-c2ccc(-c3nc(-c4ccccc4)nc(-c4ccc(-c5cccc(-c6ccc(-c7nc8ccccc8c8cc9ccccc9cc78)cc6)c5)cc4)n3)cc2)cc1]

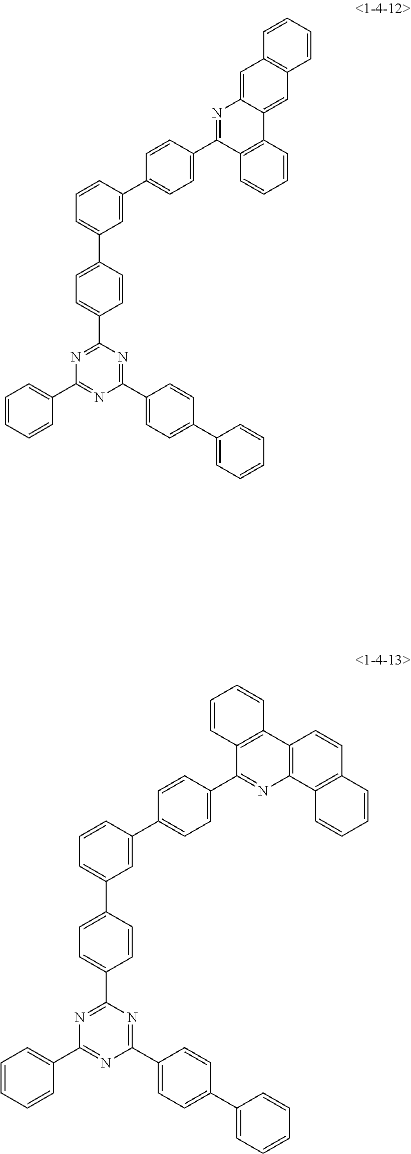 [SMILES: c1ccc(-c2ccc(-c3nc(-c4ccccc4)nc(-c4ccc(-c5cccc(-c6ccc(-c7nc8cc9ccccc9cc8c8ccccc78)cc6)c5)cc4)n3)cc2)cc1 ; c1ccc(-c2ccc(-c3nc(-c4ccccc4)nc(-c4ccc(-c5cccc(-c6ccc(-c7nc8c9ccccc9ccc8c8ccccc78)cc6)c5)cc4)n3)cc2)cc1]

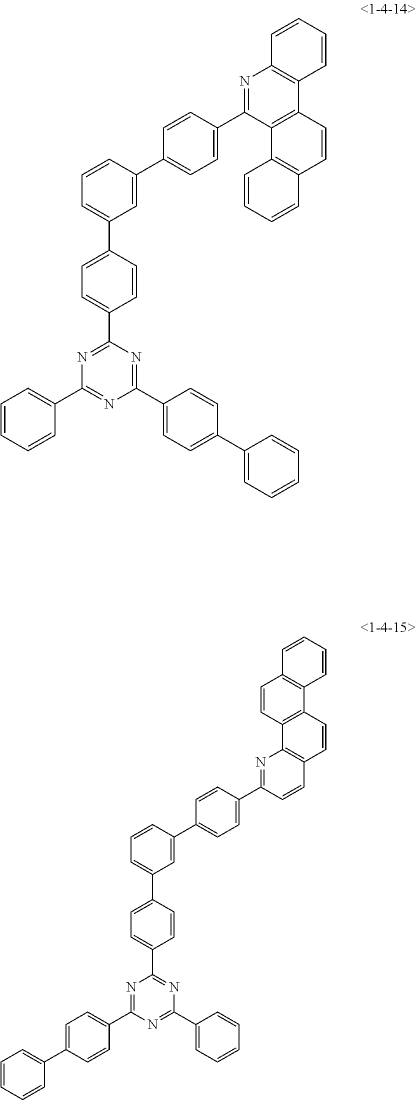 [SMILES: c1ccc(-c2ccc(-c3nc(-c4ccccc4)nc(-c4ccc(-c5cccc(-c6ccc(-c7nc8ccccc8c8ccc9ccccc9c78)cc6)c5)cc4)n3)cc2)cc1 ; c1ccc(-c2ccc(-c3nc(-c4ccccc4)nc(-c4ccc(-c5cccc(-c6ccc(-c7ccc8ccc9c%10ccccc%10ccc9c8n7)cc6)c5)cc4)n3)cc2)cc1]

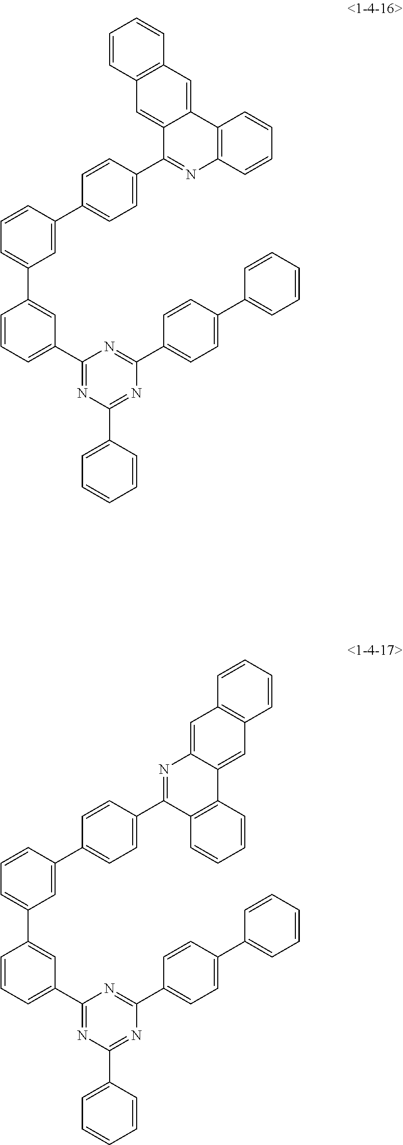 [SMILES: c1ccc(-c2ccc(-c3nc(-c4ccccc4)nc(-c4cccc(-c5cccc(-c6ccc(-c7nc8ccccc8c8cc9ccccc9cc78)cc6)c5)c4)n3)cc2)cc1 ; c1ccc(-c2ccc(-c3nc(-c4ccccc4)nc(-c4cccc(-c5cccc(-c6ccc(-c7nc8cc9ccccc9cc8c8ccccc78)cc6)c5)c4)n3)cc2)cc1]

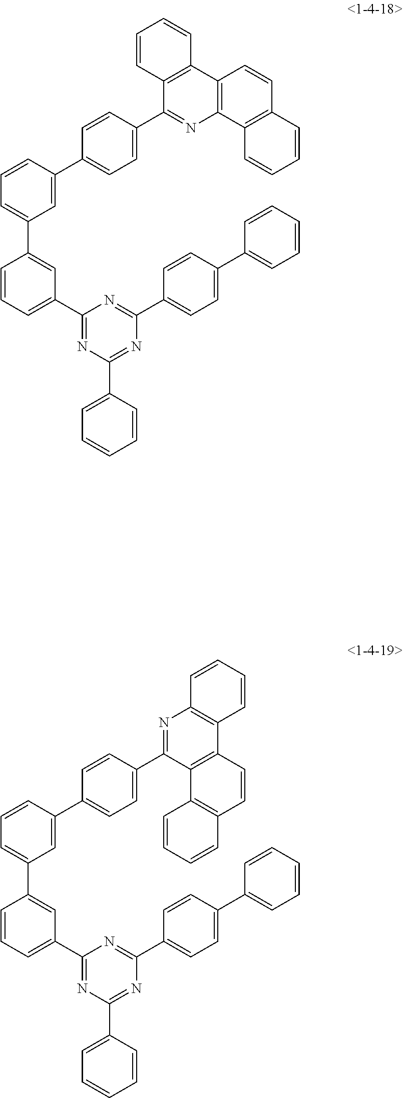 [SMILES: c1ccc(-c2ccc(-c3nc(-c4ccccc4)nc(-c4cccc(-c5cccc(-c6ccc(-c7nc8c9ccccc9ccc8c8ccccc78)cc6)c5)c4)n3)cc2)cc1 ; c1ccc(-c2ccc(-c3nc(-c4ccccc4)nc(-c4cccc(-c5cccc(-c6ccc(-c7nc8ccccc8c8ccc9ccccc9c78)cc6)c5)c4)n3)cc2)cc1]

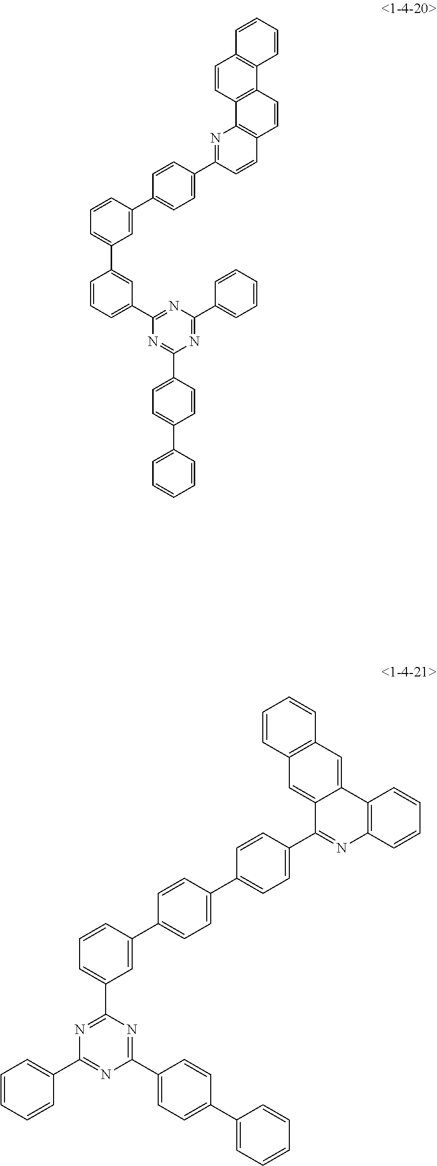 [SMILES: c1ccc(-c2ccc(-c3nc(-c4ccccc4)nc(-c4cccc(-c5cccc(-c6ccc(-c7ccc8ccc9c%10ccccc%10ccc9c8n7)cc6)c5)c4)n3)cc2)cc1 ; c1ccc(-c2ccc(-c3nc(-c4ccccc4)nc(-c4cccc(-c5ccc(-c6ccc(-c7nc8ccccc8c8cc9ccccc9cc78)cc6)cc5)c4)n3)cc2)cc1]

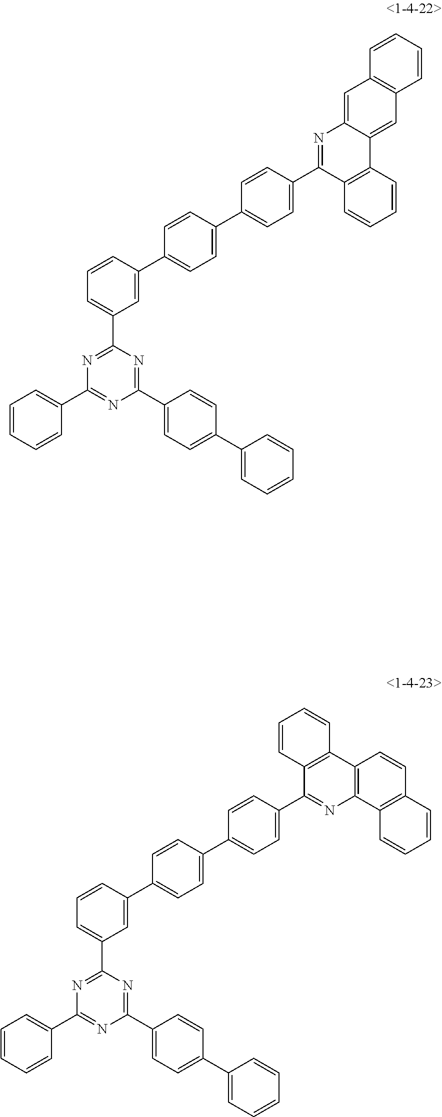 [SMILES: c1ccc(-c2ccc(-c3nc(-c4ccccc4)nc(-c4cccc(-c5ccc(-c6ccc(-c7nc8cc9ccccc9cc8c8ccccc78)cc6)cc5)c4)n3)cc2)cc1 ; c1ccc(-c2ccc(-c3nc(-c4ccccc4)nc(-c4cccc(-c5ccc(-c6ccc(-c7nc8c9ccccc9ccc8c8ccccc78)cc6)cc5)c4)n3)cc2)cc1]

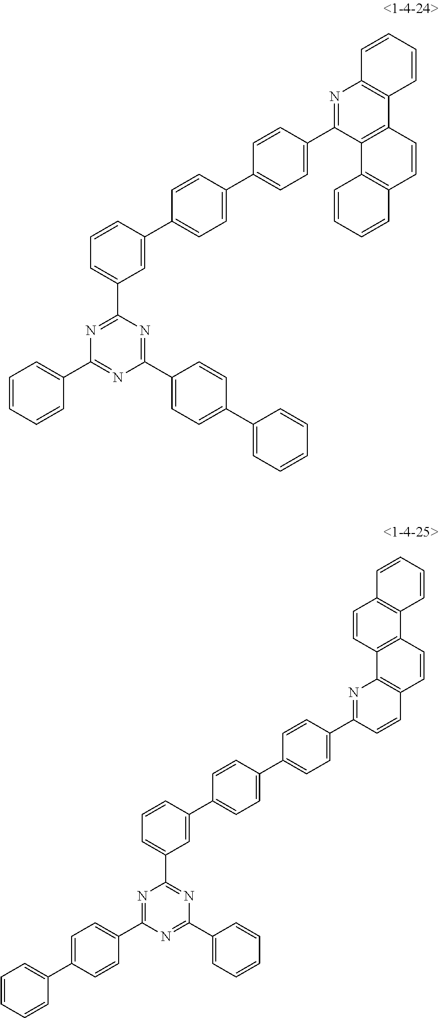 [SMILES: c1ccc(-c2ccc(-c3nc(-c4ccccc4)nc(-c4cccc(-c5ccc(-c6ccc(-c7nc8ccccc8c8ccc9ccccc9c78)cc6)cc5)c4)n3)cc2)cc1 ; c1ccc(-c2ccc(-c3nc(-c4ccccc4)nc(-c4cccc(-c5ccc(-c6ccc(-c7ccc8ccc9c%10ccccc%10ccc9c8n7)cc6)cc5)c4)n3)cc2)cc1]

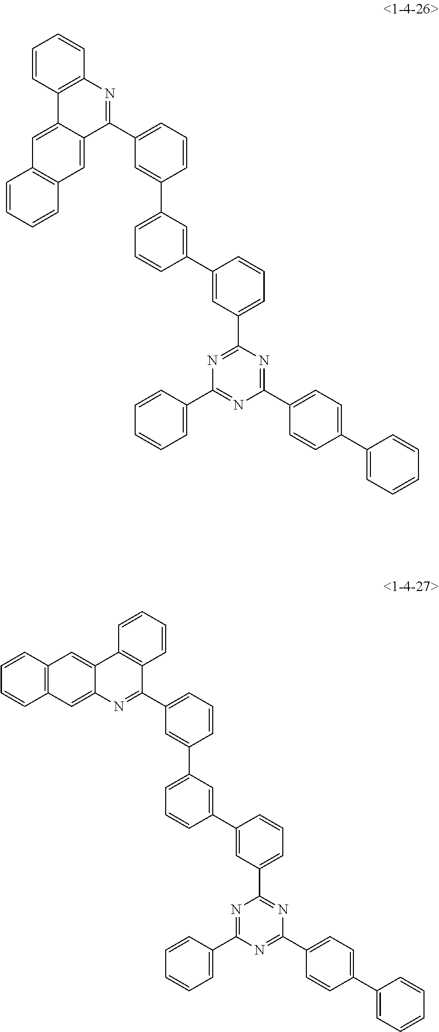 [SMILES: c1ccc(-c2ccc(-c3nc(-c4ccccc4)nc(-c4cccc(-c5cccc(-c6cccc(-c7nc8ccccc8c8cc9ccccc9cc78)c6)c5)c4)n3)cc2)cc1 ; c1ccc(-c2ccc(-c3nc(-c4ccccc4)nc(-c4cccc(-c5cccc(-c6cccc(-c7nc8cc9ccccc9cc8c8ccccc78)c6)c5)c4)n3)cc2)cc1]

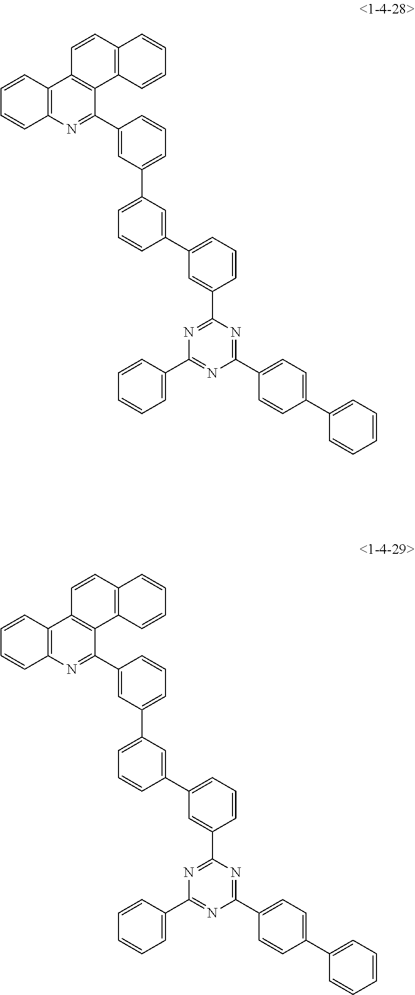 [SMILES: c1ccc(-c2ccc(-c3nc(-c4ccccc4)nc(-c4cccc(-c5cccc(-c6cccc(-c7nc8ccccc8c8ccc9ccccc9c78)c6)c5)c4)n3)cc2)cc1 ; c1ccc(-c2ccc(-c3nc(-c4ccccc4)nc(-c4cccc(-c5cccc(-c6cccc(-c7nc8ccccc8c8ccc9ccccc9c78)c6)c5)c4)n3)cc2)cc1]

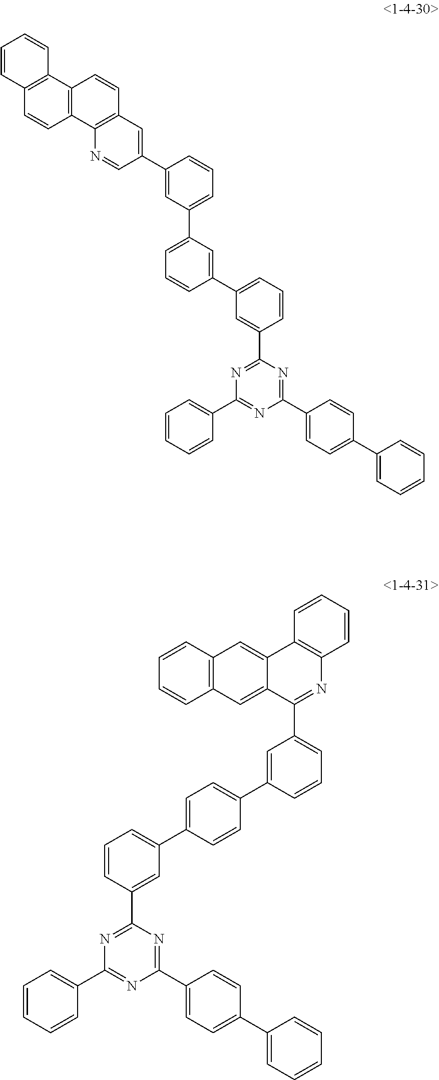 [SMILES: c1ccc(-c2ccc(-c3nc(-c4ccccc4)nc(-c4cccc(-c5cccc(-c6cccc(-c7cnc8c(ccc9c%10ccccc%10ccc98)c7)c6)c5)c4)n3)cc2)cc1 ; c1ccc(-c2ccc(-c3nc(-c4ccccc4)nc(-c4cccc(-c5ccc(-c6cccc(-c7nc8ccccc8c8cc9ccccc9cc78)c6)cc5)c4)n3)cc2)cc1]

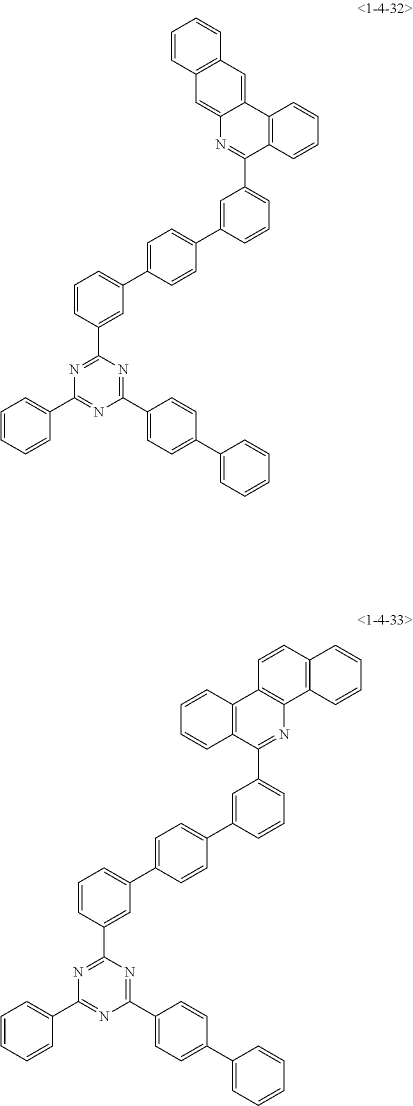 [SMILES: c1ccc(-c2ccc(-c3nc(-c4ccccc4)nc(-c4cccc(-c5ccc(-c6cccc(-c7nc8cc9ccccc9cc8c8ccccc78)c6)cc5)c4)n3)cc2)cc1 ; c1ccc(-c2ccc(-c3nc(-c4ccccc4)nc(-c4cccc(-c5ccc(-c6cccc(-c7nc8c9ccccc9ccc8c8ccccc78)c6)cc5)c4)n3)cc2)cc1]

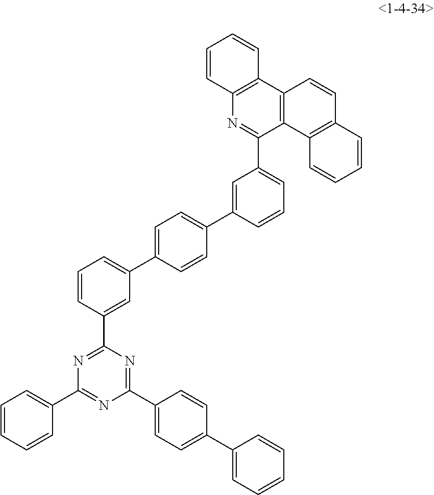 [SMILES: c1ccc(-c2ccc(-c3nc(-c4ccccc4)nc(-c4cccc(-c5ccc(-c6cccc(-c7nc8ccccc8c8ccc9ccccc9c78)c6)cc5)c4)n3)cc2)cc1]

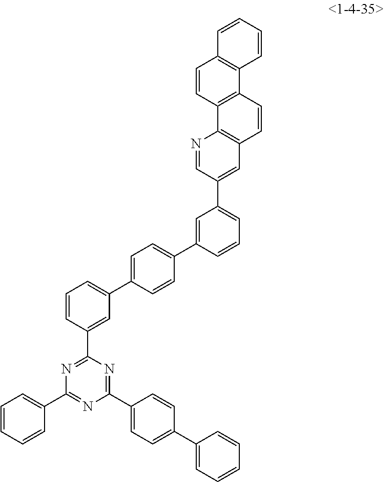 [SMILES: c1ccc(-c2ccc(-c3nc(-c4ccccc4)nc(-c4cccc(-c5ccc(-c6cccc(-c7cnc8c(ccc9c%10ccccc%10ccc98)c7)c6)cc5)c4)n3)cc2)cc1]

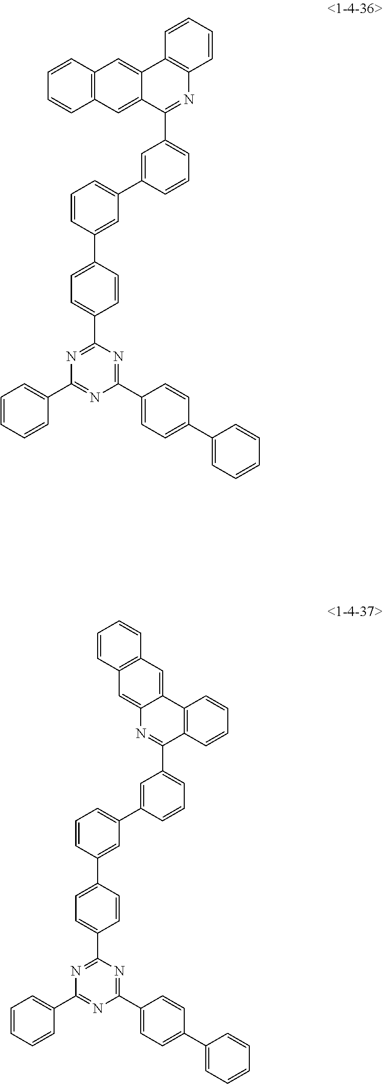 [SMILES: c1ccc(-c2ccc(-c3nc(-c4ccccc4)nc(-c4ccc(-c5cccc(-c6cccc(-c7nc8ccccc8c8cc9ccccc9cc78)c6)c5)cc4)n3)cc2)cc1 ; c1ccc(-c2ccc(-c3nc(-c4ccccc4)nc(-c4ccc(-c5cccc(-c6cccc(-c7nc8cc9ccccc9cc8c8ccccc78)c6)c5)cc4)n3)cc2)cc1]

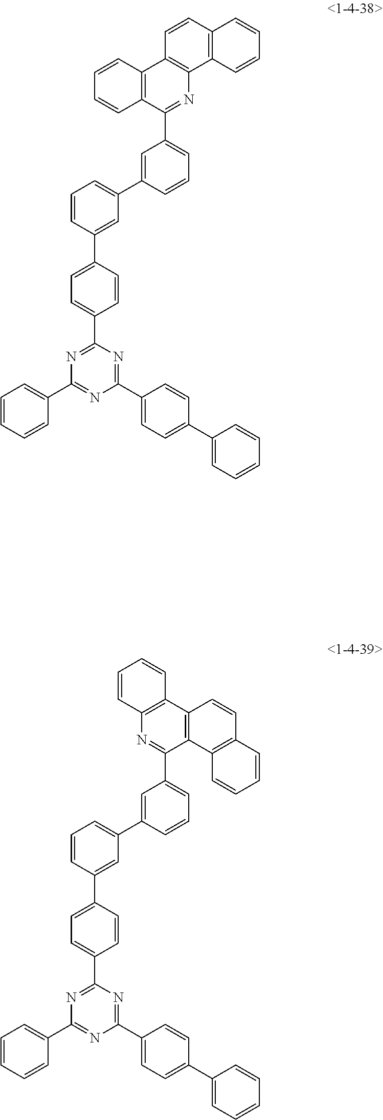 [SMILES: c1ccc(-c2ccc(-c3nc(-c4ccccc4)nc(-c4ccc(-c5cccc(-c6cccc(-c7nc8c9ccccc9ccc8c8ccccc78)c6)c5)cc4)n3)cc2)cc1 ; c1ccc(-c2ccc(-c3nc(-c4ccccc4)nc(-c4ccc(-c5cccc(-c6cccc(-c7nc8ccccc8c8ccc9ccccc9c78)c6)c5)cc4)n3)cc2)cc1]

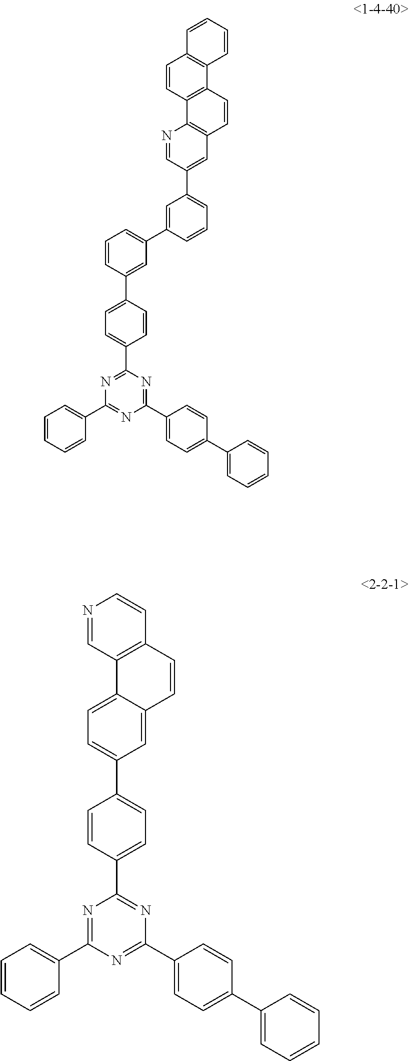 [SMILES: c1ccc(-c2ccc(-c3nc(-c4ccccc4)nc(-c4ccc(-c5cccc(-c6cccc(-c7cnc8c(ccc9c%10ccccc%10ccc98)c7)c6)c5)cc4)n3)cc2)cc1 ; c1ccc(-c2ccc(-c3nc(-c4ccccc4)nc(-c4ccc(-c5ccc6c(ccc7ccncc76)c5)cc4)n3)cc2)cc1]

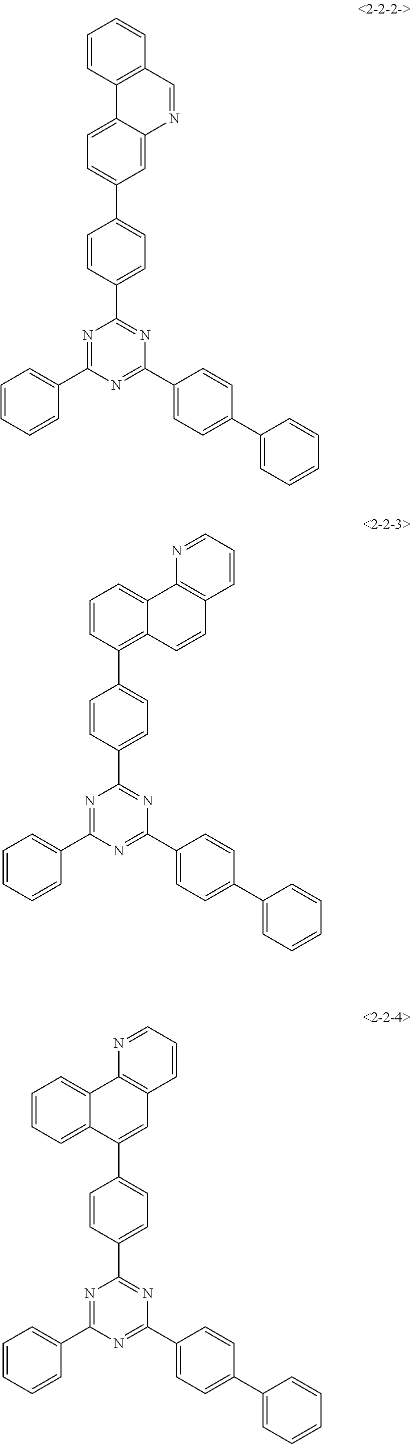 [SMILES: c1ccc(-c2ccc(-c3nc(-c4ccccc4)nc(-c4ccc(-c5ccc6c(c5)ncc5ccccc56)cc4)n3)cc2)cc1 ; c1ccc(-c2ccc(-c3nc(-c4ccccc4)nc(-c4ccc(-c5cccc6c5ccc5cccnc56)cc4)n3)cc2)cc1 ; c1ccc(-c2ccc(-c3nc(-c4ccccc4)nc(-c4ccc(-c5cc6cccnc6c6ccccc56)cc4)n3)cc2)cc1]

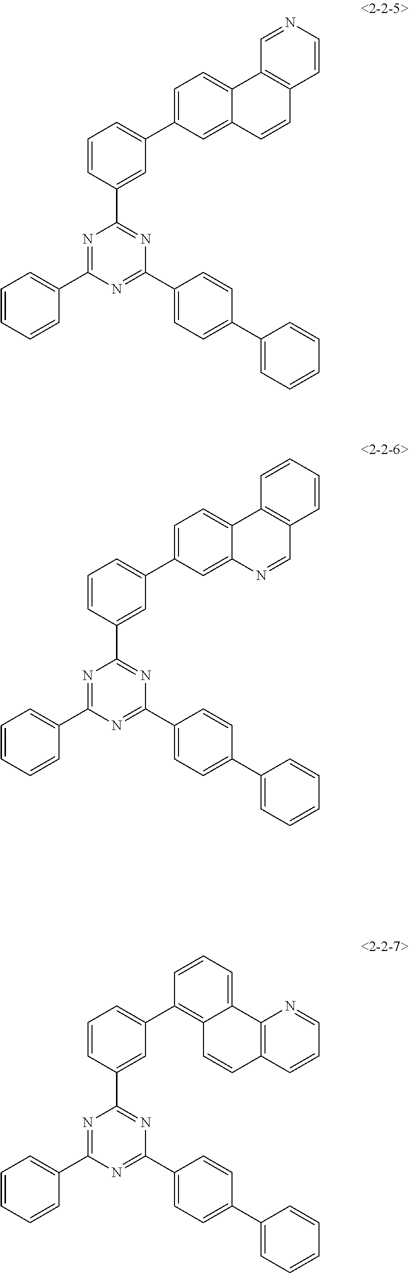 [SMILES: c1ccc(-c2ccc(-c3nc(-c4ccccc4)nc(-c4cccc(-c5ccc6c(ccc7ccncc76)c5)c4)n3)cc2)cc1 ; c1ccc(-c2ccc(-c3nc(-c4ccccc4)nc(-c4cccc(-c5ccc6c(c5)ncc5ccccc56)c4)n3)cc2)cc1 ; c1ccc(-c2ccc(-c3nc(-c4ccccc4)nc(-c4cccc(-c5cccc6c5ccc5cccnc56)c4)n3)cc2)cc1]

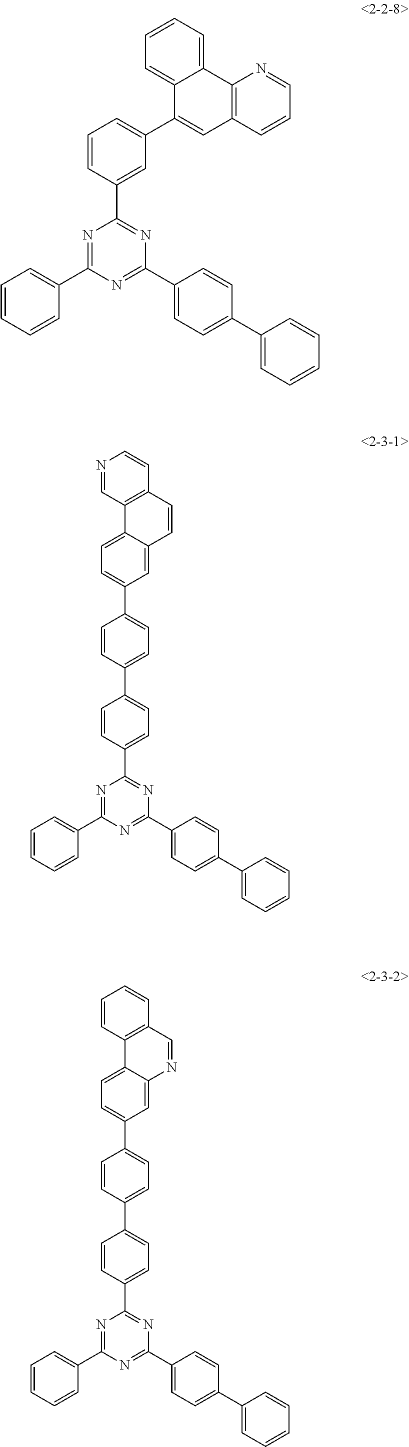 [SMILES: c1ccc(-c2ccc(-c3nc(-c4ccccc4)nc(-c4cccc(-c5cc6cccnc6c6ccccc56)c4)n3)cc2)cc1 ; c1ccc(-c2ccc(-c3nc(-c4ccccc4)nc(-c4ccc(-c5ccc(-c6ccc7c(ccc8ccncc87)c6)cc5)cc4)n3)cc2)cc1 ; c1ccc(-c2ccc(-c3nc(-c4ccccc4)nc(-c4ccc(-c5ccc(-c6ccc7c(c6)ncc6ccccc67)cc5)cc4)n3)cc2)cc1]

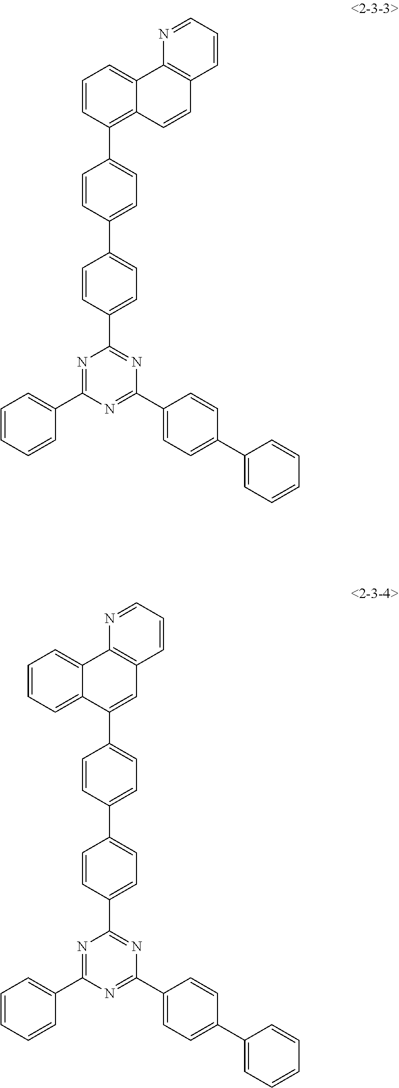 [SMILES: c1ccc(-c2ccc(-c3nc(-c4ccccc4)nc(-c4ccc(-c5ccc(-c6cccc7c6ccc6cccnc67)cc5)cc4)n3)cc2)cc1 ; c1ccc(-c2ccc(-c3nc(-c4ccccc4)nc(-c4ccc(-c5ccc(-c6cc7cccnc7c7ccccc67)cc5)cc4)n3)cc2)cc1]

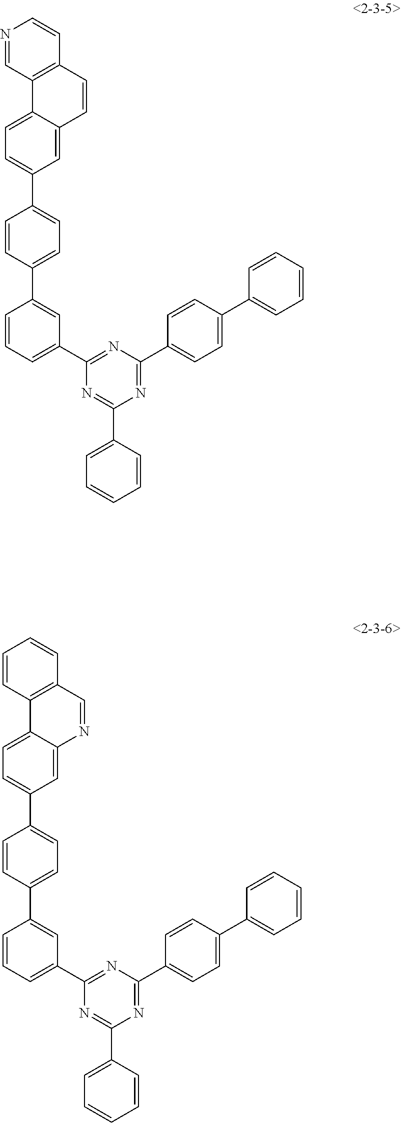 [SMILES: c1ccc(-c2ccc(-c3nc(-c4ccccc4)nc(-c4cccc(-c5ccc(-c6ccc7c(ccc8ccncc87)c6)cc5)c4)n3)cc2)cc1 ; c1ccc(-c2ccc(-c3nc(-c4ccccc4)nc(-c4cccc(-c5ccc(-c6ccc7c(c6)ncc6ccccc67)cc5)c4)n3)cc2)cc1]

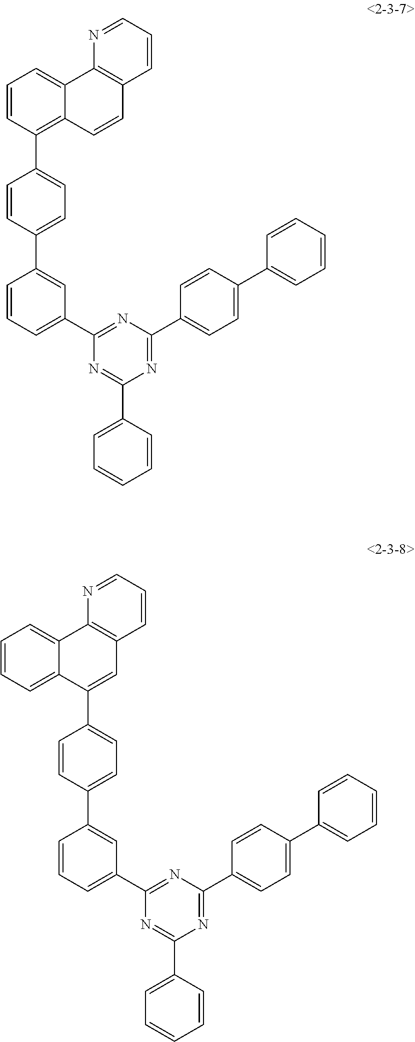 [SMILES: c1ccc(-c2ccc(-c3nc(-c4ccccc4)nc(-c4cccc(-c5ccc(-c6cccc7c6ccc6cccnc67)cc5)c4)n3)cc2)cc1 ; c1ccc(-c2ccc(-c3nc(-c4ccccc4)nc(-c4cccc(-c5ccc(-c6cc7cccnc7c7ccccc67)cc5)c4)n3)cc2)cc1]

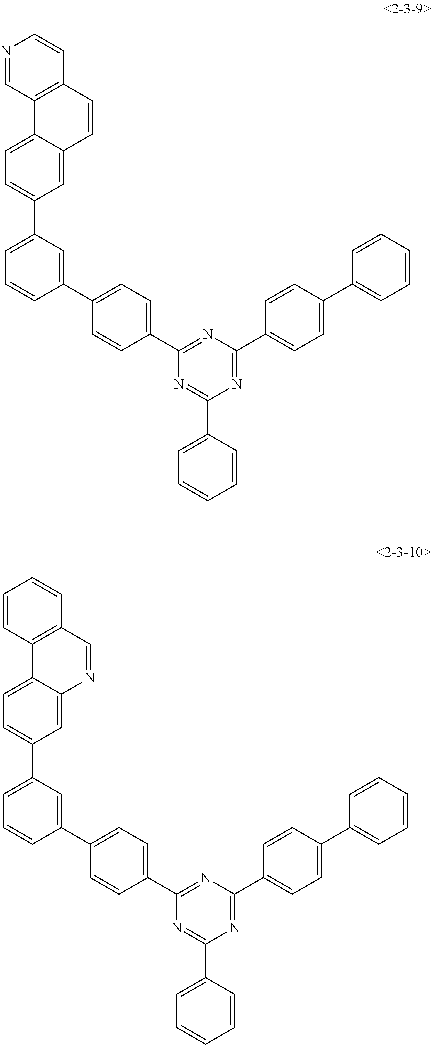 [SMILES: c1ccc(-c2ccc(-c3nc(-c4ccccc4)nc(-c4ccc(-c5cccc(-c6ccc7c(ccc8ccncc87)c6)c5)cc4)n3)cc2)cc1 ; c1ccc(-c2ccc(-c3nc(-c4ccccc4)nc(-c4ccc(-c5cccc(-c6ccc7c(c6)ncc6ccccc67)c5)cc4)n3)cc2)cc1]

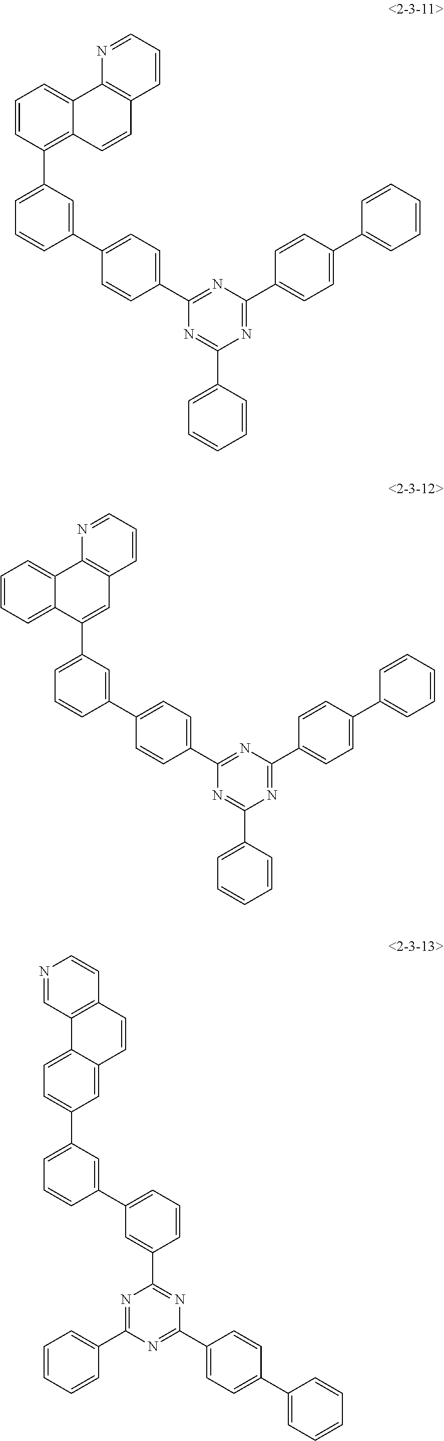 [SMILES: c1ccc(-c2ccc(-c3nc(-c4ccccc4)nc(-c4ccc(-c5cccc(-c6cccc7c6ccc6cccnc67)c5)cc4)n3)cc2)cc1 ; c1ccc(-c2ccc(-c3nc(-c4ccccc4)nc(-c4ccc(-c5cccc(-c6cc7cccnc7c7ccccc67)c5)cc4)n3)cc2)cc1 ; c1ccc(-c2ccc(-c3nc(-c4ccccc4)nc(-c4cccc(-c5cccc(-c6ccc7c(ccc8ccncc87)c6)c5)c4)n3)cc2)cc1]

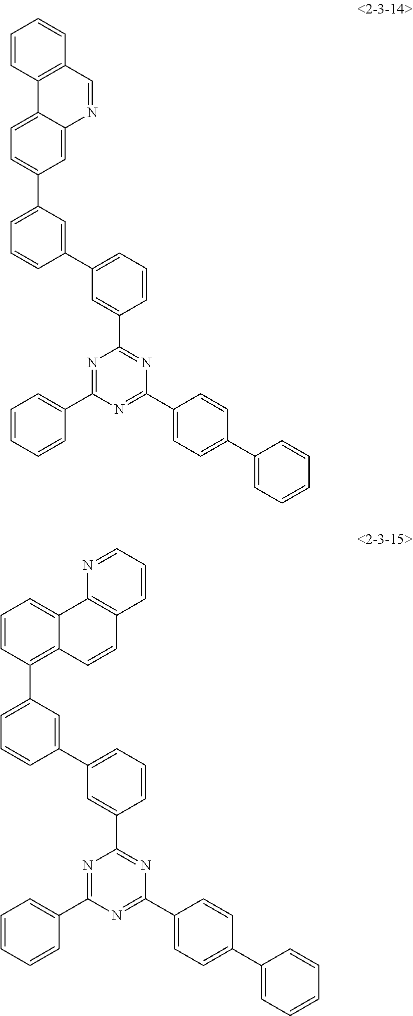 [SMILES: c1ccc(-c2ccc(-c3nc(-c4ccccc4)nc(-c4cccc(-c5cccc(-c6ccc7c(c6)ncc6ccccc67)c5)c4)n3)cc2)cc1 ; c1ccc(-c2ccc(-c3nc(-c4ccccc4)nc(-c4cccc(-c5cccc(-c6cccc7c6ccc6cccnc67)c5)c4)n3)cc2)cc1]

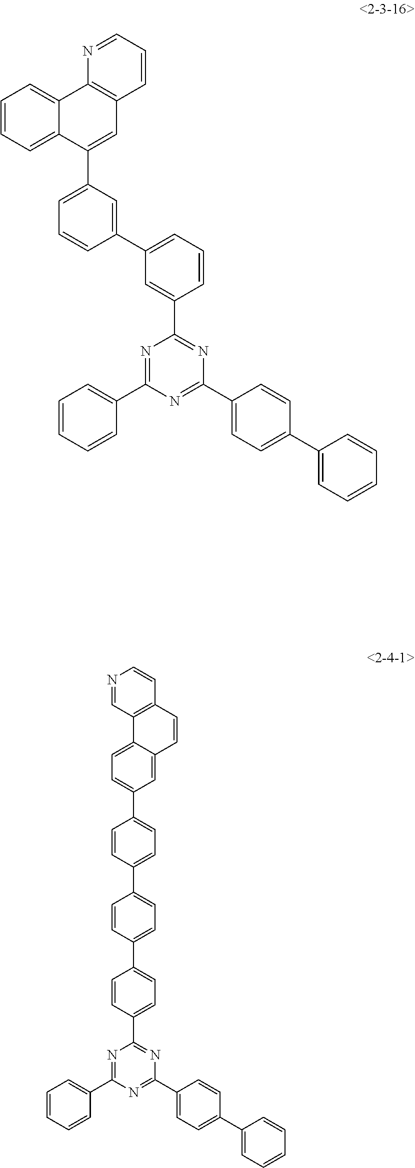 [SMILES: c1ccc(-c2ccc(-c3nc(-c4ccccc4)nc(-c4cccc(-c5cccc(-c6cc7cccnc7c7ccccc67)c5)c4)n3)cc2)cc1 ; c1ccc(-c2ccc(-c3nc(-c4ccccc4)nc(-c4ccc(-c5ccc(-c6ccc(-c7ccc8c(ccc9ccncc98)c7)cc6)cc5)cc4)n3)cc2)cc1]

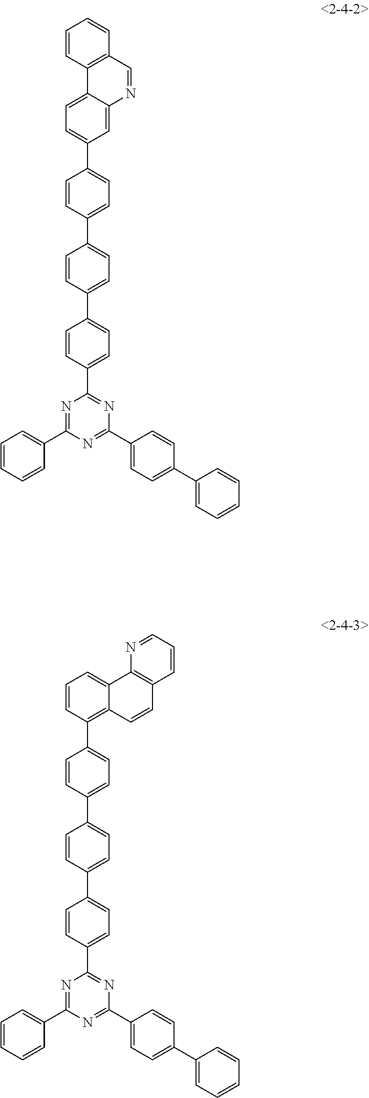 [SMILES: c1ccc(-c2ccc(-c3nc(-c4ccccc4)nc(-c4ccc(-c5ccc(-c6ccc(-c7ccc8c(c7)ncc7ccccc78)cc6)cc5)cc4)n3)cc2)cc1 ; c1ccc(-c2ccc(-c3nc(-c4ccccc4)nc(-c4ccc(-c5ccc(-c6ccc(-c7cccc8c7ccc7cccnc78)cc6)cc5)cc4)n3)cc2)cc1]

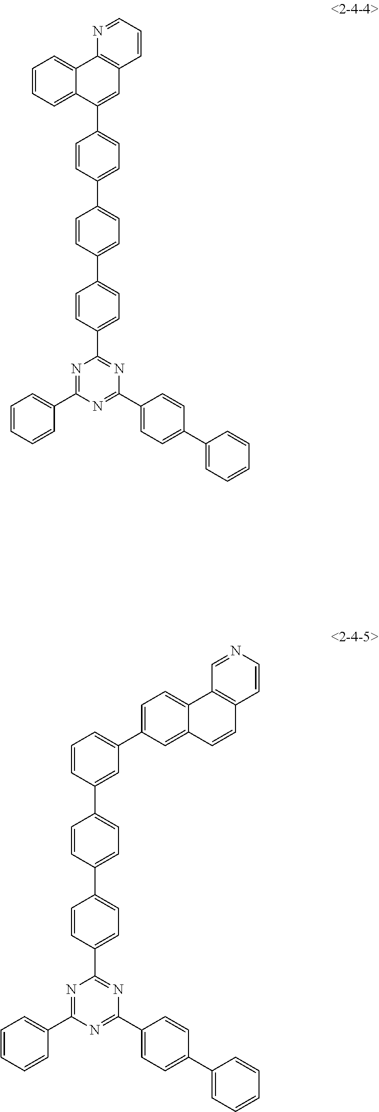 [SMILES: c1ccc(-c2ccc(-c3nc(-c4ccccc4)nc(-c4ccc(-c5ccc(-c6ccc(-c7cc8cccnc8c8ccccc78)cc6)cc5)cc4)n3)cc2)cc1 ; c1ccc(-c2ccc(-c3nc(-c4ccccc4)nc(-c4ccc(-c5ccc(-c6cccc(-c7ccc8c(ccc9ccncc98)c7)c6)cc5)cc4)n3)cc2)cc1]

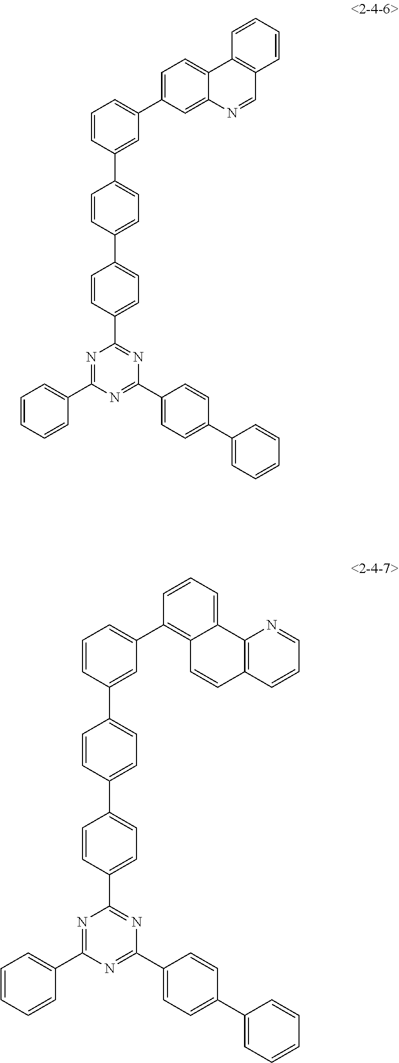 [SMILES: c1ccc(-c2ccc(-c3nc(-c4ccccc4)nc(-c4ccc(-c5ccc(-c6cccc(-c7ccc8c(c7)ncc7ccccc78)c6)cc5)cc4)n3)cc2)cc1 ; c1ccc(-c2ccc(-c3nc(-c4ccccc4)nc(-c4ccc(-c5ccc(-c6cccc(-c7cccc8c7ccc7cccnc78)c6)cc5)cc4)n3)cc2)cc1]

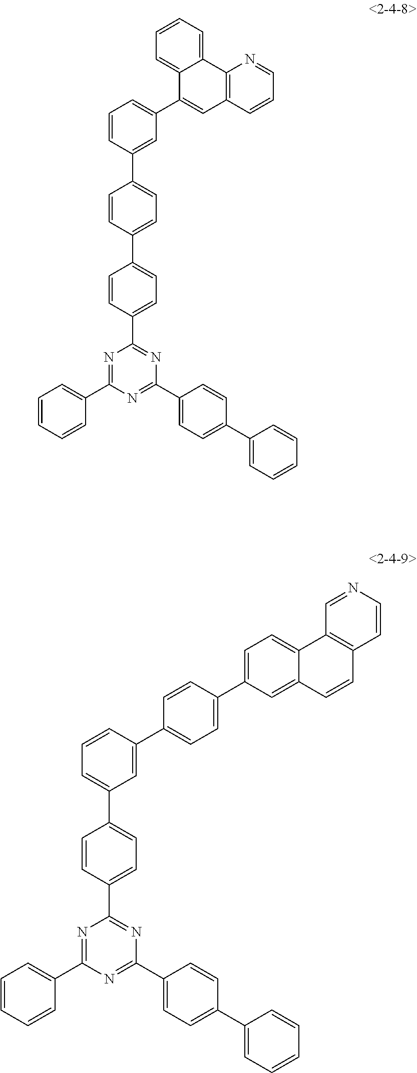 [SMILES: c1ccc(-c2ccc(-c3nc(-c4ccccc4)nc(-c4ccc(-c5ccc(-c6cccc(-c7cc8cccnc8c8ccccc78)c6)cc5)cc4)n3)cc2)cc1 ; c1ccc(-c2ccc(-c3nc(-c4ccccc4)nc(-c4ccc(-c5cccc(-c6ccc(-c7ccc8c(ccc9ccncc98)c7)cc6)c5)cc4)n3)cc2)cc1]

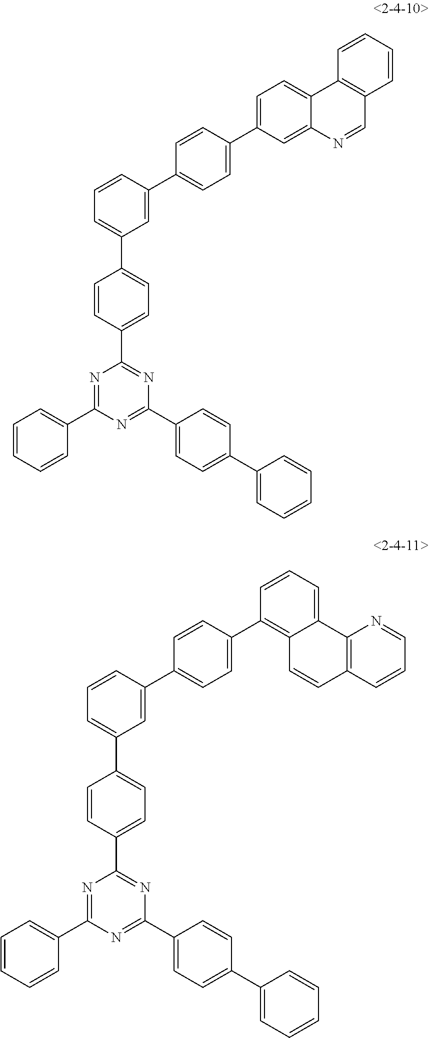 [SMILES: c1ccc(-c2ccc(-c3nc(-c4ccccc4)nc(-c4ccc(-c5cccc(-c6ccc(-c7ccc8c(c7)ncc7ccccc78)cc6)c5)cc4)n3)cc2)cc1 ; c1ccc(-c2ccc(-c3nc(-c4ccccc4)nc(-c4ccc(-c5cccc(-c6ccc(-c7cccc8c7ccc7cccnc78)cc6)c5)cc4)n3)cc2)cc1]

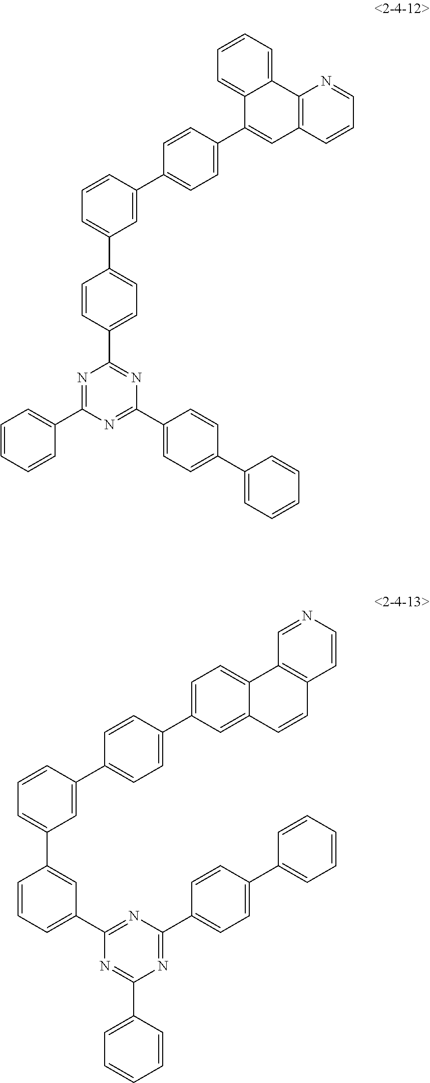 [SMILES: c1ccc(-c2ccc(-c3nc(-c4ccccc4)nc(-c4ccc(-c5cccc(-c6ccc(-c7cc8cccnc8c8ccccc78)cc6)c5)cc4)n3)cc2)cc1 ; c1ccc(-c2ccc(-c3nc(-c4ccccc4)nc(-c4cccc(-c5cccc(-c6ccc(-c7ccc8c(ccc9ccncc98)c7)cc6)c5)c4)n3)cc2)cc1]

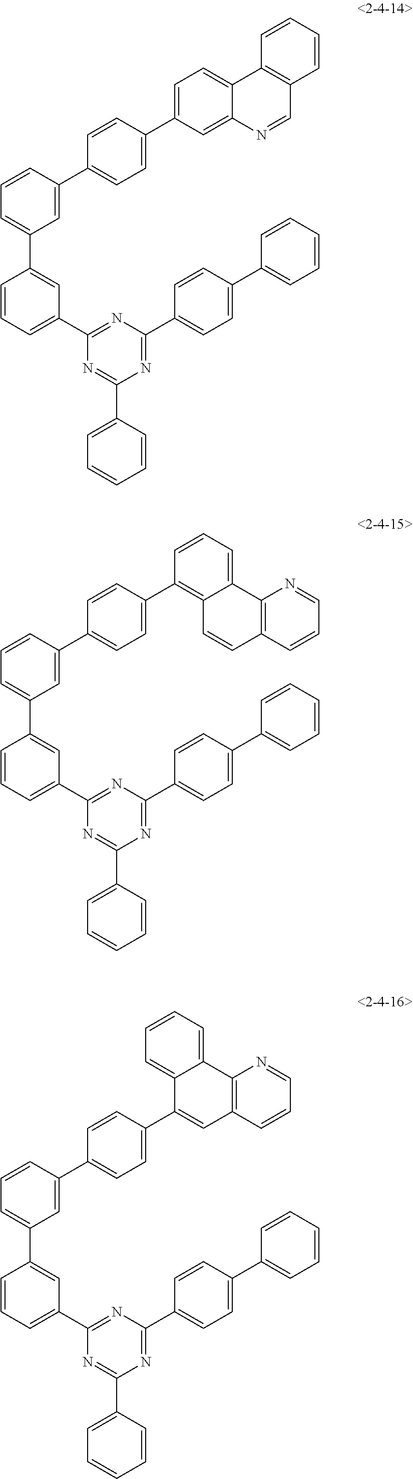 [SMILES: c1ccc(-c2ccc(-c3nc(-c4ccccc4)nc(-c4cccc(-c5cccc(-c6ccc(-c7ccc8c(c7)ncc7ccccc78)cc6)c5)c4)n3)cc2)cc1 ; c1ccc(-c2ccc(-c3nc(-c4ccccc4)nc(-c4cccc(-c5cccc(-c6ccc(-c7cccc8c7ccc7cccnc78)cc6)c5)c4)n3)cc2)cc1 ; c1ccc(-c2ccc(-c3nc(-c4ccccc4)nc(-c4cccc(-c5cccc(-c6ccc(-c7cc8cccnc8c8ccccc78)cc6)c5)c4)n3)cc2)cc1]

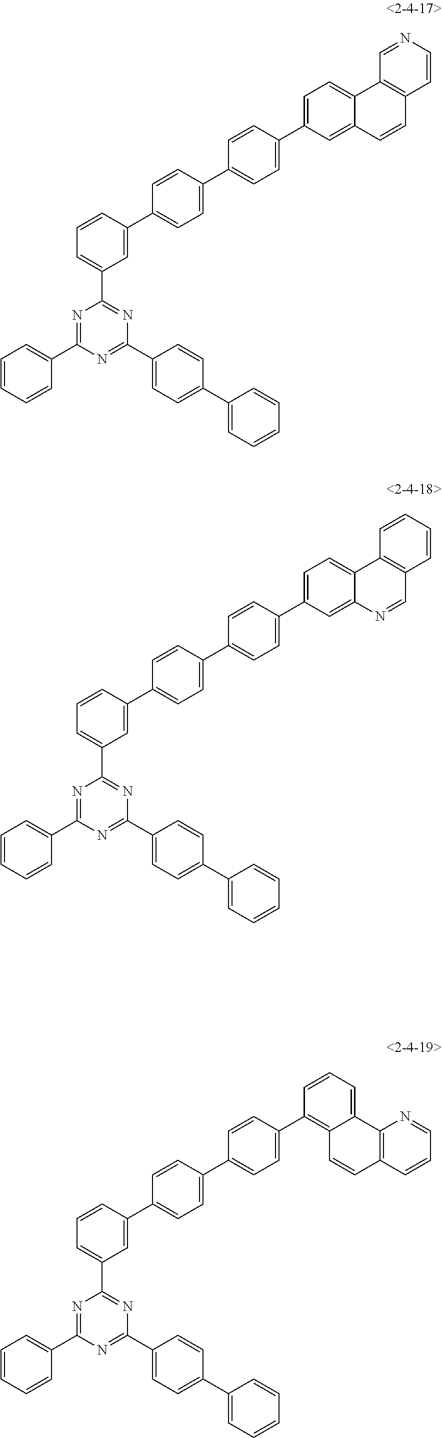 [SMILES: c1ccc(-c2ccc(-c3nc(-c4ccccc4)nc(-c4cccc(-c5ccc(-c6ccc(-c7ccc8c(ccc9ccncc98)c7)cc6)cc5)c4)n3)cc2)cc1 ; c1ccc(-c2ccc(-c3nc(-c4ccccc4)nc(-c4cccc(-c5ccc(-c6ccc(-c7ccc8c(c7)ncc7ccccc78)cc6)cc5)c4)n3)cc2)cc1 ; c1ccc(-c2ccc(-c3nc(-c4ccccc4)nc(-c4cccc(-c5ccc(-c6ccc(-c7cccc8c7ccc7cccnc78)cc6)cc5)c4)n3)cc2)cc1]

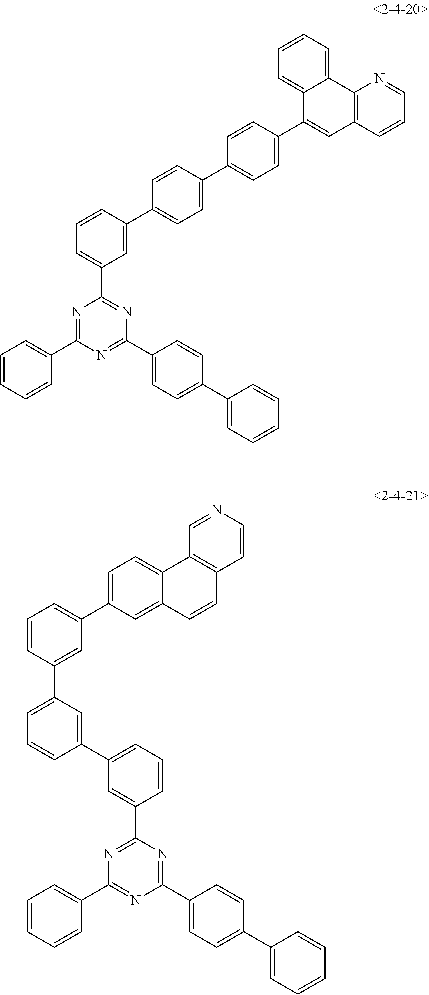 [SMILES: c1ccc(-c2ccc(-c3nc(-c4ccccc4)nc(-c4cccc(-c5ccc(-c6ccc(-c7cc8cccnc8c8ccccc78)cc6)cc5)c4)n3)cc2)cc1 ; c1ccc(-c2ccc(-c3nc(-c4ccccc4)nc(-c4cccc(-c5cccc(-c6cccc(-c7ccc8c(ccc9ccncc98)c7)c6)c5)c4)n3)cc2)cc1]

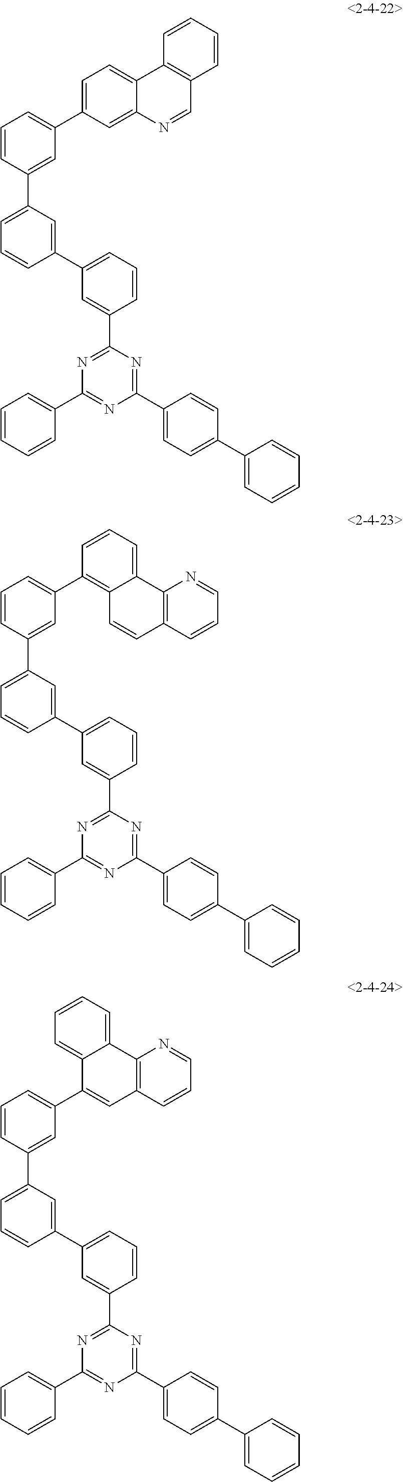 [SMILES: c1ccc(-c2ccc(-c3nc(-c4ccccc4)nc(-c4cccc(-c5cccc(-c6cccc(-c7ccc8c(c7)ncc7ccccc78)c6)c5)c4)n3)cc2)cc1 ; c1ccc(-c2ccc(-c3nc(-c4ccccc4)nc(-c4cccc(-c5cccc(-c6cccc(-c7cccc8c7ccc7cccnc78)c6)c5)c4)n3)cc2)cc1 ; c1ccc(-c2ccc(-c3nc(-c4ccccc4)nc(-c4cccc(-c5cccc(-c6cccc(-c7cc8cccnc8c8ccccc78)c6)c5)c4)n3)cc2)cc1]

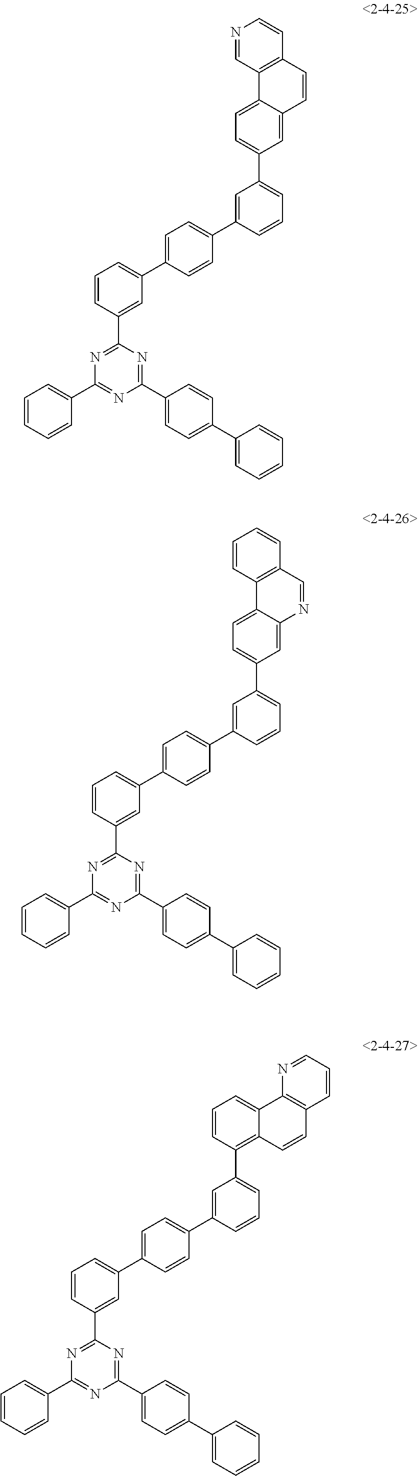 [SMILES: c1ccc(-c2ccc(-c3nc(-c4ccccc4)nc(-c4cccc(-c5ccc(-c6cccc(-c7ccc8c(ccc9ccncc98)c7)c6)cc5)c4)n3)cc2)cc1 ; c1ccc(-c2ccc(-c3nc(-c4ccccc4)nc(-c4cccc(-c5ccc(-c6cccc(-c7ccc8c(c7)ncc7ccccc78)c6)cc5)c4)n3)cc2)cc1 ; c1ccc(-c2ccc(-c3nc(-c4ccccc4)nc(-c4cccc(-c5ccc(-c6cccc(-c7cccc8c7ccc7cccnc78)c6)cc5)c4)n3)cc2)cc1]

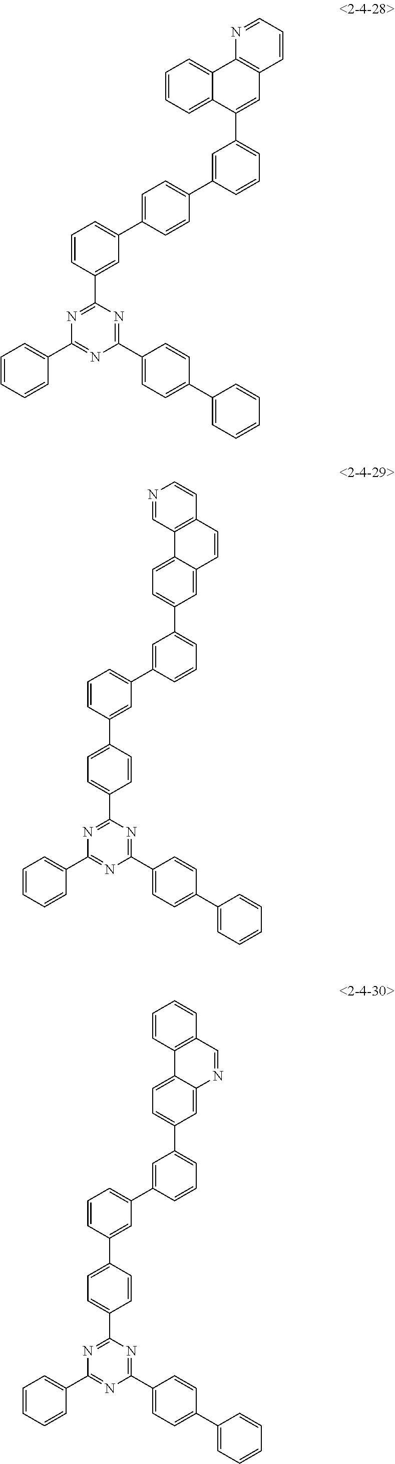 [SMILES: c1ccc(-c2ccc(-c3nc(-c4ccccc4)nc(-c4cccc(-c5ccc(-c6cccc(-c7cc8cccnc8c8ccccc78)c6)cc5)c4)n3)cc2)cc1 ; c1ccc(-c2ccc(-c3nc(-c4ccccc4)nc(-c4ccc(-c5cccc(-c6cccc(-c7ccc8c(ccc9ccncc98)c7)c6)c5)cc4)n3)cc2)cc1 ; c1ccc(-c2ccc(-c3nc(-c4ccccc4)nc(-c4ccc(-c5cccc(-c6cccc(-c7ccc8c(c7)ncc7ccccc78)c6)c5)cc4)n3)cc2)cc1]

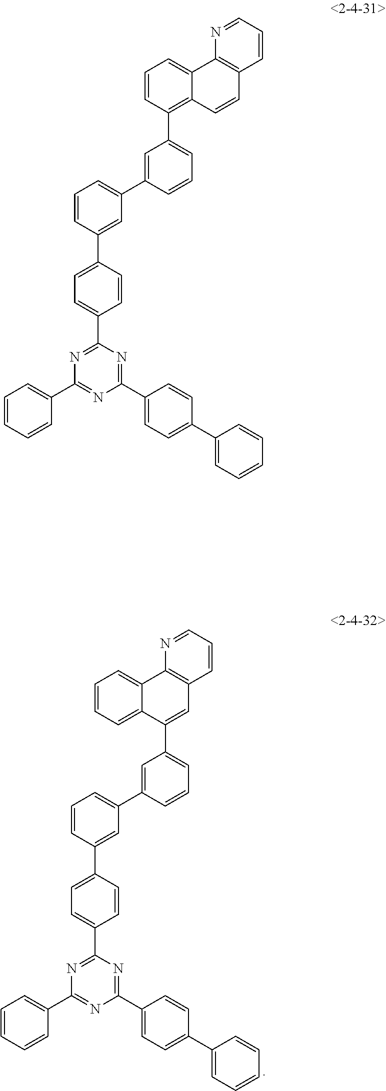 [SMILES: c1ccc(-c2ccc(-c3nc(-c4ccccc4)nc(-c4ccc(-c5cccc(-c6cccc(-c7cccc8c7ccc7cccnc78)c6)c5)cc4)n3)cc2)cc1 ; c1ccc(-c2ccc(-c3nc(-c4ccccc4)nc(-c4ccc(-c5cccc(-c6cccc(-c7cc8cccnc8c8ccccc78)c6)c5)cc4)n3)cc2)cc1]

4. The compound of claim 1, wherein the compound of Formula 1 is any one of the following compounds:

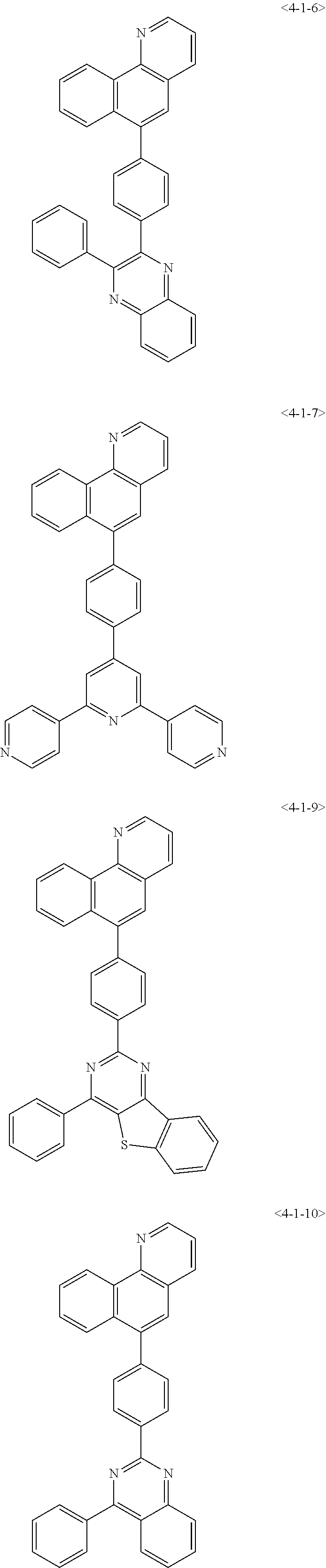 [SMILES: c1ccc(-c2nc3ccccc3nc2-c2ccc(-c3cc4cccnc4c4ccccc34)cc2)cc1 ; c1cnc2c(c1)cc(-c1ccc(-c3cc(-c4ccncc4)nc(-c4ccncc4)c3)cc1)c1ccccc12 ; c1ccc(-c2nc(-c3ccc(-c4cc5cccnc5c5ccccc45)cc3)nc3c2sc2ccccc23)cc1 ; c1ccc(-c2nc(-c3ccc(-c4cc5cccnc5c5ccccc45)cc3)nc3ccccc23)cc1]

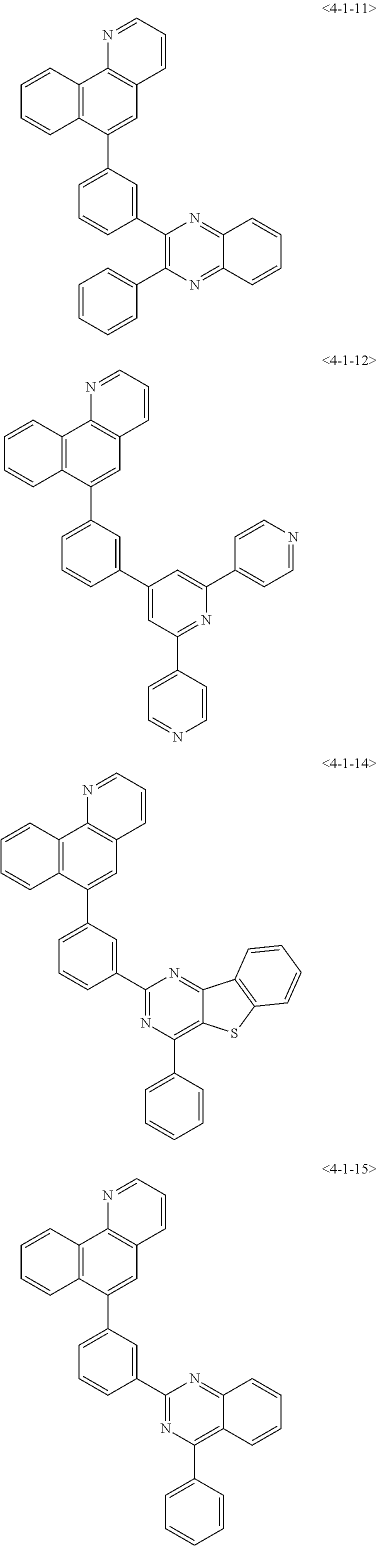

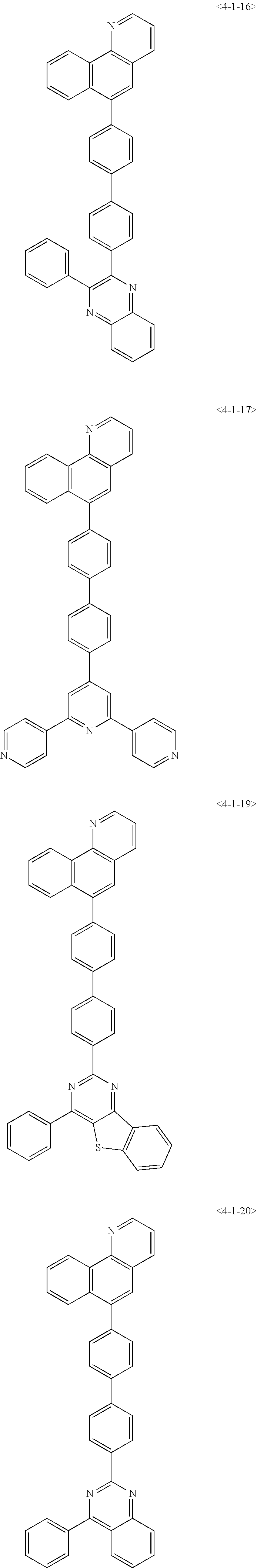 [SMILES: c1ccc(-c2nc3ccccc3nc2-c2ccc(-c3ccc(-c4cc5cccnc5c5ccccc45)cc3)cc2)cc1 ; c1cnc2c(c1)cc(-c1ccc(-c3ccc(-c4cc(-c5ccncc5)nc(-c5ccncc5)c4)cc3)cc1)c1ccccc12 ; c1ccc(-c2nc(-c3ccc(-c4ccc(-c5cc6cccnc6c6ccccc56)cc4)cc3)nc3c2sc2ccccc23)cc1 ; c1ccc(-c2nc(-c3ccc(-c4ccc(-c5cc6cccnc6c6ccccc56)cc4)cc3)nc3ccccc23)cc1]

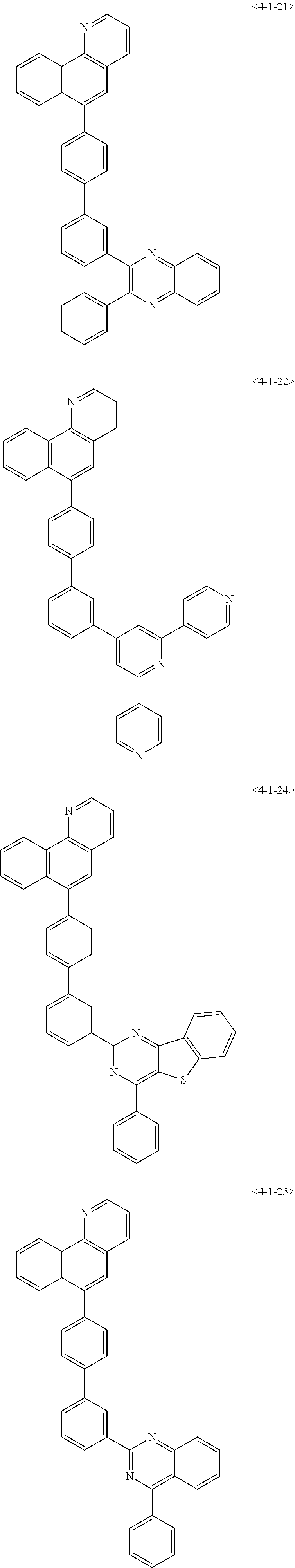 [SMILES: c1ccc(-c2nc3ccccc3nc2-c2cccc(-c3ccc(-c4cc5cccnc5c5ccccc45)cc3)c2)cc1 ; c1cc(-c2ccc(-c3cc4cccnc4c4ccccc34)cc2)cc(-c2cc(-c3ccncc3)nc(-c3ccncc3)c2)c1 ; c1ccc(-c2nc(-c3cccc(-c4ccc(-c5cc6cccnc6c6ccccc56)cc4)c3)nc3c2sc2ccccc23)cc1 ; c1ccc(-c2nc(-c3cccc(-c4ccc(-c5cc6cccnc6c6ccccc56)cc4)c3)nc3ccccc23)cc1]

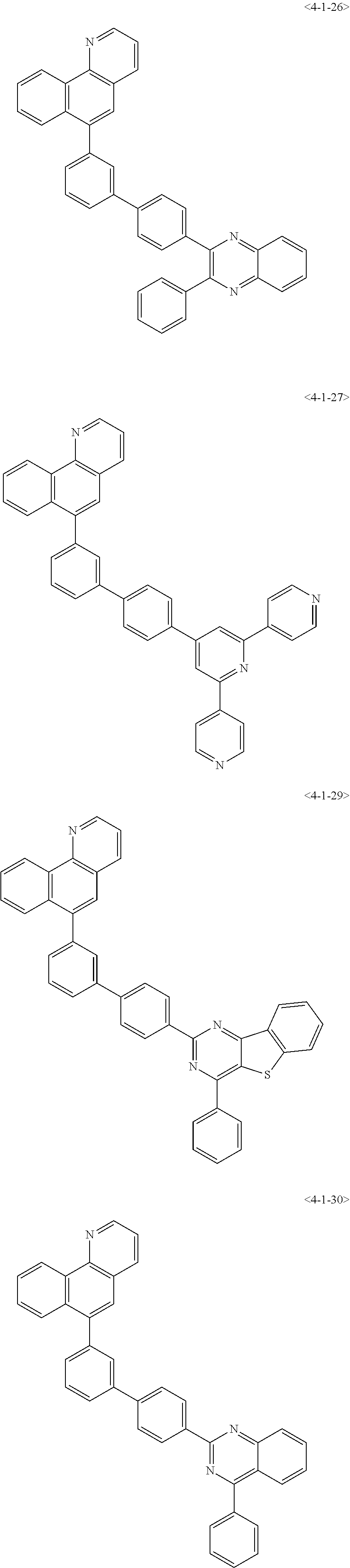 [SMILES: c1ccc(-c2nc3ccccc3nc2-c2ccc(-c3cccc(-c4cc5cccnc5c5ccccc45)c3)cc2)cc1 ; c1cc(-c2ccc(-c3cc(-c4ccncc4)nc(-c4ccncc4)c3)cc2)cc(-c2cc3cccnc3c3ccccc23)c1 ; c1ccc(-c2nc(-c3ccc(-c4cccc(-c5cc6cccnc6c6ccccc56)c4)cc3)nc3c2sc2ccccc23)cc1 ; c1ccc(-c2nc(-c3ccc(-c4cccc(-c5cc6cccnc6c6ccccc56)c4)cc3)nc3ccccc23)cc1]

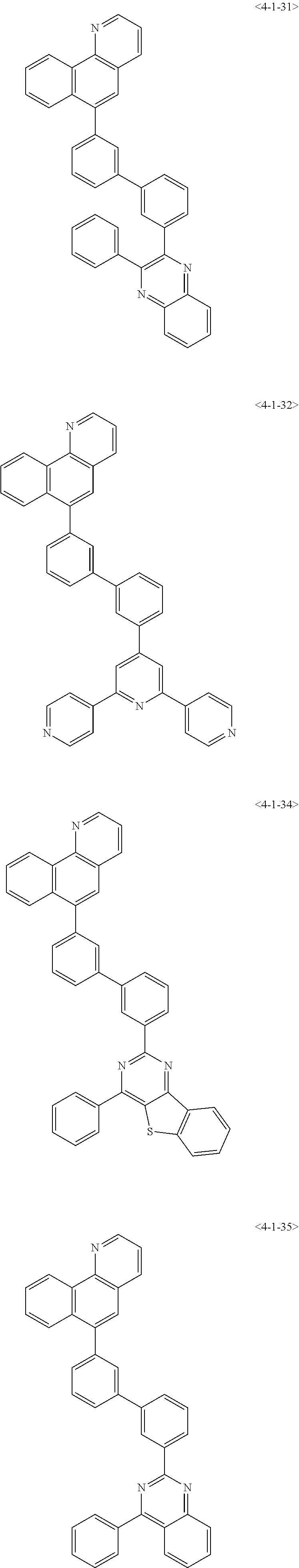 [SMILES: c1ccc(-c2nc3ccccc3nc2-c2cccc(-c3cccc(-c4cc5cccnc5c5ccccc45)c3)c2)cc1 ; c1cc(-c2cccc(-c3cc4cccnc4c4ccccc34)c2)cc(-c2cc(-c3ccncc3)nc(-c3ccncc3)c2)c1 ; c1ccc(-c2nc(-c3cccc(-c4cccc(-c5cc6cccnc6c6ccccc56)c4)c3)nc3c2sc2ccccc23)cc1 ; c1ccc(-c2nc(-c3cccc(-c4cccc(-c5cc6cccnc6c6ccccc56)c4)c3)nc3ccccc23)cc1]

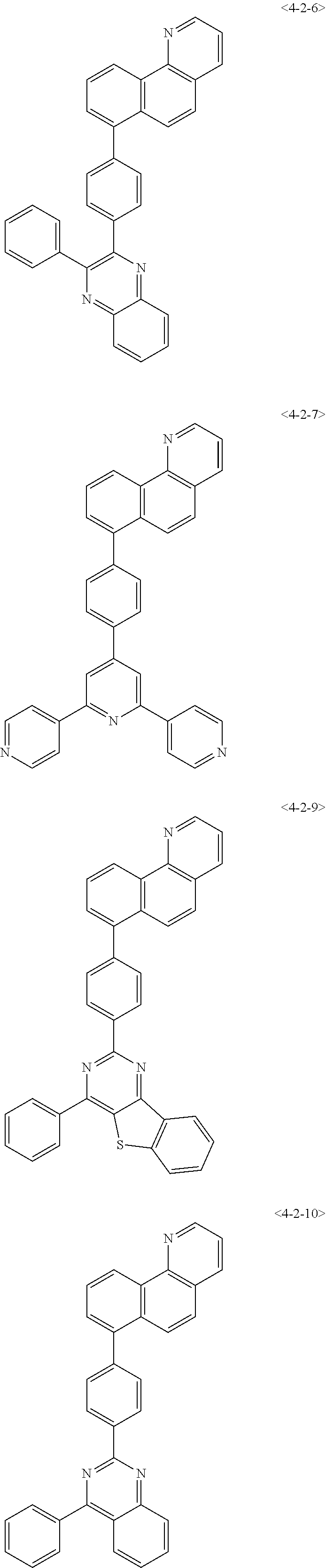 [SMILES: c1ccc(-c2nc3ccccc3nc2-c2ccc(-c3cccc4c3ccc3cccnc34)cc2)cc1 ; c1cnc2c(c1)ccc1c(-c3ccc(-c4cc(-c5ccncc5)nc(-c5ccncc5)c4)cc3)cccc12 ; c1ccc(-c2nc(-c3ccc(-c4cccc5c4ccc4cccnc45)cc3)nc3c2sc2ccccc23)cc1 ; c1ccc(-c2nc(-c3ccc(-c4cccc5c4ccc4cccnc45)cc3)nc3ccccc23)cc1]

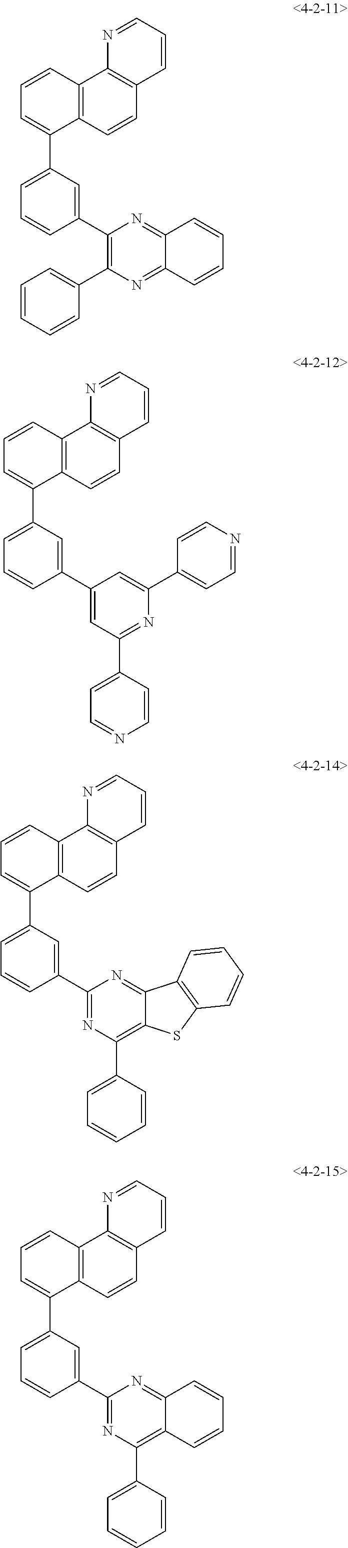

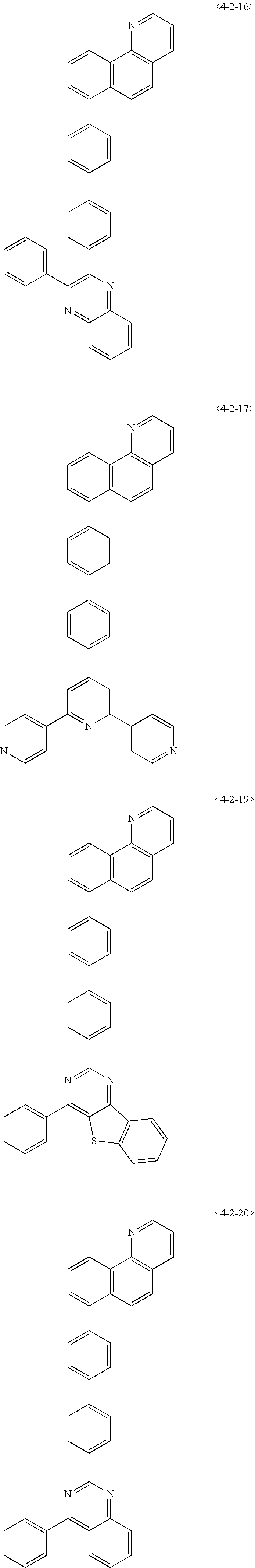 [SMILES: c1ccc(-c2nc3ccccc3nc2-c2ccc(-c3ccc(-c4cccc5c4ccc4cccnc45)cc3)cc2)cc1 ; c1cnc2c(c1)ccc1c(-c3ccc(-c4ccc(-c5cc(-c6ccncc6)nc(-c6ccncc6)c5)cc4)cc3)cccc12 ; c1ccc(-c2nc(-c3ccc(-c4ccc(-c5cccc6c5ccc5cccnc56)cc4)cc3)nc3c2sc2ccccc23)cc1 ; c1ccc(-c2nc(-c3ccc(-c4ccc(-c5cccc6c5ccc5cccnc56)cc4)cc3)nc3ccccc23)cc1]

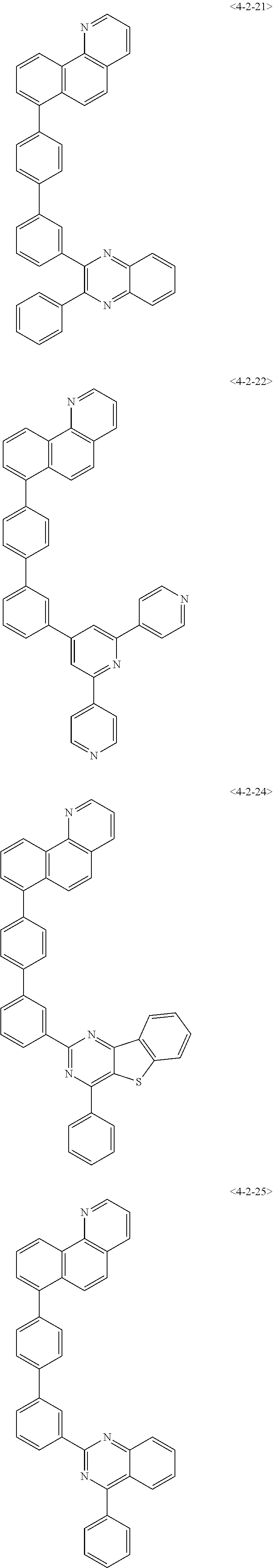 [SMILES: c1ccc(-c2nc3ccccc3nc2-c2cccc(-c3ccc(-c4cccc5c4ccc4cccnc45)cc3)c2)cc1 ; c1cc(-c2ccc(-c3cccc4c3ccc3cccnc34)cc2)cc(-c2cc(-c3ccncc3)nc(-c3ccncc3)c2)c1 ; c1ccc(-c2nc(-c3cccc(-c4ccc(-c5cccc6c5ccc5cccnc56)cc4)c3)nc3c2sc2ccccc23)cc1 ; c1ccc(-c2nc(-c3cccc(-c4ccc(-c5cccc6c5ccc5cccnc56)cc4)c3)nc3ccccc23)cc1]

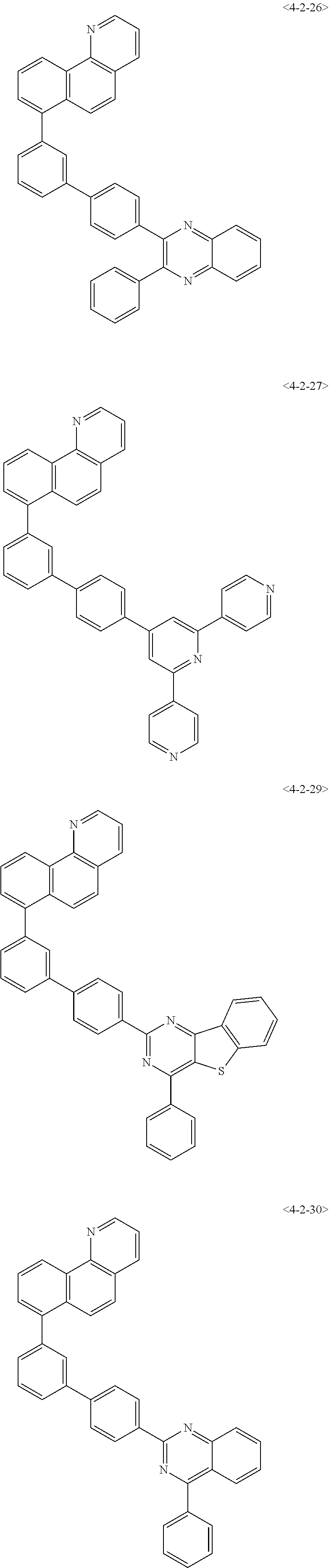 [SMILES: c1ccc(-c2nc3ccccc3nc2-c2ccc(-c3cccc(-c4cccc5c4ccc4cccnc45)c3)cc2)cc1 ; c1cc(-c2ccc(-c3cc(-c4ccncc4)nc(-c4ccncc4)c3)cc2)cc(-c2cccc3c2ccc2cccnc23)c1 ; c1ccc(-c2nc(-c3ccc(-c4cccc(-c5cccc6c5ccc5cccnc56)c4)cc3)nc3c2sc2ccccc23)cc1 ; c1ccc(-c2nc(-c3ccc(-c4cccc(-c5cccc6c5ccc5cccnc56)c4)cc3)nc3ccccc23)cc1]

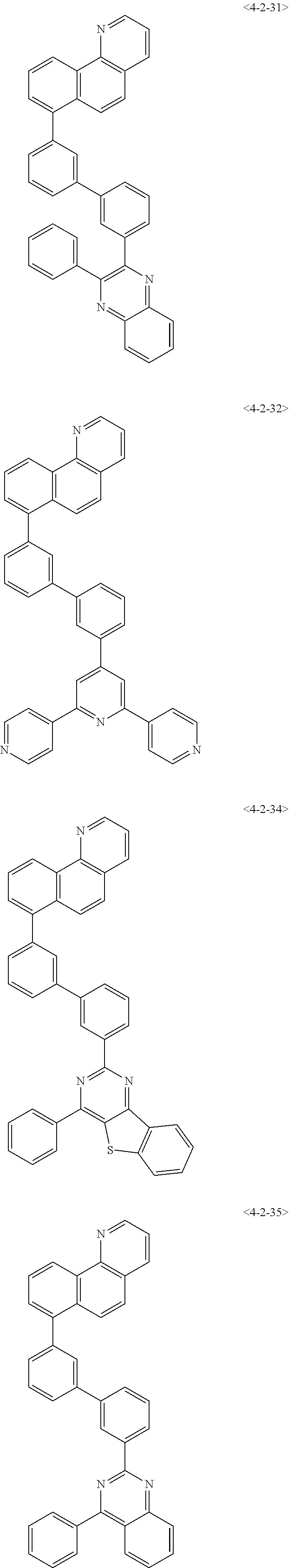 [SMILES: c1ccc(-c2nc3ccccc3nc2-c2cccc(-c3cccc(-c4cccc5c4ccc4cccnc45)c3)c2)cc1 ; c1cc(-c2cccc(-c3cccc4c3ccc3cccnc34)c2)cc(-c2cc(-c3ccncc3)nc(-c3ccncc3)c2)c1 ; c1ccc(-c2nc(-c3cccc(-c4cccc(-c5cccc6c5ccc5cccnc56)c4)c3)nc3c2sc2ccccc23)cc1 ; c1ccc(-c2nc(-c3cccc(-c4cccc(-c5cccc6c5ccc5cccnc56)c4)c3)nc3ccccc23)cc1]

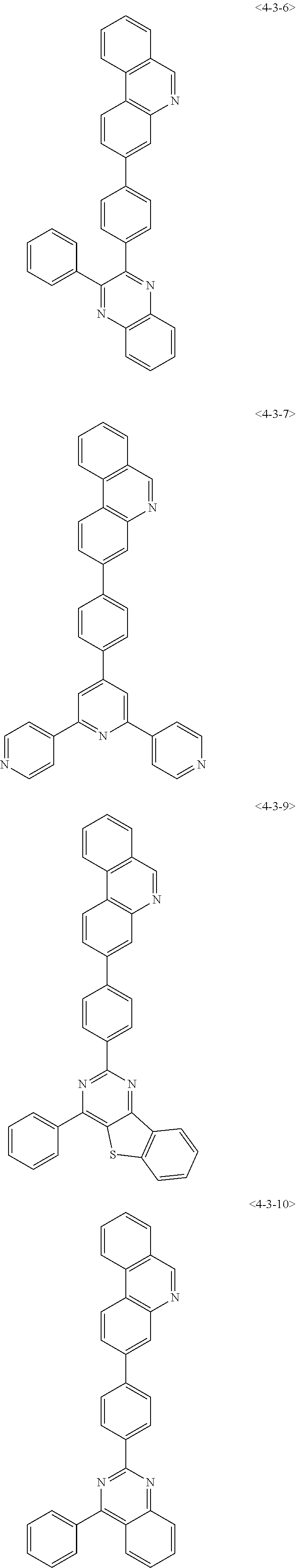 [SMILES: c1ccc(-c2nc3ccccc3nc2-c2ccc(-c3ccc4c(c3)ncc3ccccc34)cc2)cc1 ; c1ccc2c(c1)cnc1cc(-c3ccc(-c4cc(-c5ccncc5)nc(-c5ccncc5)c4)cc3)ccc12 ; c1ccc(-c2nc(-c3ccc(-c4ccc5c(c4)ncc4ccccc45)cc3)nc3c2sc2ccccc23)cc1 ; c1ccc(-c2nc(-c3ccc(-c4ccc5c(c4)ncc4ccccc45)cc3)nc3ccccc23)cc1]

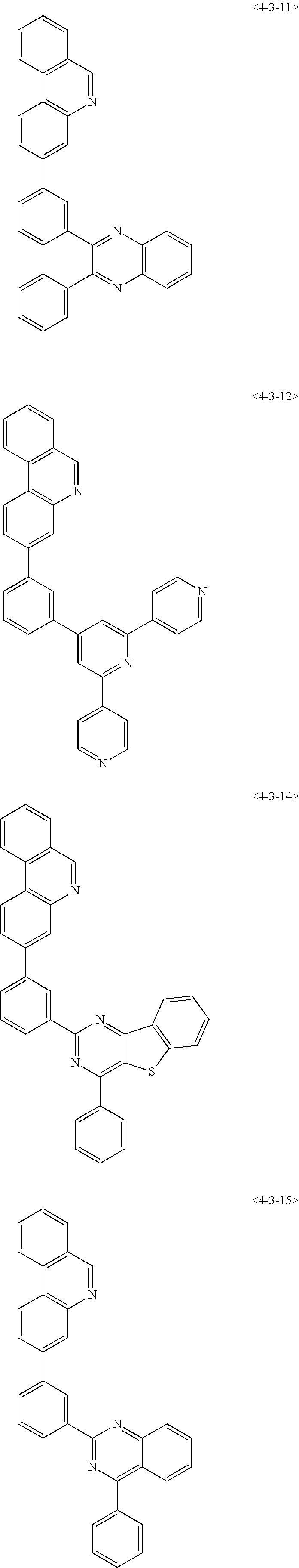 [SMILES: c1ccc(-c2nc3ccccc3nc2-c2cccc(-c3ccc4c(c3)ncc3ccccc34)c2)cc1 ; c1cc(-c2cc(-c3ccncc3)nc(-c3ccncc3)c2)cc(-c2ccc3c(c2)ncc2ccccc23)c1 ; c1ccc(-c2nc(-c3cccc(-c4ccc5c(c4)ncc4ccccc45)c3)nc3c2sc2ccccc23)cc1 ; c1ccc(-c2nc(-c3cccc(-c4ccc5c(c4)ncc4ccccc45)c3)nc3ccccc23)cc1]

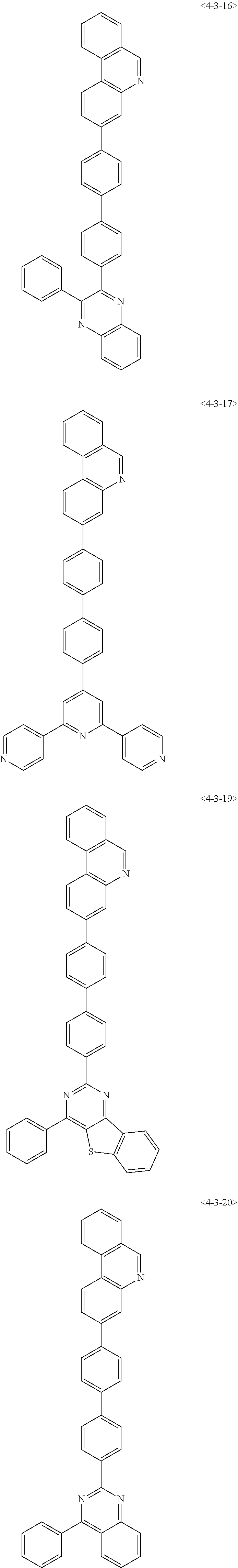 [SMILES: c1ccc(-c2nc3ccccc3nc2-c2ccc(-c3ccc(-c4ccc5c(c4)ncc4ccccc45)cc3)cc2)cc1 ; c1ccc2c(c1)cnc1cc(-c3ccc(-c4ccc(-c5cc(-c6ccncc6)nc(-c6ccncc6)c5)cc4)cc3)ccc12 ; c1ccc(-c2nc(-c3ccc(-c4ccc(-c5ccc6c(c5)ncc5ccccc56)cc4)cc3)nc3c2sc2ccccc23)cc1 ; c1ccc(-c2nc(-c3ccc(-c4ccc(-c5ccc6c(c5)ncc5ccccc56)cc4)cc3)nc3ccccc23)cc1]

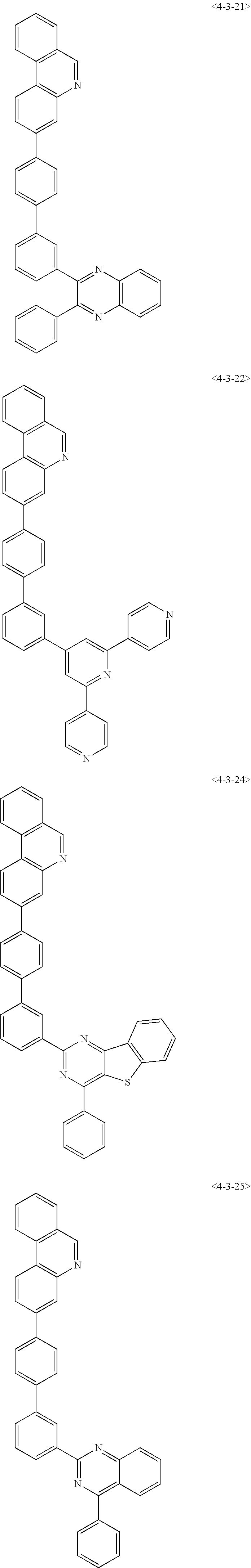 [SMILES: c1ccc(-c2nc3ccccc3nc2-c2cccc(-c3ccc(-c4ccc5c(c4)ncc4ccccc45)cc3)c2)cc1 ; c1cc(-c2ccc(-c3ccc4c(c3)ncc3ccccc34)cc2)cc(-c2cc(-c3ccncc3)nc(-c3ccncc3)c2)c1 ; c1ccc(-c2nc(-c3cccc(-c4ccc(-c5ccc6c(c5)ncc5ccccc56)cc4)c3)nc3c2sc2ccccc23)cc1 ; c1ccc(-c2nc(-c3cccc(-c4ccc(-c5ccc6c(c5)ncc5ccccc56)cc4)c3)nc3ccccc23)cc1]

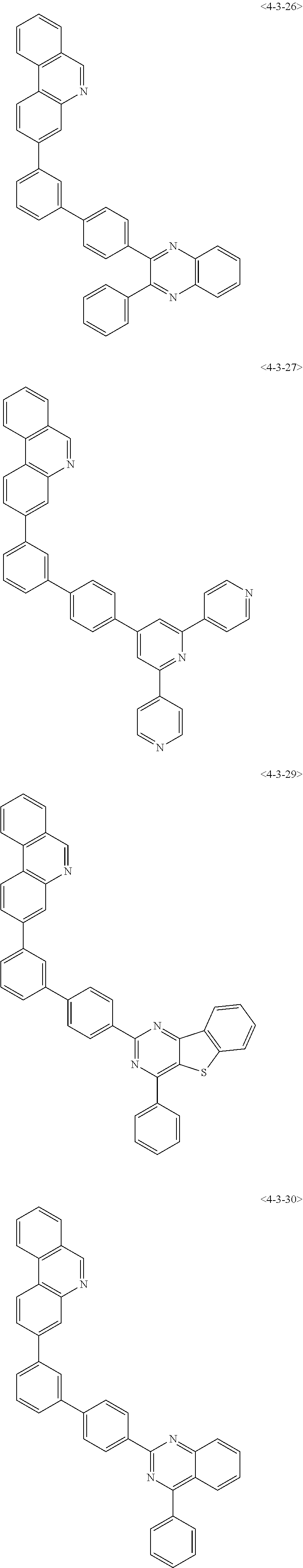 [SMILES: c1ccc(-c2nc3ccccc3nc2-c2ccc(-c3cccc(-c4ccc5c(c4)ncc4ccccc45)c3)cc2)cc1 ; c1cc(-c2ccc(-c3cc(-c4ccncc4)nc(-c4ccncc4)c3)cc2)cc(-c2ccc3c(c2)ncc2ccccc23)c1 ; c1ccc(-c2nc(-c3ccc(-c4cccc(-c5ccc6c(c5)ncc5ccccc56)c4)cc3)nc3c2sc2ccccc23)cc1 ; c1ccc(-c2nc(-c3ccc(-c4cccc(-c5ccc6c(c5)ncc5ccccc56)c4)cc3)nc3ccccc23)cc1]

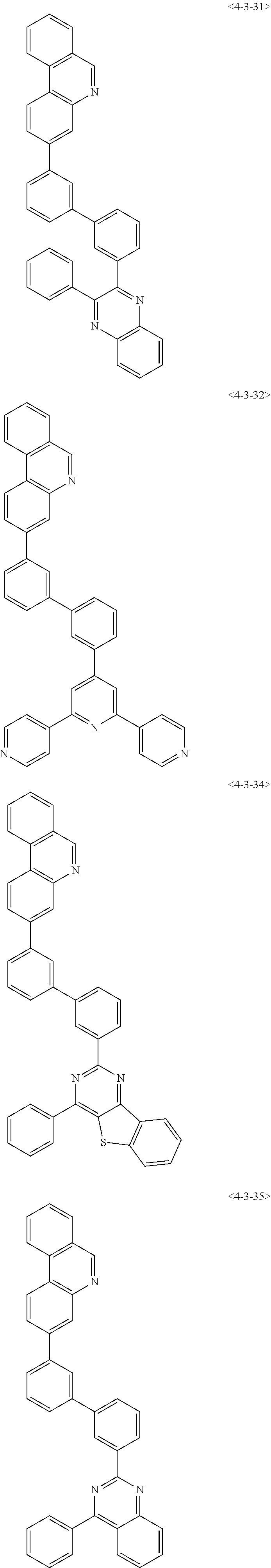

5. The compound of claim 1, wherein the compound of chemical formula 1 is any one of the following compounds:

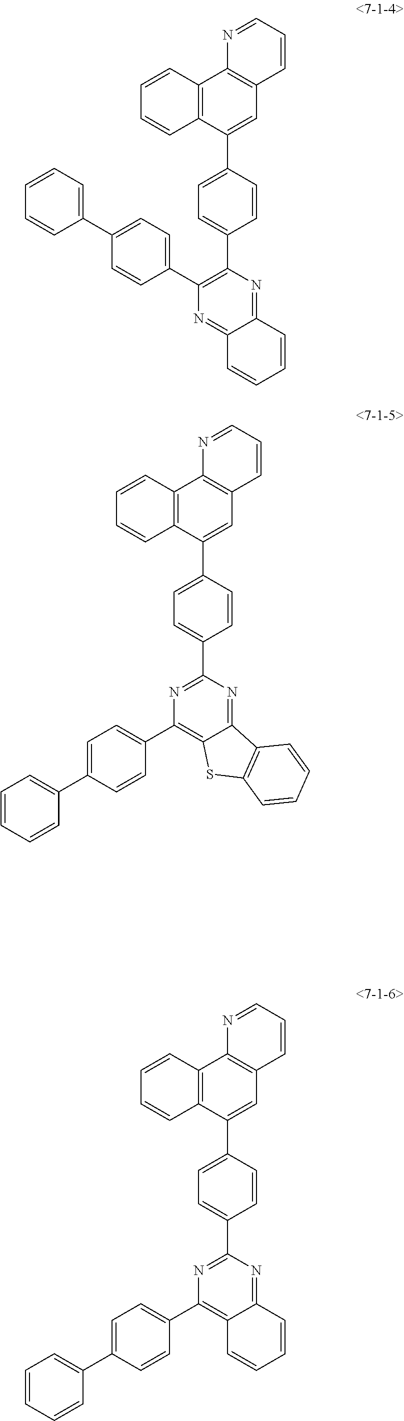 [SMILES: c1ccc(-c2ccc(-c3nc4ccccc4nc3-c3ccc(-c4cc5cccnc5c5ccccc45)cc3)cc2)cc1 ; c1ccc(-c2ccc(-c3nc(-c4ccc(-c5cc6cccnc6c6ccccc56)cc4)nc4c3sc3ccccc34)cc2)cc1 ; c1ccc(-c2ccc(-c3nc(-c4ccc(-c5cc6cccnc6c6ccccc56)cc4)nc4ccccc34)cc2)cc1]

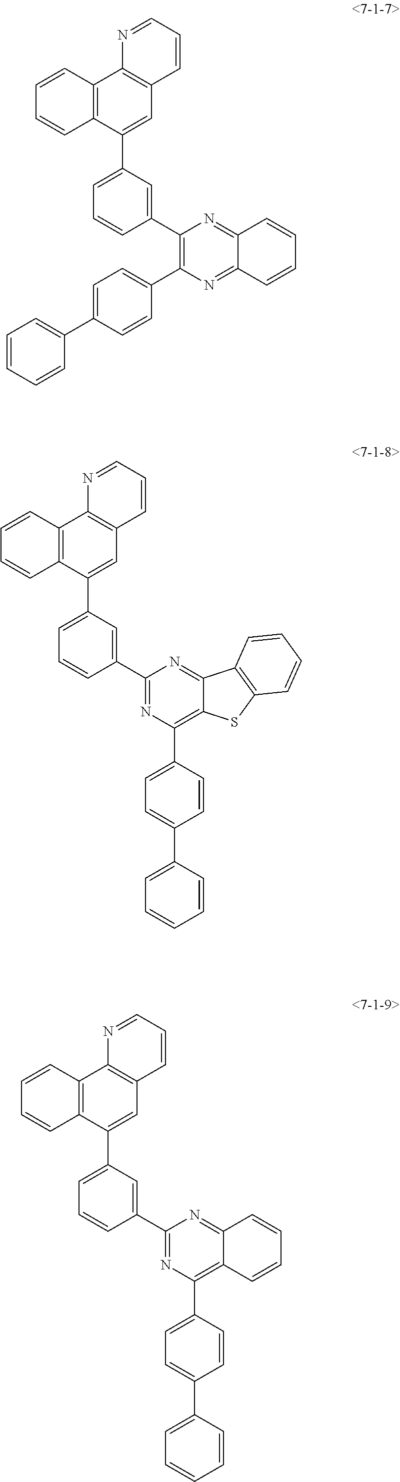 [SMILES: c1ccc(-c2ccc(-c3nc4ccccc4nc3-c3cccc(-c4cc5cccnc5c5ccccc45)c3)cc2)cc1 ; c1ccc(-c2ccc(-c3nc(-c4cccc(-c5cc6cccnc6c6ccccc56)c4)nc4c3sc3ccccc34)cc2)cc1 ; c1ccc(-c2ccc(-c3nc(-c4cccc(-c5cc6cccnc6c6ccccc56)c4)nc4ccccc34)cc2)cc1]

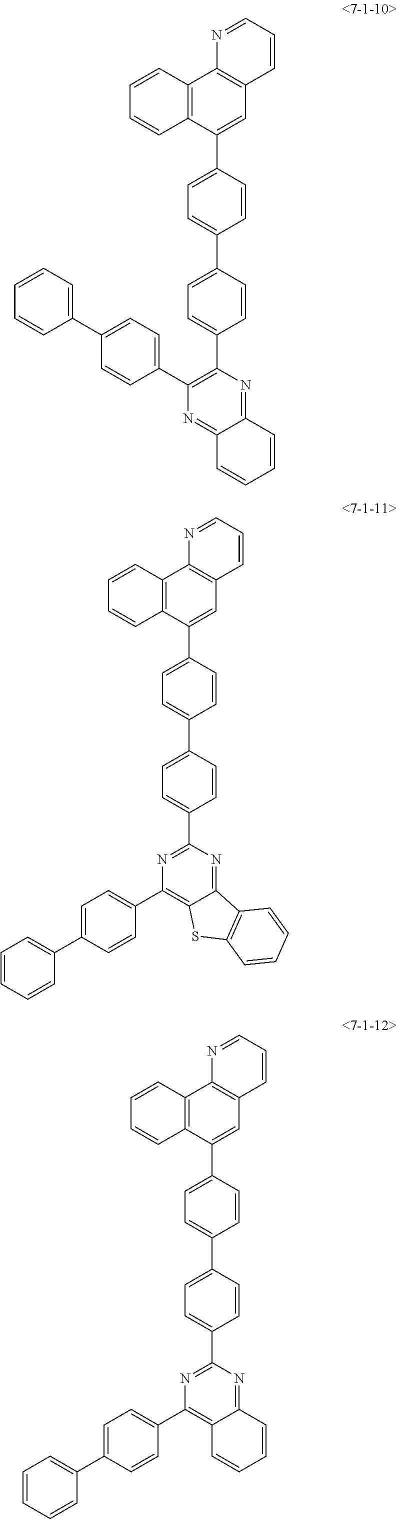 [SMILES: c1ccc(-c2ccc(-c3nc4ccccc4nc3-c3ccc(-c4ccc(-c5cc6cccnc6c6ccccc56)cc4)cc3)cc2)cc1 ; c1ccc(-c2ccc(-c3nc(-c4ccc(-c5ccc(-c6cc7cccnc7c7ccccc67)cc5)cc4)nc4c3sc3ccccc34)cc2)cc1 ; c1ccc(-c2ccc(-c3nc(-c4ccc(-c5ccc(-c6cc7cccnc7c7ccccc67)cc5)cc4)nc4ccccc34)cc2)cc1]

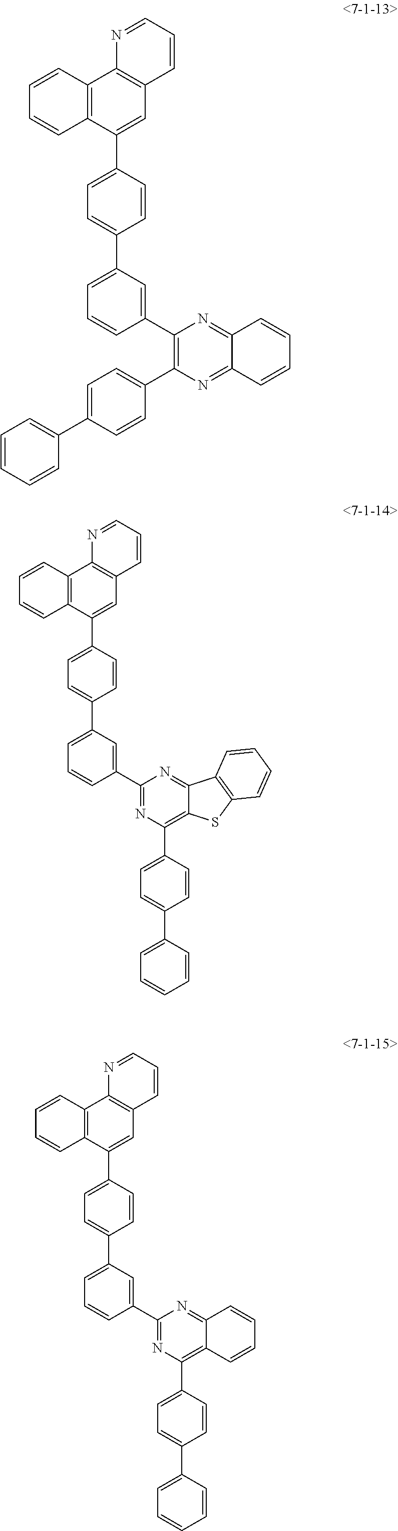 [SMILES: c1ccc(-c2ccc(-c3nc4ccccc4nc3-c3cccc(-c4ccc(-c5cc6cccnc6c6ccccc56)cc4)c3)cc2)cc1 ; c1ccc(-c2ccc(-c3nc(-c4cccc(-c5ccc(-c6cc7cccnc7c7ccccc67)cc5)c4)nc4c3sc3ccccc34)cc2)cc1 ; c1ccc(-c2ccc(-c3nc(-c4cccc(-c5ccc(-c6cc7cccnc7c7ccccc67)cc5)c4)nc4ccccc34)cc2)cc1]

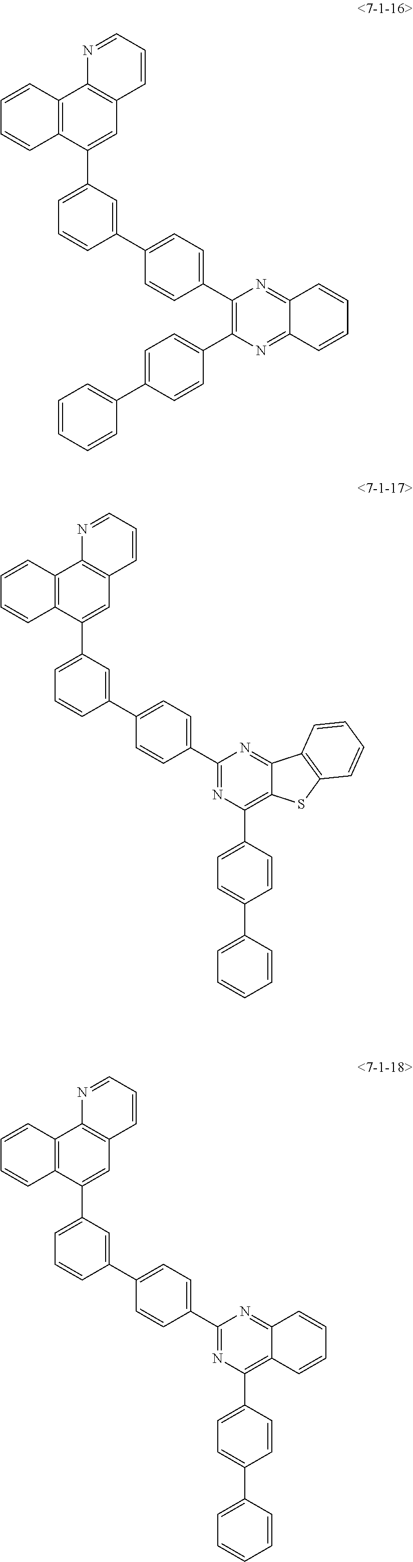 [SMILES: c1ccc(-c2ccc(-c3nc4ccccc4nc3-c3ccc(-c4cccc(-c5cc6cccnc6c6ccccc56)c4)cc3)cc2)cc1 ; c1ccc(-c2ccc(-c3nc(-c4ccc(-c5cccc(-c6cc7cccnc7c7ccccc67)c5)cc4)nc4c3sc3ccccc34)cc2)cc1 ; c1ccc(-c2ccc(-c3nc(-c4ccc(-c5cccc(-c6cc7cccnc7c7ccccc67)c5)cc4)nc4ccccc34)cc2)cc1]

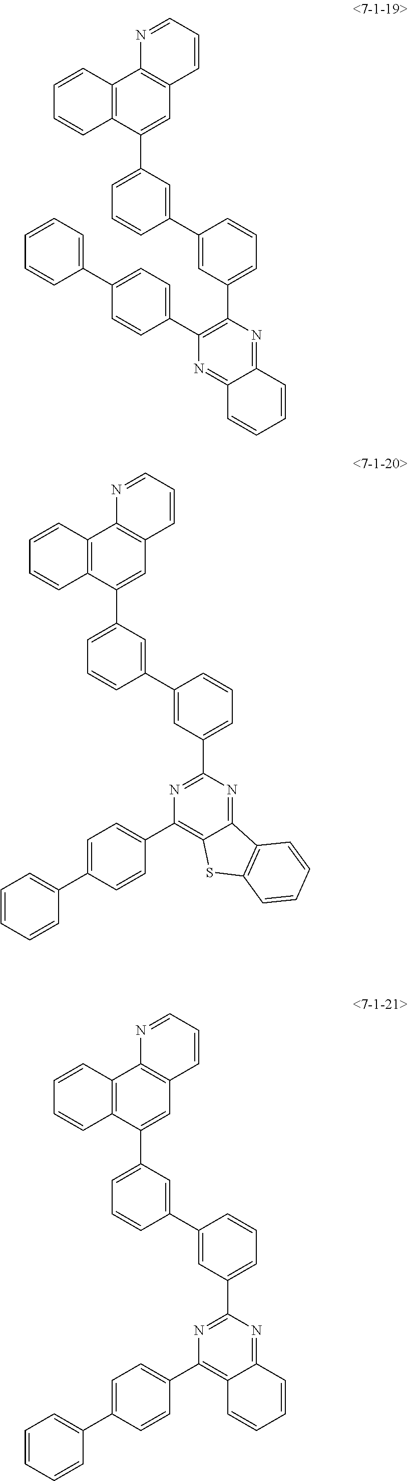 [SMILES: c1ccc(-c2ccc(-c3nc4ccccc4nc3-c3cccc(-c4cccc(-c5cc6cccnc6c6ccccc56)c4)c3)cc2)cc1 ; c1ccc(-c2ccc(-c3nc(-c4cccc(-c5cccc(-c6cc7cccnc7c7ccccc67)c5)c4)nc4c3sc3ccccc34)cc2)cc1 ; c1ccc(-c2ccc(-c3nc(-c4cccc(-c5cccc(-c6cc7cccnc7c7ccccc67)c5)c4)nc4ccccc34)cc2)cc1]

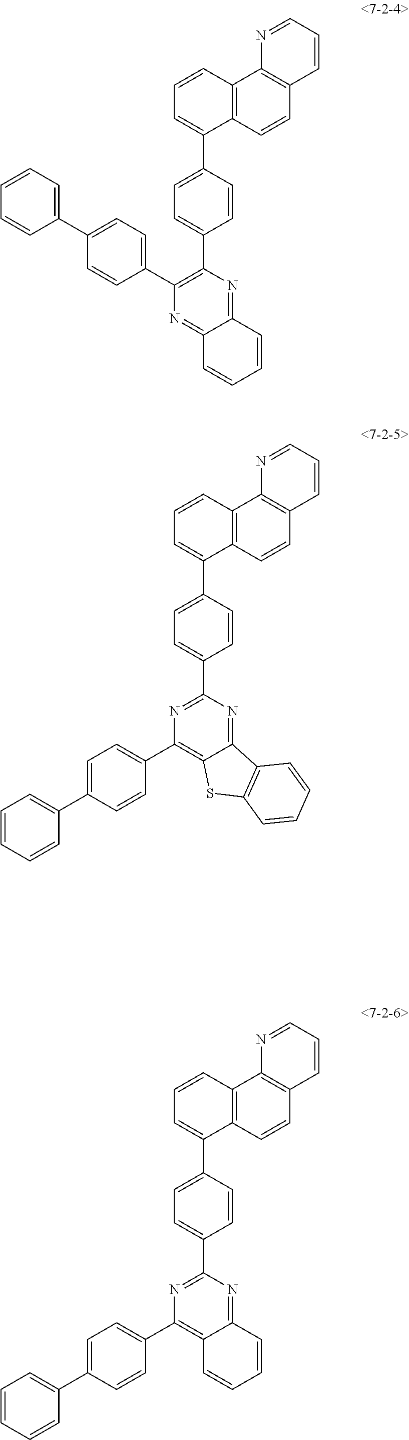 [SMILES: c1ccc(-c2ccc(-c3nc4ccccc4nc3-c3ccc(-c4cccc5c4ccc4cccnc45)cc3)cc2)cc1 ; c1ccc(-c2ccc(-c3nc(-c4ccc(-c5cccc6c5ccc5cccnc56)cc4)nc4c3sc3ccccc34)cc2)cc1 ; c1ccc(-c2ccc(-c3nc(-c4ccc(-c5cccc6c5ccc5cccnc56)cc4)nc4ccccc34)cc2)cc1]

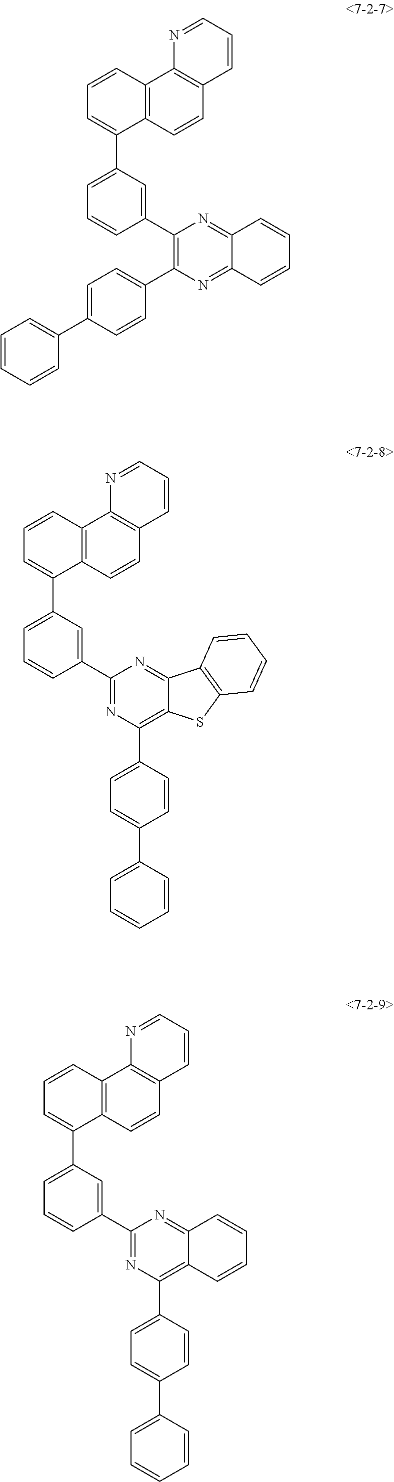 [SMILES: c1ccc(-c2ccc(-c3nc4ccccc4nc3-c3cccc(-c4cccc5c4ccc4cccnc45)c3)cc2)cc1 ; c1ccc(-c2ccc(-c3nc(-c4cccc(-c5cccc6c5ccc5cccnc56)c4)nc4c3sc3ccccc34)cc2)cc1 ; c1ccc(-c2ccc(-c3nc(-c4cccc(-c5cccc6c5ccc5cccnc56)c4)nc4ccccc34)cc2)cc1]

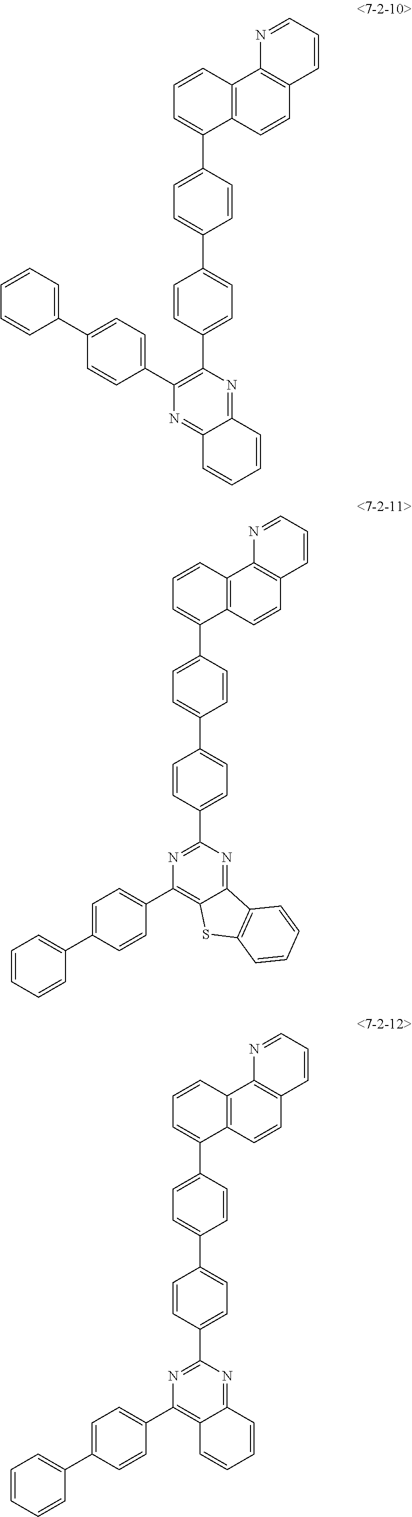 [SMILES: c1ccc(-c2ccc(-c3nc4ccccc4nc3-c3ccc(-c4ccc(-c5cccc6c5ccc5cccnc56)cc4)cc3)cc2)cc1 ; c1ccc(-c2ccc(-c3nc(-c4ccc(-c5ccc(-c6cccc7c6ccc6cccnc67)cc5)cc4)nc4c3sc3ccccc34)cc2)cc1 ; c1ccc(-c2ccc(-c3nc(-c4ccc(-c5ccc(-c6cccc7c6ccc6cccnc67)cc5)cc4)nc4ccccc34)cc2)cc1]

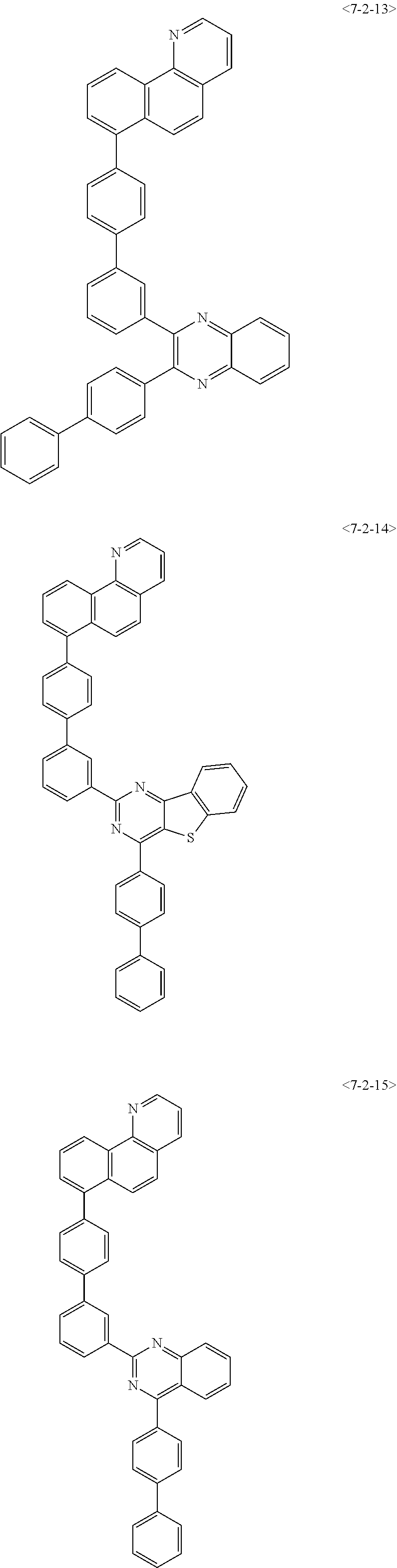 [SMILES: c1ccc(-c2ccc(-c3nc4ccccc4nc3-c3cccc(-c4ccc(-c5cccc6c5ccc5cccnc56)cc4)c3)cc2)cc1 ; c1ccc(-c2ccc(-c3nc(-c4cccc(-c5ccc(-c6cccc7c6ccc6cccnc67)cc5)c4)nc4c3sc3ccccc34)cc2)cc1 ; c1ccc(-c2ccc(-c3nc(-c4cccc(-c5ccc(-c6cccc7c6ccc6cccnc67)cc5)c4)nc4ccccc34)cc2)cc1]

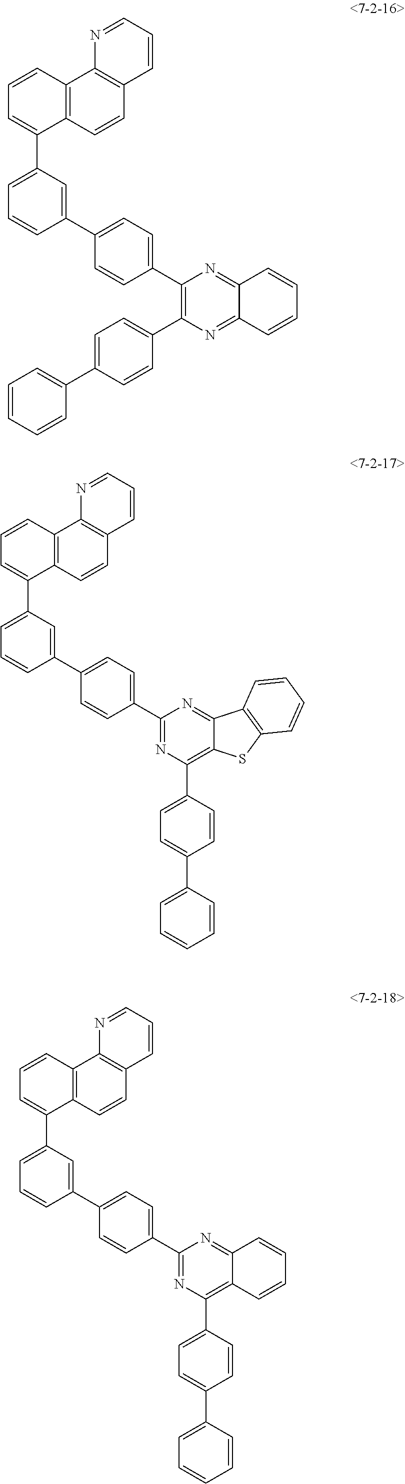 [SMILES: c1ccc(-c2ccc(-c3nc4ccccc4nc3-c3ccc(-c4cccc(-c5cccc6c5ccc5cccnc56)c4)cc3)cc2)cc1 ; c1ccc(-c2ccc(-c3nc(-c4ccc(-c5cccc(-c6cccc7c6ccc6cccnc67)c5)cc4)nc4c3sc3ccccc34)cc2)cc1 ; c1ccc(-c2ccc(-c3nc(-c4ccc(-c5cccc(-c6cccc7c6ccc6cccnc67)c5)cc4)nc4ccccc34)cc2)cc1]

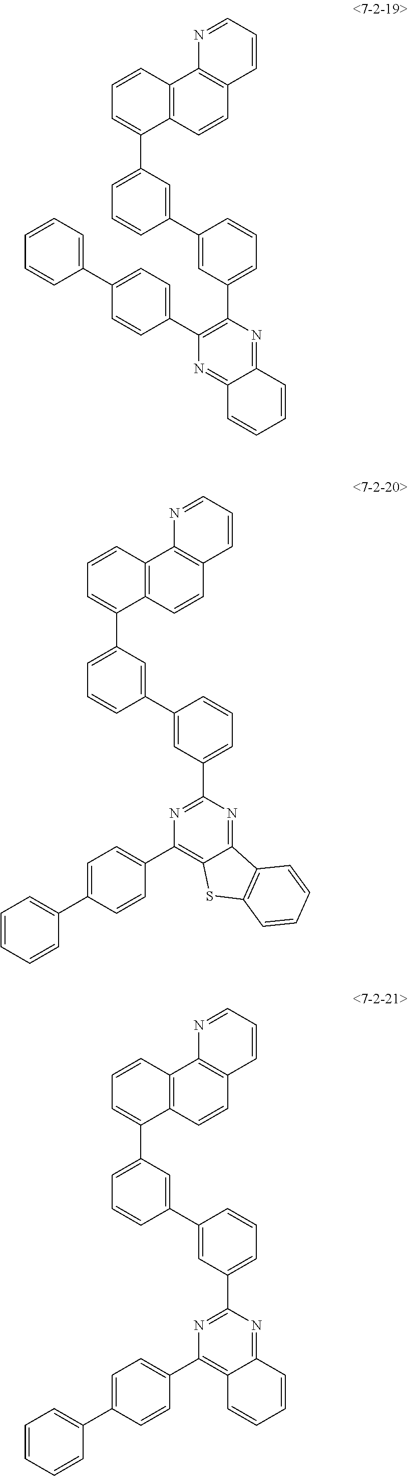 [SMILES: c1ccc(-c2ccc(-c3nc4ccccc4nc3-c3cccc(-c4cccc(-c5cccc6c5ccc5cccnc56)c4)c3)cc2)cc1 ; c1ccc(-c2ccc(-c3nc(-c4cccc(-c5cccc(-c6cccc7c6ccc6cccnc67)c5)c4)nc4c3sc3ccccc34)cc2)cc1 ; c1ccc(-c2ccc(-c3nc(-c4cccc(-c5cccc(-c6cccc7c6ccc6cccnc67)c5)c4)nc4ccccc34)cc2)cc1]

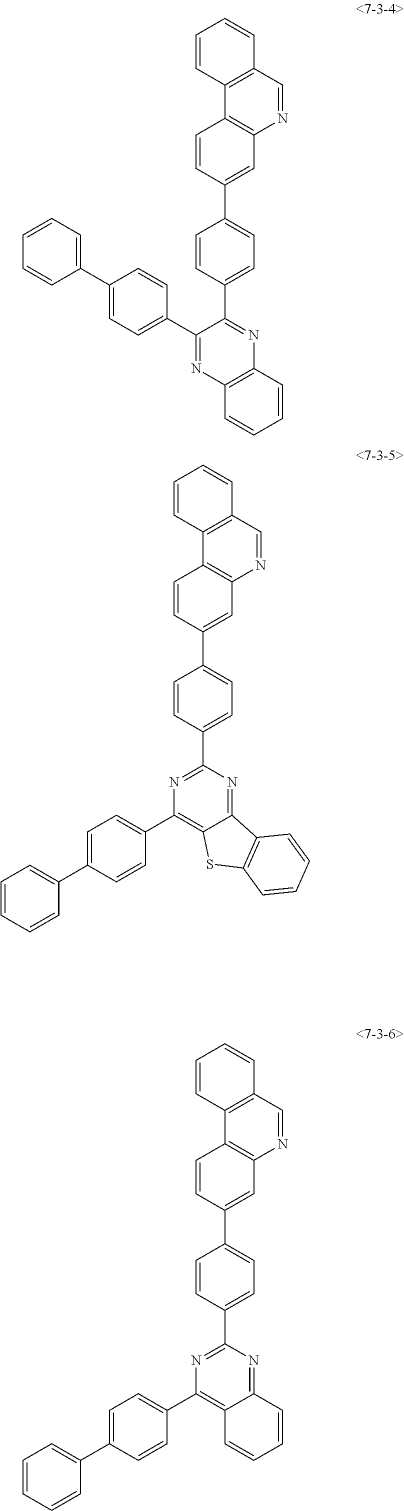 [SMILES: c1ccc(-c2ccc(-c3nc4ccccc4nc3-c3ccc(-c4ccc5c(c4)ncc4ccccc45)cc3)cc2)cc1 ; c1ccc(-c2ccc(-c3nc(-c4ccc(-c5ccc6c(c5)ncc5ccccc56)cc4)nc4c3sc3ccccc34)cc2)cc1 ; c1ccc(-c2ccc(-c3nc(-c4ccc(-c5ccc6c(c5)ncc5ccccc56)cc4)nc4ccccc34)cc2)cc1]

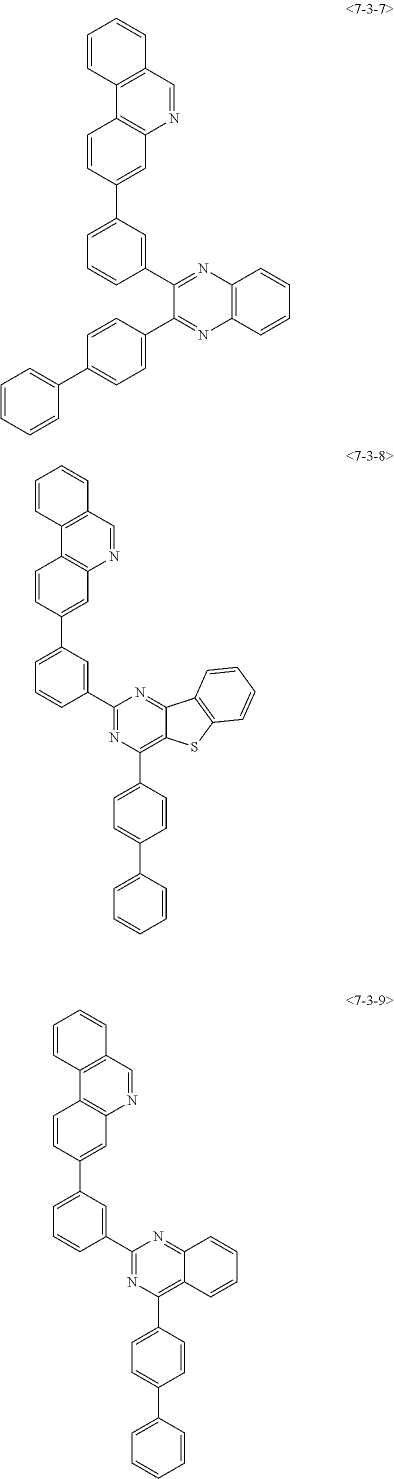 [SMILES: c1ccc(-c2ccc(-c3nc4ccccc4nc3-c3cccc(-c4ccc5c(c4)ncc4ccccc45)c3)cc2)cc1 ; c1ccc(-c2ccc(-c3nc(-c4cccc(-c5ccc6c(c5)ncc5ccccc56)c4)nc4c3sc3ccccc34)cc2)cc1 ; c1ccc(-c2ccc(-c3nc(-c4cccc(-c5ccc6c(c5)ncc5ccccc56)c4)nc4ccccc34)cc2)cc1]

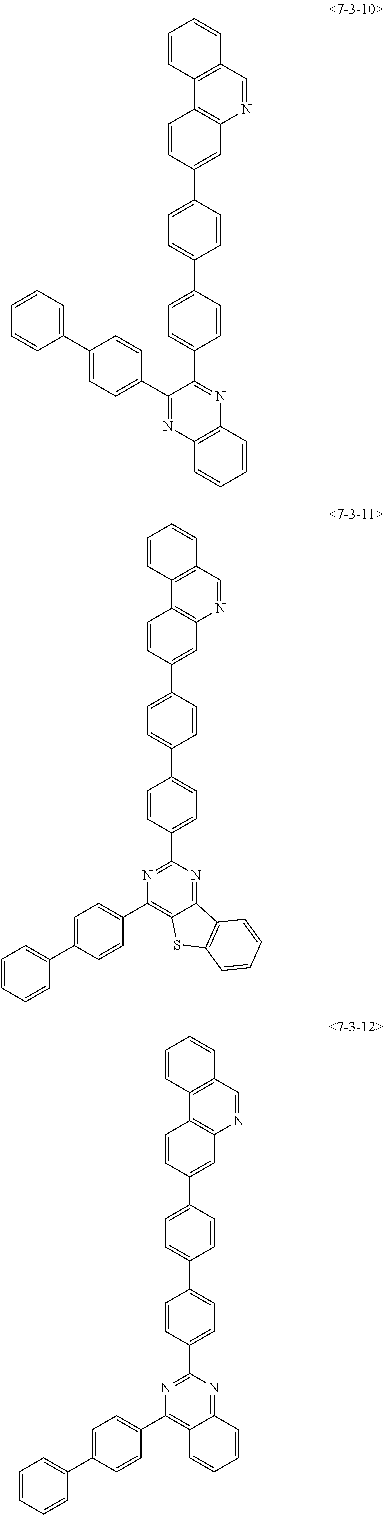 [SMILES: c1ccc(-c2ccc(-c3nc4ccccc4nc3-c3ccc(-c4ccc(-c5ccc6c(c5)ncc5ccccc56)cc4)cc3)cc2)cc1 ; c1ccc(-c2ccc(-c3nc(-c4ccc(-c5ccc(-c6ccc7c(c6)ncc6ccccc67)cc5)cc4)nc4c3sc3ccccc34)cc2)cc1 ; c1ccc(-c2ccc(-c3nc(-c4ccc(-c5ccc(-c6ccc7c(c6)ncc6ccccc67)cc5)cc4)nc4ccccc34)cc2)cc1]

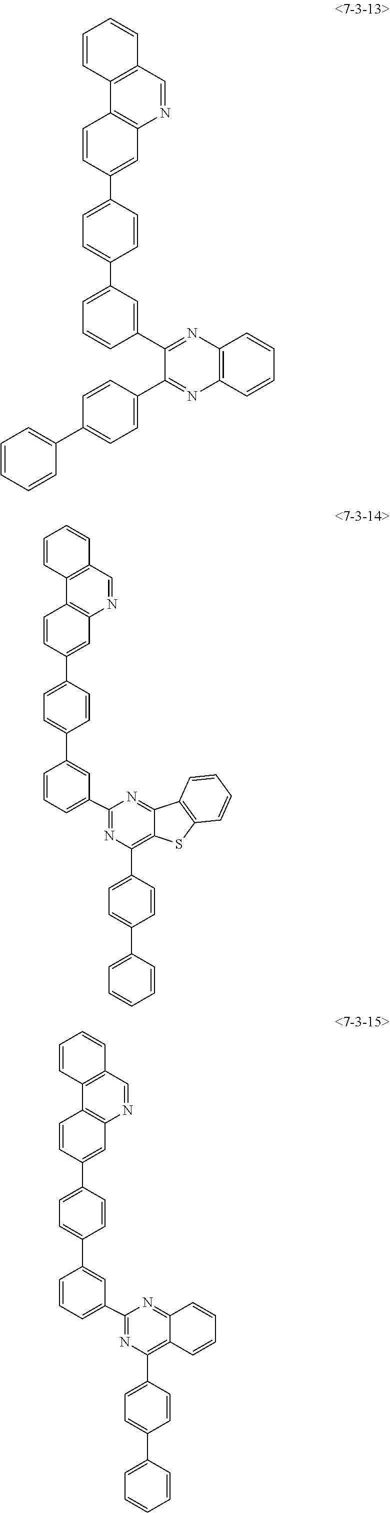 [SMILES: c1ccc(-c2ccc(-c3nc4ccccc4nc3-c3cccc(-c4ccc(-c5ccc6c(c5)ncc5ccccc56)cc4)c3)cc2)cc1 ; c1ccc(-c2ccc(-c3nc(-c4cccc(-c5ccc(-c6ccc7c(c6)ncc6ccccc67)cc5)c4)nc4c3sc3ccccc34)cc2)cc1 ; c1ccc(-c2ccc(-c3nc(-c4cccc(-c5ccc(-c6ccc7c(c6)ncc6ccccc67)cc5)c4)nc4ccccc34)cc2)cc1]

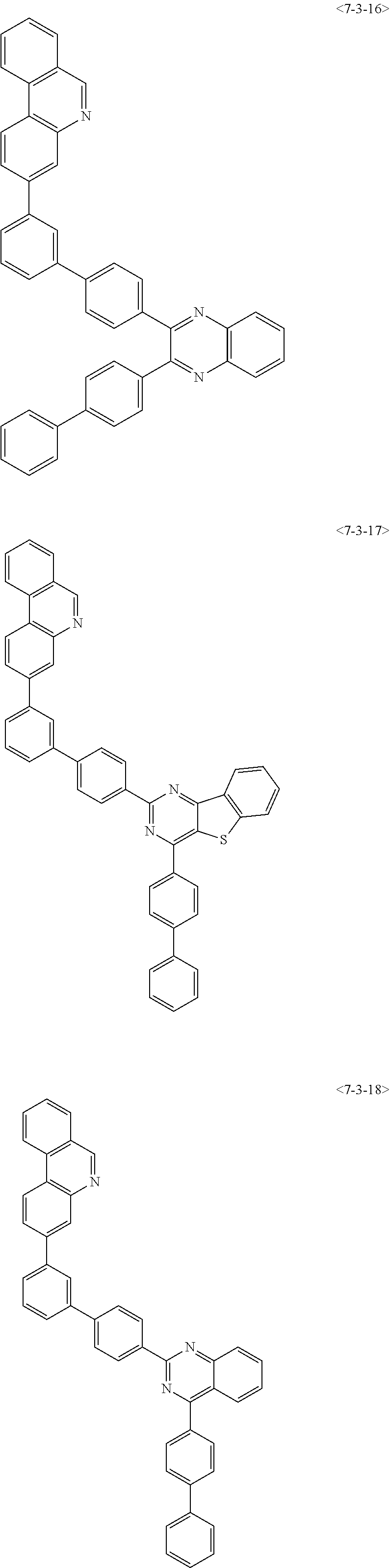 [SMILES: c1ccc(-c2ccc(-c3nc4ccccc4nc3-c3ccc(-c4cccc(-c5ccc6c(c5)ncc5ccccc56)c4)cc3)cc2)cc1 ; c1ccc(-c2ccc(-c3nc(-c4ccc(-c5cccc(-c6ccc7c(c6)ncc6ccccc67)c5)cc4)nc4c3sc3ccccc34)cc2)cc1 ; c1ccc(-c2ccc(-c3nc(-c4ccc(-c5cccc(-c6ccc7c(c6)ncc6ccccc67)c5)cc4)nc4ccccc34)cc2)cc1]

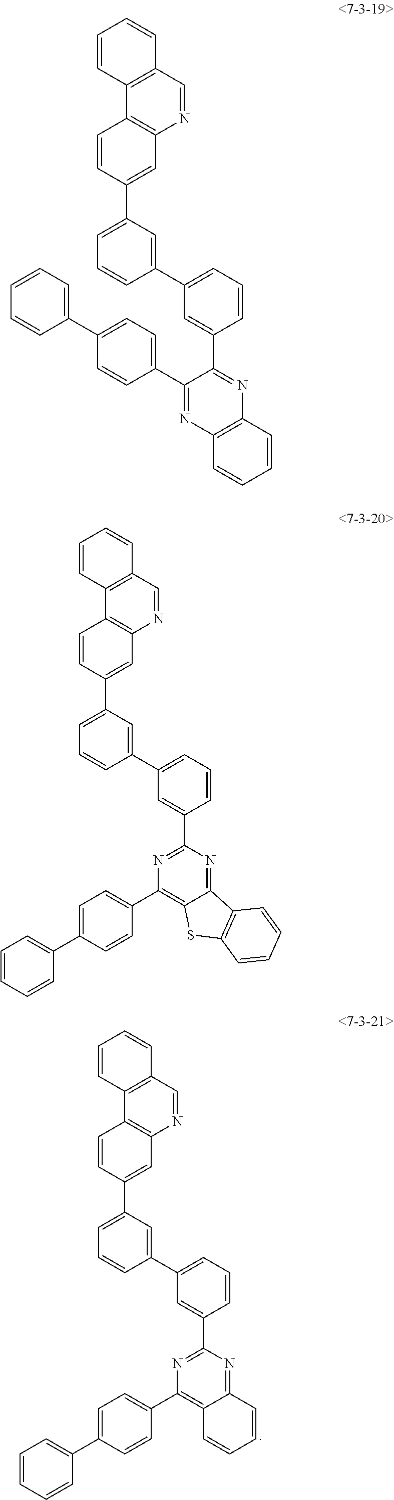 [SMILES: c1ccc(-c2ccc(-c3nc4ccccc4nc3-c3cccc(-c4cccc(-c5ccc6c(c5)ncc5ccccc56)c4)c3)cc2)cc1 ; c1ccc(-c2ccc(-c3nc(-c4cccc(-c5cccc(-c6ccc7c(c6)ncc6ccccc67)c5)c4)nc4c3sc3ccccc34)cc2)cc1 ; c1ccc(-c2ccc(-c3nc(-c4cccc(-c5cccc(-c6ccc7c(c6)ncc6ccccc67)c5)c4)nc4ccccc34)cc2)cc1]

6. An organic light emitting device comprising:
a first electrode; a second electrode facing the first electrode; and an organic material layer interposed between the first electrode and the second electrode,
wherein the organic material layer comprises i) a light emitting layer, ii) a hole transport region interposed between the first electrode and the light emitting layer and including at least one of a hole injection layer, a hole transport layer, and an electron blocking layer, and iii) an electron transport region interposed between the light emitting layer and the second electrode and including at least one of a hole blocking layer,
wherein the hole blocking layer comprises a compound represented by chemical formula 1,
A₁ [symbol] L [symbol] A₂ <Chemical Formula 1>,
wherein A₁ is a group represented by any one of the following structures: 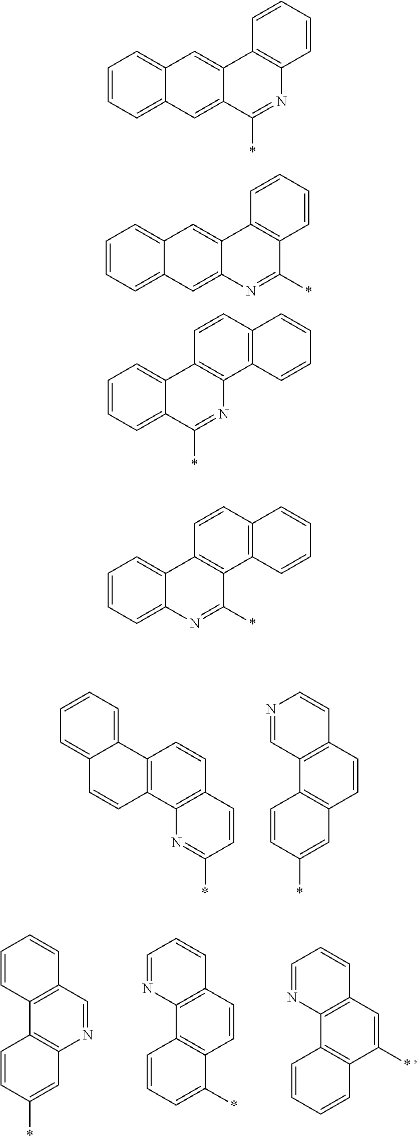
L is a direct bond; a substituted or unsubstituted arylene group; or a substituted or unsubstituted heteroarylene group; or a substituted or unsubstituted C₉-C₆₀ fused polycyclic group,
A₂ is any one of the following structures, 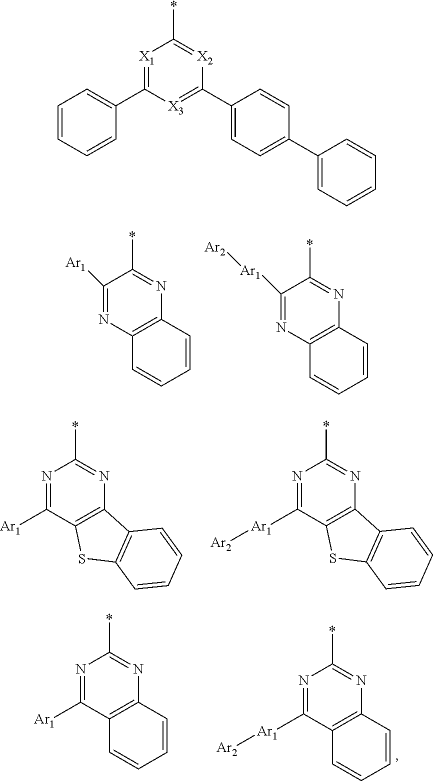 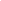
wherein X₁ to X₃ are each independently C or N, at least one of X₁ to X₃ is N, and Ar₁ and Ar₂ are each independently hydrogen, deuterium, a halogen group, a cyano group, a substituted or unsubstituted C₁ to C₆₀ alkyl group, a substituted or unsubstituted C₃ to C₁₀ cycloalkyl group, a substituted or unsubstituted C₆ to C₆₀ aryl group, a substituted or unsubstituted C₁ to C₆₀ heteroaryl group.

7. A display device including the organic light emitting device of claim 6, wherein a first electrode of the organic light emitting device is electrically connected to a source electrode or a drain electrode of a thin film transistor.
